(19) United States
(12) Patent Application Publication
(10) Pub. No.: US 2011/0034507 A1
(43) Pub. Date: Feb. 10, 2011

(54) FARNESOID X RECEPTOR AGONISTS

(75) Inventors: Adwoa Akwabi-Ameyaw, Raleigh, NC (US); David Norman Deaton, Raleigh, NC (US); Robert Blount McFadyen, Raleigh, NC (US); Frank Navas, III, Chapel Hill, NC (US)

Correspondence Address: GLAXOSMITHKLINE;GLOBAL PATENTS, FIVE MOORE DR., PO BOX 13398, MAIL STOP: C.2111F, RESEARCH TRIANGLE PARK, NC, 27709-3398, US

(73) Assignee: SMITHKLINE BEECHAM CORPORATION, Philidelphia, PA (US)

(21) Appl. No.: 12/665,772

(22) PCT Filed: Jun. 13, 2008
(86) PCT No.: PCT/US08/66817

§ 371 Date: Oct. 21, 2010

Related U.S. Application Data
(60) Provisional application No. 60/947,548, filed on Jul. 2, 2007.

(51) Int. Cl. A61K 31/4725; C07D 413/12; C07D 417/14; C07D 413/14; C07D 409/12; C07D 417/12; C07D 413/06; A61K 31/422; A61K 31/427; A61K 31/4439; A61K 31/428; A61K 31/423; A61P 3/00; A61P 1/16; A61P 35/00

(52) U.S. Cl. 514/309; 548/249; 548/187; 546/272.1; 548/178; 548/217; 546/141; 514/378; 514/369; 514/339; 514/367; 514/375

(57) ABSTRACT

The present invention relates to farnesoid X receptors (FXR, NR1H4). FXR is a member of the nuclear receptor class of ligand-activate transcription factors. More particularly, the present invention relates to compounds useful as agonists for FXR, pharmaceutical formulations comprising such compounds, and therapeutic use of the same. Novel isoxazole compounds are disclosed as part of pharmaceutical compositions for the treatment of a condition mediated by decreased FXR activity, such as obesity, diabetes, cholestatic liver disease, liver fibrosis, and metabolic syndrome.

### BACKGROUND OF THE INVENTION

The present invention relates to farnesoid X receptors (FXR, NR1H4). More particularly, the present invention relates to compounds useful as agonists for FXR, pharmaceutical formulations comprising such compounds, and therapeutic use of the same.

FXR is a member of the nuclear receptor class of ligand-activated transcription factors. Physiological concentrations of bile acids bind and activate FXR. [Parks, D. J., et al. 1999 Science 284:1365-1368; Makishima, M., et al. 1999 Science 284:1362-1365] Bile acids are amphipathic molecules that form micelles and emulsify dietary lipids. This property also makes bile acids cytotoxic if sufficient concentrations are achieved and thus mechanisms have evolved to ensure bile acid concentrations are tightly regulated. FXR plays a key role in regulating bile acid homeostasis. [Makishima, M. 2005 J. Pharmacol. Sci. 97:177-183; Kuipers, F., et al., 2004 Rev. Endocrine Metab. Disorders 5:319-326]

FXR is expressed in liver, intestine, kidney, and adrenal. [Kuipers, F., et al., 2004 Rev. Endocrine Metab. Disorders 5:319-326] FXR target genes in hepatocytes include small heterodimer partner (SHP, NR0B2) which encodes an atypical nuclear receptor that represses transcription of genes such as CYP7A1 (encoding cholesterol 7α-hydroxylase), the first and rate limiting step in the conversion of cholesterol to bile acid, CYP8B1 (encoding sterol 12α-hydroxylase) which controls the hydrophobicity of the bile pool and NTCP (encoding the sodium/taurocholate co-transporting polypeptide, SLC10A1) that imports bile acids from the portal and systemic circulation into the hepatocyte. [Goodwin, B., et al., 2000 Mol. Cell 6:517-526; del Castillo-Olivares, A., et al., 2001 Nucleic Acids Res. 29:4035-4042; Denson, L. A., et al. 2001 Gastroenterology 121:140-147] Other FXR target genes that are induced in liver include the canalicular transporter BSEP (encoding the bile salt export pump, ABCB11) that transports bile acids from the hepatocyte into the bile, multi-drug resistance P glycoprotein-3 (MDR3) (encoding the canalicular phospholipid flippase, ABCB4) that transports phospholipids from the hepatocyte into the bile and MRP2 (encoding multidrug resistance-related protein-2, ABCC2) that transports conjugated bilirubin, glutathione and glutathione conjugates into bile. [Ananthanarayanan, M., et al., 2001 J. Biol. Chem. 276:28857-28865; Huang, L., et al., 2003 J. Biol. Chem. 278:51085-51090; Kast, H. R., et al. 2002 J. Biol. Chem. 277:2908-2915.]

In the intestine, FXR also induces expression of SHP which represses transcription of the apical sodium dependent bile acid transporter (ASBT, SLC10A2) gene which encodes the high affinity apical sodium dependent bile acid transporter that moves bile acids from the intestinal lumen into the enterocyte as part of the enterohepatic recycling of bile acids. [Li, H., et al. 2005 Am. J. Physiol. Gastrointest. Liver Physiol. 288:G60-G66] Ileal bile acid binding protein (IBABP) gene expression is also induced by FXR agonists in the enterocyte. [Grober, J., et al., 1999 J. Biol. Chem. 274:29749-29754] The function of this ileal bile acid binding protein remains under investigation.

Cholestasis is a condition of reduced or arrested bile flow. Unresolved cholestasis leads to liver damage such as that seen in primary biliary cirrhosis (PBC) and primary sclerosing cholangitis (PSC), two cholestatic liver diseases. FXR agonists have been shown to protect the liver in rodent models of cholestatic liver disease. [Liu, Y., et al. 2003 J. Clin. Invest. 112:1678-1687; Fiorucci, S., et al., 2005 J. Pharmacol. Exp. Ther. 313:604-612; Pellicciari, R., et al. 2002 J. Med. Chem. 45:3569-3572]

FXR is also expressed in hepatic stellate cells (HSC) which play a role in deposition of extracellular matrix during the fibrotic process. Treatment of cultured HSCs with the FXR agonist 6-ethyl-chenodeoxycholic acid (6EtCDCA) results in decreased expression of fibrotic markers such as α-smooth muscle actin and α1(I)collagen. 6EtCDCA has also been reported to prevent development and promote resolution of hepatic fibrosis in multiple rodent models of this disease. [Fiorucci, S., et al., 2004 Gastroenterology 127:1497-1512; Fiorucci, S., et al., 2005 J. Pharmacol. Exp. Ther. 314:584-595.] According to Fiorucci et al., this anti-fibrotic effect is due to SHP inactivation of Jun and subsequent repression of tissue inhibitor of metalloproteinase 1 (TIMP1) via the activation protein 1 (AP1) binding site on the TIMP1 promoter.

S. Kliewer presented data at the Digestive Diseases Week (DDW) Conference (2005) organized by the American Association for the study of Liver Disease (AASLD) showing that activation of FXR by the agonist GW4064 resulted in improved mucosal barrier and decreased bacterial overgrowth in a bile duct-ligated mouse model of cholestasis and intestinal bacterial overgrowth. Dr. Kliewer showed data indicating decreased translocation of bacteria to mesenteric lymph nodes in mice treated with GW4064. This effect of GW4064 was lost in FXR null mice. [Inagaki, T., et al., 2006 Proc. Nat. Acad. Sci., U.S.A. 103:3920-3925.]

The FXR agonist GW4064, when administered to mice on a lithogenic diet, prevented the formation of cholesterol crystals in the bile. This effect of the compound was lost in FXR null mice. Moschetta, A., et al. 2004 Nat. Med. 10:1352-1358.

It has been suggested that GW4064 could improve lipid and glucose homeostasis and insulin sensitivity in rodent diabetic and insulin resistance models. Chen and colleagues [2006 Diabetes 55 suppl. 1: A200] demonstrated that when administered to mice on high-fat diet, GW4064 decreased body weight and body fat mass, serum glucose, insulin, triglyceride, and total cholesterol. GW4064 also corrected glucose intolerance in those animals. In addition, GW4064 decreased serum insulin concentration, improved glucose tolerance and enhanced insulin sensitivity in ob/ob mice [Cariou, B., et al., 2006 J. Biol. Chem. 281:11039-11049]. In another study, it was reported that GW4064 significantly improved hyperglycemia and hyperlipidemia in diabetic db/db mice [Zhang, Y., et al. 2006 Proc. Nat. Acad. Sci., U.S.A. 103:1006-1011].

### SUMMARY OF THE INVENTION

As a first aspect, the present invention provides compounds of formula (I):

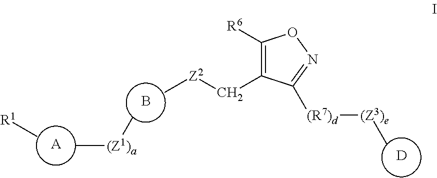

wherein:
Ring A is phenyl or a 5-6 membered heterocycle or heteroaryl comprising 1, 2 or 3 heteroatoms selected from N, O and S, wherein said phenyl, heterocycle or heteroaryl is substituted with R¹ and further optionally substituted with one or two substituents independently selected from C₁₋₆alkyl, halo and haloalkyl;
R¹ is selected from —CO₂H, —C(O)NH₂, —CO₂alkyl, —CH₂CH₂CO₂H, —CH₂CH₂CO₂alkyl, —NHC(O)CH₃, —N(C(O)CH₃)₂, —N(SO₂CF₃)₂, —OCF₃ and an acid equivalent group (for example —NHSO₂CF₃ or

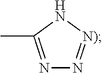

### Z¹ is —CH₂—, —CO—, —NH—, —S—, —SO— or —SO₂—;

a is 0 or 1;
Ring B is selected from

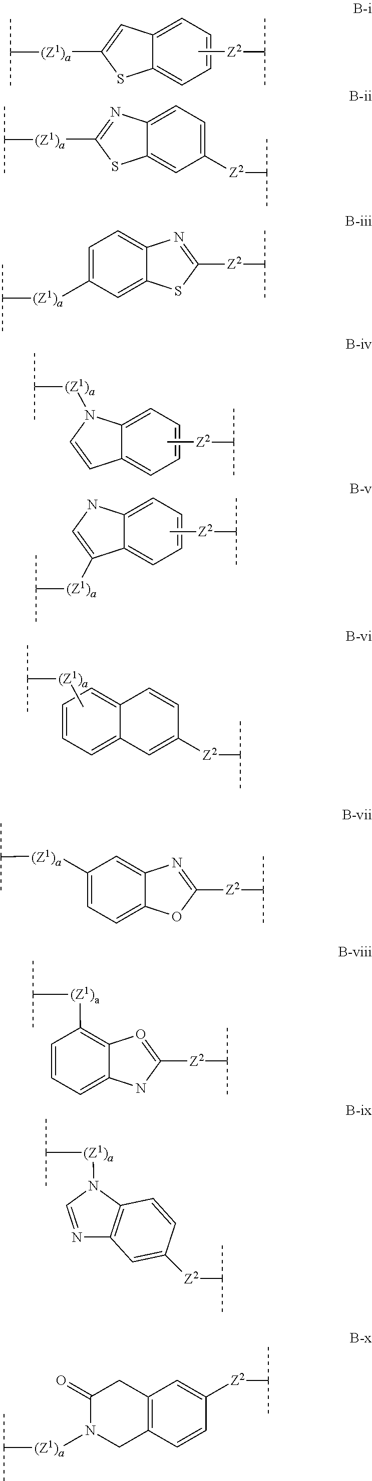

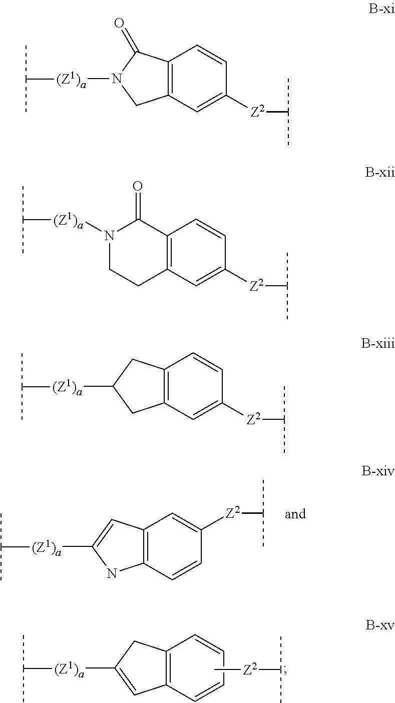

Z² is —O—, —S—, —CH₂— or —N(R⁵)—, wherein R⁵ is H or alkyl;
R⁶ is selected from alkyl, 2,2,2-trifluoroethyl, C₃₋₆cycloalkyl, alkenyl, C₃₋₆cycloalkenyl and fluoro-substituted C₃₋₆cycloalkyl;
R⁷ is —C₁₋₃alkylene-;
Z³ is —O—, —S(O)_{c}—, or —NH—, where c is 0, 1 or 2;
d and e are both 0 or d is 1 and e is 0 or 1; and
Ring D is selected from C₃₋₆cycloalkyl and a moiety of formula D-i, D-ii, D-iii, D-iv or D-v

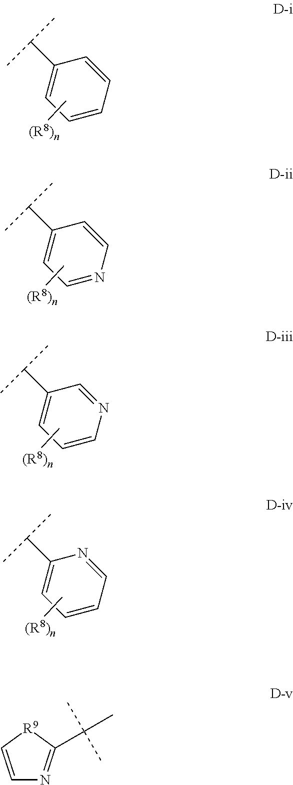

wherein
n is 0, 1, 2 or 3;
each R⁸ is the same or different and is independently selected from halo, alkyl, alkenyl, —O-alkyl, haloalkyl, hydroxyl substituted alkyl, and —OCF₃;
R⁹ is —O—, —NH— or —S—;
   and pharmaceutically acceptable salts thereof.

In a second aspect, the present invention provides a pharmaceutical composition comprising a compound of formula (I). The composition may further comprise a pharmaceutically acceptable carrier or diluent.

In a third aspect, the present invention provides a method for the treatment of a condition mediated by decreased FXR activity in a subject in need thereof. The method comprises administering to the subject a therapeutically effective amount of a compound of formula (I).

In a fourth aspect, the present invention provides a method for the treatment of obesity in a subject in need thereof. The method comprises administering to the subject a therapeutically effective amount of a compound of formula (I).

In a fifth aspect, the present invention provides a method for the treatment of diabetes mellitus in a subject in need thereof. The method comprises administering to the subject a therapeutically effective amount of a compound of formula (I).

In a sixth aspect, the present invention provides a method for the treatment of metabolic syndrome in a subject in need thereof. The method comprises administering to the subject a therapeutically effective amount of a compound of formula (I).

In a seventh aspect, the present invention provides a method for the treatment of cholestatic liver disease in a subject in need thereof. The method comprises administering to the subject a therapeutically effective amount of a compound of formula (I).

In a eighth aspect, the present invention provides a method for the treatment of organ fibrosis in a subject in need thereof. The method comprises administering to the subject a therapeutically effective amount of a compound of formula (I). In one embodiment, the organ fibrosis is liver fibrosis.

In a ninth aspect, the present invention provides a method for the treatment of liver fibrosis in a subject in need thereof. The method comprises administering to the subject a therapeutically effective amount of a compound of formula (I).

In a tenth aspect, the present invention provides a process for preparing a compound of formula (I). The process comprises the step of:

a) reacting a compound of formula (II)

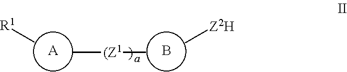

with a compound of formula (III)

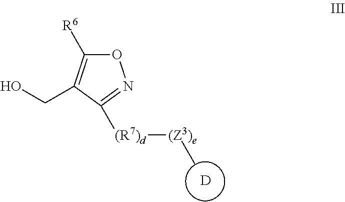

wherein R¹ is —CO₂alkyl, —CH₂CH₂CO₂alkyl, —NHC(O)CH₃, or —OCF₃;
Z² is —O—, —NH— or —S—; and
all other variables are as defined above for formula (I)
   to prepare a compound of formula (I).

In another aspect, the present invention provides another process for preparing a compound of formula (I). This process comprises the step of:

a) reacting a compound of formula (II)

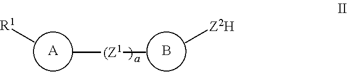

with a compound of formula (XLII)

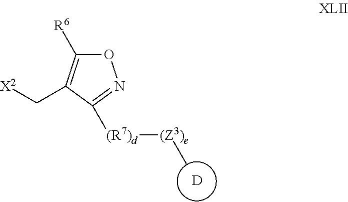

wherein:
a is 0;
Z² is —O—, —NH— or —S—;
X² is chloride, iodide, bromide, triflate, tosylate, nosylate, besylate or mesylate, (preferably chloro);
R¹ is —CO₂alkyl, —CH₂CH₂CO₂alkyl, —NHC(O)CH₃, or —OCF₃; and
all other variables are as defined above for formula (I)
   to prepare a compound of formula (I).

In another aspect, the present invention provides another process for preparing a compound of formula (I). This process comprises the step of:

a) reacting a compound of formula (XIII)

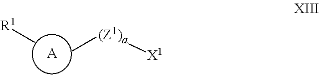

with a boronic acid or ester compound of formula (XLVII) under Suzuki coupling conditions

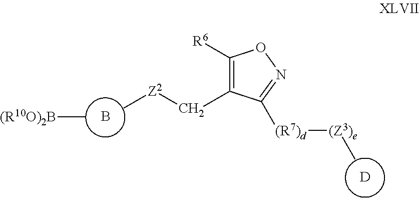

wherein:
R¹ is —CO₂alkyl;
a is 0;
X¹ is chloro, bromo, iodo or triflate;
Ring A is phenyl or a 5-6 membered heteroaryl comprising 1, 2 or 3 heteroatoms selected from N, O and S, wherein said phenyl or heteroaryl is substituted with R¹ and further optionally substituted with one or two independently selected C₁₋₆alkyl;
Ring B is B-i, B-ii, B-iii, B-iv, B-v, B-vi, B-vii, B-viii, B-ix, B-xiv, or B-xv;
R¹⁰ is H or alkyl; and
all other variables are as defined above for formula (I)
   to prepare a compound of formula (I).

In another aspect, the present invention provides another process for preparing a compound of formula (I). This process comprises the step of:

a) reacting a compound of formula (XLIX)

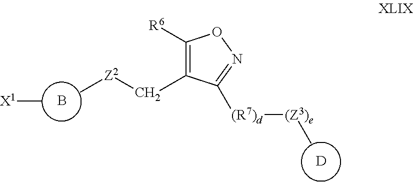

with a boronic acid or ester compound of formula (XV) under Suzuki coupling conditions

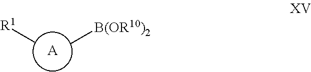

wherein:
R¹ is —CO₂alkyl, —CH₂CH₂CO₂alkyl, —NHC(O)CH₃, or —OCF₃;
Ring A is phenyl or a 5-6 membered heteroaryl comprising 1, 2 or 3 heteroatoms selected from N, O and S, wherein said phenyl or heteroaryl is substituted with R¹ and further optionally substituted with one or two independently selected C₁₋₆alkyl;
Ring B is B-i, B-ii, B-iii, B-iv, B-v, B-vi, B-vii, B-viii, B-ix, B-xiv, or B-xv;
a is 0;
R¹⁰ is H or alkyl;
X¹ is chloro, bromo, iodo or triflate; and
all other variables are as defined above for formula (I)
   to prepare a compound of formula (I).

In another aspect, the present invention provides another process for preparing a compound of formula (I). This process comprises the steps of:

a) reacting a compound of formula (LV)

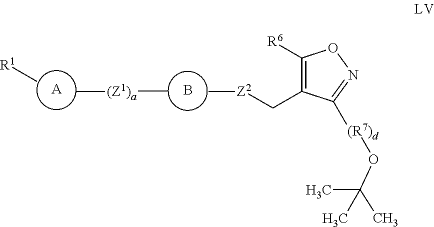

with an acid to prepare a compound of formula (LVI)

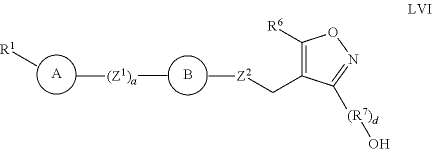

wherein: R¹ is —CO₂alkyl; and
all other variables are as defined above for formula (I);
   b) reacting a compound of formula (LVI) under Mitsunobu reaction conditions with
   a Ring D moiety of formula D-i, D-ii-a, or D-v-a

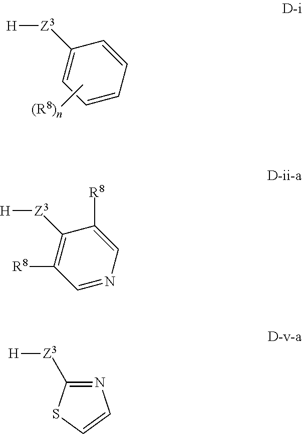

wherein:
R¹ is —CO₂alkyl;
Z³ is selected from —O—, —S—, —NH—,
e is 1;
Ring D is a moiety of formula D-i, D-ii-a or D-v-a:

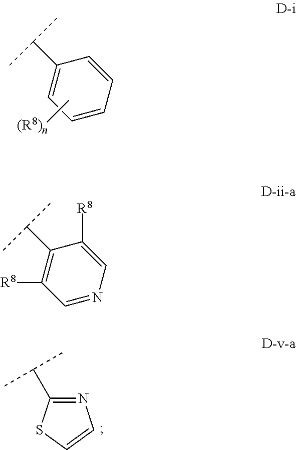

and
all other variables are as defined above for formula (I)
   to prepare a compound of formula (I).

In another aspect, the present invention provides another process for preparing a compound of formula (I). This process comprises the step of:

a) condensing a compound of formula (LXXII)

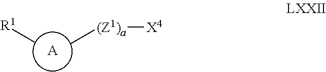

with a compound of formula (LVII) optionally with a base

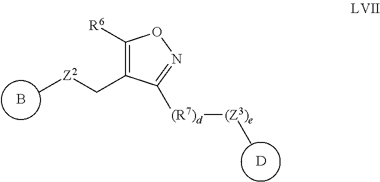

wherein:
R¹ is —CO₂alkyl;
Z¹ is —CH₂—, —CO— or —SO₂—;
a is 1;
X⁴ is iodo, chloro or bromo (preferably chloro);
Ring B is an indole or benzamidazole; and
all other variables are as defined above for formula (I)
   to prepare a compound of formula (I)

In another aspect, the present invention provides another process for preparing a compound of formula (I). This process comprises the step of:

a) condensing a compound of formula (LXI)

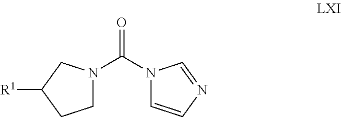

with a compound of formula (LVII-a) optionally with a base

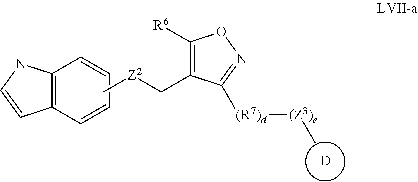

wherein: R¹ is —CO₂alkyl; and
all other variables are as defined above for formula (I)
   to prepare a compound of formula (I-c)

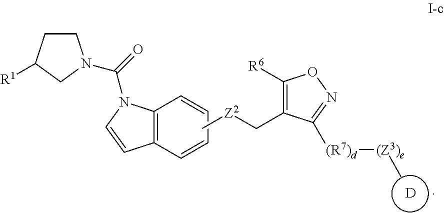

In another aspect, the present invention provides another process for preparing a compound of formula (I). This process comprises the step of:

a) reacting a compound of formula (LXIV)

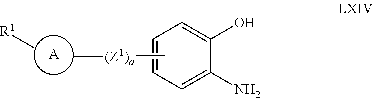

with a compound of formula (LXV)

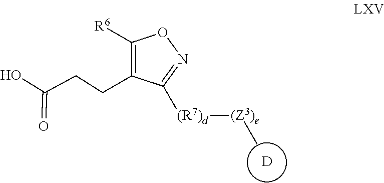

wherein:
Z¹ is —NH—;
a is 0 or 1;
R¹ is —CO₂alkyl; and
all other variables are as defined above for formula (I)
to prepare an intermediate amide, and dehydrating the intermediate to prepare a compound of formula (I-d)

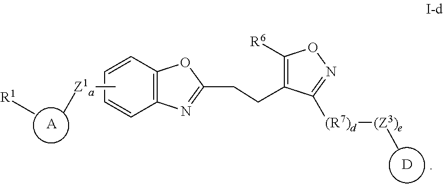

In another aspect, the present invention provides another process for preparing a compound of formula (I). This process comprises the step of:
a) reacting a compound of formula (II-b)

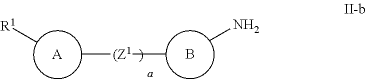

with a compound of formula (LXVI)

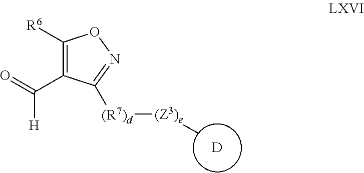

wherein:
R¹ is —CO₂alkyl;
Z² is —NH—;
all other variables are as defined above for formula (I)
   to prepare a compound of formula (I).

In another aspect, the present invention provides a compound of formula (I) for use in therapy. The present invention also provides a compound of formula (I) for use in the treatment of a condition mediated by decreased FXR activity in a subject; a compound of formula (I) for use in the treatment of obesity in a subject; a compound of formula (I) for use in the treatment of diabetes mellitus in a subject; a compound of formula (I) for use in the treatment of metabolic syndrome in a subject; a compound of formula (I) for use in the treatment of cholestatic liver disease in a subject; a compound of formula (I) for use in the treatment of organ fibrosis in a subject; and a compound of formula (I) for use in the treatment of liver fibrosis in a subject.

In another aspect, the present invention provides the use of a compound of formula (I) for the preparation of a medicament for the treatment of a condition mediated by decreased FXR activity in a subject; the use of a compound of formula (I) for the preparation of a medicament for the treatment of obesity; the use of a compound of formula (I) for the preparation of a medicament for the treatment of diabetes mellitus in a subject; the use of a compound of formula (I) for the preparation of a medicament for the treatment of metabolic syndrome in a subject; the use of a compound of formula (I) for the preparation of a medicament for the treatment of cholestatic liver disease in a subject; the use of a compound of formula (I) for the preparation of a medicament for the treatment of organ fibrosis in a subject; and the use of a compound of formula (I) for the preparation of a medicament for the treatment of liver fibrosis in a subject.

In another aspect, the present invention provides a pharmaceutical composition comprising a compound of formula (I) for use in the treatment of a condition mediated by decreased FXR activity.

In another aspect, the present invention provides a pharmaceutical composition comprising a compound of formula (I) for use in the treatment of a condition selected from diabetes mellitus, metabolic syndrome, cholestatic liver disease, and liver fibrosis.

Further aspects of the present invention are described in the description of particular embodiments, examples, and claims which follow.

### DETAILED DESCRIPTION OF THE PREFERRED EMBODIMENTS

As used herein, “a compound of the invention” or “a compound of formula (I)” or “(I-A),” etc. means a compound of formula (I) (or (I-A), etc.) or a pharmaceutically acceptable salt or solvate thereof. Similarly, with respect to isolatable intermediates such as for example, compounds of formula (II), (III), (IV), (V), (XL), (XLI) and (XLII), the phrase “a compound of formula (number)” means a compound having that formula or a pharmaceutically acceptable salt or solvate thereof.

As used herein, the term “alkyl” refers to aliphatic straight or branched saturated hydrocarbon chains containing 1-8 carbon atoms. Examples of “alkyl” groups as used herein include but are not limited to methyl, ethyl, n-propyl, isopropyl, n-butyl, isobutyl, t-butyl, pentyl, hexyl, octyl and the like.

The term “haloalkyl” as used herein refers to an alkyl as defined above substituted with one or more halogen atoms.

The term “alkylene” refers to a straight or branched alkyl bridge, i.e., the group -alkyl-, wherein alkyl is as defined above.

As used herein, the term “halo” refers to any halogen atom, i.e., fluorine, chlorine, bromine or iodine.

As used herein, the term “alkenyl” refers to an aliphatic straight or branched unsaturated hydrocarbon chain containing 2-8 carbon atoms and at least one and up to three carbon-carbon double bonds. Examples of “alkenyl” groups as used herein include but are not limited to ethenyl and propenyl.

As used herein, the term “cycloalkyl” refers to a non-aromatic monocyclic carbocyclic ring having from 3 to 8 carbon atoms (unless a different number of atoms is specified) and no carbon-carbon double bonds. “Cycloalkyl” includes by way of example cyclopropyl, cyclobutyl, cyclopentyl, cyclohexyl, cycloheptyl and cyclooctyl. Particular cycloalkyl groups include C₃₋₆cycloalkyl.

As used herein, the term “cycloalkenyl” refers to a non-aromatic monocyclic carbocyclic ring having from 3 to 8 carbon atoms (unless a different number of atoms is specified) and from 1 to 3 carbon-carbon double bonds. “Cycloalkenyl” includes by way of example cyclopropenyl, cyclobutenyl, cyclopentenyl, cyclohexenyl, cycloheptenyl and cyclooctenyl. Particular cycloalkenyl groups include C₃₋₆cycloalkenyl.

As used herein, the term “heterocycle” refers to a ring structure having one or more heteroatoms.

As used herein, the term “heteroaryl” refers to an aromatic ring having one or more heteroatoms.

As used herein, the term “optionally” means that the subsequently described event(s) may or may not occur, and includes both event(s) that occur and events that do not occur.

The present invention provides compounds of formula (I):

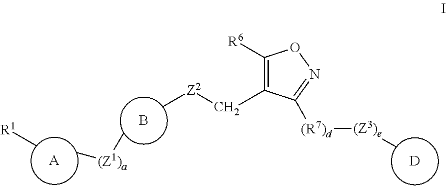

wherein:
Ring A is phenyl or a 5-6 membered heterocycle or heteroaryl comprising 1, 2 or 3 heteroatoms selected from N, O and S, wherein said phenyl, heterocycle or heteroaryl is substituted with R¹ and further optionally substituted with one or two substituents independently selected from C₁₋₆alkyl, halo and haloalkyl;
R¹ is selected from —CO₂H, —C(O)NH₂, —CO₂alkyl, —CH₂CH₂CO₂H, —CH₂CH₂CO₂alkyl, —NHC(O)CH₃, —N(C(O)CH₃)₂, —N(SO₂CF₃)₂, —OCF₃ and an acid equivalent group (for example —NHSO₂CF₃ or

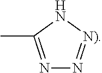

### Z¹ is —CH₂—, —CO—, —NH—, —S—, —SO— or —SO₂—;

a is 0 or 1;
Ring B is selected from

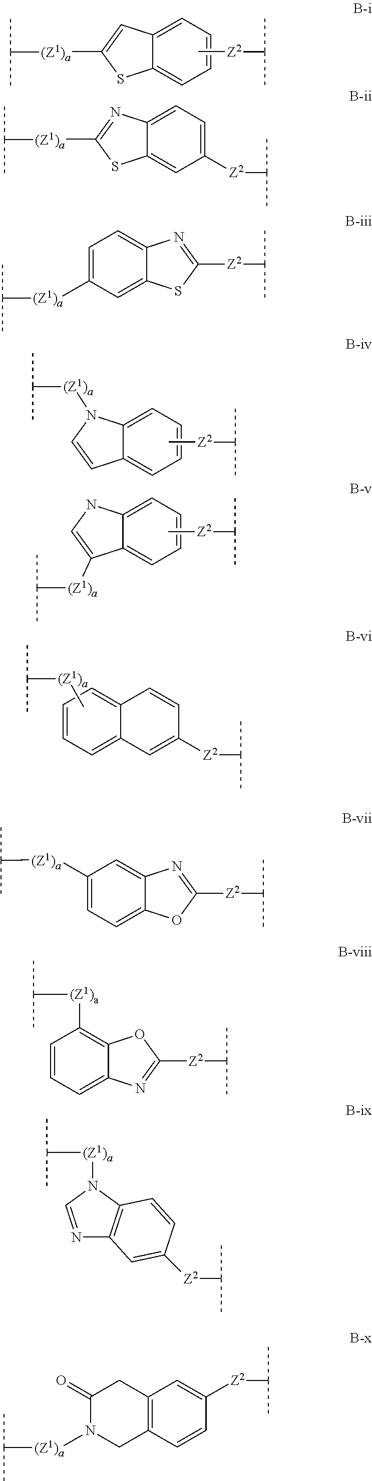

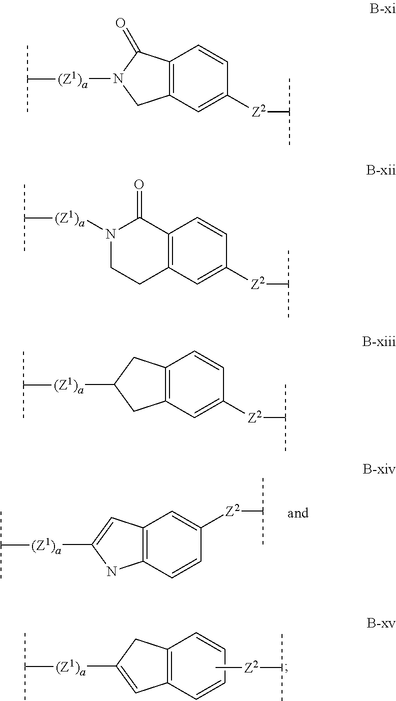

Z² is —O—, —S—, —CH₂— or —N(R⁵)—, wherein R⁵ is H or alkyl;
R⁶ is selected from alkyl, 2,2,2-trifluoroethyl, C₃₋₆cycloalkyl, alkenyl, C₃₋₆cycloalkenyl and fluoro-substituted C₃₋₆cycloalkyl;
R⁷ is —C₁₋₃alkylene-;
Z³ is —O—, —S(O)_{c}—, or —NH—, where c is 0, 1 or 2;
d and e are both 0 or d is 1 and e is 0 or 1; and
Ring D is selected from C₃₋₆cycloalkyl and a moiety of formula D-i, D-ii, D-iii, D-iv or D-v

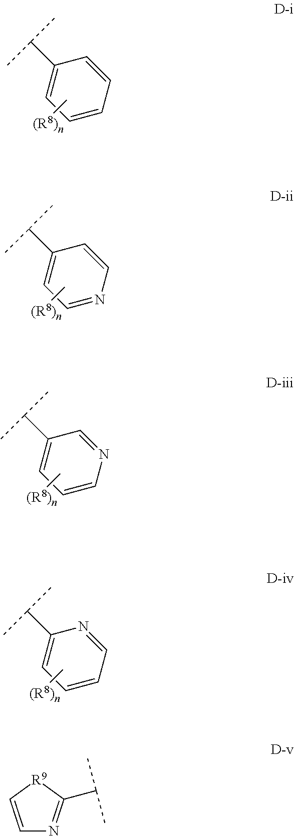

wherein
n is 0, 1, 2 or 3;
each R⁸ is the same or different and is independently selected from halo, alkyl, alkenyl, —O-alkyl, haloalkyl, hydroxyl substituted alkyl, and —OCF₃;
R⁹ is —O—, —NH— or —S—;
   and pharmaceutically acceptable salts thereof.

In one particular embodiment of the invention, the present invention provides compounds of formula (I)

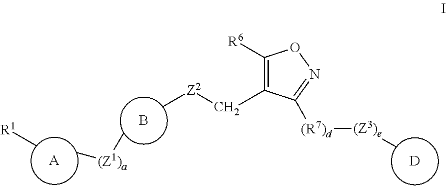

wherein:
Ring A is selected from

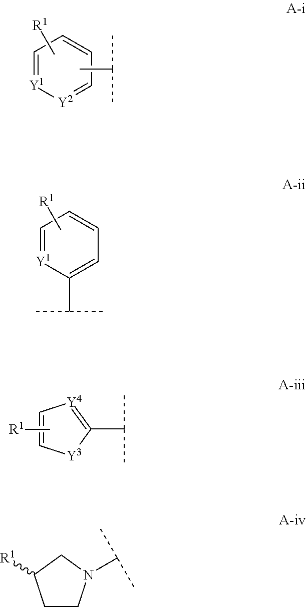

wherein R¹ is selected from —CO₂H, —C(O)NH₂, —CO₂alkyl, —CH₂CH₂CO₂H, —CH₂CH₂CO₂alkyl, —NHC(O)CH₃, —N(C(O)CH₃)₂, —N(SO₂CF₃)₂, —OCF₃ and an acid equivalent group (for example —NHSO₂CF₃ or

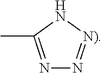

Y¹ is selected from CR², N;
Y² is selected from CR², N;
Y³ is selected from O, S, or NH;
Y⁴ is selected from CH or N;
R² is selected from H, C₁₋₆ alkyl, halo, haloalkyl;

### Z¹ is —CH₂—, —CO—, —NH—, —S—, —SO— or —SO₂—;

a is 0 or 1;
Ring B is selected from

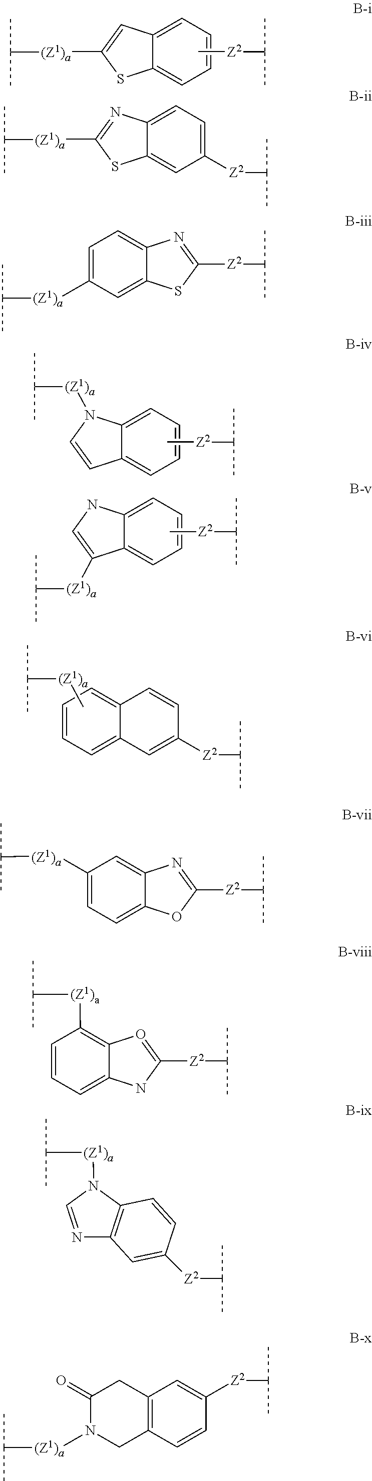

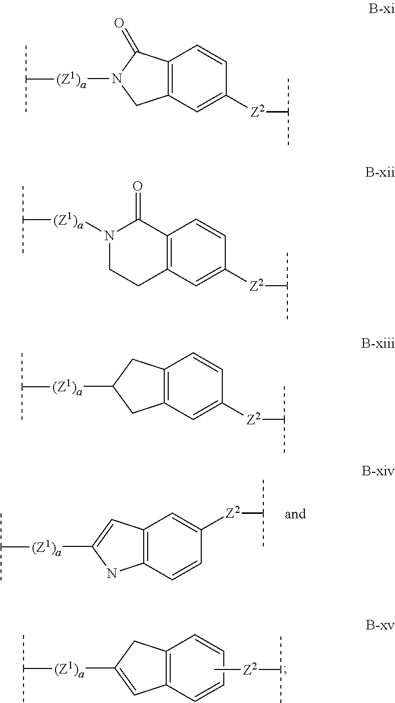

Z² is —O—, —S—, —CH₂— or —N(R⁵)—, wherein R⁵ is H or alkyl;
R⁶ is selected from alkyl, 2,2,2-trifluoroethyl, C₃₋₆cycloalkyl, alkenyl, C₃₋₆cycloalkenyl and fluoro-substituted C₃₋₆cycloalkyl;
R⁷ is —C₁₋₃alkylene-;
Z³ is —O—, —S(O)_{c}—, or —NH—, where c is 0, 1 or 2;
d and e are both 0 or d is 1 and e is 0 or 1; and
Ring D is selected from C₃₋₆cycloalkyl and a moiety of formula D-i, D-ii, D-iii, D-iv or D-v

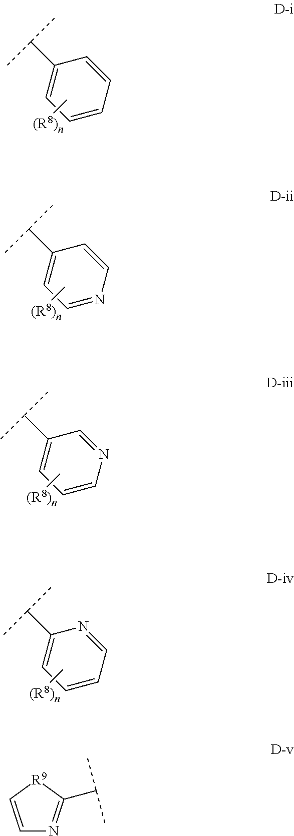

wherein
n is 0, 1, 2 or 3;
each R⁸ is the same or different and is independently selected from halo, alkyl, alkenyl, —O-alkyl, haloalkyl, hydroxyl substituted alkyl, and —OCF₃;
R⁹ is —O—, —NH— or —S—;
   and pharmaceutically acceptable salts thereof.

In one embodiment, Ring A is phenyl or a 5-6 membered heterocycle or heteroaryl comprising 1, 2 or 3 heteroatoms selected from N, O and S, wherein said phenyl, heterocycle or heteroaryl is substituted with R¹ and further optionally substituted with one or two substituents independently selected from C₁₋₆alkyl, halo and haloalkyl.

In another embodiment, Ring A is phenyl or a 5-6 membered heterocycle or heteroaryl comprising 1, 2 or 3 heteroatoms selected from N, O and S, wherein said phenyl, heterocycle or heteroaryl is substituted with R¹ and further optionally substituted with one substituent independently selected from C₁₋₆alkyl, halo and haloalkyl.

In another embodiment, Ring A is

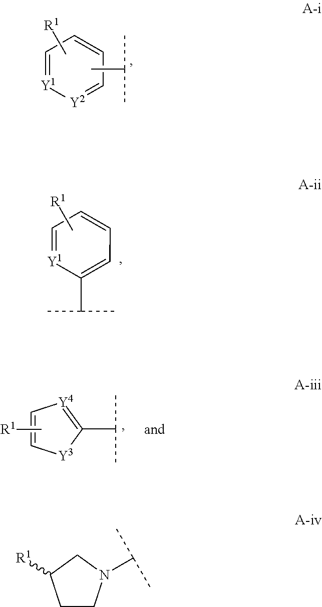

wherein R¹ is selected from —CO₂H, —C(O)NH₂, —CO₂alkyl, —CH₂CH₂CO₂H, —CH₂CH₂CO₂alkyl, —NHC(O)CH₃, —N(C(O)CH₃)₂, —N(SO₂CF₃)₂, —OCF₃ and an acid equivalent group (for example —NHSO₂CF₃ or

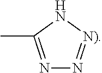

Y¹ is selected from CR², N;
Y² is selected from CR², N;
Y³ is selected from O, S, or NH;
Y⁴ is selected from CH or N;
R² is selected from H, C₁₋₆ alkyl, halo, haloalkyl.

In one particular embodiment of the invention, Ring A is A-i:

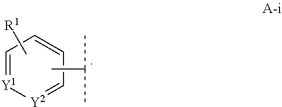

Specific examples of Ring A-i include but are not limited to

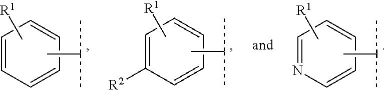

In another embodiment of the invention, Ring A is A-ii:

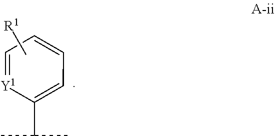

A specific example of Ring A-ii is

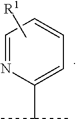

In another embodiment of the invention, Ring A is A-iii:

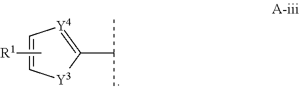

Specific examples of Ring A-iii include but are not limited to

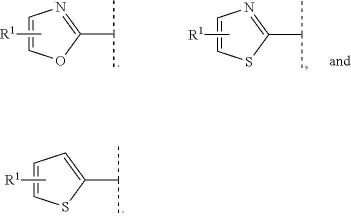

In another embodiment of the invention, Ring A is A-iv:

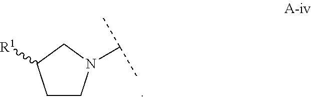

Specific examples of Ring A-iv include but are not limited to

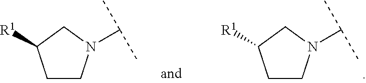

In one embodiment, R¹ is selected from —CO₂H, —C(O)NH₂, —NHC(O)CH₃, and an acid equivalent group, or any subset thereof. In one preferred embodiment R¹ is —CO₂H or an acid equivalent group. In another preferred embodiment, R¹ is —CO₂H.

In one embodiment, R² is selected from the group consisting of H and C₁₋₆alkyl, such as

—CH₃, or any subset thereof. In one preferred embodiment, R² is H.

In one embodiment of the invention, Z¹ is selected from the group consisting of —CH₂—, —CO—, —NH—, and —SO²—, or any subset thereof. In another embodiment of the invention, Z¹ is —CH₂— or —NH—. In another embodiment, Z¹ is —CH₂—. In another embodiment, Z¹ is —NH—.

In one embodiment of the invention, a is 0. In another embodiment, a is 1.

In one embodiment of the invention, Ring B is selected from the group consisting of

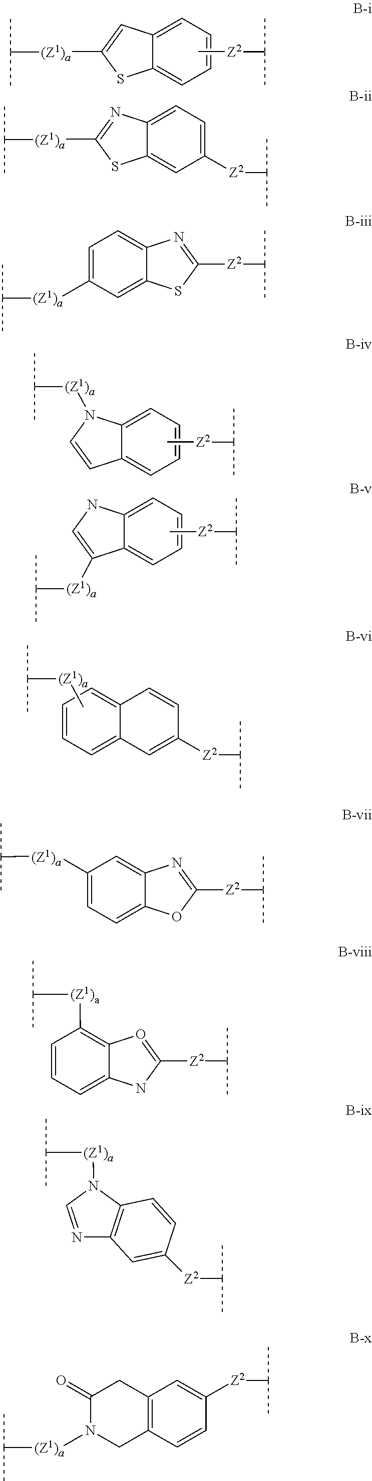

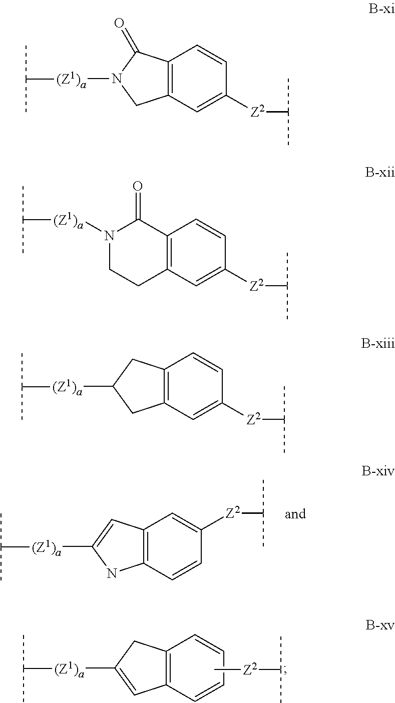

Particular embodiments are represented by each of the foregoing Ring Bs individually.

In one embodiment of the invention, Ring B is selected from the group consisting of

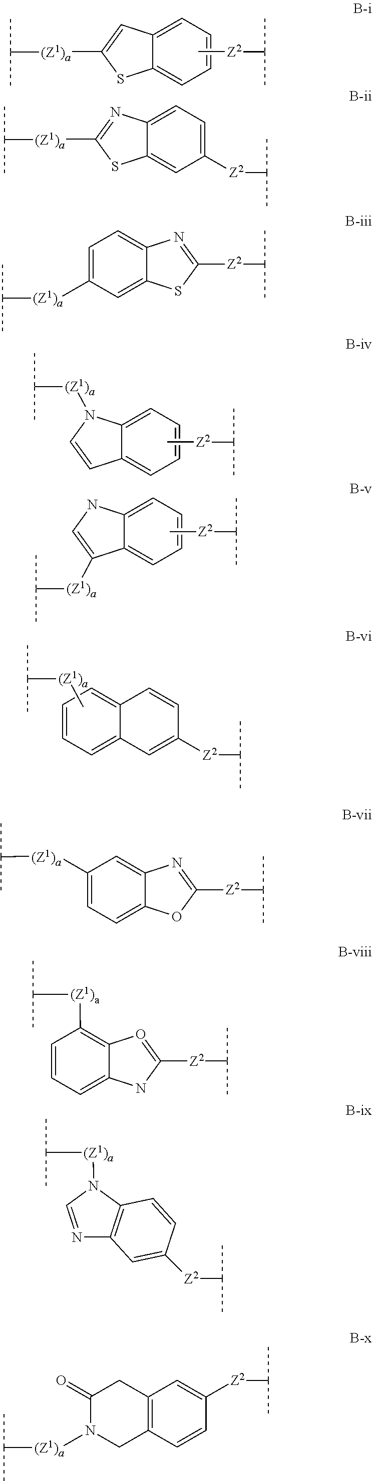

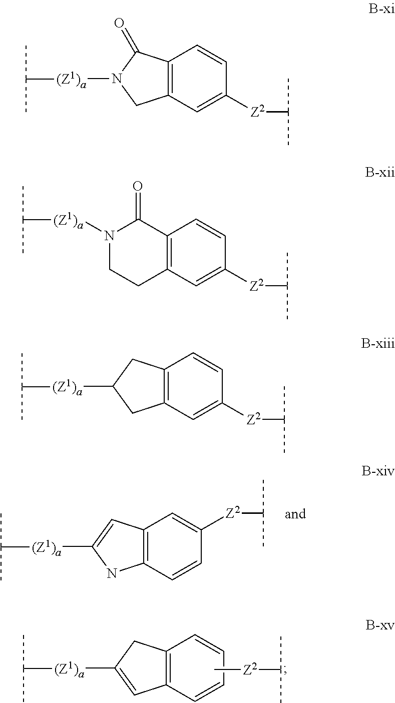

In one preferred embodiment of the invention, Ring B is B-iv:

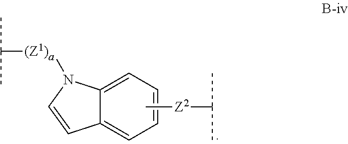

In another preferred embodiment of the invention, Ring B is B-vi:

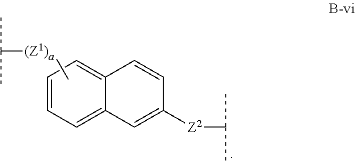

In one embodiment of the invention, Z² is selected from the group consisting of —O—, —CH₂— and —N(H)—, or any subset thereof. In one preferred embodiment, Z² is —O—.

In one embodiment, R⁶ is selected from the group consisting of alkyl, 2,2,2-trifluoroethyl and C₃₋₆cycloalkyl, or any subset thereof. Specific examples of groups defining R⁶ include but are not limited to methyl, ethyl, propyl, isopropyl, t-butyl, n-butyl, isobutyl, 2,2,2-trifluoroethyl, cyclopropyl, cyclobutyl, cyclopentyl and cyclohexyl. In one embodiment, R⁶ is selected from the group consisting of isopropyl, isobutyl, 2,2,2-trifluoroethyl, cyclopropyl, cyclobutyl and cyclopentyl, or any subset thereof. In one embodiment, R⁶ is isopropyl, isobutyl, cyclopropyl or cyclobutyl. In one particular embodiment, R⁶ is isopropyl or isobutyl. In one preferred embodiment, R⁶ is isopropyl.

In one particular embodiment, the invention includes compounds of formula I′ wherein d is 0 and e is 0 and thus Ring D is bound directly to the isoxazole ring as shown in formula (I′):

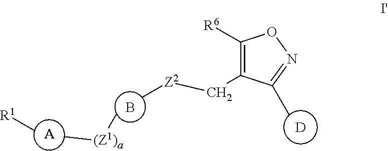

wherein all other variables are as defined above including particular and preferred embodiments thereof.

The invention also includes compounds of formula (I″) wherein d is 1 and e is 0 or 1 and thus Ring D is bound to C₁₋₃ alkylene (R⁷) (when e is 0) or Z³ (when e is 1) as shown in formula (I″).

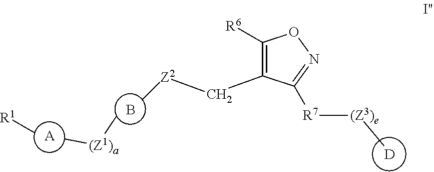

wherein all other variables are as defined above including particular and preferred embodiments thereof.

In one particular embodiment, the invention also includes compounds of formula (I) wherein d is 1 and R⁷ is preferably methylene or ethylene. In another embodiment, d and e are 1 and R⁷ is preferably methylene. In another embodiment, d is 1, e is 1 and Z³ is selected from the group consisting of —O—, —S— and —NH—, or any subset thereof. In one particular embodiment, d is 1, e is 1, R⁷ is methylene and Z³ is —O—, as in formula (I″′):

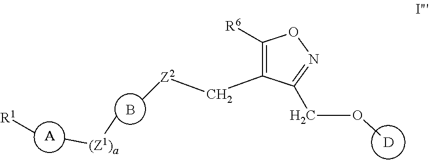

wherein all other variables are as defined above. The invention includes compounds of formula I′″.

Ring D is selected from C₃₋₆cycloalkyl and a moiety selected from formula D-i, D-ii, D-iii, D-iv, and D-v:

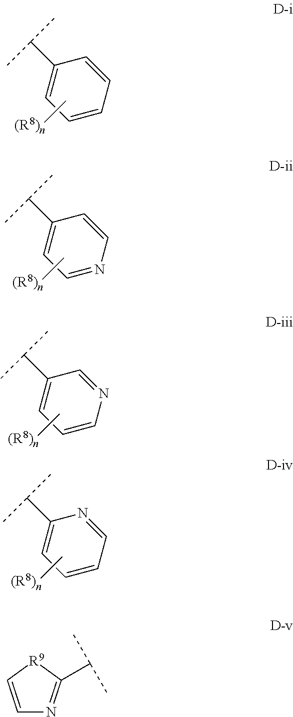

wherein:
n is 0, 1, 2 or 3;
each R⁸ is the same or different and is independently selected from halo, alkyl, alkenyl, —O-alkyl, haloalkyl, hydroxyl substituted alkyl, and —OCF₃;
R⁹ is —O—, —NH— or —S—.

In one embodiment Ring D is a moiety of formula D-i. In another embodiment, Ring D is a moiety of formula D-ii. In another embodiment, Ring D is a moiety of formula D-v. In a particular embodiment, Ring D is a moiety of formula D-v and R⁹ is —S—.

In one embodiment wherein Ring D is a moiety of formula D-i, n is 2 or 3 and each R⁸ is the same or different and is independently selected from halo and alkyl. In one particular embodiment wherein Ring D is a moiety of formula D-i, n is 2 or 3, each R⁸ is the same and is F, Cl, Br or methyl. In one preferred embodiment wherein Ring D is a moiety of formula D-i, n is 2 or 3 and each R⁸ is Cl.

In one particular embodiment wherein Ring D is a moiety of formula D-i and n is 2, each R⁸ is the same and is halo or alkyl. In one particular embodiment wherein Ring D is a moiety of formula D-i and n is 2, each R⁸ is the same and is F, Cl, or methyl.

In one preferred embodiment wherein Ring D is a moiety of formula D-i and n is 2, each R⁸ is Cl.

In one embodiment, n is 2 and each R⁸ is the same or different and is independently selected from halo, alkyl, alkenyl, —O-alkyl, haloalkyl, hydroxyl substituted alkyl, and —OCF₃,

In one embodiment, n is 1, 2 or 3 and each R⁸ is the same or different and is independently selected from halo and alkyl. In another embodiment, n is 2 and each R⁸ is the same and is halo or alkyl. In another embodiment, n is 1, 2 or 3 and each R⁸ is the same or different and is independently selected from F, Cl, Br and methyl. In another embodiment, n is 2 or 3, each R⁸ is the same and is selected from F, Cl, Br and methyl, or any subset thereof. In one preferred embodiment, n is 1, 2 or 3 and each R⁸ is Cl. In another embodiment, n is 2 or 3, each R⁸ is the same and is Cl. In another preferred embodiment, n is 2 and each R⁸ is Cl.

Specific examples of particular compounds of the present invention include those set forth in the examples below and pharmaceutically acceptable salts thereof.

One preferred compound of the invention is 3-{[5-({[3-(2,6-dichlorophenyl)-5-(1-methylethyl)-4-isoxazolyl]methyl}oxy)-1H-indol-1-yl]methyl}benzoic acid; and pharmaceutically acceptable salts thereof. One particular embodiment is 3-{[5-({[3-(2,6-dichlorophenyl)-5-(1-methylethyl)-4-isoxazolyl]methyl}oxy)-1H-indol-1-yl]methyl}benzoic acid; or a pharmaceutically acceptable salt thereof is in crystalline form. One preferred embodiment is 3-{[5-({[3-(2,6-dichlorophenyl)-5-(1-methylethyl)-4-isoxazolyl]methyl}oxy)-1H-indol-1-yl]methyl}benzoic acid (i.e. the acid).

Certain compounds of formula (I) may exist in stereoisomeric forms (e.g. they may contain one or more asymmetric carbon atoms). The individual stereoisomers (enantiomers and diastereomers) and mixtures of these are included within the scope of the present invention. The present invention also covers the individual isomers of the compounds represented by formula (I) as mixtures with isomers thereof in which one or more chiral centers are inverted.

Suitable pharmaceutically acceptable salts according to the present invention will be readily determined by one skilled in the art and will include, for example, salts prepared from inorganic bases such as lithium hydroxide, sodium hydroxide, potassium hydroxide, lithium hydride, sodium hydride, potassium hydride, lithium carbonate, lithium hydrogen carbonate, sodium carbonate, sodium hydrogen carbonate, potassium carbonate, potassium hydrogen carbonate, as well as potassium tert-butoxide and organic bases such as diethyl amine, lysine, arginine, choline, tris (hydroxymethyl)aminomethane (tromethamine), triethanolamine, diethanolamine, and ethanolamine.

When used in medicine, the salts of a compound of formula (I) should be pharmaceutically acceptable, but pharmaceutically unacceptable salts may conveniently be used to prepare the corresponding free base or pharmaceutically acceptable salts thereof.

As used herein, the term “solvate” refers to a crystal form containing the compound of formula (I) or a pharmaceutically acceptable salt thereof and either a stoichiometric or a non-stoichiometric amount of a solvent. Solvents, by way of example, include water (thus producing hydrates), methanol, ethanol, or acetic acid. Hereinafter, reference to a compound of formula (I) is to any physical form of that compound, unless a particular form, salt or solvate thereof is specified.

Processes for preparing pharmaceutically acceptable salts of the compounds of formula (I) are conventional in the art. See, e.g., Burger's Medicinal Chemistry And Drug Discovery 5th Edition, Vol 1: Principles And Practice.

As will be apparent to those skilled in the art, in the processes described below for the preparation of compounds of formula (I), certain intermediates, may be in the form of pharmaceutically acceptable salts of the compound. Those terms as applied to any intermediate employed in the process of preparing compounds of formula (I) have the same meanings as noted above with respect to compounds of formula (I). Processes for preparing pharmaceutically acceptable salts of such intermediates are known in the art and are analogous to the process for preparing pharmaceutically acceptable salts of the compounds of formula (I).

In one embodiment, the compounds of formula (I) are FXR agonists. As used herein, the term “FXR agonist” refers to compounds which exhibit a pEC₅₀ greater than 4 in the FXR Cofactor Recruitment Assay described below. More particularly, FXR agonists are compounds which exhibit a pEC₅₀ greater than 5 in the FXR Cofactor Recruitment Assay described below.

Compounds of formula (I) are useful in therapy in subjects such as mammals, and particularly humans. In particular, the compounds of formula (I) are useful in the treatment of a condition mediated by decreased FXR activity in a subject such as a mammal, particularly a human. As used herein, the term “treatment” includes the prevention of occurrence of symptoms of the condition or disease in the subject, the prevention of recurrence of symptoms of the condition or disease in the subject, the delay of recurrence of symptoms of the condition or disease in the subject, the decrease in severity or frequency of outward symptoms of the condition or disease in the subject, slowing or eliminating the progression of the condition and the partial or total elimination of symptoms of the disease or condition in the subject.

Conditions which have been reported to be mediated by a decreased FXR activity include but are not limited to dyslipidemia (Sinal, C., et al., 2000 Cell 102:731-744; Zhang, Y., et al., 2006 Proc. Nat. Acad. Sci., U.S.A., 103:1006-1011); cardiovascular diseases such as atherosclerosis (Hanniman, E. A., et al., J. Lipid Res. 2005, 46:2595-2604); obesity (Chen, L., et al., 2006 Diabetes 55 suppl. 1:A200; Cariou, B., et al., 2006 J. Biol. Chem. 281:11039-11049; Rizzo, G., et al., 2006 Mol. Pharmacol. 70:1164-1173); diabetes mellitus (Duran-Sandoval, D., et al., 2004 Diabetes 53:890-898; Bilz, S., et al., 2006 Am. J. Physiol. Endocrinol. Metab. 290:E716-E722; Nozawa, H., 2005 Biochem. Biophys. Res. Commun. 336:754-761; Duran-Sandoval, D., et al., 2005 Biochimie 87:93-98; Claudel, T., et al., 2005 Arterioscler. Thromb. Vasc. Biol. 25:2020-2030; Duran-Sandoval, D., et al., 2005 J. Biol. Chem. 280:29971-29979; Savkur, R. S., et al., 2005 Biochem. Biophys. Res. Commun., 329:391-396; Cariou, B., et al., 2006 J. Biol. Chem. 281:11039-11049; Ma, K., et al., 2006 J. Clin. Invest. 116:1102-1109; Zhang, Y., et al., 2006 Proc. Nat. Acad. Sci. U.S.A. 103:1006-1011); metabolic syndrome (Chen, L., et al., 2006 Diabetes 55 suppl. 1:A200); disorders of the liver such as cholestatic liver disease (Liu, Y. et al., 2003 J. Clin. Invest. 112:1678-1687) and cholesterol gallstone disease (Moschetta, A., et al., 2004 Nat. Med. 10:1352-1358); organ fibrosis (Fiorucci, S., et al., 2004 Gastroenterology 127:1497-1512 and Fiorucci, S., et al., 2005 J. Pharmacol. Exp. Ther. 314:584-595) including liver fibrosis (Fiorucci, S., et al., 2004 Gastroenterology 127:1497-1512); inflammatory bowel disease (Inagaki, T., et al., 2006 Proc. Nat. Acad. Sci., U.S.A. 103:3920-3925); and liver regeneration (Huang, W., et al., 2006 Science 312:233-236).

Compounds of formula (I) are believed to be useful for the treatment of dyslipidemia in a subject, such as a mammal, particularly a human. The compounds of the present invention are currently believed to increase the flow of bile acid. Increased flow of bile acids improves the flux of bile acids from the liver to the intestine. FXR null mice demonstrate that FXR not only plays a role in bile acid homeostasis, but also plays a role in lipid homeostasis by virtue of the regulation of enzymes and transporters that are involved in lipid catabolism and excretion.

Compounds of formula (I) are also believed to be useful for lowering triglycerides in a subject, such as a mammal, particularly a human. As used herein “lowering triglycerides” means lowering triglycerides in a subject in need thereof below the initial level of triglycerides in that subject before administration of a compound of formula (I). For example, the compounds of formula (I) may lower triglycerides by decreasing fat absorption, decreasing hepatic triglyceride production or decreasing hepatic triglyceride secretion. The compounds of formula (I) may also lower serum and hepatic triglycerides.

By treating dyslipidemia, compounds of formula (I) are currently believed to be useful in the treatment of hypertriglyceridemia and hypercholesteronemia related cardiovascular disease such as atherosclerosis in a subject such as a mammal, particularly a human. Compounds of formula (I) are also believed to be useful for the treatment of non-alcoholic fatty liver disease and non-alcoholic steatohepatitis in a subject, such as a mammal, particularly a human (Chen, L., et al., 2006 Diabetes 55 suppl. 1:A200; Watanabe, M., et al., 2004 J. Clin. Invest., 113:1408-1418).

The compounds of formula (I) are useful for the treatment of obesity in a subject, such as a mammal, particularly a human.

Compounds of formula (I) are also useful for the treatment of diabetes mellitus in a subject, such as a mammal, particularly a human. For example, the compounds of formula (I) are useful for the treatment of type 2 diabetes. The effects of an FXR agonist, GW4064, on body weight, glucose tolerance, serum glucose, serum insulin, serum triglyceride, and liver triglyceride contents via oral administration have been observed in an high-fat diet induced insulin resistant, glucose intolerant, and obese mouse model (Chen, L., et al., 2006 Diabetes 55 suppl. 1:A200). Male 20 to 25 g C57BL mice (Charles River, Indianapolis, Ind.) were housed at 72° F. and 50% relative humidity with a 12 h light and dark cycle and fed with standard rodent chow (Purina 5001, Harlan Teklad, Indianapolis, Ind.) or a high-fat diet (TD93075, Harlan Teklad, Indianapolis, Ind.) for seven weeks. After two weeks, mice on high-fat diet were randomized to vehicle or treatment groups. There were no significant difference in body weight, body fat mass, serum glucose and insulin, and area under the curve (AUC) for glucose in glucose tolerance test (GTT) between the vehicle group and the treatment group. Starting from the fourth week, mice were given either vehicle or GW4064 (100 mg/kg) twice a day orally. Mice on the standard rodent chow were also given vehicle as a control. At the end of the third week of compound treatment, a GTT was performed and body composition was measured using the quantitative magnetic resonance (QMR) method. At the end of the study (fourth week of compound treatment), blood samples were taken from inferior vena cava and tissue samples were collected for further analysis. Blood glucose during GTT was measured using Bayer Glucometer Elite® XL. Serum chemistry levels were measured using the Instrumentation Laboratory Ilab600™ clinical chemistry analyzer (Instrumentation Laboratory, Boston, Mass.). Liver triglyceride contents were measured using the methanolic-KOH saponification method and a triglyceride assay kit (GPO-TRINDER, Sigma Diagnostics, St. Louis, Mo.). The results indicated that GW4064 reduced the high-fat diet induced body weight gain. It is believed that the result may have been due to a decrease in fat mass. GW4064 also appeared to improve glucose tolerance, decreased serum glucose, insulin and triglyceride, and reduced liver triglyceride content. In addition, Cariou and colleagues treated male ob/ob mice with GW4064 (30 mg/kg) intraperitoneally (2006 J. Biol. Chem. 281:11039-11049). GW4064 treatment did not alter body weight as well as food intake. Whereas GW4064 had no effect on fasting blood glucose in ob/ob mice, it decreased insulin concentration in the treated group. GW4064 treated ob/ob mice also showed an improved glucose tolerance and enhanced insulin sensitivity compared to controls. In another study, it was reported that GW4064 significantly improved hyperglycemia and hyperlipidemia in diabetic db/db mice (Zhang, Y., et al., 2006 Proc. Nat. Acad. Sci. U.S.A. 103:1006-1011). Oral GW4064 (30 mg/kg, bid) treatment decreased blood glucose, serum hydroxybutyrate, triglyceride, NEFA, and total cholesterol in db/db mice. It was also demonstrated that GW4064 treatment enhanced insulin signalling and glycogen storage in the liver of db/db mice. These data suggest that FXR agonists, including the compounds of the formula (I), may be used for the treatment of obesity, insulin resistance, glucose intolerance, diabetes mellitus, fatty liver disease and metabolic syndrome.

Compounds of formula (I) are also useful for the treatment of metabolic syndrome in a subject, such as a mammal, particularly a human. Metabolic syndrome is characterized by a group of metabolic risk factors in one person. They include abdominal obesity (excessive fat tissue in and around the abdomen), atherogenic dyslipidemia (high triglycerides, low high density lipoprotein (HDL) cholesterol and high low density lipoprotein (LDL) cholesterol), elevated blood pressure, insulin resistance or glucose intolerance, prothrombotic state and proinflammatory state. People with metabolic syndrome are at increased risk of coronary heart disease and atherosclerosis-related diseases (e.g., stroke and peripheral vascular disease) and type 2 diabetes mellitus. There are several clinical criteria for metabolic syndromes including ATP III, WHO, and AACE (American Association of Clinical Endocrinologists) (see tables, for review see Grundy, S. M., et al., 2004 Circulation 109:433-438). The present invention provides a method for the treatment of metabolic syndrome characterized by abdominal obesity, atherogenic dyslipidemia and insulin resistance with or without glucose interance, and may benefit other components of metabolic syndrome in a subject.

TABLE 1
ATP III Clinical Identification of the Metabolic Syndrome

| Risk Factor | Defining Level |
|---|---|
| Abdominal obesity, given as waist circumference*^{†} | |
| Men | >102 cm (>40 in) |
| Women | >88 cm (>35 in) |
| Triglycerides | ≧150 mg/dL |
| HDL cholesterol | |
| Men | <40 mg/dL |
| Women | <50 mg/dL |
| Blood pressure | ≧130/≧85 mm Hg |
| Fasting glucose | ≧110 mg/dL^{‡} |

*Overweight and obesity are associated with insulin resistance and the metabolic syndrome. However, the presence of abdominal obesity is more highly correlated with the metabolic risk factors than is an elevated BMI. Therefore, the simple measure of waist circumference is recommended to identify the body weight component of the metabolic syndrome.
^{†}Some male patients can develop multiple metabolic risk factors when the waist circumference is only marginally increased, eg, 94 to 102 cm (37 to 39 in). Such patients may have a strong genetic contribution to insulin resistance. They should benefit from changes in life habits, similarly to men with categorical increases in waist circumference.
^{‡}The American Diabetes Association has recently established a cutpoint of ≧100 mg/dL, above which persons have either prediabetes (impaired fasting glucose) or diabetes. This new cutpoint should be applicable for identifying the lower boundary to define an elevated glucose as one criterion for the metabolic syndrome.

TABLE 2
WHO Clinical Criteria for Metabolic Syndrome

Insulin resistance, identified by 1 of the following:
Type 2 diabetes
Impaired fasting glucose
Impaired glucose tolerance
or for those with normal fasting glucose levels (<110 mg/dL), glucose
uptake below the lowest quartile for background population under
investigation under hyperinsulinemic, euglycemic conditions
Plus any 2 of the following:
Antihypertensive medication and/or high blood pressure (≧140 mm Hg
systolic or ≧90 mm Hg diastolic)
Plasma triglycerides ≧150 mg/dL (≧1.7 mmol/L)
HDL cholesterol <35 mg/dL (<0.9 mmol/L) in men or <39 mg/dL
(1.0 mmol/L) in women
BMI >30 kg/m² and/or waist:hip ratio >0.9 in men, >0.85 in women
Urinary albumin excretion rate ≧20 μg/min or albumin:creatinine
ratio ≧30 mg/g

TABLE 3
AACE Clinical Criteria for Diagnosis
of the Insulin Resistance Syndrome*

| Risk Factor Components | Cutpoints for Abnormality |
|---|---|
| Overweight/obesity | BMI ≧25 kg/m² |
| Elevated triglycerides | ≧150 mg/dL (1.69 mmol/L) |
| Low HDL cholesterol | |
| Men | <40 mg/dL (1.04 mmol/L) |
| Women | <50 mg/dL (1.29 mmol/L) |
| Elevated blood pressure | ≧130/85 mm Hg |
| 2-Hour postglucose | >140 mg/dL |
| challenge | |
| Fasting glucose | Between 110 and 126 mg/dL |
| Other risk factors | Family history of type 2 diabetes, hypertension, |
| | or CVD |
| | Polycystic ovary syndrome |
| | Sedentary lifestyle |
| | Advancing age |
| | Ethnic groups having high risk for type 2 |
| | diabetes or CVD |

*Diagnosis depends on clinical judgment based on risk factors.

Compounds of formula (I) are believed to be useful for the treatment of cholestatic liver disease. For example, the compounds of formula (I) are believed to be useful in the treatment of primary biliary cirrhosis or primary sclerosing cholangitis. FXR therefore is a target for the treatment of a number of cholestatic liver diseases and non-alcoholic steatohepatitis. The compounds of formula (I) are also believed to be useful for the treatment of gall stones. For example, the compounds of formula (I) are believed to be useful in the treatment of cholesterol gallstone disease. The compounds of formula (I) are also believed to be useful for decreasing liver lipid accumulation.

Compounds of formula (I) are also believed to be useful for the treatment of organ fibrosis. Fibrotic disorders can be characterized as acute or chronic, but share the common characteristic of excessive collagen accumulation and an associated loss of function as normal tissues are replaced or displaced by fibrotic tissues. Acute forms of fibrosis include response to trauma, infections, surgery, burns, radiation and chemotherapy. Chronic forms of fibrosis may be due to viral infection, diabetes mellitus, obesity, fatty liver, hypertension, scleroderma and other chronic conditions that induce fibrosis.

Organs that are most commonly affected by fibrosis include liver, kidney, and lung. Organ fibrosis can cause the progressive loss of organ function. Retroperitoneal fibrosis (including idiopathic retroperitoneal fibrosis) may not originate from any major organ, but can involve and adversely affect the function of organs such as the kidneys.

Accordingly, as used herein, the term fibrosis refers to all recognized fibrotic disorders, including fibrosis due to pathological conditions or diseases, fibrosis due to physical trauma (‘traumatic fibrosis’), fibrosis due to radiation damage, and fibrosis due to exposure to chemotherapeutics. As used herein, the term “organ fibrosis” includes but is not limited to liver fibrosis, fibrosis of the kidneys, fibrosis of lung, and fibrosis of the intestine. “Traumatic fibrosis” includes but is not limited to fibrosis secondary to surgery (surgical scarring), accidental physical trauma, burns, and hypertrophic scarring.

In one embodiment, compounds of formula (I) are useful for the treatment of liver fibrosis in a subject, particularly a mammal, such as a human, in need of treatment thereof. As used herein, “liver fibrosis” includes liver fibrosis due to any cause, including but not limited to virally-induced liver fibrosis such as that due to hepatitis B or C virus; exposure to alcohol (alcoholic liver disease), certain pharmaceutical compounds including but not limited to methotrexate, some chemotherapeutic agents, and chronic ingestion of arsenicals or vitamin A in megadoses, oxidative stress, cancer radiation therapy or certain industrial chemicals including but not limited to carbon tetrachloride and dimethylnitrosamine; and diseases such as primary biliary cirrhosis, primary sclerosing colangitis, fatty liver, obesity, non-alcoholic steatohepatitis, cystic fibrosis, hemochromatosis, auto-immune hepatitis, and steatohepatitis. Current therapy in liver fibrosis is primarily directed at removing the causal agent, e.g., removing excess iron (e.g., in the case of hemochromatosis), decreasing viral load (e.g., in the case of chronic viral hepatitis), or eliminating or decreasing exposure to toxins (e.g., in the case of alcoholic liver disease). Anti-inflammatory drugs such as corticosteroids and colchicine are also known for use in treating inflammation that can lead to liver fibrosis. Other strategies for treating liver fibrosis are under development (see, e.g., Murphy, F., et al., 2002 Expert Opin. Invest. Drugs 11:1575-1585; Bataller, R. and Brenner, D. A., 2001 Sem. Liver Dis. 21:437-451). Thus in another embodiment, the present invention provides a method for the treatment of liver fibrosis in a subject which comprises administering a therapeutically effective amount of a compound of formula (I) in combination with another therapeutic agent useful for the treatment of symptoms associated with liver fibrosis. Examples of therapeutic agents useful for the treatment of symptoms associated with liver fibrosis include corticosteroids and cholchicine.

The response of the liver to hepatocellular damage, similar to wound healing in other tissues, includes inflammation and tissue remodeling, with associated changes in the quantity and quality of the extracellular matrix. Progressive accumulation of extracellular matrix proteins, including collagen types I and III, eventually distorts the architecture of the liver by forming fibrous scars, resulting in disrupted blood flow and an eventual deterioration in hepatic function. (Bissell, D. M. and Maher, J. J., “Hepatic Fibrosis and Cirrhosis.” Ed. Zakim, D. and Thomas, D. B., 4 ed. 2 vols. Philadelphia: Saunders, 2003. 395-416, Hanauske-Abel, H. M., “Fibrosis of the Liver: Representative Molecular Elements and Their Emerging Role As Anti-Fibrotic Targets.” Ed. Zakim, D., and Thomas, D. B., 4 ed. 2 vols. Philadelphia: Saunders, 2003. 347-394). Hepatic stellate cells (HSC) have been identified as important mediators of the fibrotic process in the liver, and are believed to be primarily responsible for the synthesis of excess extracellular matrix seen in liver disease. Liver injury can result in quiescent HSCs converting to activated myofibroblast-like cells that proliferate, migrate, recruit inflammatory cells, and synthesize collagens and other extracellular matrix proteins. (Bissell, D. M. and Maher, J. J., “Hepatic Fibrosis and Cirrhosis.” Ed. Zakim, D. and Thomas, D. B., 4 ed. 2 vols. Philadelphia: Saunders, 2003. 395-416, Hanauske-Abel, H. M., “Fibrosis of the Liver: Representative Molecular Elements and Their Emerging Role As Anti-Fibrotic Targets.” Ed. Zakim, D., and Thomas, D. B., 4 ed. 2 vols. Philadelphia: Saunders, 2003. 347-394). Various cytokines are reported to activate HSCs, including transforming growth factor β (TGFβ). Following liver injury, HSCs synthesize α-smooth muscle actin (α-SMA) as part of the migration response, consequently a marked accumulation of α-SMA can be seen at areas of active liver fibrogenesis. (Bissell, D. M. and Maher, J. J., “Hepatic Fibrosis and Cirrhosis.” Ed. Zakim, D. and Thomas, D. B., 4 ed. 2 vols. Philadelphia: Saunders, 2003. 395-416, Hanauske-Abel, H. M., “Fibrosis of the Liver: Representative Molecular Elements and Their Emerging Role As Anti-Fibrotic Targets.” Ed. Zakim, D., and Thomas, D. B., 4 ed. 2 vols. Philadelphia: Saunders, 2003. 347-394). Derangement of the normal epithelial/mesenchymal interaction, characterised by cholangiocyte damage/proliferation, can also lead to extracellular matrix-producing and progressive fibrogenesis. (Pinzani, M., et al., 2004 Digest. Liver Dis. 36:231-242.)

As is known in the art, liver fibrosis may be clinically classified into five stages of severity (S0 to S4), usually based on histological examination of a biopsy specimen. S0 indicates no fibrosis, whereas S4 indicates cirrhosis. While various criteria for staging the severity of liver fibrosis exist, in general early stages of fibrosis are identified by discrete, localized areas of scarring in one portal (zone) of the liver, whereas later stages of fibrosis are identified by bridging fibrosis (scarring that crosses zones of the liver).

Compounds of formula (I) are also useful for the treatment of inflammatory bowel disease in a subject, such as a mammal, particularly a human. Inflammatory bowel disease (IBD) is defined as a group of idiopathic relapsing inflammatory disorders of the bowel—the large or small intestine. The pathogenesis of IBD remains obscure and may involve genetic, environmental and immunological factors. (Drossman, D. A. 1999 Aliment Pharmacol. Ther. 13(s2):3-14; Danese, S., et al., 2004 Autoimmunity Reviews 3: 394-400; Stokkers, P. C. F. and Hommes, D. W. 2004 Cytokine 28:167-173.) The most common types of inflammatory bowel disease are ulcerative colitis and Crohn disease.

Compounds of formula (I) are also believed to be useful for enhancing liver regeneration in a subject, such as a mammal, particularly a human. For example, the compounds of formula (I) are believed to be useful for enhancing liver regeneration for liver transplantation.

The present invention provides a method for the treatment of a condition mediated by decreased FXR activity, particularly a condition in which a FXR agonist may be useful, in a subject, such as a mammal, particularly a human, in need thereof. The present invention also provides the use of a compound of formula (I) for the preparation of a medicament for the treatment of a condition mediated by decreased FXR activity, particularly a condition in which a FXR agonist may be useful, in a subject, such as a mammal, particularly a human in need thereof.

The present invention also provides a method for lowering triglycerides in a subject, such as a mammal, particularly a human, in need thereof. The present invention also provides the use of a compound of formula (I) for the preparation of a medicament for lowering triglycerides in a subject. In one embodiment, the compound of formula (I) is 3-[5-({[3-(2,6-dichlorophenyl)-5-(1-methylethyl)-4-isoxazolyl]methyl}oxy)-1-benzothien-2-yl]benzoic acid or a pharmaceutically acceptable salt thereof. In another embodiment, the compound of formula (I) is 3-{[5-({[3-(2,6-dichlorophenyl)-5-(1-methylethyl)-4-isoxazolyl]methyl}oxy)-1H-indol-1-yl]methyl}benzoic acid or a pharmaceutically acceptable salt thereof.

The present invention provides a method for the treatment of obesity in a subject, such as a mammal, particularly a human, in need thereof. The present invention also provides the use of a compound of formula (I) for the preparation of a medicament for the treatment of obesity in a subject. In one embodiment, the compound of formula (I) is 3-[5-({[3-(2,6-dichlorophenyl)-5-(1-methylethyl)-4-isoxazolyl]methyl}oxy)-1-benzothien-2-yl]benzoic acid or a pharmaceutically acceptable salt thereof. In another embodiment, the compound of formula (I) is 3-{[5-({[3-(2,6-dichlorophenyl)-5-(1-methylethyl)-4-isoxazolyl]methyl}oxy)-1H-indol-1-yl]methyl}benzoic acid or a pharmaceutically acceptable salt thereof.

The present invention provides a method for the treatment of diabetes mellitus in a subject, such as a mammal, particularly a human, in need thereof. The present invention also provides the use of a compound of formula (I) for the preparation of a medicament for the treatment of diabetes mellitus in a subject. In one embodiment, the compound of formula (I) is 3-[5-({[3-(2,6-dichlorophenyl)-5-(1-methylethyl)-4-isoxazolyl]methyl}oxy)-1-benzothien-2-yl]benzoic acid or a pharmaceutically acceptable salt thereof. In another embodiment, the compound of formula (I) is 3-{[5-({[3-(2,6-dichlorophenyl)-5-(1-methylethyl)-4-isoxazolyl]methyl}oxy)-1H-indol-1-yl]methyl}benzoic acid or a pharmaceutically acceptable salt thereof.

The present invention provides a method for the treatment of metabolic syndrome in a subject, such as a mammal, particularly a human, in need thereof. The present invention also provides the use of a compound of formula (I) for the preparation of a medicament for the treatment of metabolic syndrome in a subject. In one embodiment, the compound of formula (I) is 3-[5-({[3-(2,6-dichlorophenyl)-5-(1-methylethyl)-4-isoxazolyl]methyl}oxy)-1-benzothien-2-yl]benzoic acid or a pharmaceutically acceptable salt thereof. In another embodiment, the compound of formula (I) is 3-{[5-({[3-(2,6-dichlorophenyl)-5-(1-methylethyl)-4-isoxazolyl]methyl}oxy)-1H-indol-1-yl]methyl}benzoic acid or a pharmaceutically acceptable salt thereof.

The present invention provides a method for the treatment of cholestatic liver disease in a subject, such as a mammal, particularly a human, in need thereof. The present invention also provides the use of a compound of formula (I) for the preparation of a medicament for the treatment of cholestatic liver disease in a subject. In one embodiment, the compound of formula (I) is 3-[5-({[3-(2,6-dichlorophenyl)-5-(1-methylethyl)-4-isoxazolyl]methyl}oxy)-1-benzothien-2-yl]benzoic acid or a pharmaceutically acceptable salt thereof. In another embodiment, the compound of formula (I) is 3-{[5-({[3-(2,6-dichlorophenyl)-5-(1-methylethyl)-4-isoxazolyl]methyl}oxy)-1H-indol-1-yl]methyl}benzoic acid or a pharmaceutically acceptable salt thereof.

The present invention provides a method for the treatment of organ fibrosis in a subject, such as a mammal, particularly a human, in need thereof. The present invention also provides the use of a compound of formula (I) for the preparation of a medicament for the treatment of organ fibrosis in a subject. In one embodiment, the compound of formula (I) is 3-[5-({[3-(2,6-dichlorophenyl)-5-(1-methylethyl)-4-isoxazolyl]methyl}oxy)-1-benzothien-2-yl]benzoic acid or a pharmaceutically acceptable salt thereof. In another embodiment, the compound of formula (I) is 3-{[5-({[3-(2,6-dichlorophenyl)-5-(1-methylethyl)-4-isoxazolyl]methyl}oxy)-1H-indol-1-yl]methyl}benzoic acid or a pharmaceutically acceptable salt thereof.

The present invention provides a method for the treatment of liver fibrosis in a subject, such as a mammal, particularly a human, in need thereof. The present invention also provides the use of a compound of formula (I) for the preparation of a medicament for the treatment of liver fibrosis in a subject. In one embodiment, the compound of formula (I) is 3-[5-({[3-(2,6-dichlorophenyl)-5-(1-methylethyl)-4-isoxazolyl]methyl}oxy)-1-benzothien-2-yl]benzoic acid or a pharmaceutically acceptable salt thereof. In another embodiment, the compound of formula (I) is 3-{[5-({[3-(2,6-dichlorophenyl)-5-(1-methylethyl)-4-isoxazolyl]methyl}oxy)-1H-indol-1-yl]methyl}benzoic acid or a pharmaceutically acceptable salt thereof.

All of the methods of the present invention comprise the step of administering a therapeutically effective amount of the compound of formula (I). As used herein, the term “therapeutically effective amount” refers to an amount of a compound of formula (I) which is sufficient to achieve the stated effect in the subject to which it is administered. Accordingly, a therapeutically effective amount of a compound of formula (I) used in the method for the treatment of a condition mediated by decreased FXR activity in a human will be an amount sufficient for the treatment of the condition mediated by decreased FXR activity in a human. A therapeutically effective amount of a compound of formula (I) for use in the method for the treatment of diabetes mellitus in a human will be an amount sufficient for the treatment of diabetes mellitus in a human. A therapeutically effective amount of a compound of formula (I) for use in the method for the treatment of metabolic syndrome in a human will be an amount sufficient for the treatment of metabolic syndrome in a human. A therapeutically effective amount of a compound of formula (I) for use in the method for the treatment of organ (e.g., liver) fibrosis in a human will be an amount sufficient for the treatment of organ fibrosis in a human.

The amount of a compound of formula (I) which is required to achieve the desired therapeutic or biological effect will depend on a number of factors such as the use for which it is intended, the means of administration, the recipient and the type and severity of the condition or disease being treated, and will be ultimately at the discretion of the attendant physician or veterinarian. In general, a typical daily dose for the treatment of a disease or condition mediated by decreased FXR activity in a human, for instance, may be expected to lie in the range of from about 0.01 mg/kg to about 100 mg/kg for a 70 kg human. This dose may be administered as a single unit dose or as several separate unit doses or as a continuous infusion. Similar dosages would be applicable for the treatment of other diseases, conditions and therapies including diabetes mellitus and obesity in humans.

While it is possible that, for use in therapy, a therapeutically effective amount of a compound of formula (I) may be administered as the raw chemical, it is typically presented as the active ingredient of a pharmaceutical composition or formulation. Accordingly, the invention further provides a pharmaceutical composition comprising a compound of the formula (I). The pharmaceutical composition may further comprise one or more pharmaceutically acceptable carriers or diluents. The carrier(s) and/or diluent(s) must be acceptable in the sense of being compatible with the other ingredients of the formulation and not deleterious to the recipient thereof. In one particular embodiment, the compound is in crystalline form. In accordance with another aspect of the invention there is also provided a process for the preparation of a pharmaceutical formulation including admixing a compound of the formula (I) with one or more pharmaceutically acceptable carriers and/or diluents.

Pharmaceutical formulations may be presented in unit dose form containing a predetermined amount of active ingredient per unit dose. Such a unit may contain a therapeutically effective dose of the compound of formula (I) or a fraction of a therapeutically effective dose such that multiple unit dosage forms might be administered at a given time to achieve the desired therapeutically effective dose. Preferred unit dosage formulations are those containing a daily dose or sub-dose, as herein above recited, or an appropriate fraction thereof, of an active ingredient.

Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethyl-cellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of active ingredient. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle.

Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

A compound of formula (I) can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

A compound of formula (I) may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in 1986 Pharmaceutical Research 3:318.

Pharmaceutical compositions adapted for topical administration may be formulated as ointments, creams, suspensions, lotions, powders, solutions, pastes, gels, sprays, aerosols or oils.

For treatments of the eye or other external tissues, for example mouth and skin, the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical compositions adapted for topical administrations to the eye include eye drops wherein the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or as enemas.

Pharmaceutical compositions adapted for nasal administration wherein the carrier is a solid include a coarse powder having a particle size for example in the range of about 20 microns to about 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical compositions adapted for administration by inhalation include fine particle dusts or mists, which may be generated by means of various types of metered, dose pressurised aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the compositions may include other agents conventional in the art having regard to the type of formulation in question, for example those suitable for oral administration may include flavouring agents.

In the above-described methods of treatment and uses, a compound of formula (I) may be employed alone, in combination with one or more other compounds of formula (I) or in combination with other therapeutic agents. Thus, the present invention also encompasses pharmaceutical compositions further comprising one or more therapeutic agents. In one embodiment, the pharmaceutical compositions further comprise one or more lipid-altering agents. Examples of lipid-altering agents include but are not limited to liver X receptor (LXR) agonists described in PCT Publication No. WO02/24632 to GlaxoSmithKline. Examples of other therapeutic agents include, but are not limited to, 3-Hydroxy-3-Methyl-Glutaryl-CoA reductase inhibitors such as statins (atorvastatin, fluvastatin, pravastatin, lovastatin, cerivastatin, and nisvastatin); squalene epoxidase inhibitors, squalene synthetase inhibitors, bile acid transport inhibitors (BATi), human peroxisome proliferator activated receptor (PPAR) gamma agonists such as rosiglitazone, troglitazone, and pioglitazone and thiazolidinediones; PPAR α agonists such as clofibrate, fenofibrate and gemfibronzil; PPAR dual α/γ agonists; cyclooxygenase-2 (COX-2) inhibitors such as rofecoxib and celecoxib; thrombin inhibitors; acyl-coenzyme A; cholesterol acyltransferase (ACAT) inhibitors including selective ACAT inhibitors; microsomal triglyceride transfer protein (MTP) inhibitors; probucol, niacin; cholesterol absorption inhibitors; bile acid sequestrants; LDL receptor inducers; platelet aggregation inhibitors such as glycoprotein IIb/IIIa fibrinogen receptor antagonists and aspirin; vitamin B6 and pharmaceutically acceptable salts thereof; vitamin B12; folic acid or a pharmaceutically acceptable salt or ester thereof; antioxidant vitamins such as C and E and beta carotene; beta blockers; angiotensin II antagonists such as losartan; antiotensin converting enzyme inhibitors such as enalapril and captopril; calcium channel blockers such as nifedipine and diltiazam; endothelian antagonists; agents other than LXR ligands that enhance ATP-Binding Cassette Transporter-A1 gene expression; and bisphosphonate compounds such as alendronate sodium.

The methods and uses employing these combinations may comprise the administration of the compound of formula (I) and another therapeutic agent either sequentially in any order or simultaneously in separate or combined pharmaceutical compositions. When combined in the same composition it will be appreciated that the compounds must be stable and compatible with each other and the other components of the composition and may be formulated for administration. When formulated separately they may be provided in any convenient formulation, in such a manner as are known for such compounds in the art.

When a compound of formula (I) is used in combination with another therapeutic agent, the dose of each compound may differ from that when the compound is used alone. Appropriate doses will be readily appreciated by those skilled in the art. The appropriate dose of the compound(s) of formula (I) and the other therapeutically active agent(s) and the relative timings of administration will be selected in order to achieve the desired combined therapeutic effect, and are within the expertise and discretion of the attendant clinician.

Compounds of the invention can be made according to any suitable method of organic chemistry. As will be apparent to those skilled in the art and as depicted in the schemes which follow, the order of the steps in each reaction is not critical to the practice of the processes of the present invention. The reaction steps depicted in each scheme may be carried out in any suitable order based upon the knowledge of those skilled in the art.

Further, it will be apparent to those skilled in the art that certain reaction steps may be most efficiently performed by installing protecting groups prior to the reaction, which are removed subsequently. The choice of protecting groups as well as general techniques for their installation and removal are within the skill of those in the art. It will be appreciated by those skilled in the art that certain ring systems represented in the generic ring structure of the A ring will require the use of a protective group to minimize the possibility of undesired side reactions from occurring. The protective group may be easily installed by methods contained in the literature and likewise may be removed once they are no longer needed. Examples of ring systems that would require a protective group would include benzimidazole, indazole and indole.

According to one method, a compound of formula (I) may be prepared using the process depicted in Scheme 1, below.

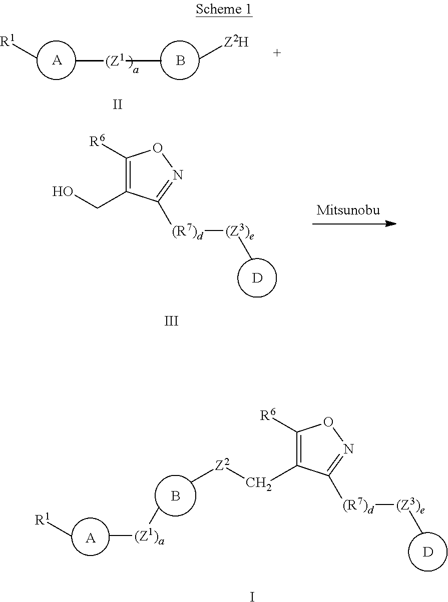

wherein:
R¹ is —CO₂alkyl, —CH₂CH₂CO₂alkyl, —NHC(O)CH₃, or —OCF₃;
Z² is —O—, —NH— or —S—; and
all other variables are as defined above for formula (I).

In general, the process for preparing a compound of formula (I) as depicted in Scheme 1 comprises the steps of:

a) reacting a compound of formula (II) with a compound of formula (III) to prepare a compound of formula (I);
b) optionally converting the compound of formula (I) into a pharmaceutically acceptable salt thereof; and
c) optionally converting the compound of formula (I) or a pharmaceutically acceptable salt thereof into a different compound of formula (I) or a pharmaceutically acceptable salt thereof.

A compound of formula (I), prepared by any suitable process, may be converted into a pharmaceutically acceptable salt thereof or may be converted to a different compound of formula (I) or a pharmaceutically acceptable salt thereof using techniques described herein below and those conventional in the art.

More particularly, the compound of formula (I) may be prepared by reacting the compound of formula (II) with a compound of formula (III) in the presence of triphenylphosphine and a dialkylazodicarbonate like diisopropylazodicarbonate at elevated temperature. It will be apparent to those that are skilled in the art that where Z² is N it may be required to first convert the compound of formula (II) to the trifluoroacetamide using known techniques prior to the Mitsunobu reaction. The trifluoroacetamide can be cleaved during the saponification of the ester to form a compound of formula (I).

The compound of formula (III) may be prepared by reducing a compound of formula (IV).

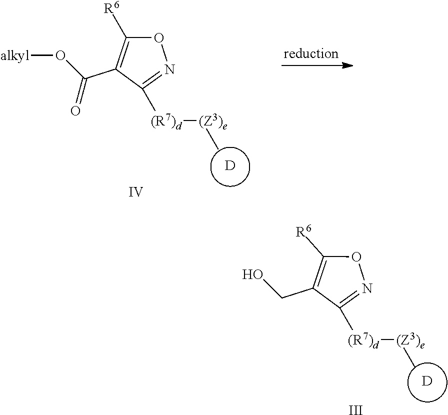

wherein all variables are as defined above.

A compound of formula (IV) may be treated with a reducing agent, such as diisobutylaluminum hydride, in a suitable solvent such as tetrahydrofuran.

In another embodiment, the compound of formula (IV) may be saponified to the corresponding carboxylic acid prior to reducing with a suitable reducing agent, such as borane, to prepare a compound of formula (III). In addition, the carboxylic acid may also converted to a mixed anhydride before reducing with a reducing agent such as sodium borohydride to prepare a compound of formula (III).

Compounds of formula (IV) may be prepared by multiple routes. In one embodiment, the compound of formula (IV) may be prepared by a process comprising the steps of:

1) chlorinating a compound of formula (V); and
2) cyclizing with a β-ketoester of formula (VI).

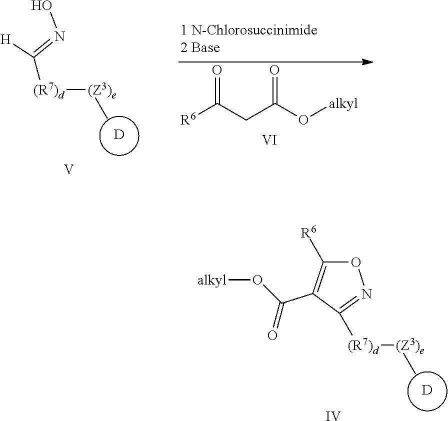

wherein all variables are as defined above.

The process may be carried out according to the method described by Doyle, F. P., et al., 1963 J. Chem. Soc. 5838-5845. Esters of formula (VI) are commercially available or can be prepared using conventional techniques.

The compound of formula (V) may be prepared by condensing a compound of formula (VII) with hydroxylamine.

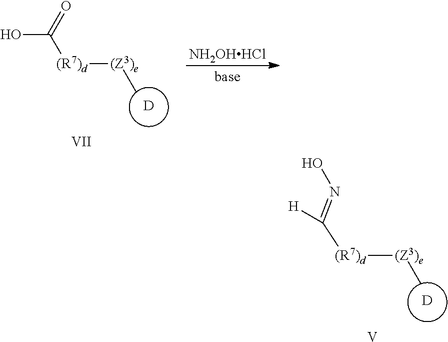

wherein all variables are as defined above.

Conditions suitable for this condensation reaction are conventional in the art.

In another embodiment, a compound of formula (IV) may be prepared by a process comprising the steps of: a) reacting a compound of formula (IX) with tin chloride in the presence of a compound of formula (VIII) to prepare a compound of formula (X) and b) reacting the compound of formula (X) with hydroxylamine to yield a compound of formula (IV). See, Singh, B. and Lesher, G. Y. 1978 Synthesis 829-830.

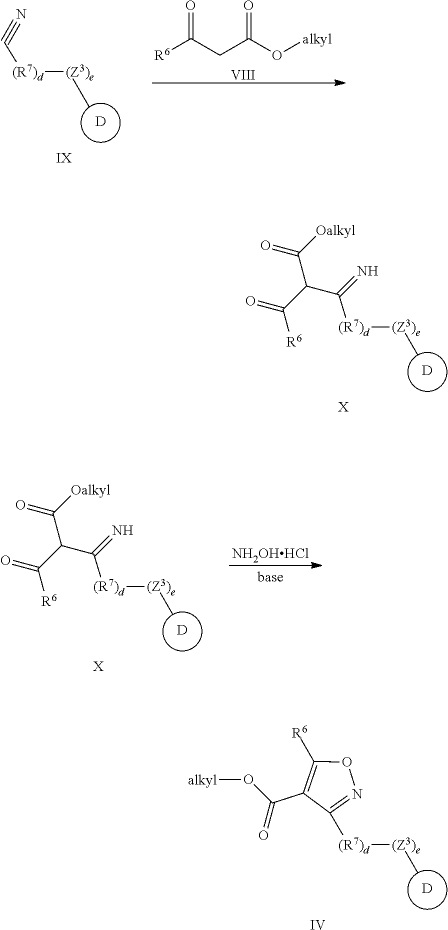

wherein all variables are as defined above.

The compound of formula (IX) may be obtained commercially or prepared by procedures in the literature. See, Guo, H. and Zhang, Y. 2000 Syn. Commun. 30:1879-1885. The compound of formula (IV) may then be reduced with a suitable reducing agent, such as diisobutylaluminum hydride, as described above, to prepare a compound of formula (III).

A compound of formula (II-a) may be prepared by reacting a compound of formula (XI) with a solution of boron tribromide in a solvent like dichloromethane. Optionally, this may be followed by submission of the material to esterification conditions, like heating in the appropriate alcoholic solvent with an acid catalyst, like sulfuric acid.

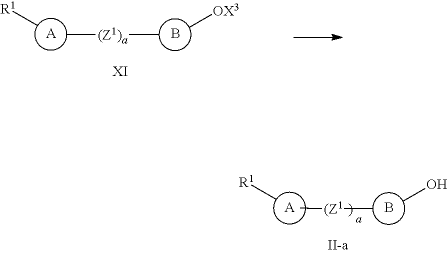

wherein:
X³ is methyl or benzyl;
R¹ is —CO₂alkyl, —CH₂CH₂CO₂alkyl, —NHC(O)CH₃, or —OCF₃; and
all other variables are as defined above.

A compound of formula (XI) may be prepared by reacting a compound of formula (XIII) with a boronic acid or ester of formula (XII) under standard Suzuki reaction conditions. Compounds of formulas (XIII) and (XII) may be purchased from commercial sources or may be prepared by those skilled in the art.

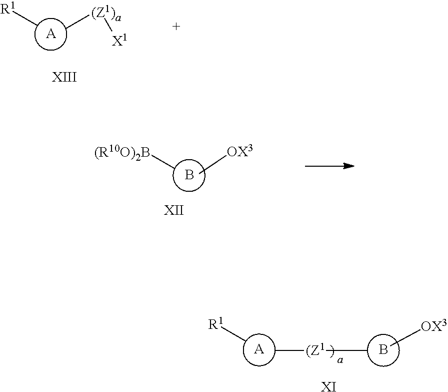

wherein:
X¹ is chloro, bromo, iodo or triflate;
Ring A is phenyl or a 5-6 membered heteroaryl comprising 1, 2 or 3 heteroatoms selected from N, O and S, wherein said phenyl or heteroaryl is substituted with R¹ and further optionally substituted with one or two independently selected C₁₋₆alkyl;

### Ring B is B-i, B-ii, B-iii, B-iv, B-v, B-vi, B-vii, B-viii, B-ix, B-xiv, or B-xv;

a is 0;
X³ is methyl or benzyl;
R¹⁰ is H or alkyl;
R¹ is —CO₂alkyl, —CH₂CH₂CO₂alkyl, —NHC(O)CH₃, or —OCF₃; and
all other variables are as defined above.

As another example of processes for preparing compounds of formula (XI), a compound of formula (XI) may be prepared by reacting a compound of formula (XIV) with a boronic acid or ester of formula (XV) under standard Suzuki reaction conditions. Compounds of formulas (XIV) and (XV) may be purchased from commercial sources or may be prepared by those skilled in the art.

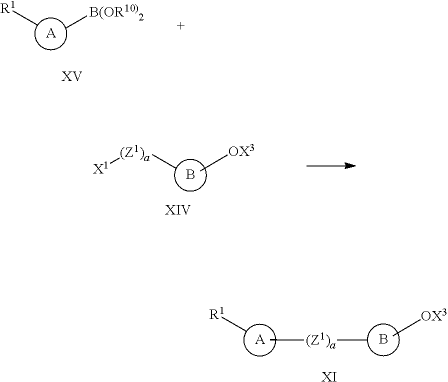

wherein:
a is 0;
Ring A is phenyl or a 5-6 membered heteroaryl comprising 1, 2 or 3 heteroatoms selected from N, O and S, wherein said phenyl or heteroaryl is substituted with R¹ and further optionally substituted with one or two independently selected C₁₋₆alkyl;

### Ring B is B-i, B-ii, B-iv, B-v, B-vi, B-vii, B-viii, B-ix, B-xiv, or B-xv;

X¹ is chloro, bromo, iodo or triflate;
X³ is methyl or benzyl;
R¹⁰ is H or alkyl;
R¹ is —CO₂alkyl, —CH₂CH₂CO₂alkyl, —NHC(O)CH₃, or —OCF₃; and
   all other variables are as defined above.

As an example of a process for preparing compounds of formula (XII), a compound of formula (XII-b) can be made by deprotonating a compound of formula (XVI) with a base like n-butyl lithium or lithium diisopropylamide and reacting the resulting anion with a trialkyl borate like triisopropyl borate. A compound of formula (XVI) can be synthesized by one skilled in the art according to literature procedures.

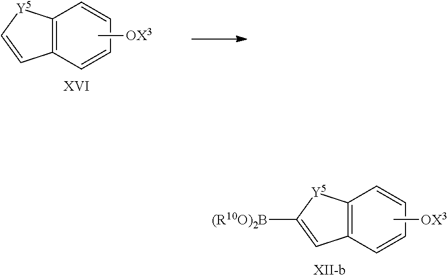

wherein:
Y⁵ is —S— or NCO₂tBu=tertButoxycarbonyl;
X³ is methyl or benzyl; and
all other variables are as defined above.

As another example of processes for preparing compounds of formula (XI), a compound of formula (XI-a) may be prepared by condensing a compound of formula (XVII) with a compound of formula (XVIII).

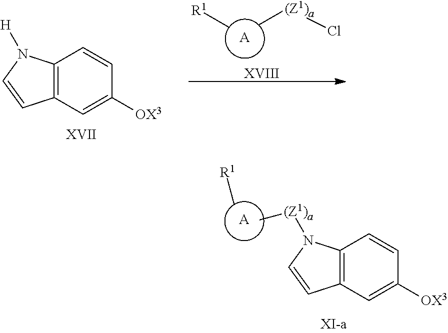

wherein:
X³ is methyl or benzyl;
R¹ is —CO₂alkyl, —CH₂CH₂CO₂alkyl, —NHC(O)CH₃, or —OCF₃;
a is 1;

### Z¹ is —CH₂—, —CO— or —SO₂—;

and all other variables are as defined above.

As another example of processes for preparing compounds of formula (XI), a compound of formula (XI-b) can be synthesized by reacting a compound of formula (XIX) with a phenyl iodide in the presence of copper (I) iodide in a solvent like N,N-dimethylformamide at elevated temperatures.

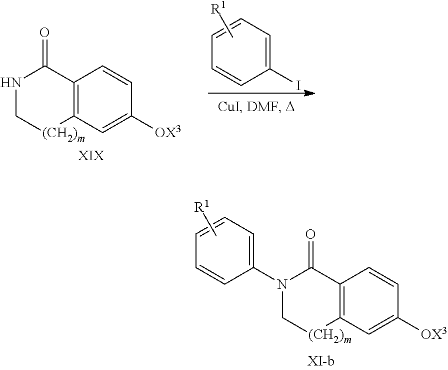

wherein:
R¹ is —CO₂alkyl, —CH₂CH₂CO₂alkyl or —OCF₃
CuI is copper (I) iodide, DMF is N,N-dimethylformamide;
m is 0 or 1; and
all other variables are as defined above.

A compound of formula (XIX) may be made by heating a compound of formula (XX) in the presence of polyphosphoric acid.

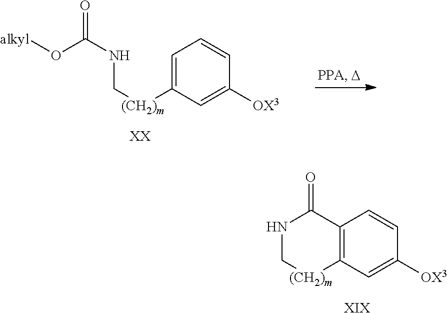

wherein:
PPA is polyphosphoric acid;
m is 0 or 1; and
all other variables are as defined above.

A compound of formula (XX) may be synthesized by condensing an amine of formula (XXI) with an alkylchloroformate, like isobutylchloroformate, in the presence of a base, like triethylamine or diisopropylethylamine in a solvent, like dichloromethane.

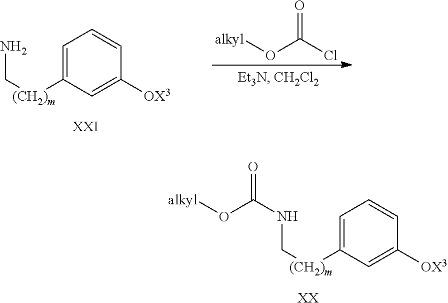

wherein:
m is 0 or 1;
Et₃N is triethylamine; and
all other variables are as defined above.

As another example of processes for preparing compounds of formula (XI), a compound of formula (XI-c) may be synthesized by condensing an aniline of formula (XXII) with a benzylbromide of formula (XXIII) in the presence of a base, like triethylamine or diisopropylethylamine, in a solvent, like toluene, at an elevated temperature. The resulting intermediate is then stirred with an acid catalyst, like trifluoroacetic acid, or p-toluenesulphonic acid in a solvent, like toluene or acetonitrile, at ambient or elevated temperature. A compound of formula (XXIII) may be made by those skilled in the art by literature procedures. A compound of formula (XXII) may be purchased from commercial sources or may be made by one skilled in the art.

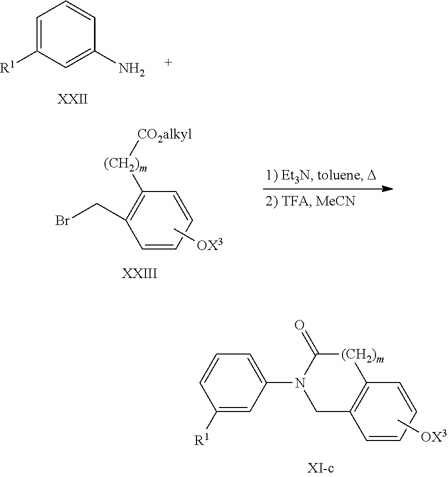

wherein:
TFA is trifluoroacetic acid, MeCN is acetonitrile;
R¹ is —CO₂alkyl, —CH₂CH₂CO₂alkyl, or —OCF₃;
m is 0 or 1; and
all other variables are as defined above.

A compound of formula (XXIII-a) may be made by reacting a compound of formula (XXIV) with thionyl bromide in a solution of toluene and an alcohol. A compound of formula (XXIV) an may be made by those skilled in the art by literature procedures.

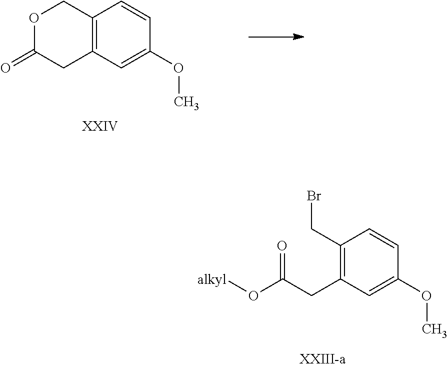

Other compounds of formula (XXIII) may be made by those skilled in the art by literature procedures.

As another example of processes for preparing compounds of formula (XI), a compound of formula (XI-d) may be synthesized by reacting the anion of an indole of formula (XXV) with a compound of formula (XXVI) employing a base, like sodium hydride, and a solvent, like N,N-dimethylformamide. A compound of formula (XXV) may be purchased from commercial sources. A compound of formula (XXVI) can be purchased from commercial sources or be synthesized by those skilled in the art.

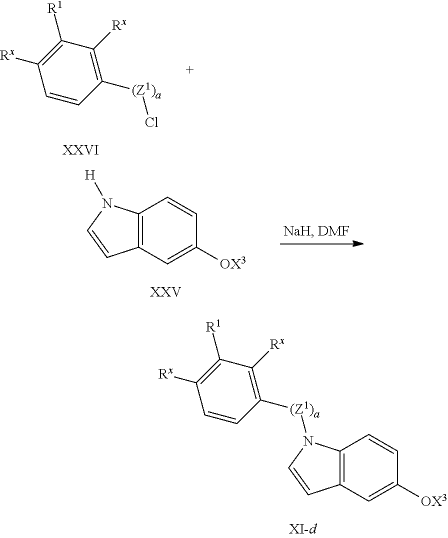

wherein:
NaH is sodium hydride;
R¹ is —CO₂alkyl or —OCF₃;
each R^{x} is the same or different and is independently selected from hydrogen and methyl and at least one R^{x} is hydrogen;

### Z¹ is —SO₂— or CH₂;

a is 1;
X³ is benzyl or methyl; and
all other variables are as defined above.

As another example of processes for preparing compounds of formula (XI), a compound of formula (XI-e) may be made by the condensing a compound of formula (XXVII) with formic acid at elevated temperature.

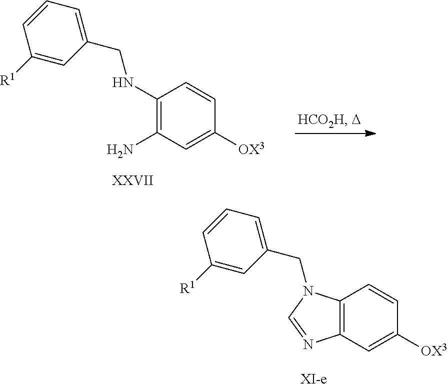

wherein: R¹ is —CO₂alkyl, —NHC(O)CH₃, or —OCF₃; and
all other variables are as defined above.

A compound of formula (XXVII) may be made by the reduction of formula (XXIX) with tin (II) chloride dehydrate in an appropriate alcohol at elevated temperatures.

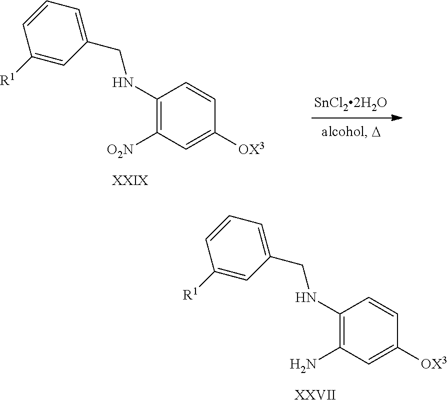

wherein: R¹ is —CO₂alkyl or —NHC(O)CH₃; and
all other variables are as defined above.

A compound of formula (XXIX) may be prepared by condensing a compound of formula (XXX) with a benzylbromide of formula (XXXI) in the presence of a base, like potassium carbonate, in a solvent, like N,N-dimethylformamide, at elevated temperature. Compounds for formula (XXX) and (XXXI) may be purchased from commercial sources or synthesized by those skilled in the art.

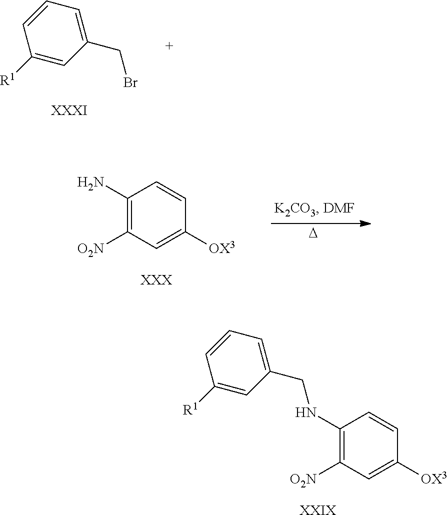

wherein: R¹ is —CO₂alkyl, —CH₂CH₂CO₂alkyl, —NHC(O)CH₃, or —OCF₃; and
all other variables are as defined above.

As another example of processes for preparing compounds of formula (XI), a compound of formula (XI-f) may be synthesized by reacting a benzylbromide of formula (XXXI) and an indole of formula (XXXII) in the presence of zinc (II) triflate, diisopropylethylamine and tetrabutylammonium iodide.

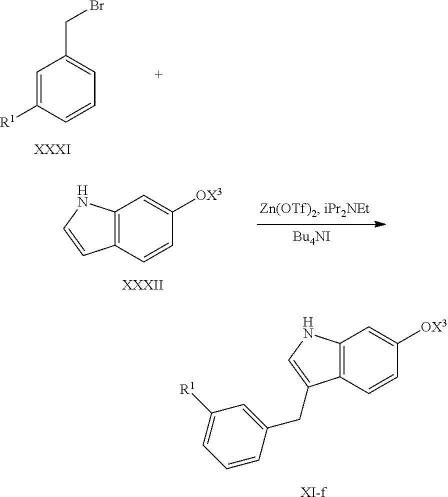

wherein:
R¹ is —CO₂alkyl, —CH₂CH₂CO₂alkyl, —NHC(O)CH₃, or —OCF₃;
X³ is benzyl or methyl;
OTf is trifluoromethane sulfonate;
Bu₄NI is tetrabutylammonium iodide;
(iPr)₂NEt is diisopropylethylamine; and
all other variables are as defined above.

As another example of processes for preparing compounds of formula (XI), a compound of formula (XI-g) may be synthesized by reacting an aryl bromide of formula (XXXIII) with a boronic acid or ester of formula (XXXIV) under standard Suzuki reaction conditions. For example, the reaction may be carried out in the presence of a suitable palladium complex such as tetrakis(triphenylphosphine)-palladium(0) and a base such as sodium carbonate in a mixture of water and ethereal solvent such as 1,2-dimethoxyethane, at an elevated temperature. A compound of formula (XXXIII) may be purchased from commercial sources or may be synthesized by those skilled in the art.

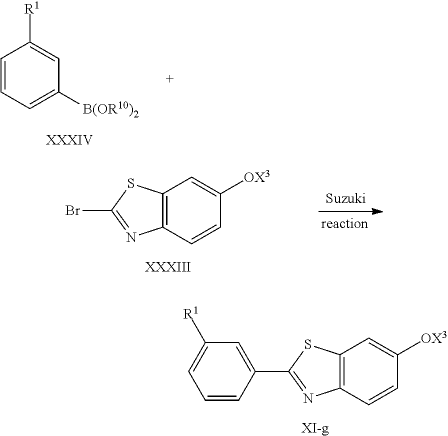

wherein:
R¹ is —CO₂alkyl, —CH₂CH₂CO₂alkyl, —NHC(O)CH₃, or —OCF₃;
X³ is benzyl or methyl; and
all other variables are as defined above.

A compound of formula (XXXVII) may be made by reacting a compound of formula (XXXV) with t-butylnitrite and copper (II) bromide in a solvent, like acetonitrile. A compound of formula (XXXV) may be purchased from commercial sources or may be synthesized by those skilled in the art.

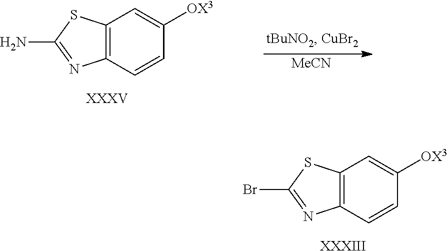

wherein: X³ is benzyl or methyl;
and other all variables are as defined above.

As another example of processes for preparing compounds of formula (XI), a compound of formula (XI-h) may be synthesized by reacting an aniline of formula (XXXVI) with a triflate or aryl halide of formula (XXXVII) in the presence of a suitable palladium catalyst and a base. For example, the reaction may be carried out in the presence cesium carbonate and a suitable palladium complex such as the one formed by the complexation of tris(diphenylideneacetone)dipalladium(0) and rac-2,2′-bis(diphenylphosphino)-1,1′-binaphthyl in a solvent like toluene at an elevated temperature. A compound of formula (XXXVI) may be purchased from commercial sources or may be synthesized by those skilled in the art.

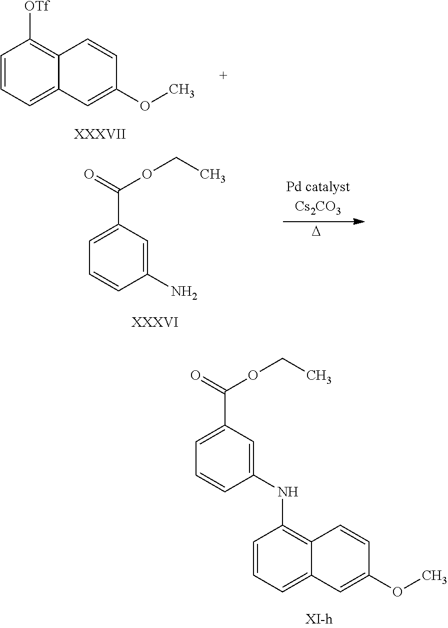

A compound of formula (XXXVII) may be synthesized by reacting a napthol of formula (XXXVIII) with trifluoromethane sulfonic anhydride in a solution of pyridine in dichloromethane.

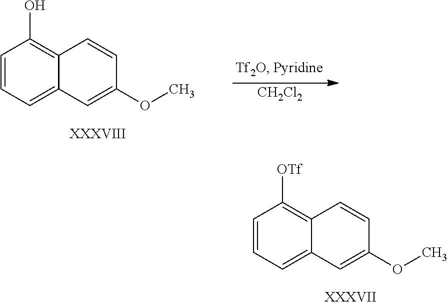

wherein:
Tf₂O is trifluoromethane sulfonic anhydride;
OTf is triflate;
and all other variables are as defined above.

According to another embodiment, a compound of formula (I) may be prepared using the process depicted in Scheme 2, below.

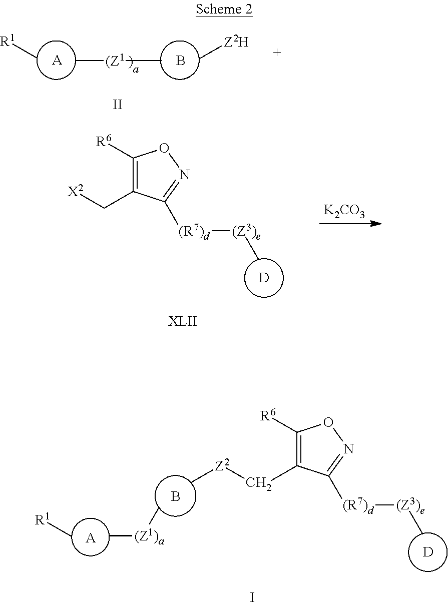

wherein:
a is 0;
Z² is —O—, —NH— or —S—;
X² is chloro, iodo, bromo, triflate, tosylate, nosylate, besylate or mesylate, (preferably chloro);
R¹ is —CO₂alkyl, —CH₂CH₂CO₂alkyl, —NHC(O)CH₃, or —OCF₃; and
all other variables are as defined above for formula (I).

In general, the process for preparing a compound of formula (I) as depicted in Scheme 2 comprises the steps of:

a) reacting a compound of formula (II) with a compound of formula (XLII) to prepare a compound of formula (I);
b) optionally converting the compound of formula (I) into a pharmaceutically acceptable salt; and
c) optionally converting the compound of formula (I) or a pharmaceutically acceptable salt thereof into a different compound of formula (I) or a pharmaceutically acceptable salt thereof.

A compound of formula (I), prepared by any suitable process, may be converted into a pharmaceutically acceptable salt thereof or may be converted to a different compound of formula (I) or a pharmaceutically acceptable salt thereof using techniques described herein below and those conventional in the art.

More particularly, the compound of formula (I) may be prepared by reacting the compound of formula (II) with a compound of formula (XLII) in the presence of a suitable base such as cesium carbonate or potassium carbonate, in a polar aprotic solvent, such as N,N-dimethylformamide, at ambient or elevated temperature.

The compound of formula (XLII) may be prepared by reacting a compound of formula (III) with the appropriate reagent to prepare a compound having the desired leaving group (X²).

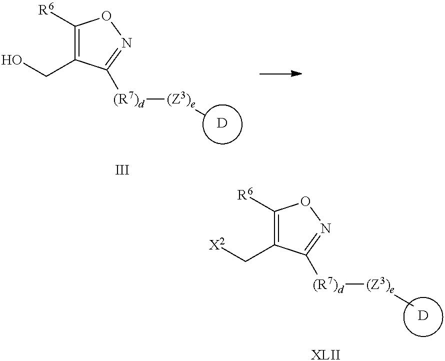

wherein all variables are as defined above.

In the embodiment wherein X² is halide, the reaction is performed by halogenating the compound of formula (III). Any suitable halogenating reagent conventional in the art may be employed in the reaction. Examples of suitable halogenating reagents include, but are not limited to, thionyl chloride and triphenylphosphine dichloride. The reaction is typically carried out in a non-polar solvent such as dichloromethane or 1,2-dichloroethane at ambient temperature.

In the embodiment wherein X² is triflate, tosylate or meslyate, the reaction process may be carried out according to the conventional methods. See, Vedejs, E., et al., 1977 J. Org. Chem. 42:3109-3113; Handy, S. T., et al., 2004 J. Org. Chem. 69:2362-2366; and Copp, F. C., et al. 1955 J. Chem. Soc. 2021-2027.

The compound of formula (III) may be prepared as previously described.

A compound of formula (XI-j) may be prepared by the reaction of an aryl bromide of formula (XLIII) with a boronic acid or ester of formula (XLIV) under standard Suzuki coupling conditions. Optionally a compound of formula (XI-j) may be reduced with hydrogen and palladium on carbon catalyst to the corresponding 1,3-dihydro-1H-indene.

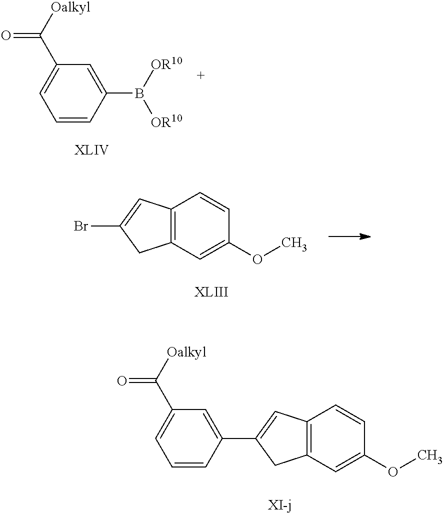

wherein: R¹⁰ is alkyl or H;
and all other variables are as defined above.

A boronic acid of formula (XLIV) may be purchased from commercial sources.

An aryl bromide of formula (XLIII) may be prepared by dehydrating a compound of formula (XLV) with an acid at elevated temperatures.

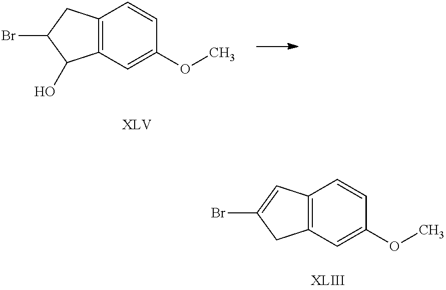

A compound of formula (XLV) may be prepared by reducing a compound of formula (XLVI) with a reducing agent, such as sodium borohydride.

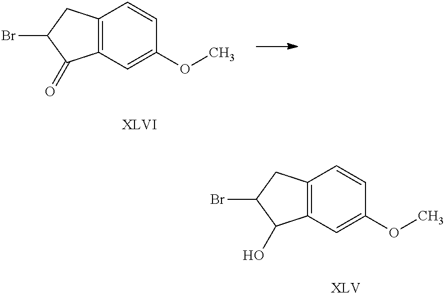

A compound of formula (XLVI) may be synthesized according to literature procedures.

It can be appreciated by those skilled in the art that a compound of formula (I) in which R¹ is —NH(SO₂CF₃) or —N(SO₂CF₃)₂ may be synthesized according to Scheme 2 by employing NH₂ in place of R¹ in a compound of formula (II). The displacement reaction shown in Scheme 2 may then be run as described to provide an intermediate aniline that can be reacted with trifluoromethanesulfonic anhydride at reduced temperature to produce a compound of formula (I) in which R¹ is —NH(SO₂CF₃) or —N(SO₂CF₃)₂. In a similar fashion a compound of formula (I) in which R¹ is —NHC(O)CH₃

or —N(C(O)CH₃)₂ may be obtained by reacting the previously described intermediate aniline with acetyl chloride.

In another embodiment, a compound of formula (I) may be prepared as depicted in Scheme 3.

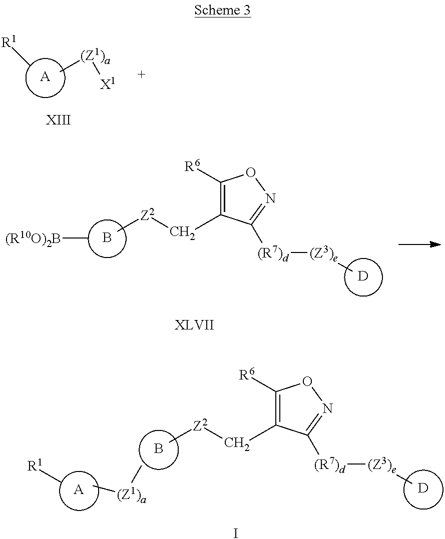

wherein:
R¹ is —CO₂alkyl;
a is 0;
X¹ is chloro, bromo, iodo, or triflate;
Ring A is phenyl or a 5-6 membered heteroaryl comprising 1, 2 or 3 heteroatoms selected from N, O and S, wherein said phenyl or heteroaryl is substituted with R¹ and further optionally substituted with one or two independently selected C₁₋₆alkyl;
Ring B is B-i, B-ii, B-iv, B-v, B-vi, B-vii, B-viii, B-ix, B-xiv, or B-xv;
R¹⁰ is H or alkyl; and
all other variables are as defined above for formula (I).

In general, the process of Scheme 3 comprises the steps of:

a) reacting a compound of formula (XIII) with a boronic acid or ester compound of formula (XLVII) under Suzuki coupling conditions to prepare a compound of formula (I);
b) optionally converting the compound of formula (I) into a pharmaceutically acceptable salt; and
c) optionally converting the compound of formula (I) or a pharmaceutically acceptable salt thereof into a different compound of formula (I) or a pharmaceutically acceptable salt thereof.

More specifically, a compound of formula (I) may prepared reacting a compound of formula (XIII) with a compound of formula (XLVII) under conventional Suzuki coupling reaction conditions. For example, the reaction may be carried out in the presence of a suitable palladium complex such as tetrakis(triphenylphosphine)-palladium(0) and a base such as sodium carbonate in a mixture of water and ethereal solvent such as 1,2-dimethoxyethane, at an elevated temperature. A compound of formula (XIII) may be purchased commercially or prepared by those skilled in the art.

A compound of formula (XLVII) may be prepared by reacting a compound of formula (XLVIII) with a compound of formula (XLII) in the presence of a base, such as cesium carbonate or potassium carbonate. The reaction may be carried out in a polar aprotic solvent, such as N,N-dimethylformamide.

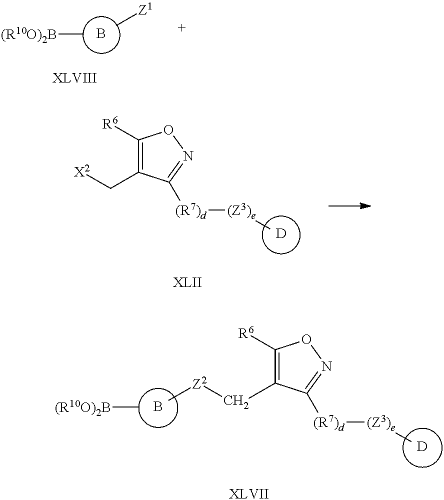

wherein:
X² is chloro, iodo, bromo, triflate, tosylate, nosylate, besylate or mesylate, (preferably chloro);
R¹⁰ is alkyl; and
all other variables are as defined above.

A compound of formula (XLVIII) may be synthesized by techniques known to those skilled in the art or purchased commercially. A compound of formula (XLII) may be prepared as described above.

In another embodiment, a compound of formula (I) may be prepared as depicted in Scheme 4.

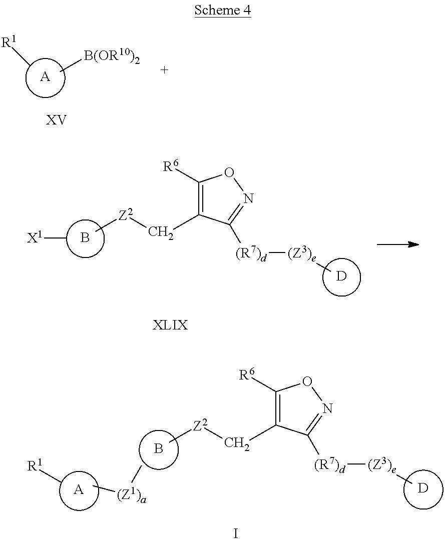

wherein:
R¹ is —CO₂alkyl, —CH₂CH₂CO₂alkyl, —NHC(O)CH₃, or —OCF₃;
Ring A is phenyl or a 5-6 membered heteroaryl comprising 1, 2 or 3 heteroatoms selected from N, O and S, wherein said phenyl or heteroaryl is substituted with R¹ and further optionally substituted with one or two independently selected C₁₋₆alkyl;

### Ring B is B-i, B-ii, B-iii, B-iv, B-v, B-vi, B-vii, B-viii, B-ix, B-xiv, or B-xv;

a is 0;
R¹⁰ is H or alkyl;
X¹ is chloro, bromo, iodo or triflate; and
all other variables are as defined above for formula (I).

In general, the process of Scheme 4 comprises the steps of:

a) reacting a compound of formula (XLIX) with a boronic acid or ester compound of formula (XV) under Suzuki coupling conditions to prepare a compound of formula (I);
b) optionally converting the compound of formula (I) into a pharmaceutically acceptable salt thereof; and
c) optionally converting the compound of formula (I) or a pharmaceutically acceptable salt thereof into a different compound of formula (I) or a pharmaceutically acceptable salt thereof.

More specifically, a compound of formula (I) may be prepared reacting a compound of formula (XLIX) with a compound of formula (XV) under conventional Suzuki coupling reaction conditions. For example, the reaction may be carried out in the presence of a suitable palladium complex such as tetrakis(triphenylphosphine)-palladium(0) and a base such as sodium carbonate in a mixture of water and ethereal solvent such as 1,2-dimethoxyethane, at an elevated temperature. A compound of formula (XV) may be purchased from commercial sources or may be prepared by those skilled in the art.

More particularly, a compound of formula (XLIX) may be prepared by reacting the compound of formula (L) with a compound of formula (III) in the presence of triphenylphosphine and a dialkylazodicarbonate like diisopropylazodicarbonate at elevated temperature.

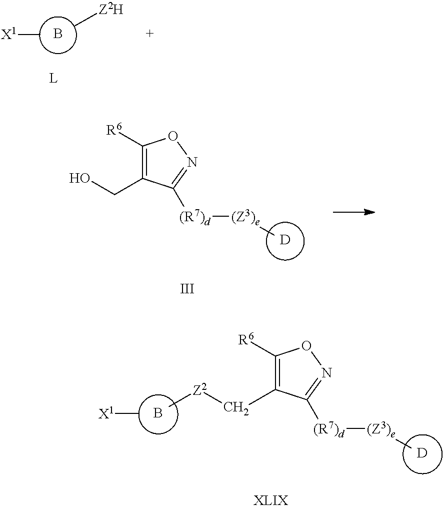

wherein:
X¹ is chloro, bromo, iodo or triflate;
Z² is —O— or —S—; and
all other variables are as defined above.

It will be apparent to those that are skilled in the art that where Z² is N it may be required to first convert the compound of formula (L) to the trifluoroacetamide using known techniques prior to the Mitsunobu reaction. The trifluoroacetamide can be cleaved during the saponification of the ester to form a compound of formula (XLIX).

A compound of formula (L) may be synthesized by techniques known to those skilled in the art or purchased commercially. A compound of formula (III) may be prepared as described above.

As another example, a compound of formula (XLIX) may be prepared by reacting a compound of formula (XLII) with a compound formula (L) in the presence of a base, such as cesium carbonate, in a solvent, such as dimethylformamide.

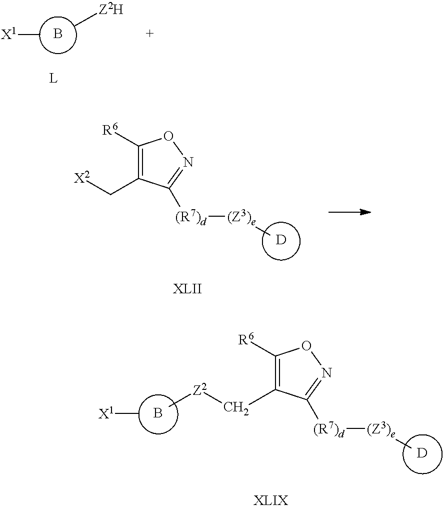

wherein all variables are as defined above.

A compound of formula (L) may be synthesized by techniques known to those skilled in the art or purchased commercially. A compound of formula (XLII) may be prepared as described above.

As another example, a compound of formula (XLIX) may be prepared by refluxing a solution of a compound of formula (LI) and an acid like p-toluene sulfonic acid in a flask fitted with a Dean Stark trap.

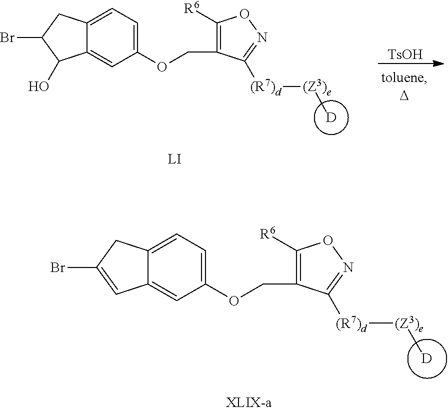

wherein all variables are as defined above.

A compound of formula (LI) may be prepared by reducing a compound of formula (LII) with a reducing agent like sodium borohydride.

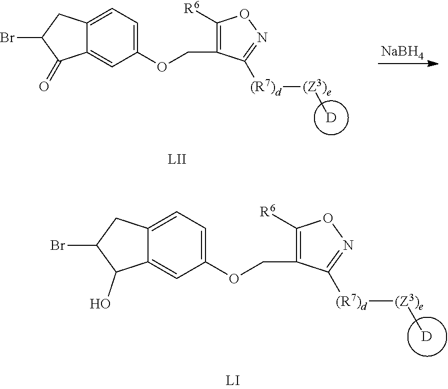

wherein all variables are as defined above.

A compound of formula (LII) may be prepared by reacting a compound of formula (LIII) with copper (II) bromide in a solvent like chloroform.

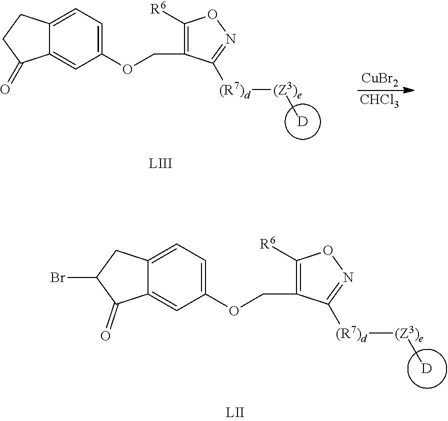

wherein all variables are as defined above.

A compound of formula (LIII) may be prepared by reacting a phenol of formula (LIV) with an alcohol of formula (III) under standard Mitsunobu coupling conditions. A compound of formula (LIV) may be purchased from commercial sources or may be synthesized by one skilled in the art. A compound of formula (III) may be prepared as described above.

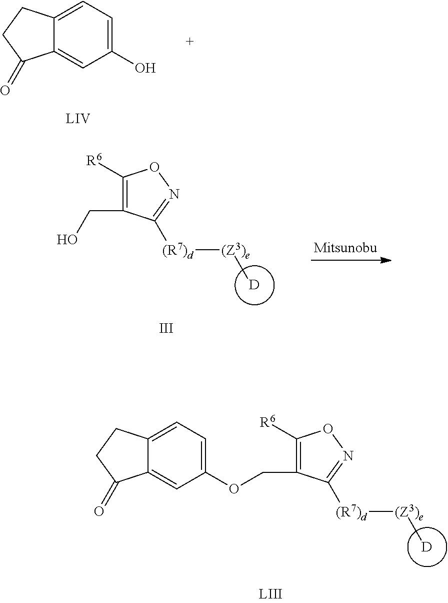

wherein all variables are as defined above.

It can be appreciated by those skilled in the art that a compound of formula (I) in which R¹ is —NH(SO₂CF₃) or —N(SO₂CF₃)₂ may be synthesized according to Scheme 4 by employing NH₂ in place of R¹ in a compound of formula (XV). The Suzuki coupling shown in Scheme 4 may then be run as described to provide an intermediate that can be reacted with trifluoromethanesulfonic anhydride at reduced temperature to produce a compound of formula (I) in which R¹ is —NH(SO₂CF₃) or —N(SO₂CF₃)₂. In a similar fashion a compound of formula (I) in which R¹ is —NHC(O)CH₃

or —N(C(O)CH₃)₂ may be obtained by reacting the previously described intermediate aniline with acetyl chloride.

In another embodiment, a compound of formula (I) may be prepared using the process depicted in Scheme 5, below.

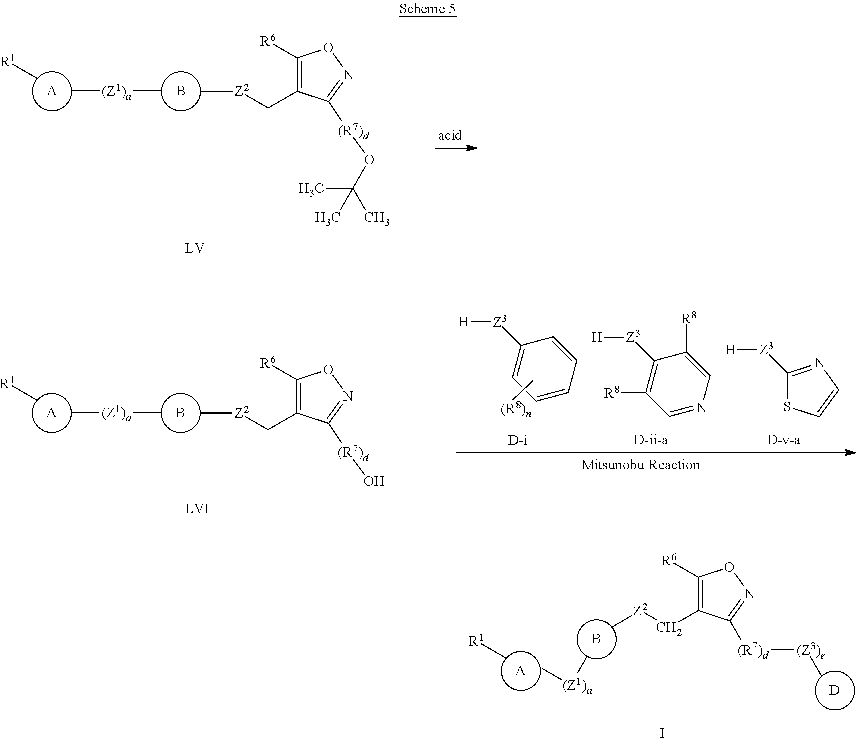

wherein:
R¹ is —CO₂alkyl;
Z³ is selected from —O—, —S—, —NH—;
e is 1;
Ring D is a moiety of formula D-i, D-ii-a or D-v-a:

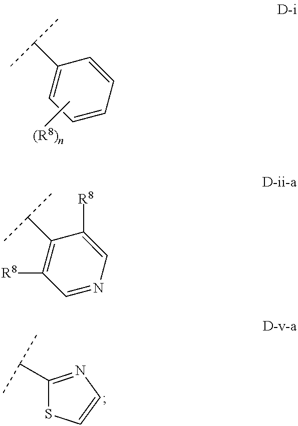

and
all other variables are as defined above.

In general, the process of preparing a compound of formula (I) according to Scheme 5 comprises the steps of:

a) reacting a compound of formula (LV) with acid to prepare a compound of formula (LVI);
b) reacting a compound of formula (LVI) under Mitsunobu reaction conditions with
a Ring D moiety of formula D-i, D-ii-a, or D-v-a to prepare a compound of formula (I);
c) optionally converting the compound of formula (I) into a pharmaceutically acceptable salt thereof; and
d) optionally converting the compound of formula (I) or a pharmaceutically acceptable salt thereof into a different compound of formula (I) or a pharmaceutically acceptable salt thereof.

More specifically, a compound of formula (LVI) may be prepared by reacting the compound of formula (LV) with an acid. The reaction may be carried out in a solvent, such as dichloromethane or 1,2-dichloroethane. Suitable acids for use in this reaction will be apparent to those skilled in the art and include, but are not limited to trifluoroacetic acid. The resulting alcohol compound of formula (LVI) may be reacted with a suitable Ring D moiety of formula of D-i, D-ii-a, or D-v-a under conventional Mitsunobu reaction conditions. For example, this reaction may be carried out in a solvent, such as dichloromethane or toluene, with triphenyl phosphine and a dialkyl azodicarboxylate like diisopropyl azodicarboxylate or di-tent-butyl azodicarboxylate to prepare a compound of formula (I). One who is skilled in the art would realize that it may be necessary to first convert an aniline Ring D moiety to the trifluoracetamide prior to the Mitsunobu reaction to form a compound of formula (I).

Upon hydrolysis of the ester to the acid the trifluoroacetamide may be hydrolysed to the corresponding amine and trifluoroacetic acid.

In another embodiment, a compound of formula (I) may be prepared as depicted in Scheme 6.

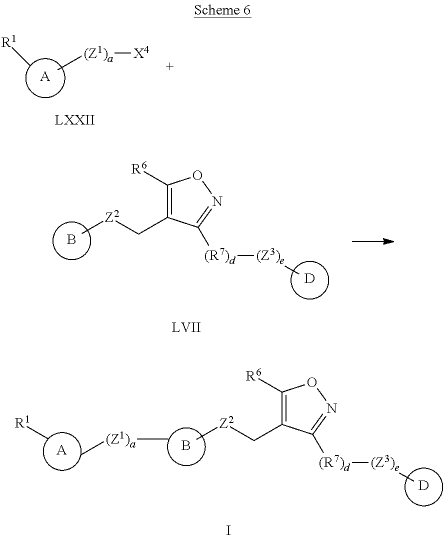

wherein:
R¹ is —CO₂alkyl;
Z¹ is —CH₂—, —CO— or —SO₂—;
a is 1;
X⁴ is iodo, chloro or bromo (preferably chloro);
Ring B is an indole or benzamidazole; and
all other variables are as defined above.

In general, the process of Scheme 6 comprises the steps of:

a) condensing a compound of formula (LXXII) with a compound of formula (LVII) optionally with a base to prepare a compound of formula (I);
b) optionally converting the compound of formula (I) into a pharmaceutically acceptable salt thereof; and
c) optionally converting the compound of formula (I) or a pharmaceutically acceptable salt thereof into a different compound of formula (I) or a pharmaceutically acceptable salt thereof.

More specifically, a compound of formula (I-a) may be prepared by reacting a compound of formula (LXXII) with a compound of formula (LVII-a). For example, the reaction may be carried out in the presence of a suitable base such as cesium carbonate in dimethyl formamide at an elevated temperature. A compound of formula (LXXII) may be purchased commercially or synthesized by those skilled in the art.

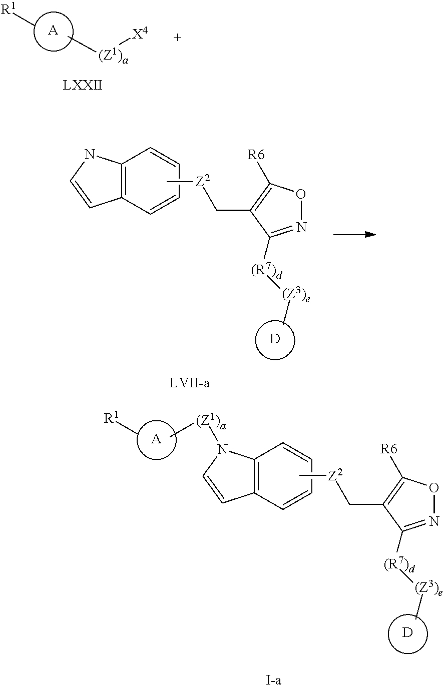

wherein:
R¹ is —CO₂alkyl;
Z¹ is —CH₂—;
a is 1;
X⁴ is chloro, bromo or iodo; and
all other variables are as defined above.

It can be appreciated by those skilled in the art that a compound of formula (I-a) in which R¹ is a tetrazole may be synthesized according to Scheme 6 by employing a nitrile in place of R¹ in a compound of formula (XIII). The resulting intermediate can then be reacted with sodium azide and ammonium chloride at elevated temperatures to form the desired tetrazole.

A compound of formula (LVII) may be prepared by reacting a compound of formula (LVIII) with a compound of formula (III) in the presence of triphenylphosphine and a dialkylazodicarbonate, like diisopropylazodicarbonate, at elevated temperature.

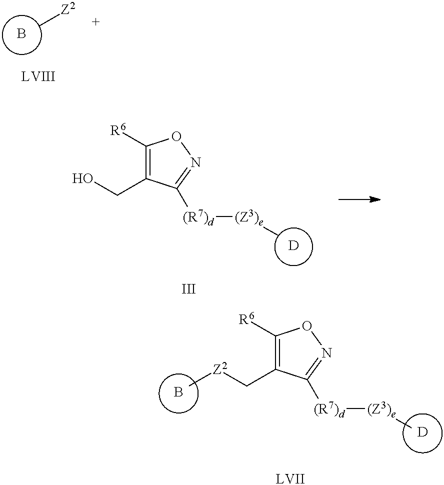

wherein:
all variables are as defined above.

A compound of formula (LVIII) may be synthesized by techniques known to those skilled in the art or purchased commercially. A compound of formula (III) may be prepared as described above.

For example, a compound of formula (XIII-a) may be synthesized by reacting a compound of formula (LIX) with sodium methoxide in methanol at a reduced temperature.

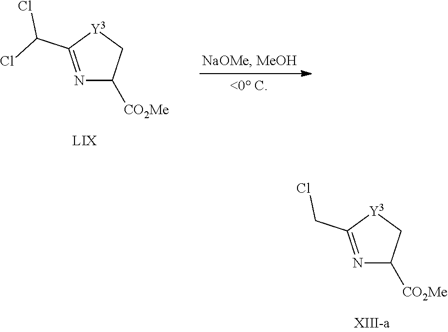

wherein: Y³ is —O— or —S—; and
all other variables are as defined above.

A compound of formula (LIX) may be synthesized by reacting a compound of formula (LX) with sodium methoxide in methanol at a reduced temperature, followed by the addition of cysteine.

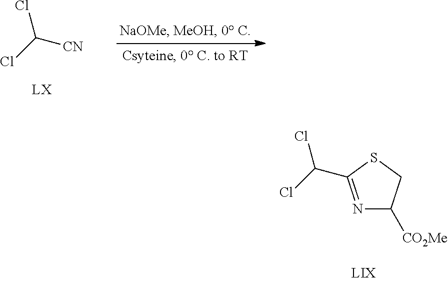

Alternatively, a compound a formula (I-b) may be made by reacting a compound of formula (LXXII) with a compound of formula (LVII-a), zinc trifluoromethansulfonate, Bu₄NI, and diisopropylethylamine.

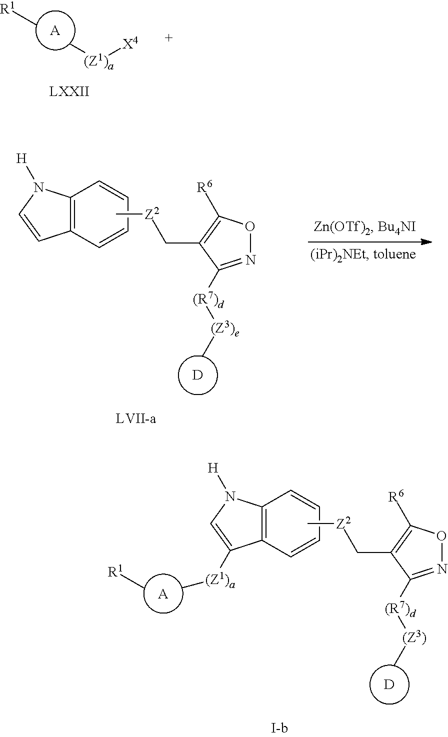

wherein:
Z¹ is —CH₂—;
a is 1;
X⁴ is chloro, bromo or iodo;
OTf is trifluoromethane sulfonate;
Bu₄NI is tetrabutylammonium iodide
(iPr)₂NEt is diisopropylethylamine; and
all other variables are as defined above.

In another embodiment, a compound of formula (I) may be prepared using the process depicted in Scheme 7, below.

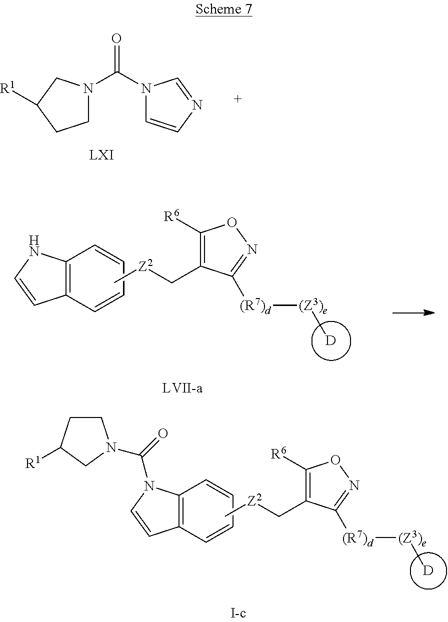

wherein:
R¹ is —CO₂alkyl; and
all other variables are as defined above.

In general, the process of Scheme 7 comprises the steps of:

a) condensing a compound of formula (LXI) with a compound of formula (LVII-a) optionally with a base to prepare a compound of formula (I-c);
b) optionally converting the compound of formula (I-c) into a pharmaceutically acceptable salt thereof and
c) optionally converting the compound of formula (I-c) or a pharmaceutically acceptable salt thereof into a different compound of formula (I-c) or a pharmaceutically acceptable salt thereof.

A compound of formula (LVII-a) can be made as described above. A compound of formula (LXI) can be made by reacting a compound of formula (LXII) with a compound of formula (LXIII). Compounds of formulas (LXII and LXIII) are available from commercial sources.

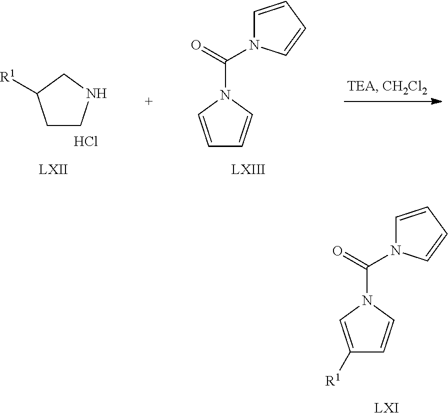

wherein R¹ is —CO₂alkyl.

In another embodiment, a compound of formula (I) may be prepared using the process depicted in Scheme 8, below.

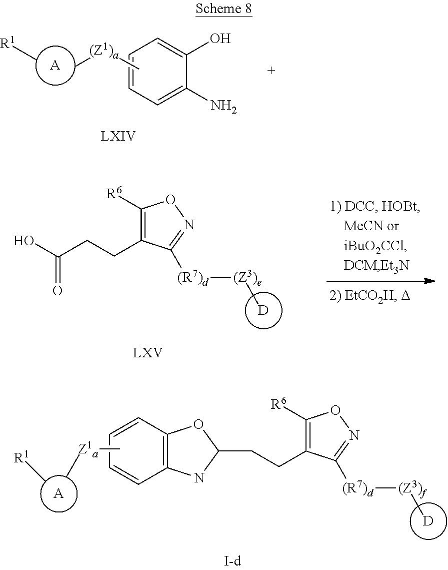

wherein:
Z¹ is —NH—;
a is 0 or 1;
R¹ is —CO₂alkyl;
DCC is N,N-dicyclohexylcarbodiimide;
HOBt is 1-hydroxybenzotriazole; and
all other variables are as defined above for formula (I),

In general, the process for preparing a compound of formula (I-d) as depicted in Scheme 8 comprises the steps of:

a) reacting a compound of formula (LXIV) with a compound of formula (LXV) to prepare an intermediate amide, and dehydrating the intermediate to prepare a compound of formula (I-d);
b) optionally converting the compound of formula (I-d) into a pharmaceutically acceptable salt thereof and
c) optionally converting the compound of formula (I-d) or a pharmaceutically acceptable salt thereof into a different compound of formula (I-d) or a pharmaceutically acceptable salt thereof.

A compound of formula (I-d), prepared by any suitable process, may be converted into a pharmaceutically acceptable salt thereof or may be converted to a different compound of formula (I-d) or a pharmaceutically acceptable salt thereof using techniques described herein below and those conventional in the art.

More particularly, the compound of formula (I-d) may be prepared by coupling the compound of formula (LXIV) with a compound of formula (LXV) through one of the many known amide bond formation reactions to produce an intermediate amide. The amide may then undergo a dehydrating ring formation by heating with an acid like propionic acid.

The compound of formula (LXV) may be prepared by reacting a compound of formula (LXVI) with triethylamine formate and 2,2-dimethyl-1.3-dioxane-4,6-dione in a solvent like N,N-dimethylformamide at elevated temperatures.

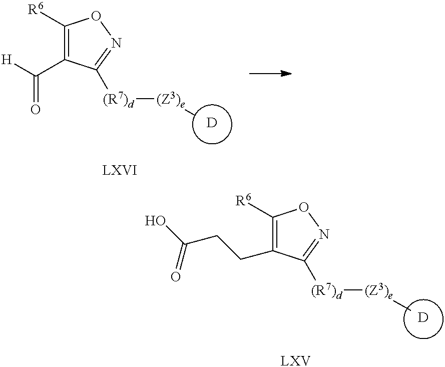

wherein all variables are as defined above.

A compound of formula (LXVI) may be synthesized by oxidizing a compound of formula (III) with an oxidizing agent like pyridium chlorochromate in a solvent like dichloromethane. A compound of formula (III) may be made as described above.

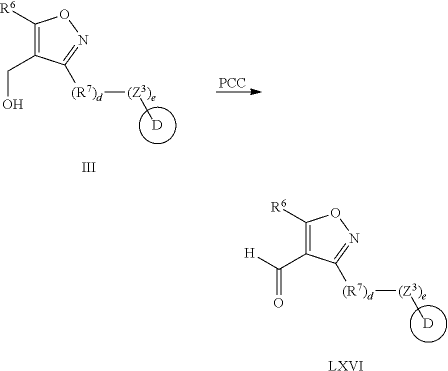

wherein: PCC is pyridinium chlorochromate; and
all other variables are as defined above.

A compound of formula (LXIV-a) can be synthesized by reducing a compound of formula (LXVII) with hydrogen and a catalyst like palladium on carbon.

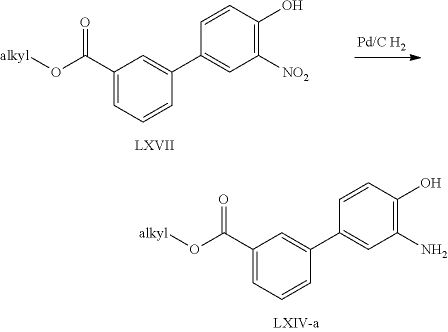

A compound of formula (LXVII) can synthesized by reacting compounds of formulas (LXIX) and (LXVIII) under standard Suzuki conditions with a palladium catalyst at elevated temperatures. Compounds of formulas (LXIX) and (LXVIII) can be purchased from commercial sources or synthesized by those skilled in the art.

More particularly, a compound of formula (LXVII) may be prepared by reacting the compounds of formula (LXVIII) and formula (LXIX) in the presence of tetrakis(triphenylphosphine)palladium(0) and an aqueous sodium carbonate solution in a solvent like 1,2-dimethoxyethane at 85-90° C.

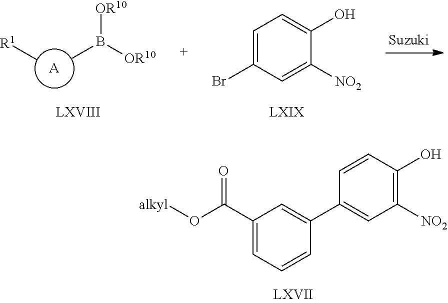

wherein:
Ring A is phenyl or a 5-6 membered heteroaryl comprising 1, 2 or 3 heteroatoms selected from N, O and S, wherein said phenyl or heteroaryl is substituted with R¹ and further optionally substituted with one or two independently selected C₁₋₆alkyl;
R¹ is —CO₂alkyl; and
all other variables are as defined above.

A compound of formula (LXIV-b) may be synthesized by reacting a aniline of formula (LXX) with a compound of formula (LXXI) in the presence of a suitable palladium catalyst in the presence of a base. For example, the reaction may be carried out in the presence of cesium carbonate and a suitable palladium complex such as the one formed by the complexation of tris(diphenylideneacetone)dipalladium(0) and rac-2,2′-bis(diphenylphosphino)-1,1′-binaphthyl in a solvent like toluene at an elevated temperature. The resulting intermediate can then be reduced with hydrogen and a palladium catalyst like palladium on carbon to afford a compound of formula (LXIV-b). Compounds of formulas (LXX and LXXI) may be purchased from commercial sources or synthesized by those skilled in the art.

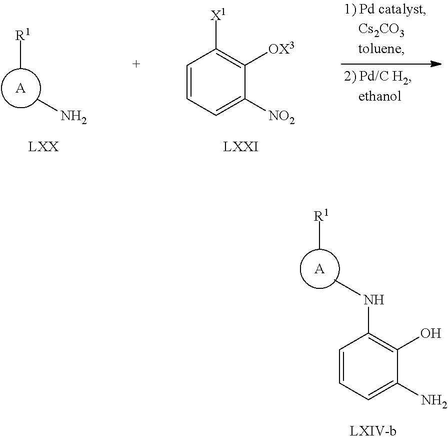

wherein:
Ring A is phenyl or a 5-6 membered heteroaryl comprising 1, 2 or 3 heteroatoms selected from N, O and S, wherein said phenyl or heteroaryl is substituted with R¹ and further optionally substituted with one or two independently selected C₁₋₆alkyl;
R¹ is —CO₂alkyl;
X³ is benzyl;
and all variables are as defined above.

In another embodiment, a compound of formula (I) may be prepared using the process depicted in Scheme 9, below.

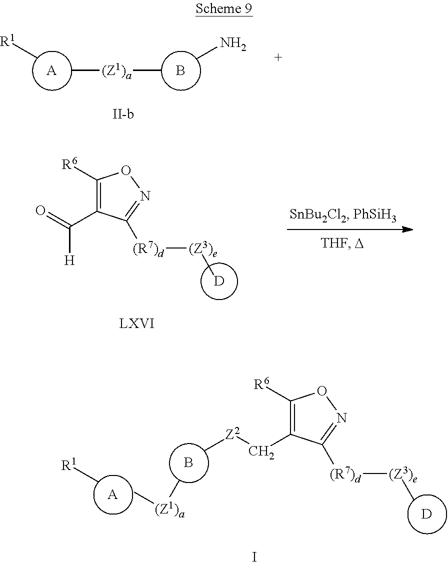

wherein:
R¹ is —CO₂alkyl;
Z² is —NH—;
SnBu₂Cl₂ is dibutyltin dichloride;
PhSiH₃ is phenyl silane;
THF is tetrahydrofuran; and
all other variables are as defined above for formula (I).

In general, the process for preparing a compound of formula (I) as depicted in Scheme 9 comprises the steps of:

a) reacting a compound of formula (II-b) with a compound of formula (LXVI) to prepare a compound of formula (I);
b) optionally converting the compound of formula (I) into a pharmaceutically acceptable salt thereof; and
c) optionally converting the compound of formula (I) or a pharmaceutically acceptable salt thereof into a different compound of formula (I) or a pharmaceutically acceptable salt thereof.

A compound of formula (I), prepared by any suitable process, may be converted into a pharmaceutically acceptable salt thereof or may be converted to a different compound of formula (I) or a pharmaceutically acceptable salt thereof using techniques described herein below and those conventional in the art.

More particularly, the compound of formula (I) may be prepared by reacting the compound of formula (II-b) with a compound of formula (LXVI) in the presence of dibutyltin dichloride and phenyl silane at room temperature or at elevated temperatures.

A compound of the formula II-b may be synthesized by reacting a boronic ester or acid of formula (XV-a) with an aryl halide or triflate of formula (LXX) under standard Suzuki coupling conditions.

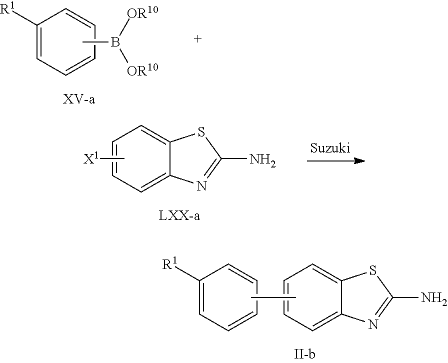

wherein: R¹ is —CO₂alkyl;
and all other variables are as defined above.

Based upon these examples and the disclosure contained herein one skilled in the art can readily convert compounds of formula (I) into other compounds of formula (I), or salts thereof. For example, an ester of a compound of formula (I) may be converted into an acid of a compound of formula (I) as in Examples 1-11, 13-25, 33-45 and 47-60.

The following examples are intended for illustration only and are not intended to limit the scope of the invention in any way, the present invention being defined by the claims.

In the examples, the following terms have the designated meaning:

Et=ethyl;
g=gram;
mg=milligram;
h=hour;
min=minute;
L=liter;
mL=milliliter;
M=molar;
mol=mole;
mmol=millimolar;
N=normal;
˜=approximately;
HPLC=high performance liquid chromatography;
NMR=nuclear magnetic resonance;
H=hydrogen atom;
Hz=Hertz; mHz=megaHertz;
DMSO=dimethylsulfoxide;

As used in the examples, 4-(chloromethyl)-3-(2,6-dichlorophenyl)-5-(1-methylethyl)isoxazole may be prepared by a procedure similar to that described below:

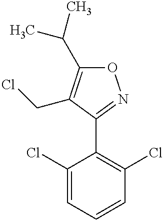

Thionyl chloride (123 mL, 202 g, 1.7 mol) was added dropwise during 30 min to a stirred suspension of benzotriazole (202 g, 1.7 mol) in dichloromethane (550 mL) at room temperature under N₂. The resulting yellow solution was transferred to an addition funnel and added dropwise during 1 hour to a stirred solution of [3-(2,6-dichlorophenyl)-5-(1-methylethyl)-4-isoxazolyl]methanol (372 g, 1.3 mol, Maloney, P. R., et al., 2000 J. Med. Chem. 43:2971-2974) in dichloromethane (975 mL). The reaction temperature gradually rose to a maximum of 28° C. After 1 hr the resulting suspension was filtered to remove the benzotriazole hydrochloride. The filtrate was washed with water (2×1 L), with 1 N sodium hydroxide (1 L), with water (1 L), dried over anhydrous sodium sulfate, filtered and concentrated to yield 4-(chloromethyl)-3-(2,6-dichlorophenyl)-5-(1-methylethyl)isoxazole as a pale yellow oil (413 g, 80%). ¹H NMR (400 MHz, DMSO-d₆); [symbol] 7.64 (m, 3H), 4.47 (s, 2H), 3.45 (m, 1H), 1.31 (d, J=7 Hz, 6H). ES-LCMS m/z 305 (M+H)⁺.

### Example 1

### 3-{[5-({[3-{[(2,6-dimethylphenyl)oxy]methyl}-5-(1-methylethyl)-4-isoxazolyl]methyl}oxy)-1H-indol-1-yl]methyl}benzoic acid

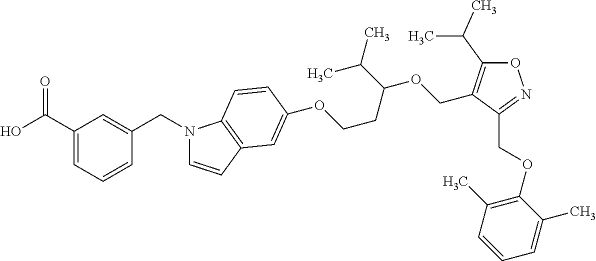

### 1a) (1,1-Dimethylethyl)oxyethanal oxime

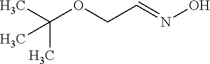

To a stirring solution of ethylene glycol tent-butyl ether (27.5 mL, 209 mmol) and triethylamine (87.5 mL, 628 mmol) in dichloromethane (600 mL) at 0° C. was added, over a period of approximately 45 minutes, a solution of sulfur trioxide-pyridine complex (100 g, 628 mmol) in dimethylsulfoxide (600 mL) that had been stirring for approximately 25 minutes. The mixture was allowed to warm to ambient temperature and stir over 6 hours and was then poured into ether, washed three times with 10% aqueous citric acid, then brine and concentrated. The residue was taken up with ethanol (2.65 L) and filtered into a stirring solution of hydroxylamine hydrochloride (16.0 g, 230 mmol) and sodium hydroxide (9.20 g, 230 mmol) in water (125 mL).

The solution was heated to approximately 90° C. and stirred for approximately 17 hours. The mixture was then concentrated and the residue was taken up with ethyl acetate and washed twice with water containing sodium chloride. The combined aqueous layers were back-extracted with ethyl acetate and the combined organic layers were dried over magnesium sulfate, concentrated and purified by chromatography (silica gel, 15% ethyl acetate in hexanes) to afford 1,1-dimethylethyl)oxyethanal oxime (8.59 g, 31%). ¹H-NMR (400 MHz, DMSO-d₆) δ 11.02 (s, 0.5H), 10.73 (s, 0.5H), 7.25 (t, J=6 Hz, 0.5H), 6.67 (t, J=4 Hz, 0.5H), 4.11 (d, J=4 Hz, 1H), 3.89 (d, J=6 Hz, 1H), 1.12 (s, 9H).

### 1b) Methyl 3-{[(1,1-dimethylethyl)oxy]methyl}-5-(1-methylethyl)-4-isoxazolecarboxylate

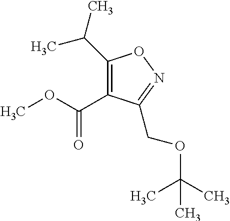

To a solution of (1,1-dimethylethyl)oxyethanal oxime (8.59 g, 65.5 mmol) in N,N-dimethylformamide (50 mL) was added N-chlorosuccinimide (8.45 g, 65.5 mmol). The solution was stirred for approximately 1 hour. The solution was poured into ether and washed twice with water. The organic layer containing the crude imidoyl chloride was then washed with brine, dried over magnesium sulfate and concentrated. Then to a solution of methyl isobutyrylacetate (8.86 mL, 78.6 mmol) in tetrahydrofuran (40 mL) at 0° C. was added a 0.5 M solution of sodium methoxide in methanol (157 mL, 78.6 mmol). After stirring for approximately 5 minutes the above imidoyl chloride was added in tetrahydrofuran (30 mL). A solid was observed to precipitate. After the addition was complete the mixture was allowed to stir and warm to ambient temperature overnight. Then the solution was poured into ether and washed with water containing sodium chloride, dried over magnesium sulfate and concentrated to afford methyl 3-{[(1,1-dimethylethyl)oxy]methyl}-5-(1-methylethyl)-4-isoxazolecarboxylate (10.6 g, 63%). ¹H-NMR (400 MHz, DMSO-d₆) δ 4.52 (s, 2H), 3.77 (s, 3H), 3.67 (septet, J=7 Hz, 1H), 1.25 (d, J=7 Hz, 6H), 1.18 (s, 9H).

### 1c) [3-{[(1,1-dimethylethyl)oxy]methyl}-5-(1-methylethyl)-4-isoxazolyl]methanol

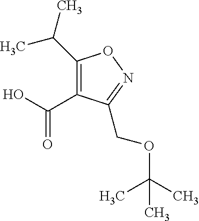

To a solution of methyl 3-{[(1,1-dimethylethyl)oxy]methyl}-5-(1-methylethyl)-4-isoxazolecarboxylate (21.5 g, 84.2 mmol, prepared by the general procedure described in Example 1b) in tetrahydrofuran (250 mL) at 0° C. was slowly added a 1.5 M solution of diisobutylaluminum hydride in toluene (185 mL, 278 mmol). The solution was allowed to warm slowly to ambient temperature overnight then was re-cooled to 0° C. and approximately 250 mL of a 10% solution of Rochelle's salt was added dropwise followed by approximately 300 mL of ethyl acetate. An additional 250 mL of a 10% solution of Rochelle's salt and 500 mL of ethyl acetate were added and the mixture was stirred at 0° C. for approximately 20 minutes, then at ambient temperature for approximately 4 hours. The mixture was filtered, the layers were separated and the aqueous layer extracted with ethyl acetate. The combined organic layers were dried over magnesium sulfate, concentrated, purified by chromatography (silica gel, 20% ethyl acetate in hexanes) to afford [3-{[(1,1-dimethylethyl)oxy]methyl}-5-(1-methylethyl)-4-isoxazolyl]methanol (15.2 g, 91%). ¹H-NMR (400 MHz, DMSO-d₆): δ 4.76 (t, J=5 Hz, 1H), 4.39 (s, 2H), 4.32 (d, J=5 Hz, 2H), 3.24 (septet, J=7 Hz, 1H), 1.21 (d, J=7 Hz, 6H), 1.18 (s, 9H).

### 1d) 4-(chloromethyl)-3-{[(1,1-dimethylethyl)oxy]methyl}-5-(1-methylethyl)isoxazole

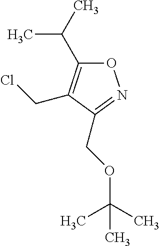

To a solution of [3-{[(1,1-dimethylethyl)oxy]methyl}-5-(1-methylethyl)-4-isoxazolyl]methanol (2.85 g, 12.5 mmol) in dichloromethane (60 mL) at 0° C. was added thionyl chloride (0.915 mL, 12.5 mmol) dropwise. The solution was allowed to stir while warming to ambient temperature for approximately 1 hour and was then concentrated. The residue was diluted with ethyl acetate, washed twice with aqueous sodium bicarbonate, once with brine, dried over magnesium sulfate and concentrated to afford 4-(chloromethyl)-3-{[(1,1-dimethylethyl)oxy]methyl}-5-(1-methylethyl)isoxazole (3.04 g, 99%). ¹H-NMR (400 MHz, DMSO-d₆): δ 4.67 (s, 2H), 4.45 (s, 2H), 3.32 (septet, J=7 Hz, 1H), 1.23 (d, J=7 Hz, 6H), 1.21 (s, 9H).

### 1e) Methyl 3-{[5-({[3-{[(1,1-dimethylethyl)oxy]methyl}-5-(1-methylethyl)-4-isoxazolyl]methyl}oxy)-1H-indol-1-yl]methyl}benzoate

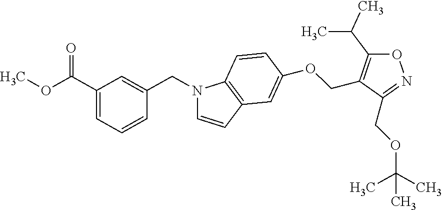

To a solution of methyl 3-[(5-hydroxy-1H-indol-1-yl)methyl]benzoate (3.31 g, 11.7 mmol, which may be prepared according to the general procedure described in Example 59b) in N,N-dimethylformamide (15 mL) was added cesium carbonate (5.74 g, 17.6 mmol) and the mixture was stirred at between 65 and 70° C. for approximately 45 minutes. Then 4-(chloromethyl)-3-{[(1,1-dimethylethyl)oxy]methyl}-5-(1-methylethyl)isoxazole (3.04 g, 12.3 mmol) in N,N-dimethylformamide (15 mL) was added at 65° C. The mixture was stirred at 65° C. overnight, then poured into a water-brine mixture and extracted three times with ethyl acetate. The combined organic layers were washed with brine, dried over magnesium sulfate and concentrated. The crude material was purified by chromatography (silica gel, 0-20% ethyl acetate in hexanes gradient elution) to afford methyl 3-{[5-({[3-{[(1,1-dimethylethyl)oxy]methyl}-5-(1-methylethyl)-4-isoxazolyl]methyl}oxy)-1H-indol-1-yl]methyl}benzoate (4.12 g, 72%). ¹H-NMR (400 MHz, DMSO-d₆): δ 7.81-7.75 (m, 2H), 7.47-7.39 (m, 3H), 7.28 (d, J=9 Hz, 1H), 7.14 (s, 1H), 6.75-6.73 (m 1H), 6.39 (s, 1H), 5.44 (s, 2H), 4.90 (s, 2H), 4.41 (s, 2H), 3.79 (s, 3H), 3.26 (septet, J=7 Hz, 1H), 1.16 (d, J=7 Hz, 6H), 1.10 (s, 9H).

### 1f) Methyl 3-{[5-({[3-(hydroxymethyl)-5-(1-methylethyl)-4-isoxazolyl]methyl}oxy)-1H-indol-1-yl]methyl}benzoate

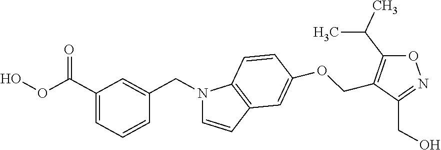

To a solution of methyl 3-{[5-({[3-{[(1,1-dimethylethyl)oxy]methyl}-5-(1-methylethyl)-4-isoxazolyl]methyl}oxy)-1H-indol-1-yl]methyl}benzoate (1.34 g, 2.73 mmol) in dichloromethane (135 mL) was added trifluoroacetic acid (135 mL, 640 mmol) and the solution was stirred for 1 hour. The solution was then concentrated and the residue was diluted with ethyl acetate and stirred with aqueous sodium bicarbonate. Solid sodium bicarbonate was added until the pH was slightly basic. The layers were separated and the organic layer was washed once more with aqueous sodium bicarbonate, dried over magnesium sulfate and concentrated. The crude material was purified by chromatography (silica gel, 0-40% ethyl acetate in hexanes gradient elution) to afford methyl 3-{[5-({[3-(hydroxymethyl)-5-(1-methylethyl)-4-isoxazolyl]methyl}oxy)-1H-indol-1-yl]methyl}benzoate (381 mg, 32%). ¹H-NMR (400 MHz, DMSO-d₆): δ 7.82-7.75 (m, 2H), 7.48-7.38 (m, 3H), 7.28 (d, J=9 Hz, 1H), 7.14 (s, 1H), 6.76-6.74 (m, 1H), 6.40 (s, 1H), 5.45 (s, 2H), 5.38 (br s, 1H), 4.93 (s, 2H), 4.51 (br s, 2H), 3.79 (s, 3H), 3.25 (septet, J=7 Hz, 1H), 1.16 (d, J=7 Hz, 6H). APCI-LCMS m/z 457 (M+Na)⁺.

### 1g) Methyl 3-{[5-({[3-{[(2,6-dimethylphenyl)oxy]methyl}-5-(1-methylethyl)-4-isoxazolyl]methyl}oxy)-1H-indol-1-yl]methyl}benzoate

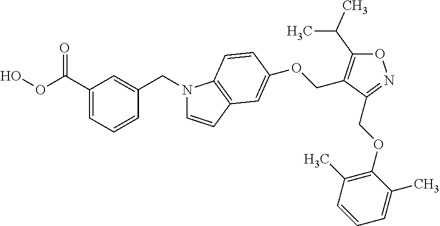

To a solution of 2,6-dimethylphenol (20 mg, 0.16 mmol), triphenylphosphine (43 mg, 0.16 mmol) and methyl 3-{[5-({[3-(hydroxymethyl)-5-(1-methylethyl)-4-isoxazolyl]methyl}oxy)-1H-indol-1-yl]methyl}benzoate (71 mg, 0.16 mmol) in toluene (2.5 mL) was added diisopropyl azodicarboxylate (0.029 mL, 0.16 mmol). The solution was heated in a microwave reactor at 90° C. for 10 minutes. The solution was adsorbed onto silica gel and purified by chromatography (silica gel, 0-15% ethyl acetate in hexanes gradient elution) to afford methyl 3-{[5-({[3-{[(2,6-dimethylphenyl)oxy]methyl}-5-(1-methylethyl)-4-isoxazolyl]methyl}oxy)-1H-indol-1-yl]methyl}benzoate (34 mg, 39%). ¹H-NMR (400 MHz, DMSO-d₆): δ 7.95-7.90 (m, 2H), 7.35 (t, J=8 Hz, 1H), 7.20-7.11 (m, 4H), 6.99-6.81 (m, 4H), 6.49 (s, 1H), 5.33 (s, 2H), 5.00 (s, 2H), 4.94 (s, 2H), 3.89 (s, 3H), 3.25 (septet, J=7 Hz, 1H), 2.23 (s, 6H), 1.33 (d, J=7 Hz, 6H). APCI-LCMS m/z 539 (M+H)⁺.

### 1h) 3-{[5-({[3-{[(2,6-dimethylphenyl)oxy]methyl}-5-(1-methylethyl)-4-isoxazolyl]methyl}oxy)-1H-indol-1-yl]methyl}benzoic acid

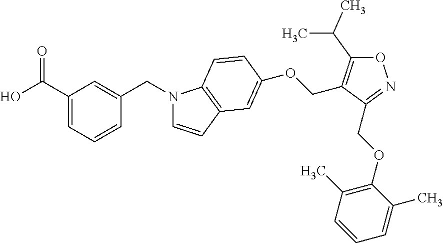

To a solution of methyl 3-{[5-({[3-{[(2,6-dimethylphenyl)oxy]methyl}-5-(1-methylethyl)-4-isoxazolyl]methyl}oxy)-1H-indol-1-yl]methyl}benzoate (34 mg, 0.063 mmol) in 1:1 tetrahydrofuran-methanol (1.5 mL) was added 1 N sodium hydroxide (0.11 mL, 0.11 mmol). The solution was heated in a microwave reactor at 120° C. for 500 seconds. Additional 1 N sodium hydroxide (0.11 mL) was added and the solution was heated in a microwave reactor at 120° C. for 500 seconds. The solution was concentrated, and water followed by 1 N hydrochloric acid (0.22 mL, 0.22 mmol) was added. The solution was extracted with ethyl acetate and the organic layer was dried over magnesium sulfate and concentrated to afford 3-{[5-({[3-{[(2,6-dimethylphenyl)oxy]methyl}-5-(1-methylethyl)-4-isoxazolyl]methyl}oxy)-1H-indol-1-yl]methyl}benzoic acid (29 mg, 88%). ¹H-NMR (400 MHz, CDCl₃): δ 8.01-7.74 (m, 2H), 7.39 (t, J=8 Hz, 1H), 7.26-7.24 (m, 1H), 7.18-7.12 (m, 3H), 6.99-6.82 (m, 4H), 6.50 (s, 1H), 5.35 (s, 2H), 5.01 (s, 2H), 4.94 (s, 2H), 3.26 (septet, J=7 Hz, 1H), 2.23 (s, 6H), 1.33 (d, J=7 Hz, 6H). HRMS (ESI) C₃₂H₃₂N₂O₅ calculated: 525.2389 (M+H)⁺, found: 525.2394 (M+H)⁺.

### Example 2

### 3-[(5-{[(5-(1-methylethyl)-3-{[(2,4,6-trifluorophenyl)oxy]methyl}-4-isoxazolyl)methyl]oxy}-1H-indol-1-yl)methyl]benzoic acid

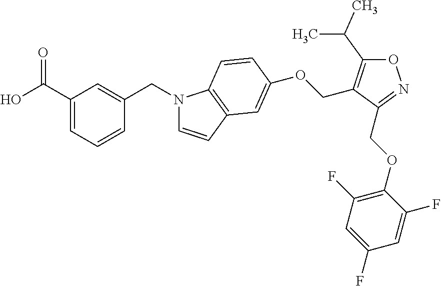

### 2a) Methyl 3-[(5-{[(5-(1-methylethyl)-3-{[(2,4,6-trifluorophenyl)oxy]methyl}-4-isoxazolyl)methyl]oxy}-1H-indol-1-yl)methyl]benzoate

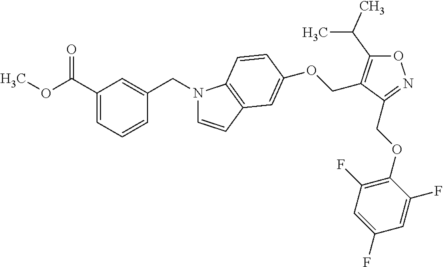

Prepared by a procedure similar to that used to prepare as methyl 3-{[5-({[3-{[(2,6-dimethylphenyl)oxy]methyl}-5-(1-methylethyl)-4-isoxazolyl]methyl}oxy)-1H-indol-1-yl]methyl}benzoate in Example 1g above using 2,4,6-trifluorophenol (24 mg, 0.16 mmol) to afford methyl 3-[(5-{[(5-(1-methylethyl)-3-{[(2,4,6-trifluorophenyl)oxy]methyl}-4-isoxazolyl)methyl]oxy}-1H-indol-1-yl)methyl]benzoate (43 mg, 47%). ¹H-NMR (400 MHz, CDCl₃): δ 7.94-7.90 (m, 2H), 7.34 (t, J=8 Hz, 1H), 7.20-7.11 (m, 4H), 6.83-6.80 (m, 1H), 6.62 (t, J=8 Hz, 2H), 6.49 (s, 1H), 5.32 (s, 2H), 5.19 (s, 2H), 5.05 (s, 2H), 3.89 (s, 3H), 3.25 (septet, J=7 Hz, 1H), 1.31 (d, J=7 Hz, 6H). APCI-LCMS m/z 565 (M+H)⁺.

### 2b) 3-[(5-{[(5-(1-methylethyl)-3-{[(2,4,6-trifluorophenyl)oxy]methyl}-4-isoxazolyl)methyl]oxy}-1H-indol-1-yl)methyl]benzoic acid

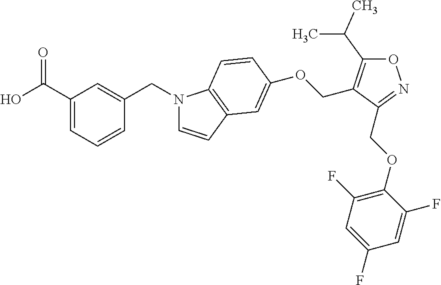

Prepared by a procedure similar to that used to prepare as 3-{[5-({[3-{[(2,6-dimethylphenyl)oxy]methyl}-5-(1-methylethyl)-4-isoxazolyl]methyl}oxy)-1H-indol-1-yl]methyl}benzoic acid in Example 1h above using methyl 3-[(5-{[(5-(1-methylethyl)-3-{[(2,4,6-trifluorophenyl)oxy]methyl}-4-isoxazolyl)methyl]oxy}-1H-indol-1-yl)methyl]benzoate (43 mg, 0.076 mmol) to afford 3-[(5-{[(5-(1-methylethyl)-3-{[(2,4,6-trifluorophenyl)oxy]methyl}-4-isoxazolyl)methyl]oxy}-1H-indol-1-yl)methyl]benzoic acid (18 mg, 43%). ¹H-NMR (400 MHz, CDCl₃): δ 8.01-7.94 (m, 2H), 7.38 (t, J=8 Hz, 1H), 7.23-7.11 (m, 4H), 6.83-6.80 (m, 1H), 6.61 (t, J=8 Hz, 2H), 6.50 (s, 1H), 5.34 (s, 2H), 5.19 (s, 2H), 5.05 (s, 2H), 3.24 (septet, J=7 Hz, 1H), 1.31 (d, J=7 Hz, 6H). HRMS (ESI) C₃₀H₂₅F₃N₂O₅ calculated: 551.1794 (M+H)⁺, found: 551.1790 (M+H)⁺.

### Example 3

### 3-[(5-{[(5-(1-methylethyl)-3-{[(2,4,6-trichlorophenyl)oxy]methyl}-4-isoxazolyl)methyl]oxy}-1H-indol-1-yl)methyl]benzoic acid

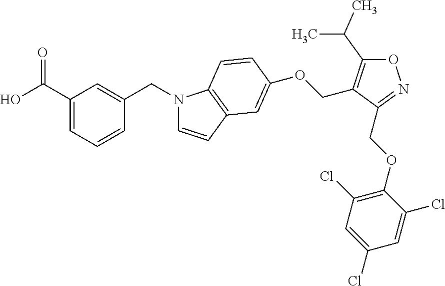

### 3a) Methyl 3-[(5-{[(5-(1-methylethyl)-3-{[(2,4,6-trichlorophenyl)oxy]methyl}-4-isoxazolyl)methyl]oxy}-1H-indol-1-yl)methyl]benzoate

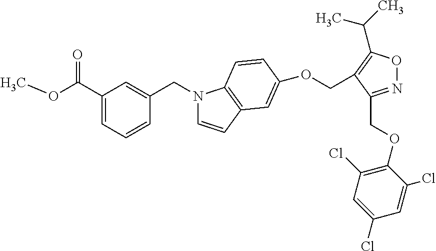

Prepared by a procedure similar to that used to prepare methyl 3-{[5-({[3-{[(2,6-dimethylphenyl)oxy]methyl}-5-(1-methylethyl)-4-isoxazolyl]methyl}oxy)-1H-indol-1-yl]methyl}benzoate in Example 1g above using 2,4,6-trichlorophenol (32 mg, 0.16 mmol) to afford methyl 3-[(5-{[(5-(1-methylethyl)-3-{[(2,4,6-trichlorophenyl)oxy]methyl}-4-isoxazolyl)methyl]oxy}-1H-indol-1-yl)methyl]benzoate (23 mg, 23%). ¹H-NMR (400 MHz, CDCl₃): δ 7.94-7.90 (m, 2H), 7.34 (t, J=8 Hz, 1H), 7.27-7.11 (m, 6H), 6.84-6.81 (m, 1H), 6.49 (s, 1H), 5.32 (s, 2H), 5.15 (s, 2H), 5.09 (s, 2H), 3.89 (s, 3H), 3.26 (septet, J=7 Hz, 1H), 1.33 (d, J=7 Hz, 6H). APCI-LCMS m/z 613 (M+H)⁺.

### 3b) 3-[(5-{[(5-(1-methylethyl)-3-{[(2,4,6-trichlorophenyl)oxy]methyl}-4-isoxazolyl)methyl]oxy}-1H-indol-1-yl)methyl]benzoic acid

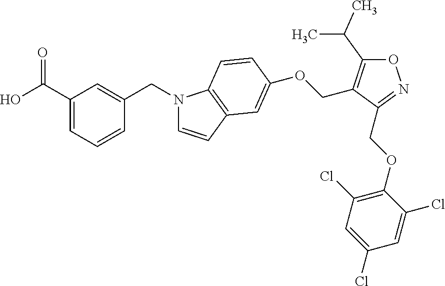

To a solution of methyl 3-[(5-{[(5-(1-methylethyl)-3-{[(2,4,6-trichlorophenyl)oxy]methyl}-4-isoxazolyl)methyl]oxy}-1H-indol-1-yl)methyl]benzoate (23 mg, 0.037 mmol) in 1:1 tetrahydrofuran-methanol (1.5 mL) was added 1 N sodium hydroxide (0.11 mL, 0.11 mmol). The solution was heated in a microwave reactor at 120° C. for 500 seconds. The solution was concentrated, and water followed by 1 N hydrochloric acid (0.11 mL, 0.11 mmol) were added. The solution was extracted with ethyl acetate and the organic layer was dried over magnesium sulfate and concentrated to afford 3-[(5-{[(5-(1-methylethyl)-3-{[(2,4,6-trichlorophenyl)oxy]methyl}-4-isoxazolyl)methyl]oxy}-1H-indol-1-yl)methyl]benzoic acid (21 mg, 88%). ¹H-NMR (400 MHz, CDCl₃): δ 8.01-7.94 (m, 2H), 7.39 (t, J=8 Hz, 1H), 7.26-7.12 (m, 6H), 6.85-6.82 (m, 1H), 6.49 (s, 1H), 5.35 (s, 2H), 5.16 (s, 2H), 5.09 (s, 2H), 3.26 (septet, J=7 Hz, 1H), 1.33 (d, J=7 Hz, 6H). HRMS (ESI) C₃₀H₂₅Cl₃N₂O₅ calculated: 599.0907 (M+H)⁺, found: 599.0903 (M+H)⁺.

### Example 4

### 3-{[5-({[3-{[(2,6-dichlorophenyl)amino]methyl}-5-(1-methylethyl)-4-isoxazolyl]methyl}oxy)-1H-indol-1-yl]methyl}benzoic acid

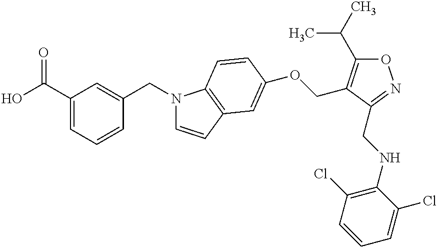

### 4a) N-(2,6-dichlorophenyl)-2,2,2-trifluoroacetamide

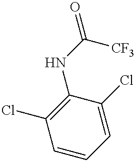

To a solution of 2,6-dichloroaniline (243 mg, 1.50 mmol) in dichloromethane (50 mL) at 0° C. was added trifluoroacetic anhydride (0.254 mL, 1.80 mmol), dropwise. The solution was stirred while the flask was in the cold bath for approximately 2 hours, then at ambient temperature for approximately 1 hour. The solution was concentrated, and the residue was diluted with ethyl acetate, washed with aqueous sodium bicarbonate, dried over magnesium sulfate and concentrated to afford N-(2,6-dichlorophenyl)-2,2,2-trifluoroacetamide (359 mg, 93%). ¹H-NMR (400 MHz, DMSO-d₆): δ 11.58 (s, 1H), 7.62 (d, J=8 Hz, 1H), 7.48-7.44 (m, 2H).

### 4b) Methyl 3-{[5-({[3-{[(2,6-dichlorophenyl)(trifluoroacetyl)amino]methyl}-5-(1-methylethyl)-4-isoxazolyl]methyl}oxy)-1H-indol-1-yl]methyl}benzoate

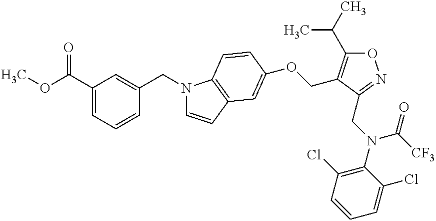

To a solution of N-(2,6-dichlorophenyl)-2,2,2-trifluoroacetamide (42 mg, 0.16 mmol), triphenylphosphine (42 mg, 0.16 mmol) and methyl 3-{[5-({[3-(hydroxymethyl)-5-(1-methylethyl)-4-isoxazolyl]methyl}oxy)-1H-indol-1-yl]methyl}benzoate (70 mg, 0.16 mmol) in toluene (2.5 mL) was added diisopropyl azodicarboxylate (0.029 mL, 0.16 mmol). The solution was heated in a microwave reactor at 90° C. for 10 minutes. The solution was adsorbed onto silica gel and purified by chromatography (silica gel, 0-25% ethyl acetate in hexanes gradient elution) to afford methyl 3-{[5-({[3-{[(2,6-dichlorophenyl)(trifluoroacetyl)amino]methyl}-5-(1-methylethyl)-4-isoxazolyl]methyl}oxy)-1H-indol-1-yl]methyl}benzoate (56 mg, 0.083 mmol). APCI-LCMS m/z 674 (M+H)⁺.

### 4c) 3-{[5-({[3-{[(2,6-dichlorophenyl)amino]methyl}-5-(1-methylethyl)-4-isoxazolyl]methyl}oxy)-1H-indol-1-yl]methyl}benzoic acid

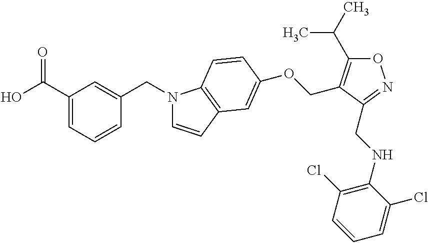

To a solution of methyl 3-{[5-({[3-{[(2,6-dichlorophenyl)(trifluoroacetyl)amino]methyl}-5-(1-methylethyl)-4-isoxazolyl]methyl}oxy)-1H-indol-1-yl]methyl}benzoate (56 mg, 0.083 mmol) in 1:1 tetrahydrofuran-methanol (1.5 mL) was added 1 N sodium hydroxide (0.25 mL, 0.25 mmol). The solution was heated in a microwave reactor at 120° C. for 500 seconds.

The solution was concentrated and 1 N hydrochloric acid (0.25 mL, 0.25 mmol) was added followed by ethyl acetate. The mixture was washed with brine and concentrated to afford 3-{[5-({[3-{[(2,6-dichlorophenyl)amino]methyl}-5-(1-methylethyl)-4-isoxazolyl]methyl}oxy)-1H-indol-1-yl]methyl}benzoic acid (29 mg, 58% as 0.4 ethyl acetate). ¹H-NMR (400 MHz, CDCl₃): δ 8.94 (br s, 2H), 8.00 (d, J=8 Hz, 1H), 7.93 (s, 1H), 7.38 (t, J=8 Hz, 1H), 7.24 (d, J=7 Hz, 1H), 7.19 (d, J=8 Hz, 2H), 7.15-7.11 (m, 3H), 6.84-6.77 (m, 2H), 6.50 (d, J=4 Hz, 1H), 5.34 (s, 2H), 4.89 (s, 2H), 4.60 (s, 2H), 3.20 (septet, J=7 Hz, 1H), 1.31 (d, J=7 Hz, 6H). HRMS (ESI) C₃₀H₂₇Cl₂N₃O₄ calculated: 564.1457 (M+H)⁺, found: 564.1453 (M+H)⁺.

### Example 5

### 3-{[5-({[3-{[(2,6-dibromophenyl)oxy]methyl}-5-(1-methylethyl)-4-isoxazolyl]methyl}oxy)-1H-indol-1-yl]methyl}benzoic acid

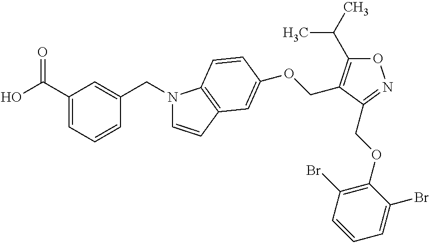

### 5a) Methyl 3-{[5-({[3-{[(2,6-dibromophenyl)oxy]methyl}-5-(1-methylethyl)-4-isoxazolyl]methyl}oxy)-1H-indol-1-yl]methyl}benzoate

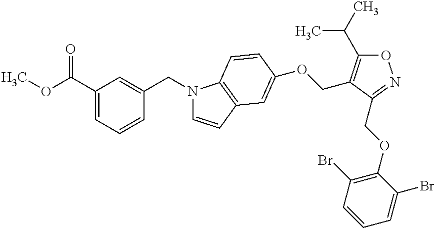

Prepared by a procedure similar to that used to prepare methyl 3-{[5-({[3-{[(2,6-dimethylphenyl)oxy]methyl}-5-(1-methylethyl)-4-isoxazolyl]methyl}oxy)-1H-indol-1-yl]methyl}benzoate in Example 1g above using 2,6-dibromophenol (41 mg, 0.16 mmol). The resulting material from the purification was purified by chromatography (5% methanol, 20% dichloromethane and 75% hexanes isocratic elution) to afford methyl 3-{[5-({[3-{[(2,6-dibromophenyl)oxy]methyl}-5-(1-methylethyl)-4-isoxazolyl]methyl}oxy)-1H-indol-1-yl]methyl}benzoate (49 mg, 0.073 mmol). ¹H-NMR (400 MHz, CDCl₃): δ 7.93 (d, J=8 Hz, 1H), 7.90 (s, 1H), 7.47 (d, J=8 Hz, 2H), 7.36 (d, J=3 Hz, 1H), 7.22-7.16 (m, 2H), 7.14-7.10 (m, 2H), 6.88-6.82 (m, 2H), 6.48 (d, J=3 Hz, 1H), 5.32 (s, 2H), 5.18 (s, 2H), 5.15 (s, 2H), 3.89 (s, 3H), 3.28 (septet, J=7 Hz, 1H), 1.34 (d, J=7 Hz, 6H). APCI-LCMS m/z 669 (M+H)⁺.

### 5b) 3-{[5-({[3-{[(2,6-dibromophenyl)oxy]methyl}-5-(1-methylethyl)-4-isoxazolyl]methyl}oxy)-1H-indol-1-yl]methyl}benzoic acid

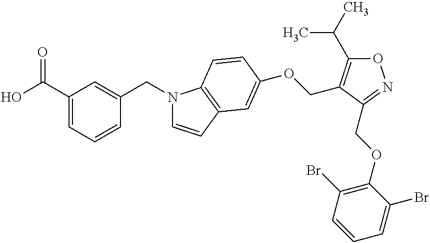

Prepared by a procedure similar to that used to prepare 3-{[5-({[3-{[(2,6-dimethylphenyl)oxy]methyl}-5-(1-methylethyl)-4-isoxazolyl]methyl}oxy)-1H-indol-1-yl]methyl}benzoic acid in Example 1h above using methyl 3-{[5-({[3-{[(2,6-dibromophenyl)oxy]methyl}-5-(1-methylethyl)-4-isoxazolyl]methyl}oxy)-1H-indol-1-yl]methyl}benzoate (49 mg, 0.073 mmol), 0.22 mL of 1 N sodium hydroxide and neutralized at the end with 0.22 mL of 1 N hydrochloric acid to afford 3-{[5-({[3-{[(2,6-dibromophenyl)oxy]methyl}-5-(1-methylethyl)-4-isoxazolyl]methyl}oxy)-1H-indol-1-yl]methyl}benzoic acid (33 mg, 69%). ¹H-NMR (400 MHz, CDCl₃): δ 7.99 (d, J=8 Hz, 1H), 7.93 (s, 1H), 7.46 (d, J=8 Hz, 2H), 7.38 (t, J=8 Hz, 1H) 7.25-7.21 (m, 2H), 7.14-7.11 (m, 2H), 6.85 (t, J=8 Hz, 2H), 6.50 (d, J=3 Hz, 1H), 5.34 (s, 2H), 5.18 (s, 2H), 5.15 (s, 2H), 3.28 (septet, J=7 Hz, 1H), 1.34 (d, J=7 Hz, 6H). HRMS (ESI) C₃₀H₂₆Br₂N₂O₅ calculated: 653.0287 (M+H)⁺, found: 653.0283 (M+H)⁺.

### Example 6

### 3-({5-[({5-(1-methylethyl)-3-[(1,3-thiazol-2-ylthio)methyl]-4-isoxazolyl}methyl)oxy]-1H-indol-1-yl}methyl)benzoic acid

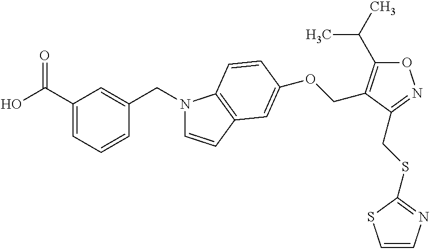

### 6a) Methyl 3-({5-[({5-(1-methylethyl)-3-[(1,3-thiazol-2-ylthio)methyl]-4-isoxazolyl}methyl)oxy]-1H-indol-1-yl}methyl)benzoate

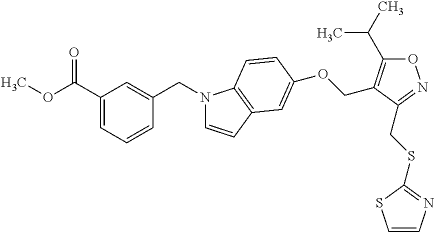

Prepared by a procedure similar to that used to prepare methyl 3-{[5-({[3-{[(2,6-dimethylphenyl)oxy]methyl}-5-(1-methylethyl)-4-isoxazolyl]methyl}oxy)-1H-indol-1-yl]methyl}benzoate in Example 1g above using 1,3-thiazole-2(3H)-thione (19 mg, 0.16 mmol) to afford methyl 3-({5-[({5-(1-methylethyl)-3-[(1,3-thiazol-2-ylthio)methyl]-4-isoxazolyl}methyl)oxy]-1H-indol-1-yl}methyl)benzoate (43 mg, 50%). ¹H-NMR (400 MHz, CDCl₃): δ 7.93 (d, J=8 Hz, 1H), 7.89 (s, 1H), 7.60 (d, J=3 Hz, 1H), 7.34 (t, J=8 Hz, 1H), 7.20-7.10 (m, 5H), 6.80 (dd, J=2, 9 Hz, 1H), 6.48 (d, J=3 Hz, 1H), 5.32 (s, 2H), 4.93 (s, 2H), 4.51 (s, 2H), 3.89 (s, 3H), 3.18 (septet, J=7 Hz, 1H), 1.29 (d, J=7 Hz, 6H). LRMS APCI-LCMS m/z 534 (M+H)⁺.

### 6b) 3-({5-[({5-(1-methylethyl)-3-[(1,3-thiazol-2-ylthio)methyl]-4-isoxazolyl}methyl)oxy]-1H-indol-1-yl}methyl)benzoic acid

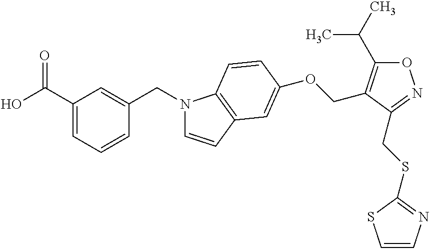

Prepared by a procedure similar to that used to prepare 3-{[5-({[3-{[(2,6-dimethylphenyl)oxy]methyl}-5-(1-methylethyl)-4-isoxazolyl]methyl}oxy)-1H-indol-1-yl]methyl}benzoic acid in Example 1h above using methyl 3-({5-[({5-(1-methylethyl)-3-[(1,3-thiazol-2-ylthio)methyl]-4-isoxazolyl}methyl)oxy]-1H-indol-1-yl}methyl)benzoate (43 mg, 0.081 mmol), 0.24 mL of 1 N sodium hydroxide and neutralized at the end with 0.24 mL of hydrochloric acid to afford 3-({5-[({5-(1-methylethyl)-3-[(1,3-thiazol-2-ylthio)methyl]-4-isoxazolyl}methyl)oxy]-1H-indol-1-yl}methyl)benzoic acid (22 mg, 49% as 0.35 ethyl acetate). ¹H-NMR (400 MHz, CDCl₃): δ 7.99 (d, J=8 Hz, 1H), 7.91 (s, 1H), 7.64 (d, J=3 Hz, 1H), 7.37 (t, J=8 Hz, 1H), 7.25-7.22 (m, 1H), 7.17-7.10 (m, 4H), 6.80 (d, J=2, 9 Hz, 1H), 6.48 (d, J=3 Hz, 1H), 5.33 (s, 2H), 4.93 (s, 2H), 4.49 (s, 2H), 3.18 (septet, J=7 Hz, 1H), 1.28 (d, J=7 Hz, 6H). HRMS (ESI) C₂₇H₂₅N₃O₄S₂ calculated: 520.1365 (M+H)⁺, found: 520.1364 (M+H)⁺.

### Example 7

### 3-[(5-{[(5-(1-Methylethyl)-3-{2-[(trifluoromethyl)oxy]phenyl}-4-isoxazolyl)methyl]oxy}-1H-indol-1-yl)methyl]benzoic acid

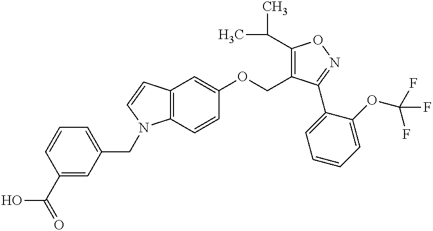

### 7a) 2-[(Trifluoromethyl)oxy]benzaldehyde oxime

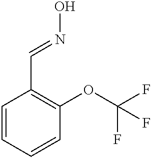

A solution of hydroxylamine hydrochloride (1.07 g, 15.40 mmol) and sodium hydroxide (0.67 g, 16.75 mmol) in water (10 mL) was added dropwise to a solution of 2-trifluoromethoxybenzaldehyde (2.00 mL, 14.01 mmol) in ethanol (20 mL). The mixture was stirred at 35° C. for 6 hours. Upon cooling, the mixture was concentrated. Water was added, and the mixture was extracted with ethyl acetate. The organic layer was dried over anhydrous magnesium sulfate, filtered, and concentrated to give 2.55 g (89%) of 2-[(trifluoromethyl)oxy]benzaldehyde oxime as a solid. ¹H NMR (400 MHz, CDCl₃): δ 8.41 (s, 1H), 7.88 (dd, J=8, 2 Hz, 1H), 7.45-7.40 (m, 1H), 7.33-7.27 (3H).

### 7b) N-Hydroxy-2-[(trifluoromethyl)oxy]benzenecarboximidoyl chloride

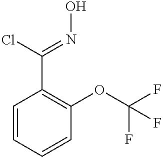

N-chlorosuccinimide (0.65 g, 4.90 mmol) was added to a solution of 2-[(trifluoromethyl)oxy]benzaldehyde oxime (1.00 g, 4.87 mmol) in N,N-dimethylformamide (3 mL). The mixture was stirred at room temperature overnight. The mixture was poured into water, and extracted with diethyl ether. The combined organics were dried with anhydrous magnesium sulfate, filtered, and concentrated to give 0.93 g (79%) of N-hydroxy-2-[(trifluoromethyl)oxy]benzenecarboximidoyl chloride as a solid. ¹H NMR (400 MHz, CDCl₃): δ 9.32 (s, 1H), 7.60 (dd, J=8, 2 Hz, 1H), 7.50-7.45 (m, 1H), 7.38-7.31 (m, 2H).

### 7c) Methyl 5-(1-methylethyl)-3-{2-[(trifluoromethyl)oxy]phenyl}-4-isoxazolecarboxylate

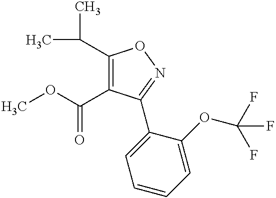

A 0.5 M solution of sodium hydroxide in methanol (10.5 mL) was added dropwise to a 0° C. solution of methyl isobutyryl acetate (0.75 mL, 5.27 mmol) in tetrahydrofuran (2 mL). After 5 minutes of stirring, a solution of N-hydroxy-2-[(trifluoromethyl)oxy]benzene-carboximidoyl chloride (0.93 g, 3.88 mmol) in tetrahydrofuran (5 mL) was added. The mixture was stirred at room temperature overnight. The mixture was concentrated. Water was added, and the resulting precipitate was filtered and dried under high vacuum to give 1.07 g (67%) of methyl 5-(1-methylethyl)-3-{2-[(trifluoromethyl)oxy]phenyl}-4-isoxazolecarboxylate as a solid. ¹H NMR (400 MHz, CDCl₃): δ 7.54-7.49 (m, 2H), 7.39-7.31 (m, 2H), 3.85-3.75 (m, 1H), 3.67 (s, 3H), 1.40 (d, J=7 Hz, 6H).

### 7d) (5-(1-Methylethyl)-3-{2-[(trifluoromethyl)oxy]phenyl}-4-isoxazolyl)methanol

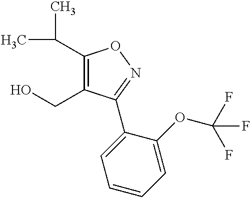

A 1.5 M solution of diisobutylaluminum hydride in toluene (2.2 mL, 3.30 mmol) was added, dropwise, to a 0° C. solution of methyl 5-(1-methylethyl)-3-{2-[(trifluoromethyl)oxy]phenyl}-4-isoxazolecarboxylate (0.51 g, 1.56 mmol) in tetrahydrofuran (4 mL). The mixture was warmed to room temperature and stirred for 4 hours. Rochelle's salt was added, and the mixture was stirred overnight. The mixture was extracted with ethyl acetate. The organics were dried over anhydrous magnesium sulfate, filtered, and concentrated. The residue was purified by silica gel chromatography eluting with a 10% to 35% ethyl acetate:hexanes gradient to give 0.178 g (38%) of (5-(1-methylethyl)-3-{2-[(trifluoromethyl)oxy]phenyl}-4-isoxazolyl)methanol as an oil. ¹H NMR (400 MHz, CDCl₃): δ 7.58 (dd, J=7, 2 Hz, 1H), 7.55-7.49 (m, 1H), 7.43-7.36 (m, 2H), 4.42 (s, 2H), 3.38-3.27 (m, 1H), 1.41 (d, J=7 Hz, 6H).

### 7e) Methyl 3-[(5-{[(5-(1-methylethyl)-3-{2-[(trifluoromethyl)oxy]phenyl}-4-isoxazolyl)methyl]oxy}-1H-indol-1-yl)methyl]benzoate

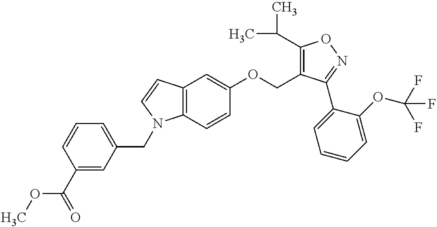

A mixture of methyl 3-[(5-hydroxy-1H-indol-1-yl)methyl]benzoate (0.167 g, 0.59 mmol, which may be prepared according to the general procedure described in Example 59b), (5-(1-methylethyl)-3-{2-[(trifluoromethyl)oxy]phenyl}-4-isoxazolyl)methanol (0.171 g, 0.567 mmol), polymer-bound triphenylphosphine (0.30 g, 0.63 mmol), and diisopropyl azodicarboxylate (0.11 mL, 0.56 mmol) in dichloromethane (8 mL) was stirred at room temperature for 3 days. The polymer was filtered off, and the resin was washed with dichloromethane. The filtrate was concentrated. The residue was purified by silica gel chromatography eluting with a 25% to 50% ethyl acetate:hexanes gradient to give 0.21 g (67%) of methyl 3-[(5-{[(5-(1-methylethyl)-3-{2-[(trifluoromethyl)oxy]phenyl}-4-isoxazolyl)methyl]oxy}-1H-indol-1-yl)methyl]benzoate as an oil. ¹H NMR (400 MHz, CDCl₃): δ 7.92 (d, J=7.81 Hz, 1H), 7.88 (s, 1H), 7.59-7.54 (m, 1H), 7.50-7.44 (m, 1H), 7.38-7.29 (m, 3H), 7.17 (d, J=8 Hz, 1H), 7.10 (d, J=3 Hz, 1H), 7.06 (d, J=9 Hz, 1H), 6.99 (d, J=2 Hz, 1H), 6.67 (dd, J=9, 2 Hz, 1H), 6.42 (d, J=3 Hz, 1H), 5.30 (s, 2H), 4.79 (s, 2H), 3.89 (s, 3H), 3.36-3.25 (m, 1H), 1.37 (d, J=7 Hz, 6H).

### 7f) 3-[(5-{[(5-(1-Methylethyl)-3-{2-[(trifluoromethyl)oxy]phenyl}-4-isoxazolyl)methyl]oxy}-1H-indol-1-yl)methyl]benzoic acid

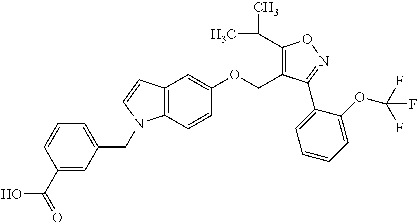

A 1N solution of aqueous sodium hydroxide (0.65 mL, 0.65 mmol) was added to a solution of methyl 3-[(5-{[(5-(1-methylethyl)-3-{2-[(trifluoromethyl)oxy]phenyl}-4-isoxazolyl)methyl]oxy}-1H-indol-1-yl)methyl]benzoate (0.18 g, 0.33 mmol) in a mixture of tetrahydrofuran and methanol (4:2 mL). The mixture was heated at 65° C. for 3 hours. Upon cooling, the mixture was acidified with 1 M aqueous hydrochloric acid, and extracted with ethyl acetate. The organics were washed with water and brine, dried over anhydrous magnesium sulfate, filtered, and concentrated. The residue was purified by silica gel chromatography eluting with ethyl acetate and hexanes to give 0.118 g (65%) of 3-[(5-{[(5-(1-methylethyl)-3-{2-[(trifluoromethyl)oxy]phenyl}-4-isoxazolyl)methyl]oxy}-1H-indol-1-yl)methyl]benzoic acid as a solid. ¹H NMR (400 MHz, CDCl₃): δ 7.99 (d, J=8 Hz, 1H), 7.93 (s, 1H), 7.61-7.54 (m, 1H), 7.51-7.44 (m, 1H), 7.41-4.30 (m, 3H), 7.25-7.19 (m, 1H), 7.12 (d, J=3 Hz, 1H), 7.06 (d, J=9 Hz, 1H), 7.00 (d, J=2 Hz, 1H), 6.68 (dd, J=9, 2 Hz, 1H), 6.43 (d, J=3 Hz, 1H), 5.32 (s, 2H), 4.80 (s, 2H), 3.36-3.25 (m, 1H), 1.37 (d, J=7 Hz, 6H). ESI-LCMS m/z 551 (M+H)⁺.

### Example 8

### 3-{[6-({[3-(3,5-dichloro-4-pyridinyl)-5-(1-methylethyl)-4-isoxazolyl]methyl}oxy)-1H-indol-1-yl]methyl}benzoic acid

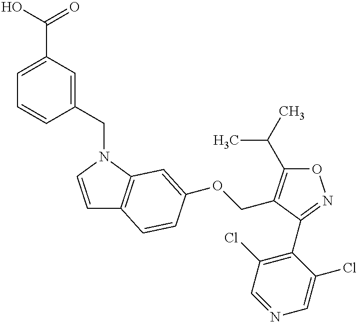

### 8a) 3-(3,5-Dichloro-4-pyridinyl)-5-(1-methylethyl)-4-isoxazolecarboxylic acid

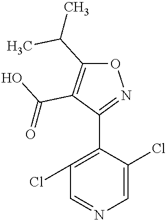

N-chlorosuccinimide (1.36 g, 10.2 mmol) was added to a stirred solution of 3,5-dichloro-4-pyridinecarbaldehyde oxime (1.94 g, 10.2 mmol) in N,N-dimethylformamide (8 mL) and the solution was heated in a 65° C. oil bath for 1.5 hours. The solution was poured into water and extracted with ether. The organic layer was dried with magnesium sulfate, filtered, and concentrated to yield a crude carboximidoyl chloride. A solution of methylisobutyrylacetate (1.7 mL, 12.3 mmol) in tetrahydrofuran (2.5 mL) was stirred at 0° C. as 0.5 N solution of sodium methoxide in methanol (24.6 mL, 12.3 mmol) was added. The solution was allowed to stir for ten minutes before the addition of the crude 3,5-dichloro-N-hydroxy-4-pyridinecarboximidoyl chloride in tetrahydrofuran (8.1 mL). The solution was allowed to stir at room temperature overnight. The solution was then concentrated and the residue was partitioned between water and ethyl acetate. The organic layer was dried with magnesium sulfate, filtered and concentrated. The residue was purified by chromatography (silica gel, hexane to 1:9 ethyl acetate:hexanes). Fractions containing the intermediate were combined and concentrated. The residue was azetroped with methanol then was diluted with tetrahydrofuran (11 mL) and methanol (5.5 mL). A 1 N solution of sodium hydroxide (3.3 mL) was added and the solution of heated to 100° C. for 500 seconds in a microwave reactor. The solution was neutralized with 1 N hydrochloric acid and concentrated to yield a white solid. The residue was slurried in water and filtered to yield 3-(3,5-dichloro-4-pyridinyl)-5-(1-methylethyl)-4-isoxazolecarboxylic acid (0.57 g, 18%). ¹H NMR (400 MHz, DMSO-d6): δ 13.39 (s, 1H), 8.81 (s, 2H), 3.82 (septet, J=7 Hz, 1H), 1.34 (d, J=7 Hz, 6H).

### 8b) [3-(3,5-Dichloro-4-pyridinyl)-5-(1-methylethyl)-4-isoxazolyl]methanol

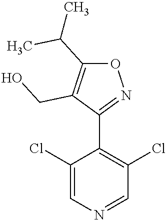

A solution of 3-(3,5-dichloro-4-pyridinyl)-5-(1-methylethyl)-4-isoxazolecarboxylic acid (0.54 g, 1.8 mmol) in tetrahydrofuran (9 mL), was stirred as triethylamine (0.25 mL, 1.8 mmol) was added. The solution was cooled in an ice bath before the addition of a 1 N solution of isopropylchloroformate in toluene (1.8 mL, 1.8 mmol). The solution was allowed to stir for 30 minutes before being filtered into a solution of sodium borohydride (91 mg, 2.4 mmol) in water (0.62 mL). The mixture was allowed to warm to room temperature and stir for 3 days. The mixture was filtered and the filtrate was partitioned between brine and ethyl acetate. The organic layer was dried with magnesium sulfate, filtered and concentrated. The residue was purified by chromatography (silica gel, hexane to 2:3 ethyl acetate:hexanes) to provide [3-(3,5-dichloro-4-pyridinyl)-5-(1-methylethyl)-4-isoxazolyl]methanol (0.32 g, 57% as 0.3 ethyl acetate). ¹H NMR (400 MHz, DMSO-d₆): δ 8.79 (s, 2H), 4.96 (t, J=5 Hz, 1H), 4.20 (d, J=5 Hz, 2H), 3.35 (septet, J=7 Hz, 1H), 1.29 (d, J=7 Hz, 6H).

### 8c) Methyl 3-{[6-({[3-(3,5-dichloro-4-pyridinyl)-5-(1-methylethyl)-4-isoxazolyl]methyl}oxy)-1H-indol-1-yl]methyl}benzoate

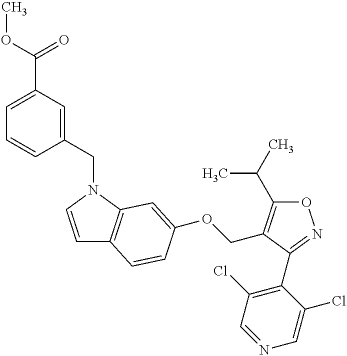

A mixture of [3-(3,5-dichloro-4-pyridinyl)-5-(1-methylethyl)-4-isoxazolyl]methanol (152 mg, 0.53 mmol), triphenylphosphine (139 mg, 0.53 mmol), methyl 3-[(6-hydroxy-1H-indol-1-yl)methyl]benzoate (150 mg, 0.53 mmol), in toluene (4.5 mL) and diisopropyl azodicarboxylate (0.104 mL, 0.53 mmol) was heated to 85° C. in a microwave reaction tube for 500 seconds. The mixture was reheated to 85° C. for another 500 seconds. The reaction mixture was concentrated and the residue was purified by chromatography (silica gel, hexane to 2:3 ethyl acetate:hexane). Fractions containing the product were combined and concentrated to yield methyl 3-{[6-({[3-(3,5-dichloro-4-pyridinyl)-5-(1-methylethyl)-4-isoxazolyl]methyl}oxy)-1H-indol-1-yl]methyl}benzoate

(65 mg, 22%). ¹H NMR (400 MHz, d₆-DMSO): δ 8.76 (s, 2H), 7.80 (d, J=7 Hz, 1H), 7.73 (s, 1H), 7.45-7.39 (m, 3H), 7.20 (d, J=9 Hz, 1H), 6.92 (d, J=2 Hz, 1H), 6.42 (dd, J=2, 9 Hz, 1H), 6.32 (d, J=3 Hz, 1H), 5.41 (s, 2H), 4.83 (s, 2H), 3.78 (s, 3H), 3.40 (septet, J=7 Hz, 1H), 1.27 (d, J=7 Hz, 6H).

### 8d) 3-{[6-({[3-(3,5-dichloro-4-pyridinyl)-5-(1-methylethyl)-4-isoxazolyl]methyl}oxy)-1H-indol-1-yl]methyl}benzoic acid

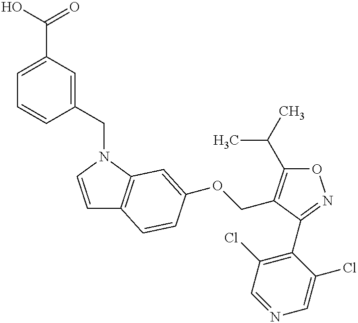

A mixture of 1 N solution of sodium hydroxide (200 μL, 200 μmol) and methyl 3-{[6-({[3-(3,5-dichloro-4-pyridinyl)-5-(1-methylethyl)-4-isoxazolyl]methyl}oxy)-1H-indol-1-yl]methyl}benzoate (61 mg, 11 mmol) in methanol (0.5 mL) and tetrahydrofuran (1 mL) was heated in the microwave at 100° C. for 1000 seconds. The mixture neutralized with 1 N hydrochloric acid was concentrated. The residue was extracted with ethyl acetate and concentrated to yield 61 mg (99% as 0.25 ethyl acetate) of 3-{[6-({[3-(3,5-dichloro-4-pyridinyl)-5-(1-methylethyl)-4-isoxazolyl]methyl}oxy)-1H-indol-1-yl]methyl}benzoic acid. ¹H NMR (400 MHz, d₆-DMSO): δ 12.32 (br s, 1H), 8.76 (s, 2H), 7.77 (d, J=7 Hz, 1H), 7.68 (s, 1H), 7.44 (d, J=3 Hz, 1H), 7.42-7.35 (m, 2H) 7.20 (d, J=9 Hz, 1H), 6.92 (d, J=2 Hz, 1H), 6.42 (dd, J=2, 9 Hz, 1H), 6.32 (d, J=3 Hz, 1H), 5.40 (s, 2H), 4.83 (s, 2H), 3.40 (septet, J=7 Hz, 1H), 1.27 (d, J=7 Hz, 6H). HRMS C₂₈H₂₃Cl₂N₃O₄ m/z 536.1144 (M+H)⁺_{Cal}; 536.1140 (M+H)⁺_{Obs}.

### Example 9

### 4-[6-({[3-(2,6-Dichlorophenyl)-5-(1-methylethyl)-4-isoxazolyl]methyl}oxy)-1-benzothien-2-yl]benzoic acid

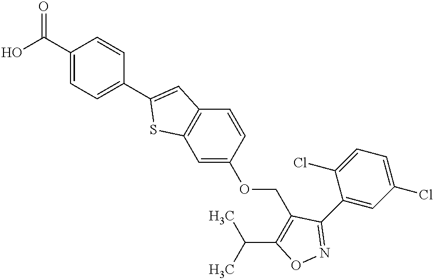

### 9a) Ethyl 4-[6-(methyloxy)-1-benzothien-2-yl]benzoate

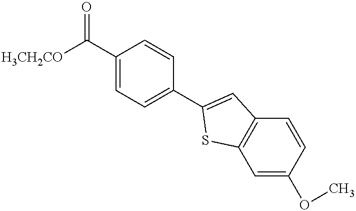

[6-(Methyloxy)-1-benzothien-2-yl]boronic acid (prepared according to the general procedure described in Example 36c) (0.35 g, 1.68 mmol), ethyl-4-iodobenzoate (0.32 mL, 1.92 mmol), sodium carbonate (2 M) (2 mL, 4 mmol), tetrakistriphenylphosphinepalladium(0) (0.080 g, 0.07 mmol), and toluene (10 mL) were combined and heated overnight at reflux with stirring under a nitrogen atmosphere. The reaction mixture was partitioned between ethyl acetate and water. The organic phase was separated, dried over magnesium sulfate, filtered, and the filtrate was concentrated to give the crude product. The crude product was purified by flash chromatography over silica with a hexanes:ethyl acetate gradient (100:0 to 80:20) to give 0.19 g (36%) of ethyl 4-[6-(methyloxy)-1-benzothien-2-yl]benzoate as a white solid. The solid became pale pink upon standing. ¹H NMR (CDCl₃; 400 MHz): δ 8.07 (d, J=8 Hz, 2H), 7.72 (d, J=8 Hz, 2H), 7.67 (d, J=9 Hz, 1H), 7.57 (s, 1H), 7.31 (d, J=2 Hz, 1H), 6.99 (dd, J=9, 2 Hz, 1H), 4.40 (q, J=7 Hz, 2H), 3.89 (s, 3H), 1.41 (t, J=7 Hz, 3H). ES-LCMS: m/z 313 (M+H)⁺.

### 9b) Ethyl 4-(6-hydroxy-1-benzothien-2-yl)benzoate

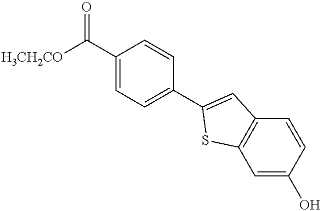

To a stirred ice-water cooled solution of ethyl 4-[6-(methyloxy)-1-benzothien-2-yl]benzoate (0.19 g, 0.61 mmol) in dichloromethane (10 mL) was slowly added, dropwise, boron tribromide (1M in dichloromethane) (2.4 mL, 2.4 mmol) under a nitrogen atmosphere. The reaction mixture was stirred at 0° C. for 1.5 h. The reaction mixture was poured onto ice and the mixture was partitioned between water and ethyl acetate. The organic phase was separated, dried over magnesium sulfate, filtered, and the filtrate was concentrated to give a pale yellow solid. The crude product was purified by flash chromatography over silica with a hexanes:ethyl acetate gradient (100:0 to 80:20) to give 0.090 g (49%) of ethyl 4-(6-hydroxy-1-benzothien-2-yl)benzoate as a white solid. ¹H NMR (DMSO-d₆; 400 MHz): δ 9.79 (s, 1H), 7.98 (d, J=8 Hz, 2H), 7.87 (s, 1H), 7.82 (d, J=8 Hz, 2H), 7.66 (d, J=9 Hz, 1H), 7.28 (d, J=2 Hz, 1H), 6.88 (dd, J=9, 2 Hz, 1H), 4.31 (q, J=7 Hz, 2H), 1.31 (t, J=7 Hz, 3H).

### 9c) Ethyl 4-[6-({[3-(2,6-dichlorophenyl)-5-(1-methylethyl)-4-isoxazolyl]methyl}oxy)-1-benzothien-2-yl]benzoate

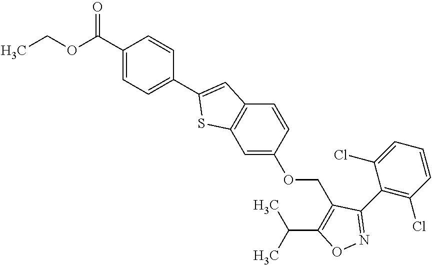

To a stirred mixture of ethyl 4-(6-hydroxy-1-benzothien-2-yl)benzoate (0.086 g, 0.288 mmol), [3-(2,6-dichlorophenyl)-5-(1-methylethyl)-4-isoxazolyl]methanol (0.0825 g, 0.288 mmol) and triphenylphosphine (0.076 g, 0.288 mmol) in dichloromethane (5 mL) at 0° C. was slowly added a solution of diisopropyl azodicarboxylate (0.057 mL, 0.288 mmol) in dichloromethane (2 mL). The reaction mixture was stirred at 0° C. for 10 min, then it was taken out of the ice-bath and stirred at room temperature. After stirring for 1 day at room temperature, the reaction mixture was concentrated to give an oil. The crude oil was purified using a hexanes:ethyl acetate gradient of 0 to 20% ethyl acetate to afford 0.092 g (56%) of ethyl 4-[6-({[3-(2,6-dichlorophenyl)-5-(1-methylethyl)-4-isoxazolyl]methyl}oxy)-1-benzothien-2-yl]benzoate. ¹H NMR (400 MHz, DMSO-d₆): δ 7.99 (d, J=9 Hz, 2H), 7.90 (s, 1H), 7.83 (d, J=9 Hz, 2H), 7.67 (d, J=9 Hz, 1H), 7.61 (m, 2H), 7.53 (dd, J=9, 7 Hz, 1H), 7.48 (d, J=2 Hz, 1H), 6.78 (dd, J=9, 2 Hz, 1H), 4.89 (s, 2H), 4.31 (q, J=7 Hz, 2H), 3.47 (septet, J=7 Hz, 1H), 1.34-1.29 (m, 9H)

### 9d) 4-[6-({[3-(2,6-Dichlorophenyl)-5-(1-methylethyl)-4-isoxazolyl]methyl}oxy)-1-benzothien-2-yl]benzoic acid

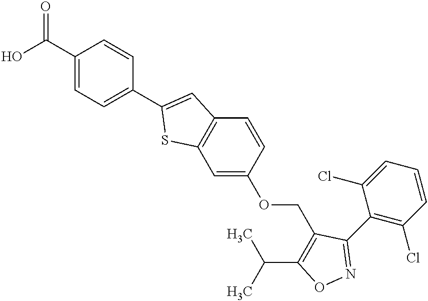

Ethyl 4-[6-({[3-(2,6-dichlorophenyl)-5-(1-methylethyl)-4-isoxazolyl]methyl}oxy)-1-benzothien-2-yl]benzoate (0.086 g, 0.15 mmol) and 1 N lithium hydroxide (1 mL) were stirred in tetrahydrofuran (1.5 mL) at room temperature for 24 h. 1,4-Dioxane (1.5 mL) was added to the reaction mixture and the reaction mixture was stirred for 4 days. The reaction mixture was concentrated, then diluted with saturated sodium hydrogensulfate and ethyl acetate. The layers were separated and the ethyl acetate layer was washed with water, followed by brine. The aqueous layer was extracted with ethyl acetate and the organic layers were washed with brine. The organic fractions were combined, dried over magnesium sulfate, filtered, and concentrated to a powder. The powder was dried at 60° C. overnight to afford 0.0696 g (85%) of 4-[6-({[3-(2,6-dichlorophenyl)-5-(1-methylethyl)-4-isoxazolyl]methyl}oxy)-1-benzothien-2-yl]benzoic acid. ¹H NMR (400 MHz, DMSO-d₆): δ 7.97 (d, J=8 Hz, 2H), 7.88 (s, 1H), 7.81 (d, J=9 Hz, 2H), 7.67 (d, J=9 Hz, 1H), 7.61 (m, 2H), 7.53 (dd, J=9, 7 Hz, 1H), 7.48 (d, J=2 Hz, 1H), 6.78 (dd, J=9, 2 Hz, 1H), 4.89 (s, 2H), 3.47 (septet, J=7 Hz, 1H), 1.32 (d, J=7 Hz, 6H). HRMS C₂₈H₂₁Cl₂NO₄S m/z 538.0647 (M+H)⁺_{Cal}; 538.0660 (M+H)⁺_{Obs}.

### Example 10

### 3-{[5-({[3-(2,6-Dichlorophenyl)-5-(1-methylethyl)-4-isoxazoly]methyl}oxy)-1H-indol-1-yl]carbonyl}benzoic acid

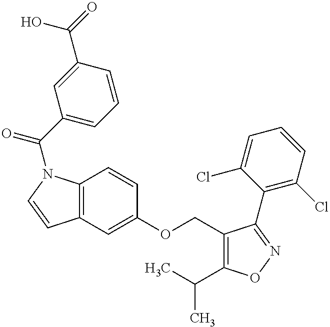

### 10a) Methyl 3-{[5-(methyloxy)-1H-indol-1-yl]carbonyl}benzoate

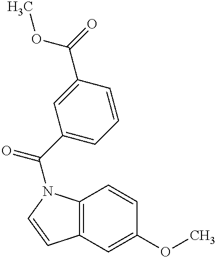

To a slurry of 3-[(methyloxy)carbonyl]benzoic acid (1 g, 5.55 mmol) in dichloromethane (16 mL) was slowly added oxalyl chloride (0.97 mL, 11.1 mmol), followed by N,N-dimethylformamide (2 drops) at room temperature. The reaction mixture was stirred for 1 h at room temperature and concentrated to afford methyl 3-(chlorocarbonyl)benzoate which was approximately 80% pure as determined by ¹H NMR. The impure methyl 3-(chlorocarbonyl)benzoate was used without purification.

Sodium hydride (60% dispersion in oil) (0.25 g, 6.18 mmol) was washed with hexanes, and N,N-dimethylformamide (10 mL) was added. The slurry was cooled to 0° C. A solution of 5-(methyloxy)-1H-indole (0.65 g, 4.43 mmol) in N,N-dimethylformamide (2 mL) was added slowly to the suspension of sodium hydride and the mixture was stirred for approximately 15 min. A solution of methyl 3-(chlorocarbonyl)benzoate (1.1 g, 5.54 mmol) in N,N-dimethylformamide (2 mL) was slowly added to the reaction mixture and the reaction mixture was stirred at 0° C. for 30 min, then at room temperature for 3 h. The reaction mixture was diluted with water, followed by ethyl acetate. The organic layer was separated, washed several times with water, followed by brine, dried over magnesium sulfate, filtered, and concentrated. The crude material was purified by flash chromatography over silica using a hexanes:ethyl acetate gradient of 0 to 35% ethyl acetate to afford 0.86 g (63%) of methyl 3-{[5-(methyloxy)-1H-indol-1-yl]carbonyl}benzoate. ¹H NMR (400 MHz, DMSO-d₆): δ 8.21 (m, 2H), 8.16 (d, J=9 Hz, 1H), 8.00 (d, J=8 Hz, 1H), 7.73 (t, J=8 Hz, 1H), 7.34 (d, J=4 Hz, 1H), 7.19 (d, J=3 Hz, 1H), 6.97 (dd, J=9, 2 Hz, 1H), 6.68 (d, J=4 Hz, 1H), 3.87 (s, 3H), 3.79 (s, 3H).

### 10b) Methyl 3-[(5-hydroxy-1H-indol-1-yl)carbonyl]benzoate

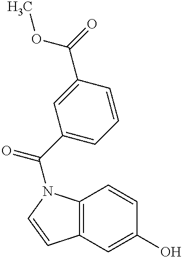

To a solution of methyl 3-{[5-(methyloxy)-1H-indol-1-yl]carbonyl}benzoate (0.86 g, 2.78 mmol) in dichloromethane (25 mL) at −60° C. was slowly added 1 M boron tribromide (11 mL, 11.1 mmol). The reaction mixture was stirred at −60° C. for 30 min then stirred at 0° C. for 3.5 h. The reaction mixture was poured into ice, stirred for several minutes, and extracted with ethyl acetate. The ethyl acetate was washed with brine, dried over magnesium sulfate, filtered, and concentrated. The crude material was purified by flash chromatography over silica using a hexanes:ethyl acetate gradient of 0 to 50% ethyl acetate to afford 0.618 g (75%) of methyl 3-[(5-hydroxy-1H-indol-1-yl)carbonyl]benzoate. ¹H NMR (400 MHz, DMSO-d₆): δ 9.38 (s, 1H), 8.22 (m, 2H), 8.11 (d, J=9 Hz, 1H), 8.02 (d, J=8 Hz, 1H), 7.76 (dd, J=8, 8 Hz, 1H), 7.29 (d, J=4 Hz, 1H), 6.99 (d, J=2 Hz, 1H), 6.84 (dd, J=9, 2 Hz, 1H), 6.64 (d, J=4 Hz, 1H), 3.90 (s, 3H).

### 10c) 5-({[3-(2,6-dichlorophenyl)-5-(1-methylethyl)-4-isoxazolyl]methyl}oxy)-1H-indole

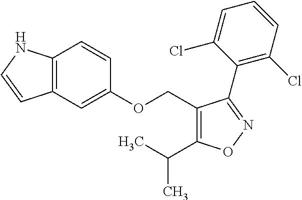

To a stirred mixture of methyl 3-[(5-hydroxy-1H-indol-1-yl)carbonyl]benzoate (0.3 g, 1.02 mmol), [3-(2,6-dichlorophenyl)-5-(1-methylethyl)-4-isoxazolyl]methanol (prepared by the general procedure described in Maloney, P. R., et al., 2000 J. Med. Chem. 43:2971-2974) (0.29 g, 1.02 mmol) and triphenylphosphine (0.266 g, 1.02 mmol) in dichloromethane (15 mL) at 0° C. was slowly added diisopropyl azodicarboxylate (0.2 mL, 1.02 mmol). After stirring for 2 days at room temperature, the reaction mixture was concentrated. The crude oil was purified by flash chromatography over silica using a hexanes:ethyl acetate gradient of 0 to 25% ethyl acetate to afford impure methyl 3-{[5-({[3-(2,6-dichlorophenyl)-5-(1-methylethyl)-4-isoxazolyl]methyl}oxy)-1H-indol-1-yl]carbonyl}benzoate. The ester was used without further purification. Methyl 3-{[5-({[3-(2,6-dichlorophenyl)-5-(1-methylethyl)-4-isoxazolyl]methyl}oxy)-1H-indol-1-yl]carbonyl}benzoate (0.35 g, 0.621 mmol) and 1 N lithium hydroxide (0.9 mL, 0.9 mmol) were stirred in 1,4-dioxane (2 mL) for 40 min. The reaction mixture was concentrated and ethyl acetate was added to the residue. The ethyl acetate phase was washed with water, dried over magnesium sulfate, filtered and concentrated. The crude material was purified by flash chromatography over silica using a hexanes:dichloromethane gradient of 0 to 70% dichloromethane to afford 0.137 g (55%) of 5-({[3-(2,6-dichlorophenyl)-5-(1-methylethyl)-4-isoxazolyl]methyl}oxy)-1H-indole. [Note: The intended product was 3-{[5-({[3-(2,6-dichlorophenyl)-5-(1-methylethyl)-4-isoxazoly]methyl}oxy)-1H-indol-1-yl]carbonyl}benzoic acid. The material obtained herein was acylated and hydrolyzed as described in the following steps.] ¹H NMR (400 MHz, DMSO-d₆) δ 10.87 (s, 1H), 7.61 (m, 2H), 7.52 (dd, J=9, 7 Hz, 1H), 7.23 (t, J=3 Hz, 1H), 7.16 (d, J=9 Hz, 1H), 6.90 (d, J=2 Hz, 1H), 6.48 (dd, J=9, 2 Hz, 1H), 6.24 (m, 1H), 4.73 (s, 2H), 3.39 (septet, J=7 Hz, 1H), 1.28 (d, J=7 Hz, 6H).

### 10d) 1,1-Dimethylethyl methyl 1,3-benzenedicarboxylate

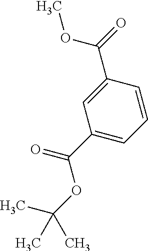

N,N-dimethylformamide di-tert-butyl acetal (10.6 mL, 44.4 mmol) was added over a period of 1 h to a stirred solution of mono-methyl isophthalate (2 g, 11.1 mmol) in toluene (21 mL) at room temperature. The reaction mixture was stirred at room temperature overnight, then it was heated at reflux for 24 hours. N,N-dimethylformamide di-tert-butyl acetal (5 mL, 21 mmol) was added to the reaction mixture and reflux was continued for another 24 hours. The reaction mixture was cooled to room temperature then diluted with 5% sodium carbonate, followed by ethyl acetate. The ethyl acetate layer was washed twice with 5% sodium carbonate, followed by brine, dried over magnesium sulfate, filtered and concentrated to an oil. The crude material was purified by flash chromatography over silica using a hexanes:ethyl acetate gradient of 0 to 20% ethyl acetate to afford 0.5 g (19%) of 1,1-dimethylethyl methyl 1,3-benzenedicarboxylate. ¹H NMR (400 MHz, DMSO-d₆): δ 8.40 (s, 1H), 8.13 (m, 2H), 7.64 (t, J=8 Hz, 1H), 3.86 (s, 3H), 1.54 (s, 9H).

### 10e) 3-{[(1,1-Dimethylethyl)oxy]carbonyl}benzoic acid

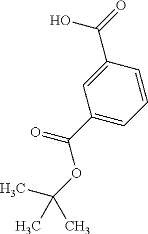

1,1-Dimethylethyl methyl 1,3-benzenedicarboxylate (0.5 g, 2.12 mmol) and 1 N lithium hydroxide (2 mL, 2 mmol) were stirred in 1,4-dioxane (2 mL) at room temperature overnight. The reaction mixture was heated at 60° C. for 30 min. Lithium hydroxide (1 N) (2 mL, 2 mmol) was added to the reaction mixture and heating was continued at 60° C. for another 1.5 hours. The reaction mixture was concentrated and diluted with ethyl acetate followed by saturated sodium hydrogensulfate. The organic layer was separated, washed with brine, dried over magnesium sulfate, filtered, and concentrated to afford 0.3 g (64%) of 3-{[(1,1-dimethylethyl)oxy]carbonyl}benzoic acid. ¹H NMR (400 MHz, DMSO-d₆): δ 13.25 (s, 1H), 8.40 (s, 1H), 8.12 (dd, J=8 Hz, 2H), 7.62 (t, J=8 Hz, 1H), 1.54 (s, 9H).

### 10f) 1,1-Dimethylethyl 3-{[5-({[3-(2,6-dichlorophenyl)-5-(1-methylethyl)-4-isoxazolyl]methyl}oxy)-1H-indol-1-yl]carbonyl}benzoate

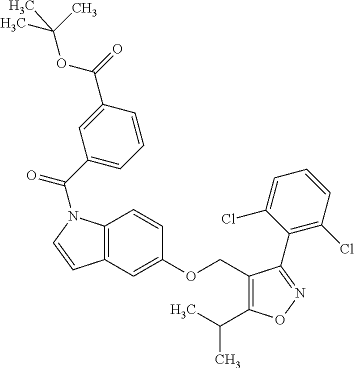

A solution of oxalyl chloride (0.088 mL, 1.01 mmol) in dichloromethane (0.5 mL) was added slowly to a solution of 3-{[(1,1-dimethylethyl)oxy]carbonyl}benzoic acid (0.15 g, 0.675 mmol) in dichloromethane (7 mL) at 0° C. To the reaction mixture was added N,N-dimethylformamide (2 drops). The reaction mixture was stirred at 0° C. for 30 min and concentrated to afford 0.13 g of 1,1-dimethylethyl 3-(chlorocarbonyl)benzoate. 1,1-dimethylethyl 3-(chlorocarbonyl)benzoate was used without purification. Sodium hydride (60% dispersion in oil) (0.021 g, 0.518 mmol) was washed with hexanes and N,N-dimethylformamide (1 mL) was added. The slurry of sodium hydride in N,N-dimethylformamide was cooled to 0° C. and a solution of 5-({[3-(2,6-dichlorophenyl)-5-(1-methylethyl)-4-isoxazolyl]methyl}oxy)-1H-indole (0.13 g, 0.324 mmol) in N,N-dimethylformamide (0.5 mL) was added slowly and stirred for approximately 15 min. To the reaction mixture was added a solution of 1,1-dimethylethyl 3-(chlorocarbonyl)benzoate (0.097 g, 0.405 mmol) in N,N-dimethylformamide (0.5 mL) and the reaction mixture was stirred at 0° C. for 30 min, and then at room temperature for 48 h. The reaction mixture was diluted with water, followed by ethyl acetate. The organic layer was separated, washed with brine, dried over magnesium sulfate, filtered, and concentrated. The crude material was purified by flash chromatography over silica using a hexanes:ethyl acetate gradient of 0 to 30% ethyl acetate to afford 0.037 g (19%) of 1,1-dimethylethyl 3-{[5-({[3-(2,6-dichlorophenyl)-5-(1-methylethyl)-4-isoxazolyl]methyl}oxy)-1H-indol-1-yl]carbonyl}benzoate. ¹H NMR (400 MHz, DMSO-d₆): δ 8.14 (m, 2H), 8.08 (d, J=9 Hz, 1H), 7.94 (m, 1H), 7.69 (m, 1H), 7.61 (m, 2H), 7.53 (dd, J=9, 7 Hz, 1H), 7.30 (d, J=4 Hz, 1H), 7.07 (d, J=3 Hz, 1H), 6.76 (dd, J=9, 3 Hz, 1H), 6.61 (dd, J=4, 1 Hz, 1H), 4.85 (s, 2H), 3.45 (septet, J=7 Hz, 1H), 1.53 (s, 9H), 1.31 (d, J=7 Hz, 6H).

### 10g) 3-{[5-({[3-(2,6-Dichlorophenyl)-5-(1-methylethyl)-4-isoxazoly]methyl}oxy)-1H-indol-1-yl]carbonyl}benzoic acid

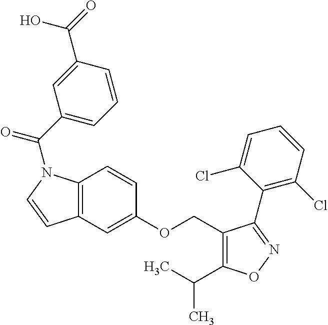

Trifluoroacetic acid (1 mL) was added slowly to a solution of 1,1-dimethylethyl 3-{[5-({[3-(2,6-dichlorophenyl)-5-(1-methylethyl)-4-isoxazolyl]methyl}oxy)-1H-indol-1-yl]carbonyl}benzoate (0.035 g, 0.058 mmol) in dichloromethane (3 mL) at 0° C. The reaction mixture was stirred for 3 h, then concentrated. The crude product was dissolved in toluene and the solution was concentrated. The gummy material was dissolved in methanol and the solution was concentrated. The crude material was purified by reverse phase preparative HPLC using an acetonitrile:water gradient (50-100% acetonitrile) with 0.05% trifluoroacetic acid as a modifier to afford 0.016 g (50%) of 3-{[5-({[3-(2,6-dichlorophenyl)-5-(1-methylethyl)-4-isoxazoly]methyl}oxy)-1H-indol-1-yl]carbonyl}benzoic acid. ¹H NMR (400 MHz, DMSO-d₆): δ 13.32 (s, 1H), 8.18 (m, 2H), 8.07 (d, J=9 Hz, 1H), 7.95 (d, J=8 Hz, 1H), 7.71 (m, 1H), 7.62 (m, 2H), 7.53 (dd, J=9, 7 Hz, 1H), 7.33 (d, J=4 Hz, 1H), 7.07 (d, J=2 Hz, 1H), 6.76 (dd, J=9, 3 Hz, 1H), 6.61 (d, J=4 Hz, 1H), 4.85 (s, 2H), 3.45 (septet, J=7 Hz, 1H), 1.31 (d, J=7 Hz, 6H). HRMS C₂₉H₂₂Cl₂N₂O₅ m/z 549.0984 (M+H)⁺_{Cal}; 549.0987 (M+H)⁺_{Obs}.

### Example 11

### 3-[6-({[3-(2,6-Dichlorophenyl)-5-(1-methylethyl)-4-isoxazolyl]methyl}oxy)-1,3-benzothiazol-2-yl]benzoic acid

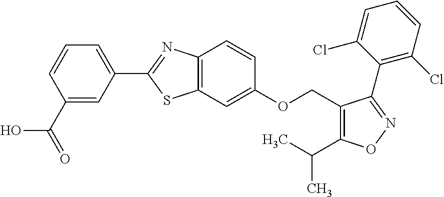

### 11a) 2-Bromo-6-(methyloxy)-1,3-benzothiazole

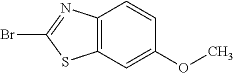

To a solution of copper (II) bromide (0.74 g, 3.33 mmol) in acetonitrile (12.5 mL) at 0° C. was added t-butylnitrite (0.495 mL, 4.16 mmol). To this mixture was added 2-amino-6-methoxybenzothiazole (0.5 g, 2.77 mmol) portion-wise via an addition funnel. The reaction mixture was stirred at room temperature for 24 hours. The reaction mixture was filtered through a pad of Celite® and the Celite® pad was washed with diethyl ether, followed by ethyl acetate. The filtrate was washed with 1N hydrochloric acid, followed by brine, dried over magnesium sulfate, filtered and concentrated to afford 0.57 g (84%) of 2-bromo-6-(methyloxy)-1,3-benzothiazole. ¹H NMR (400 MHz, DMSO-d₆): δ 7.85 (d, J=9 Hz, 1H), 7.68 (d, J=3 Hz, 1H), 7.10 (dd, J=9, 3 Hz, 1H), 3.80 (s, 3H).

### 11b) Methyl 3-[6-(methyloxy)-1,3-benzothiazol-2-yl]benzoate

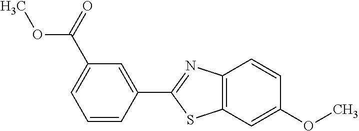

To a slurry of 2-bromo-6-(methyloxy)-1,3-benzothiazole (0.57 g, 2.34 mmol), tetrakis(triphenylphosphine)palladium(0) (0.108 g, 0.093 mmol), ethylene glycol dimethyl ether (14 mL) and 2 N sodium carbonate (11 mL, 22 mmol) was added {3-[(methyloxy)carbonyl]phenyl}boronic acid (0.5 g, 2.80 mmol) and the reaction mixture was heated at 80° C. for 1 h. The reaction mixture was cooled to room temperature then diluted with ethyl acetate, followed by water. The ethyl acetate layer was separated, washed with brine, dried over magnesium sulfate, filtered, and concentrated. The crude material was purified by flash chromatography over silica using a hexanes:ethyl acetate gradient of 0 to 30% ethyl acetate to afford 0.37 g (53%) of methyl 3-[6-(methyloxy)-1,3-benzothiazol-2-yl]benzoate. ¹H NMR (400 MHz, DMSO-d₆): δ 8.57 (t, J=2 Hz, 1H), 8.27 (d, J=8 Hz, 1H), 8.08 (d, J=8 Hz, 1H), 7.98 (d, J=9 Hz, 1H), 7.74 (d, J=3 Hz, 1H), 7.70 (t, J=8 Hz, 1H), 7.15 (dd, J=9, 3 Hz, 1H), 3.90 (s, 3H), 3.84 (s, 3H).

### 11c) 3-(6-hydroxy-1,3-benzothiazol-2-yl)benzoic acid

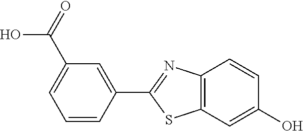

Boron tribromide (1 M in dichloromethane) (4.94 mL, 4.94 mmol) was added slowly to a solution of methyl 3-[6-(methyloxy)-1,3-benzothiazol-2-yl]benzoate (0.37 g, 1.24 mmol) in dichloromethane (12 mL) at −12° C. (ice-acetone bath). The reaction mixture was stirred for 4 hours as the bath warmed to 0° C. The bath was removed and the reaction mixture was stirred at room temperature overnight. The reaction mixture was poured into ice water and extracted with ethyl acetate. The aqueous and organic layers were independently filtered to provide a solid which was dried at 75° C. overnight to afford 0.236 g (67%) of 3-(6-hydroxy-1,3-benzothiazol-2-yl)benzoic acid. ¹H NMR (400 MHz, DMSO-d₆): δ 13.33 (s, 1H), 9.93 (s, 1H), 8.52 (s, 1H), 8.20 (d, J=7 Hz, 1H), 8.04 (d, J=8 Hz, 1H), 7.87 (d, J=9 Hz, 1H), 7.66 (t, J=8 Hz, 1H), 7.42 (d, J=2 Hz, 1H), 7.00 (d, J=9 Hz, 1H).

### 11d) Methyl 3-(6-hydroxy-1,3-benzothiazol-2-yl)benzoate

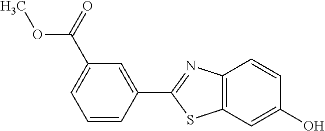

Thionyl chloride (0.13 mL, 1.74 mmol) was added slowly to a slurry of 3-(6-hydroxy-1,3-benzothiazol-2-yl)benzoic acid (0.236 g, 0.870 mmol) in methanol (8 mL) and the reaction mixture was heated at 75° C. overnight. The reaction mixture was cooled to room temperature and concentrated. The crude material was diluted with 5% sodium bicarbonate and extracted with ethyl acetate. The ethyl acetate layer was dried over magnesium sulfate, filtered, and concentrated to afford 0.24 g (97%) of methyl 3-(6-hydroxy-1,3-benzothiazol-2-yl)benzoate. ¹H NMR (400 MHz, DMSO-d₆): δ 9.95 (s, 1H), 8.54 (t, J=2 Hz, 1H), 8.23 (d, J=8 Hz, 1H), 8.07 (d, J=8 Hz, 1H), 7.88 (d, J=9 Hz, 1H), 7.69 (t, J=8 Hz, 1H), 7.43 (d, J=2 Hz, 1H), 7.00 (dd, J=9, 2 Hz, 1H), 3.90 (s, 3H).

### 11e) 3-[6-({[3-(2,6-Dichlorophenyl)-5-(1-methylethyl)-4-isoxazolyl]methyl}oxy)-1,3-benzothiazol-2-yl]benzoic acid

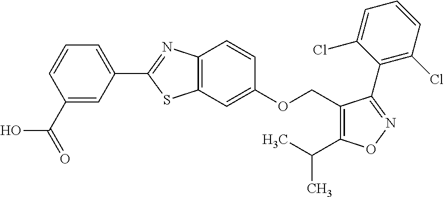

Methyl 3-(6-hydroxy-1,3-benzothiazol-2-yl)benzoate (0.12 g, 0.421 mmol), [3-(2,6-dichlorophenyl)-5-(1-methylethyl)-4-isoxazolyl]methanol (0.12 g, 0.421 mmol) (prepared by the general procedure described in Maloney, P. R., et al., 2000 J. Med. Chem. 43:2971-2974) and triphenylphosphine (0.11 g, 0.421 mmol) were stirred in dichloromethane (8 mL) at 0° C., then diisopropyl azodicarboxylate (0.083 mL, 0.421 mmol) was added slowly to the reaction mixture. After stirring for 4 days at room temperature, the reaction mixture was concentrated to give an oil. The crude oil was purified by flash chromatography over silica using a hexanes:ethyl acetate gradient of 0 to 30% ethyl acetate to afford 0.17 g (73%) of impure methyl 3-[6-({[3-(2,6-dichlorophenyl)-5-(1-methylethyl)-4-isoxazolyl]methyl}oxy)-1,3-benzothiazol-2-yl]benzoate. The impure ester was used without further purification. Methyl 3-[6-({[3-(2,6-dichlorophenyl)-5-(1-methylethyl)-4-isoxazolyl]methyl}oxy)-1,3-benzothiazol-2-yl]benzoate (0.17 g, 0.307 mmol) and 1 N lithium hydroxide (0.43 mL, 0.43 mmol) were stirred in 1,4-dioxane (2 mL) at room temperature overnight. The reaction mixture was concentrated and diluted with ethyl acetate followed by water and 5% sodium hydrogensulfate. The layers were separated and the organic layer was washed with brine, dried over magnesium sulfate, filtered, and concentrated. To this material was added 5% sodium bicarbonate followed by 1 N sodium hydroxide then this mixture was washed with diethyl ether. The aqueous layer was acidified using 6 N hydrochloric acid and the precipitate was filtered. The material obtained from the filtration was purified by reverse phase preparative HPLC using an acetonitrile:water gradient of 0% to 50% acetonitrile with 0.05% trifluoroacetic acid as a modifier to afford 0.028 g (12%) of 3-[6-({[3-(2,6-dichlorophenyl)-5-(1-methylethyl)-4-isoxazolyl]methyl}oxy)-1,3-benzothiazol-2-yl]benzoic acid. ¹H NMR (400 MHz, DMSO-d₆): δ 13.32 (s, 1H), 8.52 (t, J=1 Hz, 1H), 8.23 (d, J=8 Hz, 1H), 8.06 (d, J=8 Hz, 1H), 7.89 (d, J=9 Hz, 1H), 7.67 (t, J=8 Hz, 1H), 7.63 (d, J=3 Hz, 1H), 7.62 (s, 1H), 7.60 (s, 1H), 7.53 (m, J=9, 7 Hz, 1H), 6.91 (dd, J=9, 3 Hz, 1H), 4.91 (s, 2H), 3.47 (septet, J=7 Hz, 1H), 1.32 (d, J=7 Hz, 6H). HRMS C₂₇H₂₀Cl₂N₂O₄S m/z 559.14100 (M+H)⁺_{Cal}; 559.14088 (M+H)⁺_{Obs}.

### Example 12

### 5-({[3-(2,6-Dichlorophenyl)-5-(1-methylethyl)-4-isoxazolyl]methyl}oxy)-1-{[3-(1H-tetrazol-5-yl)phenyl]methyl}-1H-indole

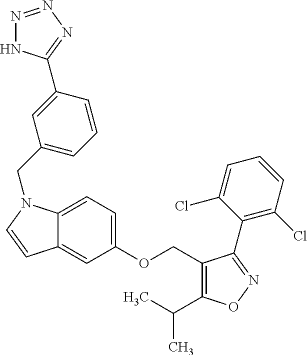

### 12a) 5-({[3-(2,6-Dichlorophenyl)-5-(1-methylethyl)-4-isoxazolyl]methyl}oxy)-1H-indole

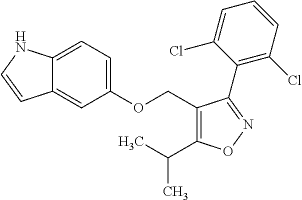

1H-Indol-5-ol (0.58 g, 4.4 mmol), [3-(2,6-dichlorophenyl)-5-(1-methylethyl)-4-isoxazolyl]methanol (prepared by the general procedure described in Maloney, P. R., et al., 2000 J. Med. Chem. 43:2971-2974) (1.25 g, 4.4 mmol) and polymer-bound triphenylphosphine (1 mmol/g) (4.36 g, 4.4 mmol) were stirred in dichloromethane (35 mL) at 0° C. in an ice bath, then a solution of diisopropyl azodicarboxylate (0.86 mL, 4.4 mmol) in dichloromethane (3 mL) was added slowly to the reaction mixture. After stirring at 0° C. for approximately 10 min, the ice bath was removed and the reaction mixture was stirred at room temperature. After stirring at room temperature for 3 days, the reaction mixture was filtered, and concentrated. The crude oil was purified by flash chromatography over silica using a hexanes:ethyl acetate gradient of 0 to 25% ethyl acetate to afford impure material, which was purified again by flash chromatography over silica using a hexanes:dichloromethane gradient of 0 to 50% dichloromethane to afford 0.72 g (41%) of 5-({[3-(2,6-dichlorophenyl)-5-(1-methylethyl)-4-isoxazolyl]methyl}oxy)-1H-indole. ¹H NMR (400 MHz, DMSO-d₆): δ 10.87 (s, 1H), 7.62 (m, 2H), 7.52 (dd, J=9, 7 Hz, 1H), 7.23 (t, J=3 Hz, 1H), 7.16 (d, J=9 Hz, 1H), 6.90 (d, J=2 Hz, 1H), 6.48 (dd, J=9, 2 Hz, 1H), 6.23 (s, 1H), 4.73 (s, 2H), 3.39 (septet, J=7 Hz, 1H), 1.27 (d, J=7 Hz, 6H).

### 12b) 3-{[5-({[3-(2,6-Dichlorophenyl)-5-(1-methylethyl)-4-isoxazolyl]methyl}oxy)-1H-indol-1-yl]methyl}benzonitrile

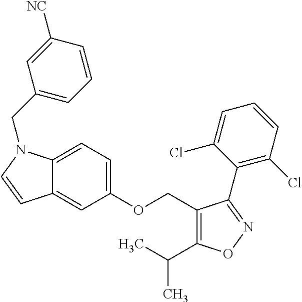

Sodium hydride (60% dispersion in oil) (0.017 g, 0.430 mmol) was washed with hexanes. To a suspension of sodium hydride in N,N-dimethylformamide (1 mL) was added a solution of 5-({[3-(2,6-dichlorophenyl)-5-(1-methylethyl)-4-isoxazolyl]methyl}oxy)-1H-indole (0.15 g, 0.374 mmol) in N,N-dimethylformamide (1 mL). The reaction mixture was stirred for several minutes, then a solution of 3-(bromomethyl)benzonitrile (0.081 g, 0.415 mmol) in N,N-dimethylformamide (1 mL) was added to the reaction mixture and stirring was continued at room temperature for 24 h. The reaction mixture was diluted with water, followed by ethyl acetate. The layers were separated and the ethyl acetate layer was washed with brine, dried over magnesium sulfate, filtered, and concentrated. The crude product was purified by flash chromatography over silica using a hexanes:ethyl acetate gradient of 0 to 30% ethyl acetate to afford 0.143 g (75%) of 3-{[5-({[3-(2,6-dichlorophenyl)-5-(1-methylethyl)-4-isoxazolyl]methyl}oxy)-1H-indol-1-yl]methyl}benzonitrile. ¹H NMR (400 MHz, DMSO-d₆): δ 7.69 (d, J=8 Hz, 1H), 7.60 (d, J=1 Hz, 1H), 7.58 (s, 2H), 7.50 (m, 2H), 7.45 (d, J=3 Hz, 1H), 7.40 (m, 1H), 7.24 (d, J=9 Hz, 1H), 6.94 (d, J=2 Hz, 1H), 6.51 (dd, J=9, 2 Hz, 1H), 6.33 (d, J=4 Hz, 1H), 5.39 (s, 2H), 4.74 (s, 2H), 3.35 (septet, J=7 Hz, 1H), 1.24 (d, J=7 Hz, 6H).

### 12c) 5-({[3-(2,6-Dichlorophenyl)-5-(1-methylethyl)-4-isoxazolyl]methyl}oxy)-1-{[3-(1H-tetrazol-5-yl)phenyl]methyl}-1H-indole

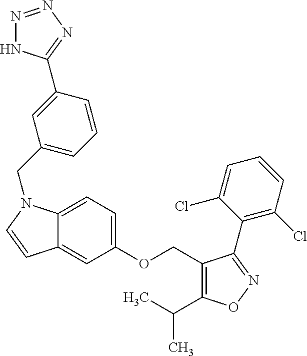

A mixture of 3-{[5-({[3-(2,6-Dichlorophenyl)-5-(1-methylethyl)-4-isoxazolyl]methyl}oxy)-1H-indol-1-yl]methyl}benzonitrile (0.14 g, 0.271 mmol), sodium azide (0.0194 g, 0.298 mmol) and ammonium chloride (0.016 g, 0.298 mmol) in N,N-dimethylformamide (2 mL) was heated at 100° C. in a sealed tube for 48 h. The reaction mixture was diluted with water, followed by 5 N hydrochloric acid to pH of 4 (litmus paper). The acidic mixture was extracted with ethyl acetate and the ethyl acetate layer was washed with brine, dried over magnesium sulfate, filtered, and concentrated. The crude material was purified by reverse phase preparative HPLC using an acetonitrile:water gradient of 0% to 50% acetonitrile with 0.05% trifluoroacetic acid as a modifier to afford 0.012 g (7.9%) of 5-({[3-(2,6-dichlorophenyl)-5-(1-methylethyl)-4-isoxazolyl]methyl}oxy)-1-{[3-(1H-tetrazol-5-yl)phenyl]methyl}-1H-indole. ¹H NMR (400 MHz, DMSO-d₆): δ 7.87 (m, 2H), 7.59 (m, 2H), 7.49 (m, 3H), 7.29 (d, J=8 Hz, 1H), 7.24 (d, J=9 Hz, 1H), 6.94 (d, J=3 Hz, 1H), 6.50 (dd, J=9, 2 Hz, 1H), 6.33 (dd, J=3, 1 Hz, 1H), 5.44 (s, 2H), 4.73 (s, 2H), 3.36 (septet, J=7 Hz, 1H), 1.24 (d, J=7 Hz, 6H). HRMS C₂₉H₂₄Cl₂N₆O₂ m/z 559.14100 (M+H)⁺_{Cal}; 559.14088 (M+H)⁺_{Obs}.

### Example 13

### 4-{[5-({[3-(2,6-Dichlorophenyl)-5-(1-methylethyl)-4-isoxazolyl]methyl}oxy)-1H-indol-1-yl]methyl}benzoic acid

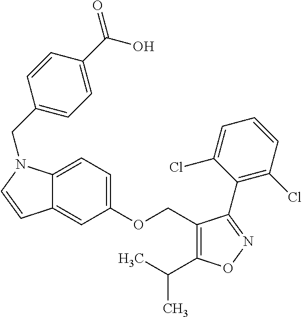

### 13a) Methyl 4-{[5-({[3-(2,6-dichlorophenyl)-5-(1-methylethyl)-4-isoxazolyl]methyl}oxy)-1H-indol-1-yl]methyl}benzoate

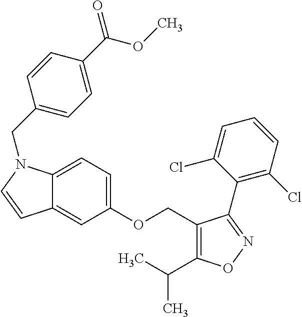

Sodium hydride (60% dispersion in oil) (0.017 g, 0.430 mmol) was washed with hexanes. To the sodium hydride was added N,N-dimethylformamide (1 mL), followed by a solution of 5-({[3-(2,6-dichlorophenyl)-5-(1-methylethyl)-4-isoxazolyl]methyl}oxy)-1H-indole (0.15 g, 0.374 mmol) in N,N-dimethylformamide (1 mL). The reaction mixture was stirred for several minutes, then a solution of methyl 4-(bromomethyl)benzoate (0.095 g, 0.415 mmol) in N,N-dimethylformamide (1 mL) was added and the reaction mixture was stirred at room temperature for 24 hours. The reaction mixture was diluted with water, followed by ethyl acetate. The layers were separated and the ethyl acetate layer was washed with water followed by brine, dried over magnesium sulfate, filtered, and concentrated to afford 0.205 (100%) of methyl 4-{[5-({[3-(2,6-dichlorophenyl)-5-(1-methylethyl)-4-isoxazolyl]methyl}oxy)-1H-indol-1-yl]methyl}benzoate. ¹H NMR (400 MHz, DMSO-d₆): δ 7.84 (d, J=8 Hz, 2H), 7.59 (m, 2H), 7.51 (dd, J=9, 7 Hz, 1H), 7.42 (d, J=3 Hz, 1H), 7.18 (t, J=9 Hz, 3H), 6.95 (d, J=2 Hz, 1H), 6.49 (dd, J=9, 2 Hz, 1H), 6.33 (dd, J=3, 1 Hz, 1H), 5.42 (s, 2H), 4.73 (s, 2H), 3.79 (s, 3H), 3.36 (septet, J=7 Hz, 1H), 1.25 (d, J=7 Hz, 6H).

### 13b) 4-{[5-({[3-(2,6-Dichlorophenyl)-5-(1-methylethyl)-4-isoxazolyl]methyl}oxy)-1H-indol-1-yl]methyl}benzoic acid

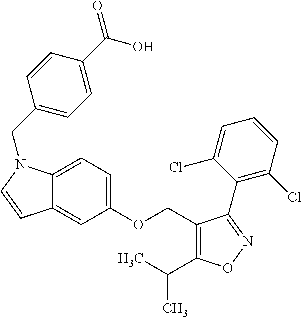

To a solution of methyl 4-{[5-({[3-(2,6-dichlorophenyl)-5-(1-methylethyl)-4-isoxazolyl]methyl}oxy)-1H-indol-1-yl]methyl}benzoate (0.205 g, 0.374 mmol) in tetrahydrofuran (3 mL) was added 1 N lithium hydroxide (0.52 mL). The reaction mixture was stirred at room temperature for 24 hours. 1,4-Dioxane (1 mL) was added to the reaction mixture and stirring was continued for another 24 hours. A portion of the reaction mixture (0.3 mL) was heated in the microwave at 100° C. for 500 seconds, then 1 N lithium hydroxide (0.1 mL) was added and the reaction mixture was heated in the microwave again for another 500 seconds at 100° C. Lithium hydroxide (1 N) (0.7 mL) was added to the remaining reaction mixture and it was heated in the microwave at 100° C. for 500 seconds. The two reaction mixtures were combined and heated for another 500 seconds. Lithium hydroxide (1 N) (0.75 mL, 0.75 mmol) was added to the reaction mixture and heating was continued at 100° C. for 1000 seconds. The reaction mixture was concentrated and diluted with ethyl acetate, water and 5% sodium hydrogensulfate. The ethyl acetate layer was separated, washed with brine, dried over magnesium sulfate, filtered, and concentrated. The crude material was purified by flash chromatography over silica using a dichloromethane:methanol gradient of 0 to 5% methanol to afford partially purified material. The impure product was purified again by flash chromatography over silica using a dichloromethane:methanol gradient of 0 to 1% methanol to afford 0.080 g (40%) of 4-{[5-({[3-(2,6-dichlorophenyl)-5-(1-methylethyl)-4-isoxazolyl]methyl}oxy)-1H-indol-1-yl]methyl}benzoic acid. ¹H NMR (400 MHz, DMSO-d₆): δ 12.87 (s, 1H), 7.82 (d, J=8 Hz, 2H), 7.59 (m, 2H), 7.51 (dd, J=9, 7 Hz, 1H), 7.43 (d, J=3 Hz, 1H), 7.18 (m, 3H), 6.95 (d, J=2 Hz, 1H), 6.49 (dd, J=9, 2 Hz, 1H), 6.33 (d, J=4 Hz, 1H), 5.41 (s, 2H), 4.73 (s, 2H), 3.37 (septet, J=7 Hz, 1H), 1.26 (d, J=7 Hz, 6H). HRMS C₂₉H₂₄Cl₂N₂O₄ m/z 535.11900 (M+H)⁺_{Cal}; 535.11861 (M+H)⁺_{Obs}.

### Example 14

### 3-{[5-({[3-{[(2,6-Dichloro-4-fluorophenyl)oxy]methyl}-5-(1-methylethyl)-4-isoxazolyl]methyl}oxy)-1H-indol-1-yl]methyl}benzoic acid

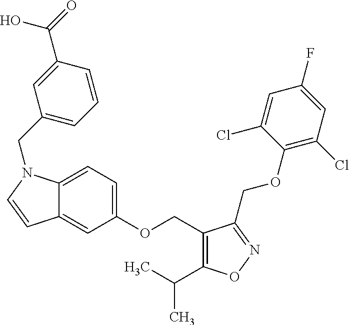

### 14a) Methyl 3-{[5-({[3-{[(2,6-dichloro-4-fluorophenyl)oxy]methyl}-5-(1-methylethyl)-4-isoxazolyl]methyl}oxy)-1H-indol-1-yl]methyl}benzoate

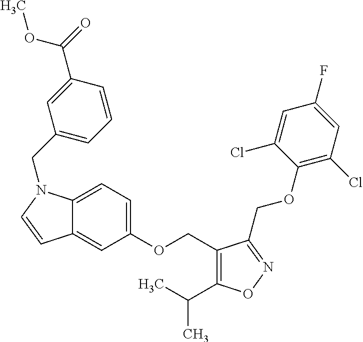

Methyl 3-{[5-({[3-(hydroxymethyl)-5-(1-methylethyl)-4-isoxazolyl]methyl}oxy)-1H-indol-1-yl]methyl}benzoate (prepared by the general procedure described in Example 1f) (0.1 g, 0.230 mmol), 2,6-dichloro-4-fluorophenol (0.0417 g, 0.230 mmol) and triphenylphosphine (0.06 g, 0.23 mmol) were stirred in toluene (2.5 mL), then diisopropyl azodicarboxylate (0.045 mL, 0.23 mmol) was added to the reaction mixture. The reaction mixture was heated in the microwave at 90° C. for 10 min. The reaction mixture was concentrated and the crude oil was purified by flash chromatography over silica using a hexanes:ethyl acetate gradient of 0 to 30% ethyl acetate to afford 0.090 g (65%) of methyl 3-{[5-({[3-{[(2,6-dichloro-4-fluorophenyl)oxy]methyl}-5-(1-methylethyl)-4-isoxazolyl]methyl}oxy)-1H-indol-1-yl]methyl}benzoate. ¹H NMR (400 MHz, DMSO-d₆): δ 7.81 (d, J=7 Hz, 1H), 7.75 (s, 1H), 7.52 (d, J=8 Hz, 2H), 7.47 (m, 1H), 7.41 (m, 2H), 7.29 (d, J=9 Hz, 1H), 7.15 (d, J=2 Hz, 1H), 6.75 (dd, J=9, 3 Hz, 1H), 6.39 (d, J=3 Hz, 1H), 5.45 (s, 2H), 5.07 (s, 2H), 5.03 (s, 2H), 3.78 (s, 3H), 3.35 (m, 1H), 1.21 (d, J=7 Hz, 6H).

### 14b) 3-{[5-({[3-{[(2,6-Dichloro-4-fluorophenyl)oxy]methyl}-5-(1-methylethyl)-4-isoxazolyl]methyl}oxy)-1H-indol-1-yl]methyl}benzoic acid

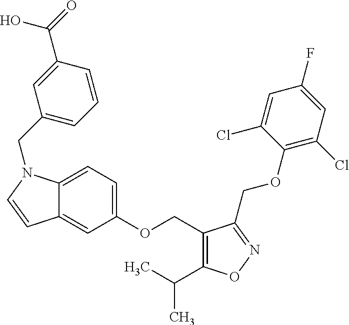

To a solution of methyl 3-{[5-({[3-{[(2,6-dichloro-4-fluorophenyl)oxy]methyl}-5-(1-methylethyl)-4-isoxazolyl]methyl}oxy)-1H-indol-1-yl]methyl}benzoate (0.0867 g, 0.145 mmol), tetrahydrofuran (2.3 mL) and methanol (1.1 mL) was added 1 N sodium hydroxide (0.435 mL, 0.435 mmol). The reaction mixture was heated in the microwave at 120° C. for 500 seconds. The reaction mixture was concentrated and diluted with water, followed by 1 N hydrochloric acid (0.5 mL). The acidic solution was extracted with diethyl ether. The organic layer was separated and concentrated to afford 0.0774 g (91%) of 3-{[5-({[3-{[(2,6-dichloro-4-fluorophenyl)oxy]methyl}-5-(1-methylethyl)-4-isoxazolyl]methyl}oxy)-1H-indol-1-yl]methyl}benzoic acid. ¹H NMR (400 MHz, DMSO-d₆): δ 12.94 (s, 1H), 7.78 (d, J=7 Hz, 1H), 7.70 (s, 1H), 7.52 (d, J=8 Hz, 2H), 7.47 (d, J=3 Hz, 1H), 7.40 (m, 2H), 7.30 (d, J=9 Hz, 1H), 7.15 (d, J=2 Hz, 1H), 6.75 (dd, J=9, 2 Hz, 1H), 6.39 (d, J=3 Hz, 1H), 5.44 (s, 2H), 5.07 (s, 2H), 5.03 (s, 2H), 3.35 (m, 1H), 1.21 (d, J=7 Hz, 6H). HRMS C₃₀H₂₅Cl₂FN₂O₅ m/z 583.1203 (M+H)⁺_{Cal}; 583.1187 (M+H)⁺_{Obs}.

### Example 15

### 3-{[5-({[3-{[(2,6-Dichlorophenyl)oxy]methyl}-5-(1-methylethyl)-4-isoxazolyl]methyl}oxy)-1H-indol-1-yl]methyl}benzoic acid

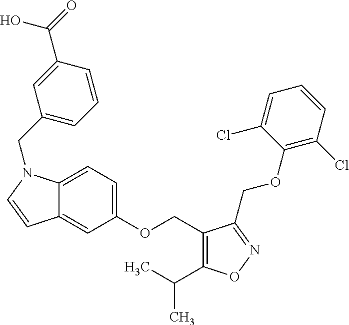

### 15a) Methyl 3-{[5-({[3-{[(2,6-dichlorophenyl)oxy]methyl}-5-(1-methylethyl)-4-isoxazolyl]methyl}oxy)-1H-indol-1-yl]methyl}benzoate

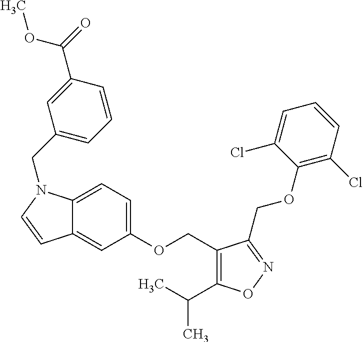

Methyl 3-{[5-({[3-(hydroxymethyl)-5-(1-methylethyl)-4-isoxazolyl]methyl}oxy)-1H-indol-1-yl]methyl}benzoate (prepared by the general procedure described in Example 1f) (0.1 g, 0.230 mmol), 2,6-dichlorophenol (0.038 g, 0.230 mmol) and triphenylphosphine (0.06 g, 0.23 mmol) were stirred in toluene (2.5 mL), then diisopropyl azodicarboxylate (0.045 mL, 0.23 mmol) was added to the reaction mixture. The reaction mixture was heated in the microwave at 90° C. for 10 min. The reaction was concentrated and the crude oil was purified by flash chromatography over silica using a hexanes:ethyl acetate gradient of 0 to 30% ethyl acetate to afford 0.090 g (68%) of methyl 3-{[5-({[3-{[(2,6-dichlorophenyl)oxy]methyl}-5-(1-methylethyl)-4-isoxazolyl]methyl}oxy)-1H-indol-1-yl]methyl}benzoate. ¹H NMR (400 MHz, DMSO-d₆): δ 7.81 (m, 1H), 7.75 (s, 1H), 7.43 (m, 5H), 7.29 (d, J=9 Hz, 1H), 7.17 (m, 2H), 6.75 (dd, J=9, 3 Hz, 1H), 6.39 (d, J=3 Hz, 1H), 5.45 (s, 2H), 5.09 (s, 2H), 5.04 (s, 2H), 3.78 (s, 3H), 3.35 (m, 1H), 1.21 (d, J=7 Hz, 6H).

### 15b) 3-{[5-({[3-{[(2,6-Dichlorophenyl)oxy]methyl}-5-(1-methylethyl)-4-isoxazolyl]methyl}oxy)-1H-indol-1-yl]methyl}benzoic acid

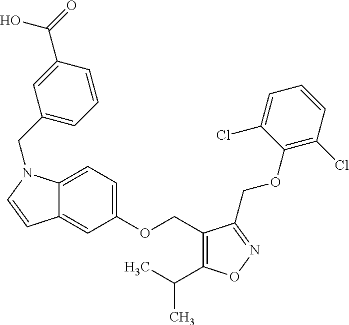

To a solution of methyl 3-{[5-({[3-{[(2,6-dichlorophenyl)oxy]methyl}-5-(1-methylethyl)-4-isoxazolyl]methyl}oxy)-1H-indol-1-yl]methyl}benzoate (0.09 g, 0.155 mmol), tetrahydrofuran (2.5 mL) and methanol (1.2 mL) was added 1 N sodium hydroxide (0.47 mL, 0.466 mmol). The reaction mixture was heated in the microwave at 120° C. for 500 seconds. The reaction mixture was concentrated, then diluted with water, followed by 1 N hydrochloric acid (0.5 mL). This solution was extracted with diethyl ether. The organic phase was separated and concentrated to afford 0.0771 g (88%) of 3-{[5-({[3-{[(2,6-dichlorophenyl)oxy]methyl}-5-(1-methylethyl)-4-isoxazolyl]methyl}oxy)-1H-indol-1-yl]methyl}benzoic acid. ¹H NMR (400 MHz, DMSO-d₆): δ 7.77 (d, J=7 Hz, 1H), 7.70 (s, 1H), 7.46 (m, 3H), 7.38 (m, 2H), 7.30 (d, J=9 Hz, 1H), 7.17 (m, 2H), 6.75 (dd, J=9, 2 Hz, 1H), 6.39 (d, J=3 Hz, 1H), 5.43 (s, 2H), 5.10 (s, 2H), 5.04 (s, 2H), 3.36 (m, 1H), 1.21 (d, J=7 Hz, 6H). HRMS C₃₀H₂₆Cl₂N₂O₅ m/z 565.1297 (M+H)⁺_{Cal}; 565.1295 (M+H)⁺_{Obs}.

### Example 16

### 4-[5-({[3-(2,6-Dichlorophenyl)-5-(1-methylethyl)-4-isoxazolyl]methyl}oxy)-1-benzothien-2-yl]benzoic acid

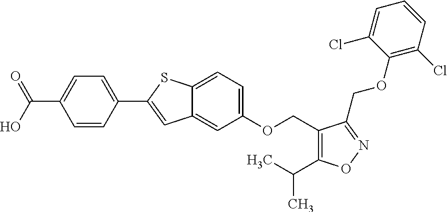

### 16a) 5-(Methyloxy)-1-benzothiophene

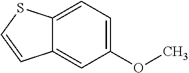

To a slurry of 5-bromo-1-benzothiophene (3 g, 14.1 mmol) and methanol (32 mL) was added sodium methoxide (25% wt/wt in Methanol) (32 mL, 140 mmol) followed by copper (I) bromide (0.2 g, 1.4 mmol). The reaction mixture was stirred at reflux for 1.5 hours. The reaction mixture was cooled to room temperature then copper (0.087 g, 1.37 mmol) was added. The reaction mixture was heated at reflux for 4 days then it was cooled to room temperature and concentrated. The reaction mixture was diluted with ice-water followed by diethyl ether. The diethyl ether layer was separated, washed with water followed by brine, dried over magnesium sulfate, filtered and concentrated. The crude material was purified by flash chromatography over silica using a hexanes:ethyl acetate gradient of 0 to 10% ethyl acetate to afford 1.2 g (52%) of 5-(methyloxy)-1-benzothiophene. ¹H NMR (400 MHz, DMSO-d₆): δ 7.83 (d, J=9 Hz, 1H), 7.71 (d, J=5 Hz, 1H), 7.39 (d, J=2 Hz, 1H), 7.35 (d, J=6 Hz, 1H), 6.97 (dd, J=9, 2 Hz, 1H), 3.78 (s, 3H)

### 16b) [5-(Methyloxy)-1-benzothien-2-yl]boronic acid

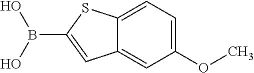

To a solution of 5-(methyloxy)-1-benzothiophene (1.13 g, 6.88 mmol) in tetrahydrofuran (38 mL) at −60° C. was slowly added n-butyl lithium (2.5 M in hexanes) (3.05 mL, 7.57 mmol). The reaction mixture was stirred at −60° C. for approximately 15 min then triisopropylborate (1.8 mL, 7.91 mmol) was added to the reaction mixture over a period of 35 min. The cooling bath was removed and the reaction mixture was stirred at room temperature for 1 h. The reaction mixture was diluted with 1 N hydrochloric acid (30 mL) followed by ethyl acetate. The ethyl acetate layer was separated, washed with brine, dried over magnesium sulfate, filtered and concentrated to afford 1.3 g (91%) of [5-(methyloxy)-1-benzothien-2-yl]boronic acid. ¹H NMR (400 MHz, DMSO-d₆): δ 7.83 (d, J=9 Hz, 1H), 7.77 (s, 1H), 7.45 (s, 1H), 6.99 (dd, J=9, 2 Hz, 1H), 3.79 (t, J=2 Hz, 3H).

### 16c) Ethyl 4-[5-(methyloxy)-1-benzothien-2-yl]benzoate

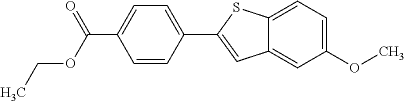

To a solution of [5-(methyloxy)-1-benzothien-2-yl]boronic acid (1.3 g, 6.25 mmol) and toluene (30 mL) was added ethyl 4-iodobenzoate (1.59 mL, 9.37 mmol), tetrakis(triphenylphosphine)palladium(0) (0.29 g, 0.25 mmol) and 2 M sodium carbonate (7.1 mL, 13.75 mmol). The reaction mixture was heated at reflux for 4 h, then stirred at room temperature for 3 days, and heated for another 2 h. The reaction mixture was cooled to room temperature, diluted with ethyl acetate, followed by water. The ethyl acetate layer was separated, washed with brine, dried over magnesium sulfate, filtered, and concentrated. The crude material was purified by flash chromatography over silica using a hexanes:ethyl acetate gradient of 0 to 10% ethyl acetate to afford 0.32 g (16%) of ethyl 4-[5-(methyloxy)-1-benzothien-2-yl]benzoate. ¹H NMR (400 MHz, DMSO-d₆): δ 8.02 (d, J=9 Hz, 2H), 7.93 (s, 1H), 7.88 (m, 3H), 7.38 (d, J=2 Hz, 1H), 7.02 (dd, J=9, 2 Hz, 1H), 4.32 (q, J=7 Hz, 2H), 3.81 (s, 3H), 1.32 (t, J=7 Hz, 3H).

### 16d) Ethyl 4-(5-hydroxy-1-benzothien-2-yl)benzoate

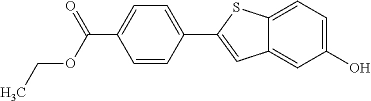

To a solution of ethyl 4-[5-(methyloxy)-1-benzothien-2-yl]benzoate (0.23 g, 0.736 mmol) in dichloromethane (10 mL) at 0° C. was slowly added boron tribromide (1 M in dichloromethane) (2.95 mL, 2.95 mmol). The reaction mixture was stirred at 0° C. for 5 hours. The reaction mixture was poured into ice-water and stirred for several minutes. Ethyl acetate was added to the mixture and the layers were separated. The ethyl acetate layer was washed with brine, dried over magnesium sulfate, filtered, and concentrated. The crude material was purified by flash chromatography over silica using a hexanes:ethyl acetate gradient of 0 to 40% ethyl acetate to afford 0.14 g (64%) of ethyl 4-(5-hydroxy-1-benzothien-2-yl)benzoate. ¹H NMR (400 MHz, DMSO-d₆): δ 9.52 (s, 1H), 8.00 (d, J=9 Hz, 2H), 7.87 (m, 3H), 7.75 (d, J=9 Hz, 1H), 7.18 (d, J=2 Hz, 1H), 6.88 (dd, J=9, 2 Hz, 1H), 4.31 (q, J=7 Hz, 2H), 1.32 (t, J=7 Hz, 3H).

### 16e) Ethyl 4-[5-({[3-(2,6-dichlorophenyl)-5-(1-methylethyl)-4-isoxazolyl]methyl}oxy)-1-benzothien-2-yl]benzoate

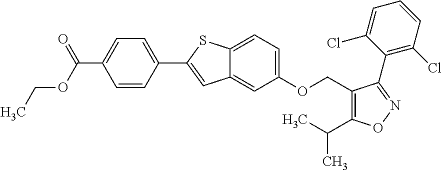

Ethyl 4-(5-hydroxy-1-benzothien-2-yl)benzoate (0.14 g, 0.469 mmol), [3-(2,6-dichlorophenyl)-5-(1-methylethyl)-4-isoxazolyl]methanol (prepared by the general procedure described in Maloney, P. R., et al., 2000 J. Med. Chem. 43:2971-2974) (0.134 g, 0.469 mmol) and polymer-bound triphenylphosphine (3 mmol/g) (0.16 g, 0.469 mmol) were stirred in dichloromethane (5 mL) at 0° C., then diisopropyl azodicarboxylate (0.094 mL, 0.469 mmol) was added slowly to the reaction mixture. The reaction mixture was allowed to warm to room temperature. After stirring for 3 days at room temperature, the reaction mixture was filtered and concentrated. The crude oil was purified by flash chromatography over silica using a hexanes:ethyl acetate gradient of 0 to 30% ethyl acetate to afford 0.116 g (44%) of ethyl 4-[5-({[3-(2,6-dichlorophenyl)-5-(1-methylethyl)-4-isoxazolyl]methyl}oxy)-1-benzothien-2-yl]benzoate. ¹H NMR (400 MHz, DMSO-d₆): δ 8.02 (m, 2H), 7.86 (m, 3H), 7.79 (d, J=9 Hz, 1H), 7.61 (m, 2H), 7.52 (dd, J=9, 7 Hz, 1H), 7.28 (d, J=3 Hz, 1H), 6.80 (dd, J=9, 3 Hz, 1H), 4.87 (s, 2H), 4.31 (q, J=7 Hz, 2H), 3.46 (septet, J=7 Hz, 1H), 1.32 (d, J=7 Hz, 6H), 1.32 (t, J=7 Hz, 3H).

### 16f) 4-[5-({[3-(2,6-Dichlorophenyl)-5-(1-methylethyl)-4-isoxazolyl]methyl}oxy)-1-benzothien-2-yl]benzoic acid

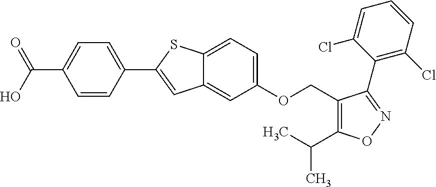

To a solution of ethyl 4-[5-({[3-(2,6-dichlorophenyl)-5-(1-methylethyl)-4-isoxazolyl]methyl}oxy)-1-benzothien-2-yl]benzoate (0.116 g, 0.205 mmol) in tetrahydrofuran (2 mL) and methanol (1.2 mL) was added 1 N sodium hydroxide (0.43 mL, 0.43 mmol). The reaction mixture was stirred at room temperature for 24 h. The reaction mixture was concentrated, then diluted with 1 N hydrochloric acid, followed by ethyl acetate. The ethyl acetate layer was separated, washed with brine, dried over magnesium sulfate, filtered, and concentrated to afford 0.103 g (94%) of 4-[5-({[3-(2,6-dichlorophenyl)-5-(1-methylethyl)-4-isoxazolyl]methyl}oxy)-1-benzothien-2-yl]benzoic acid. ¹H NMR (400 MHz, DMSO-d₆): δ 13.02 (s, 1H), 7.99 (d, J=9 Hz, 2H), 7.84 (m, 3H), 7.79 (d, J=9 Hz, 1H), 7.61 (m, 2H), 7.52 (dd, J=9, 7 Hz, 1H), 7.27 (d, J=2 Hz, 1H), 6.79 (dd, J=9, 2 Hz, 1H), 4.87 (s, 2H), 3.46 (septet, J=7 Hz, 1H), 1.32 (d, J=7 Hz, 6H). HRMS C₂₈H₂₁Cl₂NO₄S m/z 538.0647 (M+H)⁺_{Cal}; 538.0642 (M+H)⁺_{Obs}.

### Example 17

### 5-[6-({[3-(2,6-Dichlorophenyl)-5-(1-methylethyl)-4-isoxazolyl]methyl}oxy)-2-naphthalenyl]-3-pyridinecarboxylic acid

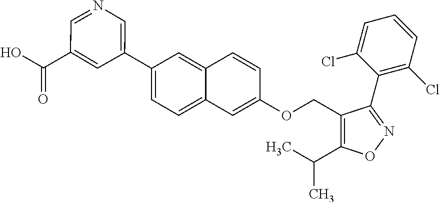

### 17a) Methyl 5-[6-(methyloxy)-2-naphthalenyl]-3-pyridinecarboxylate

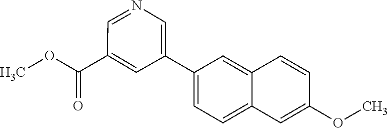

To a slurry of methyl 5-bromo-3-pyridinecarboxylate (1.12 g, 5.55 mmol), tetrakis(triphenylphosphine)palladium(0) (0.21 g, 0.185 mmol), ethylene glycol dimethyl ether (25 mL) and 2 N sodium carbonate (22 mL, 44 mmol) was added [6-(methyloxy)-2-naphthalenyl]boronic acid (1 g, 4.6 mmol) and the reaction mixture was heated at 80° C. for 1 h. The reaction mixture was cooled to room temperature and diluted with water, followed by ethyl acetate. The layers were separated and the ethyl acetate layer was washed with brine, dried over magnesium sulfate, filtered, and concentrated. The crude material was triturated with hot dichloromethane, filtered, and the filtrate was concentrated to give a solid, which was triturated with hot dichloromethane. The filtrate was concentrated and the resulting solid was purified by flash chromatography over silica using a hexanes:ethyl acetate gradient of 0 to 30% ethyl acetate. The solid collected from the two triturations combined with the product obtained from flash chromatography afforded 1.14 g (84%) of methyl 5-[6-(methyloxy)-2-naphthalenyl]-3-pyridinecarboxylate. ¹H NMR (400 MHz, DMSO-d₆): δ 9.25 (d, J=2 Hz, 1H), 9.06 (d, J=2 Hz, 1H), 8.58 (t, J=2 Hz, 1H), 8.33 (s, 1H), 7.95 (m, 2H), 7.90 (dd, J=9, 2 Hz, 1H), 7.38 (d, J=3 Hz, 1H), 7.21 (dd, J=9, 3 Hz, 1H), 3.92 (s, 3H), 3.88 (s, 3H).

### 17b) Methyl 5-(6-hydroxy-2-naphthalenyl)-3-pyridinecarboxylate

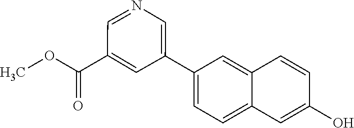

Boron tribromide (1 M in dichloromethane) (16 mL, 16 mmol) was added slowly to a solution of methyl 5-[6-(methyloxy)-2-naphthalenyl]-3-pyridinecarboxylate (1.14 g, 3.89 mmol) in dichloromethane (45 mL) at 0° C. The reaction mixture was stirred for 3 h at 0° C. The reaction mixture was poured into ice water and extracted with ethyl acetate. The ethyl acetate layer was washed with brine, dried over magnesium sulfate, filtered, and concentrated. To the resulting solid was added ethyl acetate and saturated sodium bicarbonate. The mixture was stirred for several minutes, then the layers were separated and the ethyl acetate layer was washed with brine, dried over magnesium sulfate, filtered, and concentrated. To the solid was added ethyl acetate and saturated sodium bicarbonate and the mixture was stirred overnight at room temperature. The ethyl acetate layer was separated, concentrated and to the resulting solid was added a mixture of hot dichloromethane, methanol and ethyl acetate. The precipitate, which formed upon standing, was filtered and the filtrate was purified by flash chromatography over silica using a dichloromethane:methanol gradient of 0 to 1% methanol. The impure fractions from the chromatography were purified again using a dichloromethane:methanol gradient of 0 to 1% methanol. The combined pure product fractions from the two flash columns afforded 0.29 g (27%) of methyl 5-(6-hydroxy-2-naphthalenyl)-3-pyridinecarboxylate. ¹H NMR (400 MHz, DMSO-d₆): δ 9.90 (s, 1H), 9.23 (d, J=2 Hz, 1H), 9.04 (d, J=2 Hz, 1H), 8.56 (t, J=2 Hz, 1H), 8.25 (s, 1H), 7.88 (d, J=9 Hz, 1H), 7.81 (d, J=1 Hz, 2H), 7.16 (m, 1H), 7.13 (m, 1H), 3.92 (s, 3H).

### 17c) 4-(Chloromethyl)-3-(2,6-dichlorophenyl)-5-(1-methylethyl)isoxazole

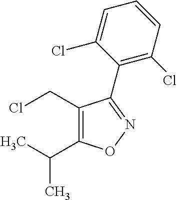

A solution of [3-(2,6-dichlorophenyl)-5-(1-methylethyl)-4-isoxazolyl]methanol (prepared by the general procedure described in Maloney, P. R., et al., 2000 J. Med. Chem. 43:2971-2974) (0.14 g, 0.489 mmol) in dichloroethane (2 mL) was added to a solution of thionyl chloride (0.18 mL, 2.45 mmol) in dichloroethane (1 mL) and the reaction mixture was stirred for 2 h. The reaction mixture was concentrated to afford 0.142 g (95%) of 4-(chloromethyl)-3-(2,6-dichlorophenyl)-5-(1-methylethyl)isoxazole. ¹H NMR (400 MHz, DMSO-d₆): δ 7.66 (m, 2H), 7.59 (dd, J=9, 7 Hz, 1H), 4.47 (s, 2H), 3.45 (septet, J=7 Hz, 1H), 1.31 (d, J=7 Hz, 6H).

### 17d) Methyl 5-[6-({[3-(2,6-dichlorophenyl)-5-(1-methylethyl)-4-isoxazolyl]methyl}oxy)-2-naphthalenyl]-3-pyridinecarboxylate

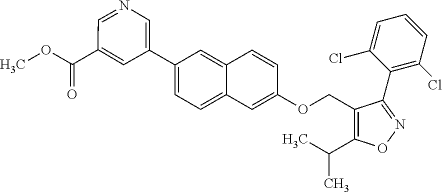

Methyl 5-(6-hydroxy-2-naphthalenyl)-3-pyridinecarboxylate (0.13 g, 0.465 mmol) and cesium carbonate (0.21 g, 0.652 mmol) in N,N-dimethylformamide (1 mL) were heated at 65° C. for 45 min. A solution of 4-(chloromethyl)-3-(2,6-dichlorophenyl)-5-(1-methylethyl)isoxazole (0.142 g, 0.465 mmol) in N,N-dimethylformamide (1 mL) was added to the reaction mixture and heating at 65° C. was continued for 24 h. The reaction mixture was cooled to room temperature, then diluted with water, followed by ethyl acetate. The ethyl acetate layer was separated, washed several times with water, followed by brine, dried over magnesium sulfate, filtered, and concentrated. The crude material was purified by flash chromatography over silica using a dichloromethane:methanol gradient of 0 to 1% methanol to afford 0.21 g (84%) of methyl 5-[6-({[3-(2,6-dichlorophenyl)-5-(1-methylethyl)-4-isoxazolyl]methyl}oxy)-2-naphthalenyl]-3-pyridinecarboxylate. ¹H NMR (400 MHz, DMSO-d₆): δ 9.23 (d, J=2 Hz, 1H), 9.05 (d, J=2 Hz, 1H), 8.56 (t, J=2 Hz, 1H), 8.29 (s, 1H), 7.88 (m, 3H), 7.61 (m, 2H), 7.52 (dd, J=9, 7 Hz, 1H), 7.33 (d, J=2 Hz, 1H), 6.95 (dd, J=9, 2 Hz, 1H), 4.95 (s, 2H), 3.92 (s, 3H), 3.51 (septet, J=7 Hz, 1H), 1.34 (d, J=7 Hz, 6H).

### 17e) 5-[6-({[3-(2,6-Dichlorophenyl)-5-(1-methylethyl)-4-isoxazolyl]methyl}oxy)-2-naphthalenyl]-3-pyridinecarboxylic acid

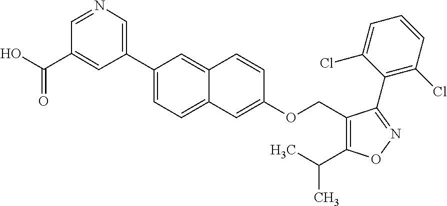

To a solution of methyl 5-[6-({[3-(2,6-dichlorophenyl)-5-(1-methylethyl)-4-isoxazolyl]methyl}oxy)-2-naphthalenyl]-3-pyridinecarboxylate (0.21 g, 0.384 mmol) in tetrahydrofuran (4 mL) and methanol (2 mL) was added 1 N sodium hydroxide (0.79 mL) and the reaction mixture was heated at 65° C. for 1.5 h. The reaction mixture was cooled to room temperature and concentrated. The crude material was acidified with 1N hydrochloric acid to pH 3 (litmus paper) and extracted with ethyl acetate. The ethyl acetate layer was separated, washed with brine, dried over magnesium sulfate, filtered, and concentrated. To the product was added hot dichloromethane and methanol and the solvent was removed in vacuo to afford 0.166 g (81%) of 5-[6-({[3-(2,6-dichlorophenyl)-5-(1-methylethyl)-4-isoxazolyl]methyl}oxy)-2-naphthalenyl]-3-pyridinecarboxylic acid. ¹H NMR (400 MHz, DMSO-d₆): δ 13.54 (s, 1H), 9.20 (d, J=2 Hz, 1H), 9.03 (d, J=2 Hz, 1H), 8.54 (t, J=2 Hz, 1H), 8.28 (s, 1H), 7.86 (m, 3H), 7.61 (m, 2H), 7.52 (dd, J=9, 7 Hz, 1H), 7.32 (d, J=3 Hz, 1H), 6.95 (dd, J=9, 3 Hz, 1H), 4.95 (s, 2H), 3.51 (septet, J=7 Hz, 1H), 1.34 (d, J=7 Hz, 6H). HRMS C₂₉H₂₂Cl₂N₂O₄ m/z 533.1035 (M+H)⁺_{Cal}; 533.1037 (M+H)⁺_{Obs}.

### Example 18

### 6-[6-({[3-(2,6-Dichlorophenyl)-5-(1-methylethyl)-4-isoxazolyl]methyl}oxy)-2-naphthalenyl]-2-pyridinecarboxylic acid

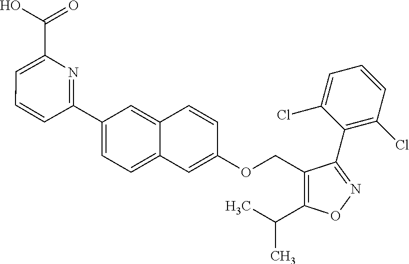

### 18a) Methyl 6-[6-(methyloxy)-2-naphthalenyl]-2-pyridinecarboxylate

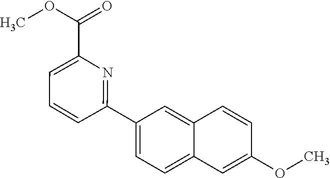

To a slurry of methyl 6-bromo-2-pyridinecarboxylate (1.12 g, 5.55 mmol), tetrakis(triphenylphosphine)palladium(0) (0.21 g, 0.185 mmol), ethylene glycol dimethyl ether (25 mL) and 2 N sodium carbonate (22 mL, 44 mmol) was added [6-(methyloxy)-2-naphthalenyl]boronic acid (1 g, 4.6 mmol) and the reaction mixture was heated at 80° C. for 1 h. The reaction mixture was cooled to room temperature and diluted with water, followed by ethyl acetate. The layers were separated and the ethyl acetate layer was washed with brine, dried over magnesium sulfate, filtered, and concentrated. The crude product was triturated with hot dichloromethane and filtered to give the product. The filtrate was concentrated and the resulting solid was triturated with hot dichloromethane and filtered to give a second batch of product for a total of 0.32 g of product from the two triturations. The filtrate from the second trituration was concentrated and purified by flash chromatography over silica using a hexanes:ethyl acetate gradient of 0 to 30% ethyl acetate to afford additional product for a total yield of 0.936 (58%) of methyl 6-[6-(methyloxy)-2-naphthalenyl]-2-pyridinecarboxylate. ¹H NMR (400 MHz, DMSO-d₆): δ 8.61 (s, 1H), 8.32 (dd, J=8, 1 Hz, 1H), 8.24 (dd, J=9, 2 Hz, 1H), 8.08 (t, J=8 Hz, 1H), 7.96 (m, 3H), 7.37 (d, J=2 Hz, 1H), 7.20 (dd, J=9, 2 Hz, 1H), 3.92 (s, 3H), 3.89 (s, 3H).

### 18b) Methyl 6-(6-hydroxy-2-naphthalenyl)-2-pyridinecarboxylate

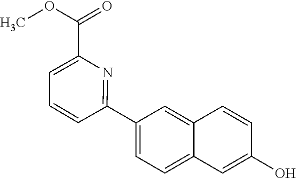

To a solution of methyl 6-[6-(methyloxy)-2-naphthalenyl]-2-pyridinecarboxylate (0.4 g, 1.36 mmol) in dichloromethane (19 mL) at 0° C. was added boron tribromide (1 M in dichloromethane) (5.6 mL, 5.6 mmol). The reaction mixture was stirred at 0° C. for 50 min. The reaction mixture was poured into ice-water and stirred for several minutes, then ethyl acetate was added, followed by saturated sodium bicarbonate. The layers were separated and the ethyl acetate layer was washed with brine, dried over magnesium sulfate, filtered, and concentrated. The resulting solid was triturated with hot dichloromethane, followed by ethyl acetate then methanol. The resulting solid was filtered and dried to afford 0.56 g of a mixture of methyl 6-(6-hydroxy-2-naphthalenyl)-2-pyridinecarboxylate and 6-(6-hydroxy-2-naphthalenyl)-2-pyridinecarboxylic acid. To a solution of this mixture (0.56 g, 2.11 mmol) and methanol (19 mL) was added thionyl chloride (0.31 g, 4.22 mmol) slowly. The reaction mixture was heated at 75° C. for 24 h. The reaction mixture was cooled to room temperature and concentrated. Ethyl acetate was added to the crude material, followed by saturated sodium bicarbonate and 1 N sodium hydroxide to pH 8 (litmus paper). The ethyl acetate layer was separated, washed with brine, dried over magnesium sulfate, filtered, and concentrated. The crude material was purified by flash chromatography over silica using a hexanes:ethyl acetate gradient of 0 to 50% ethyl acetate to afford 0.3 g (79%) of methyl 6-(6-hydroxy-2-naphthalenyl)-2-pyridinecarboxylate. ¹H NMR (400 MHz, DMSO-d₆): δ 9.92 (s, 1H), 8.55 (s, 1H), 8.29 (d, J=8 Hz, 1H), 8.17 (dd, J=9, 2 Hz, 1H), 8.06 (t, J=8 Hz, 1H), 7.97 (d, J=8 Hz, 1H), 7.89 (d, J=9 Hz, 1H), 7.80 (d, J=9 Hz, 1H), 7.13 (td, J=8, 2 Hz, 2H), 3.92 (s, 3H).

### 18c) Methyl 6-[6-({[3-(2,6-dichlorophenyl)-5-(1-methylethyl)-4-isoxazolyl]methyl}oxy)-2-naphthalenyl]-2-pyridinecarboxylate

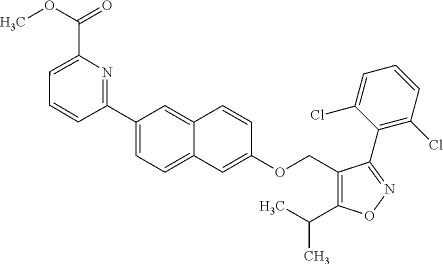

Methyl 6-(6-hydroxy-2-naphthalenyl)-2-pyridinecarboxylate (0.15 g, 0.537 mmol) and cesium carbonate (0.245 g, 0.752 mmol) in N,N-dimethylformamide (1.5 mL) were heated at 65° C. for 45 min. A solution of 4-(chloromethyl)-3-(2,6-dichlorophenyl)-5-(1-methylethyl)isoxazole (0.164 g, 0.537 mmol) in N,N-dimethylformamide (1 mL) was added to the reaction mixture and heating at 65° C. was continued for 24 h. The reaction mixture was cooled to room temperature, then diluted with water, followed by ethyl acetate. The ethyl acetate layer was separated, washed several times with water, followed by brine, dried over magnesium sulfate, filtered, and concentrated. The crude material was purified by flash chromatography over silica using a hexanes:ethyl acetate gradient of 0 to 50% ethyl acetate to afford 0.165 g (56%) of methyl 6-[6-({[3-(2,6-dichlorophenyl)-5-(1-methylethyl)-4-isoxazolyl]methyl}oxy)-2-naphthalenyl]-2-pyridinecarboxylate. ¹H NMR (400 MHz, DMSO-d₆): δ 8.57 (s, 1H), 8.30 (d, J=8 Hz, 1H), 8.22 (dd, J=9, 2 Hz, 1H), 8.08 (t, J=8 Hz, 1H), 7.99 (m, 1H), 7.89 (d, J=9 Hz, 1H), 7.84 (d, J=9 Hz, 1H), 7.62 (m, 2H), 7.52 (dd, J=9, 7 Hz, 1H), 7.32 (d, J=2 Hz, 1H), 6.94 (dd, J=9, 3 Hz, 1H), 4.95 (s, 2H), 3.92 (s, 3H), 3.50 (septet, J=7 Hz, 1H), 1.34 (d, J=7 Hz, 6H).

### 18d) 6-[6-({[3-(2,6-Dichlorophenyl)-5-(1-methylethyl)-4-isoxazolyl]methyl}oxy)-2-naphthalenyl]-2-pyridinecarboxylic acid

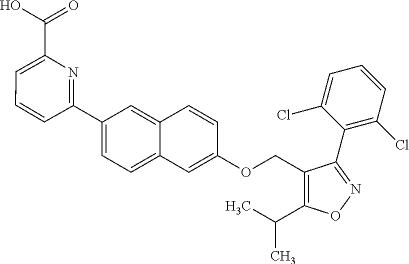

To a solution of methyl 6-[6-({[3-(2,6-dichlorophenyl)-5-(1-methylethyl)-4-isoxazolyl]methyl}oxy)-2-naphthalenyl]-2-pyridinecarboxylate (0.157 g, 0.287 mmol) in tetrahydrofuran (3 mL) and methanol (1.5 mL) was added 1 N sodium hydroxide (0.6 mL, 0.6 mmol) and the reaction mixture was stirred at 65° C. for 1 h. The reaction mixture was cooled to room temperature and concentrated. The crude material was acidified with 1N hydrochloric acid to pH 6 (litmus paper) and extracted with ethyl acetate. The ethyl acetate layer was separated, washed with brine, dried over magnesium sulfate, filtered, and concentrated to afford 0.16 g (100%) of 6-[6-({[3-(2,6-dichlorophenyl)-5-(1-methylethyl)-4-isoxazolyl]methyl}oxy)-2-naphthalenyl]-2-pyridinecarboxylic acid. ¹H NMR (400 MHz, DMSO-d₆): δ 13.17 (s, 1H), 8.63 (s, 1H), 8.28 (m, 2H), 8.06 (t, J=8 Hz, 1H), 7.97 (d, J=8 Hz, 1H), 7.88 (d, J=9 Hz, 1H), 7.83 (d, J=9 Hz, 1H), 7.62 (m, 2H), 7.52 (dd, J=9, 7 Hz, 1H), 7.32 (d, J=2

Hz, 1H), 6.94 (dd, J=9, 2 Hz, 1H), 4.95 (s, 2H), 3.50 (septet, J=8 Hz, 1H), 1.34 (d, J=7 Hz, 6H). HRMS C₂₉H₂₂Cl₂N₂O₄ m/z 533.10294 (M+H)⁺_{Cal}; 533.10299 (M+H)⁺_{Obs}.

### Example 19

### 5-[6-({[3-(2,6-Dichlorophenyl)-5-(1-methylethyl)-4-isoxazolyl]methyl}oxy)-2-naphthalenyl]-2-pyridinecarboxylic acid

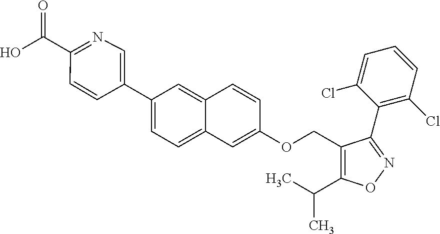

### 19a) Methyl 5-[6-(methyloxy)-2-naphthalenyl]-2-pyridinecarboxylate

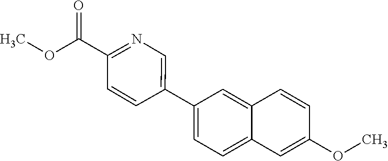

To a slurry of methyl 5-bromo-2-pyridinecarboxylate (1 g, 4.6 mmol), tetrakis(triphenylphosphine)palladium(0) (0.21 g, 0.185 mmol), ethylene glycol dimethyl ether (25 mL) and 2 N sodium carbonate (22 mL, 44 mmol) was added [6-(methyloxy)-2-naphthalenyl]boronic acid (1.12 g, 5.55 mmol) and the reaction mixture was heated at 80° C. for 1 h. The reaction mixture was cooled to room temperature, then diluted with water, followed by ethyl acetate. The layers were separated and the ethyl acetate layer was washed with brine, dried over magnesium sulfate, filtered, and concentrated. The crude material was partially dissolved in dichloromethane and filtered. The filtrate was purified by flash chromatography over silica using a hexanes:ethyl acetate gradient of 0 to 30% ethyl acetate to afford 0.127 g of methyl 5-[6-(methyloxy)-2-naphthalenyl]-2-pyridinecarboxylate. The aqueous layer was acidified with 1N hydrochloric acid to pH 6 (litmus paper) and extracted with ethyl acetate. The ethyl acetate layer was separated, washed with brine, dried over magnesium sulfate, filtered, and concentrated. This crude material was triturated with hot dichloromethane to afford 0.22 g of 5-[6-(methyloxy)-2-naphthalenyl]-2-pyridinecarboxylic acid. To a solution of 5-[6-(methyloxy)-2-naphthalenyl]-2-pyridinecarboxylic acid (0.219 g, 0.784 mmol) in methanol (7 mL) was slowly added thionyl chloride (0.114 mL, 1.57 mmol). The reaction mixture was heated at 75° C. for 2 days. The reaction mixture was cooled to room temperature, then concentrated. The crude material was diluted with saturated sodium bicarbonate, followed by ethyl acetate. The layers were separated and the ethyl acetate layer was washed with brine, dried over magnesium sulfate, filtered, and concentrated to afford 0.16 g of methyl 5-[6-(methyloxy)-2-naphthalenyl]-2-pyridinecarboxylate. This afforded the combined total of 0.287 g (21%) of methyl 5-[6-(methyloxy)-2-naphthalenyl]-2-pyridinecarboxylate. ¹H NMR (400 MHz, DMSO-d₆): δ 9.16 (d, J=2 Hz, 1H), 8.38 (dd, J=8, 2 Hz, 1H), 8.35 (s, 1H), 8.14 (d, J=8 Hz, 1H), 7.94 (m, 3H), 7.38 (d, J=2 Hz, 1H), 7.22 (dd, J=9, 3 Hz, 1H), 3.89 (s, 3H), 3.89 (s, 3H).

### 19b) Methyl 5-(6-hydroxy-2-naphthalenyl)-2-pyridinecarboxylate

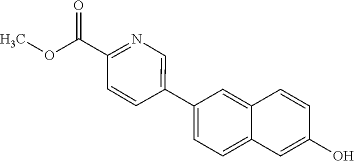

Boron tribromide (1 M in dichloromethane) (5 mL, 5 mmol) was added slowly to a slurry of methyl 5-[6-(methyloxy)-2-naphthalenyl]-2-pyridinecarboxylate (0.28 g, 0.955 mmol) in dichloromethane (18 mL) at 0° C. The reaction mixture was stirred for 1 h at 0° C. The reaction mixture was poured into ice water, then ethyl acetate and saturated sodium bicarbonate were added and the mixture was stirred for several minutes. The layers were separated and the ethyl acetate layer was washed with brine, dried over magnesium sulfate, filtered, and concentrated. The crude material was purified by flash chromatography over silica using a hexanes:ethyl acetate gradient of 0 to 50% ethyl acetate to afford 0.14 g (53%) of methyl 5-(6-hydroxy-2-naphthalenyl)-2-pyridinecarboxylate. ¹H NMR (400 MHz, DMSO-d₆): δ 9.94 (s, 1H), 9.13 (d, J=2 Hz, 1H), 8.35 (dd, J=8, 2 Hz, 1H), 8.28 (s, 1H), 8.12 (d, J=8 Hz, 1H), 7.87 (d, J=9 Hz, 1H), 7.82 (s, 2H), 7.16-7.15 (m, 1H), 7.13 (dd, J=9, 2 Hz, 1H), 3.89 (s, 3H).

### 19c) Methyl 5-[6-({[3-(2,6-dichlorophenyl)-5-(1-methylethyl)-4-isoxazolyl]methyl}oxy)-2-naphthalenyl]-2-pyridinecarboxylate

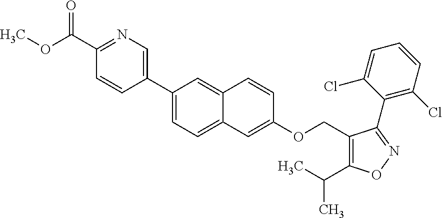

Methyl 5-(6-hydroxy-2-naphthalenyl)-2-pyridinecarboxylate (0.14 g, 0.501 mmol) and cesium carbonate (0.23 g, 0.702 mmol) in N,N-dimethylformamide (1.3 mL) were heated at 65° C. for 45 min. A solution of 4-(chloromethyl)-3-(2,6-dichlorophenyl)-5-(1-methylethyl)isoxazole (0.153 g, 0.501 mmol) in N,N-dimethylformamide (1 mL) was added to the reaction mixture and the reaction mixture was heated at 65° C. for 3 h. The reaction mixture was cooled to room temperature and diluted with water, followed by ethyl acetate. The ethyl acetate layer was separated, washed several times with water, followed by brine, dried over magnesium sulfate, filtered, and concentrated. The crude material was purified by flash chromatography over silica using a hexanes:ethyl acetate gradient of 0 to 50% ethyl acetate. This material was dissolved in ethyl acetate and washed several times with water. The ethyl acetate layer was dried over magnesium sulfate, filtered, and concentrated to afford 0.118 g (43%) of methyl 5-[6-({[3-(2,6-dichlorophenyl)-5-(1-methylethyl)-4-isoxazolyl]methyl}oxy)-2-naphthalenyl]-2-pyridinecarboxylate. ¹H NMR (400 MHz, DMSO-d₆): δ 9.14 (d, J=3 Hz, 1H), 8.36 (dd, J=8, 2 Hz, 1H), 8.31 (s, 1H), 8.13 (d, J=8 Hz, 1H), 7.88 (m, 3H), 7.61 (m, 2H), 7.52 (dd, J=7, 2 Hz, 1H), 7.33 (d, J=2 Hz, 1H), 6.96 (dd, J=9, 2 Hz, 1H), 4.95 (s, 2H), 3.89 (s, 3H), 3.50 (septet, J=7 Hz, 1H), 1.34 (d, J=7 Hz, 6H).

### 19d) 5-[6-({[3-(2,6-Dichlorophenyl)-5-(1-methylethyl)-4-isoxazolyl]methyl}oxy)-2-naphthalenyl]-2-pyridinecarboxylic acid

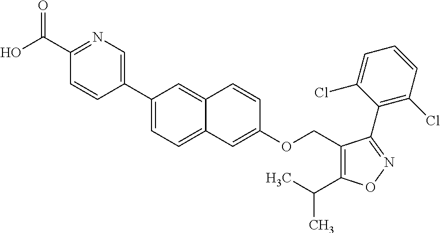

Methyl 5-[6-({[3-(2,6-dichlorophenyl)-5-(1-methylethyl)-4-isoxazolyl]methyl}oxy)-2-naphthalenyl]-2-pyridinecarboxylate (0.116 g, 0.212 mmol) and 1 N sodium hydroxide (0.45 mL, 0.45 mmol) were stirred in tetrahydrofuran (2.2 mL) and methanol (1.1 mL) at 65° C. for 1 h. The reaction mixture was cooled to room temperature, then concentrated. The crude material was diluted with 1 N hydrochloric acid and extracted with ethyl acetate. The ethyl acetate layer was separated, washed with brine, dried over magnesium sulfate, filtered, and concentrated. The crude material was triturated with hot dichloromethane, followed by hexanes, then methanol, and then it was then filtered and dried to afford 0.044 g (39%) of 5-[6-({[3-(2,6-dichlorophenyl)-5-(1-methylethyl)-4-isoxazolyl]methyl}oxy)-2-naphthalenyl]-2-pyridinecarboxylic acid. ¹H NMR (400 MHz, DMSO-d₆): δ 13.18 (br s, 1H), 9.11 (d, J=2 Hz, 1H), 8.34 (dd, J=8, 2 Hz, 1H), 8.29 (s, 1H), 8.11 (d, J=8 Hz, 1H), 7.88 (m, 3H), 7.61 (m, 2H), 7.52 (dd, J=9, 7 Hz, 1H), 7.33 (d, J=2 Hz, 1H), 6.96 (dd, J=9, 2 Hz, 1H), 4.95 (s, 2H), 3.51 (septet, J=7 Hz, 1H), 1.34 (d, J=7 Hz, 6H). HRMS C₂₉H₂₂Cl₂N₂O₄ m/z 533.10294 (M+H)⁺_{Cal}; 533.10279 (M+H)⁺_{Obs}.

### Example 20

### 4-[6-({[3-(2,6-Dichlorophenyl)-5-(1-methylethyl)-4-isoxazolyl]methyl}oxy)-2-naphthalenyl]-2-pyridinecarboxylic acid

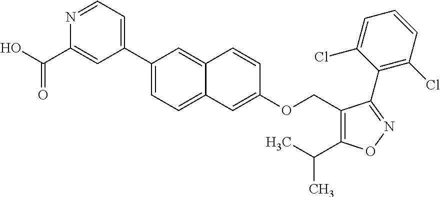

### 20a) 4-[6-(Methyloxy)-2-naphthalenyl]-2-pyridinecarboxylic acid

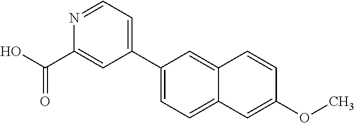

To a slurry of 4-bromo-2-pyridinecarboxylic acid (1 g, 4.95 mmol), tetrakis(triphenylphosphine)palladium(0) (0.23 g, 0.198 mmol), ethylene glycol dimethyl ether (27 mL) and 2 N sodium carbonate (24 mL) was added [6-(methyloxy)-2-naphthalenyl]boronic acid (1.2 g, 5.94 mmol) and the reaction mixture was heated at 80° C. for 4 days. The reaction mixture was cooled to room temperature, then diluted with water, followed by ethyl acetate. The layers were separated and the aqueous layer was acidified to pH 5 (litmus paper) then extracted with ethyl acetate. The ethyl acetate layer was washed with brine, dried over magnesium sulfate, filtered, and concentrated. The crude material was washed with hot dichloromethane to afford 0.85 g (62%) of 4-[6-(methyloxy)-2-naphthalenyl]-2-pyridinecarboxylic acid. ¹H NMR (400 MHz, DMSO-d₆): δ 8.73 (d, J=5 Hz, 1H), 8.41 (s, 2H), 8.03 (d, J=5 Hz, 1H), 7.98 (d, J=9 Hz, 1H), 7.94 (d, J=3 Hz, 2H), 7.38 (s, 1H), 7.22 (dd, J=9, 2 Hz, 1H), 3.88 (s, 3H).

### 20b) Methyl 4-[6-(methyloxy)-2-naphthalenyl]-2-pyridinecarboxylate

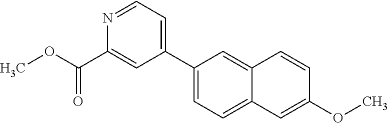

Thionyl chloride (0.44 mL, 6.09 mmol) was added slowly to a slurry of 4-[6-(methyloxy)-2-naphthalenyl]-2-pyridinecarboxylic acid (0.85 g, 3.04 mmol) in methanol (27 mL) and the reaction mixture was heated at 75° C. for 2 days. The reaction mixture was cooled to room temperature and concentrated. The crude material was diluted with saturated sodium bicarbonate and ethyl acetate. The mixture was stirred for several minutes, then the layers were separated and the ethyl acetate layer was washed with brine, dried over magnesium sulfate, filtered, and concentrated to afford 0.26 g (29%) of methyl 4-[6-(methyloxy)-2-naphthalenyl]-2-pyridinecarboxylate. ¹H NMR (400 MHz, DMSO-d₆): δ 8.76 (d, J=5 Hz, 1H), 8.42 (s, 2H), 8.08 (dd, J=5, 2 Hz, 1H), 7.96 (m, 3H), 7.39 (d, J=2 Hz, 1H), 7.23 (dd, J=9, 2 Hz, 1H), 3.91 (s, 3H), 3.89 (s, 3H).

### 20c) Methyl 4-(6-hydroxy-2-naphthalenyl)-2-pyridinecarboxylate

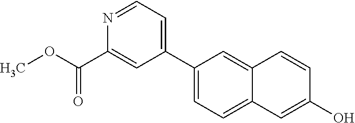

Boron tribromide (1 M in dichloromethane) (3.55 mL, 3.55 mmol) was added slowly to a solution of methyl 4-[6-(methyloxy)-2-naphthalenyl]-2-pyridinecarboxylate (0.26 g, 0.886 mmol) in dichloromethane (13 mL) at 0° C. The reaction mixture was stirred for 24 h at room temperature. The reaction mixture was poured into ice-water and extracted with ethyl acetate. The ethyl acetate layer was separated, washed with brine, dried over magnesium sulfate, filtered, and concentrated to afford impure 0.4 g of 4-(6-hydroxy-2-naphthalenyl)-2-pyridinecarboxylic acid. Thionyl chloride (0.22 mL, 3.02 mmol) was added slowly to a slurry of 4-(6-hydroxy-2-naphthalenyl)-2-pyridinecarboxylic acid (0.4 g, 1.51 mmol) in methanol (14 mL) and the reaction mixture was heated at 75° C. for 24 h, then at room temperature for 3 days. The reaction mixture was concentrated, then the crude material was diluted with saturated sodium bicarbonate and ethyl acetate. The layers were separated and the ethyl acetate layer was washed with brine, dried over magnesium sulfate, filtered, and concentrated. The crude material was purified by flash chromatography over silica using a hexanes:ethyl acetate gradient of 0 to 50% ethyl acetate to afford 0.10 g (40%) of methyl 4-(6-hydroxy-2-naphthalenyl)-2-pyridinecarboxylate. ¹H NMR (400 MHz, DMSO-d₆): δ 9.98 (s, 1H), 8.75 (d, J=5 Hz, 1H), 8.39 (s, 1H), 8.35 (s, 1H), 8.05 (d, J=5 Hz, 1H), 7.92 (d, J=9 Hz, 1H), 7.85 (m, 2H), 7.15 (m, 2H), 3.91 (s, 3H).

### 20d) Methyl 4-[6-({[3-(2,6-dichlorophenyl)-5-(1-methylethyl)-4-isoxazolyl]methyl}oxy)-2-naphthalenyl]-2-pyridinecarboxylate

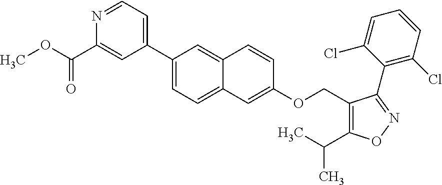

Methyl 4-(6-hydroxy-2-naphthalenyl)-2-pyridinecarboxylate (0.10 g, 0.365 mmol) and cesium carbonate (0.168 g, 0.511 mmol) in N,N-dimethylformamide (1.7 mL) were heated at 65° C. for 1 h. A solution of 4-(chloromethyl)-3-(2,6-dichlorophenyl)-5-(1-methylethyl)isoxazole (0.111 g, 0.365 mmol) in N,N-dimethylformamide (0.5 mL) was added to the reaction mixture and heating at 65° C. was continued for 24 h. The reaction mixture was cooled to room temperature and diluted with water, followed by ethyl acetate. The ethyl acetate layer was separated, washed several times with water, followed by brine, dried over magnesium sulfate, filtered, and concentrated to afford 0.17 g (85%) of methyl 4-[6-({[3-(2,6-dichlorophenyl)-5-(1-methylethyl)-4-isoxazolyl]methyl}oxy)-2-naphthalenyl]-2-pyridinecarboxylate. ¹H NMR (400 MHz, DMSO-d₆): δ 8.76 (d, J=5 Hz, 1H), 8.39 (d, J=6 Hz, 2H), 7.87 (t, J=9 Hz, 1H), 7.92 (dd, J=5, 3 Hz, 2H), 7.60 (m, 3H), 7.57-7.49 (m, 1H), 7.34 (d, J=2 Hz, 1H), 6.96 (dd, J=9, 3 Hz, 1H), 4.96 (s, 2H), 3.91 (s, 3H), 3.51 (septet, J=7 Hz, 1H), 1.34 (d, J=7 Hz, 6H).

### 20e) 4-[6-({[3-(2,6-Dichlorophenyl)-5-(1-methylethyl)-4-isoxazolyl]methyl}oxy)-2-naphthalenyl]-2-pyridinecarboxylic acid

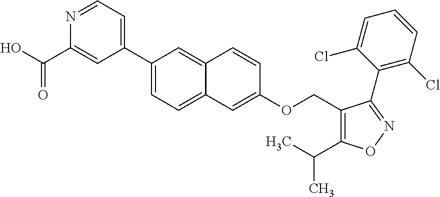

Methyl 4-[6-({[3-(2,6-dichlorophenyl)-5-(1-methylethyl)-4-isoxazolyl]methyl}oxy)-2-naphthalenyl]-2-pyridinecarboxylate (0.17 g, 0.311 mmol) and 1 N sodium hydroxide (0.66 mL) were stirred in tetrahydrofuran (3.2 mL) and methanol (1.6 mL) at room temperature overnight. The reaction mixture was concentrated and the crude material was diluted with 1 N hydrochloric acid to pH 4 (litmus paper). The acidic mixture was extracted with ethyl acetate. The layers were separated and the pH of the aqueous layer was adjusted to approximately 6 (litmus paper) with 1N sodium hydroxide and filtered. The solid was dried to afford 0.010 g (6%) of 4-[6-({[3-(2,6-dichlorophenyl)-5-(1-methylethyl)-4-isoxazolyl]methyl}oxy)-2-naphthalenyl]-2-pyridinecarboxylic acid. ¹H NMR (400 MHz, DMSO-d₆): δ 8.75 (d, J=5.13 Hz, 1H), 8.40 (d, J=5 Hz, 2H), 8.05 (d, J=5 Hz, 1H), 7.92 (t, J=8 Hz, 2H), 7.86 (m, 1H), 7.64-7.58 (m, 2H), 7.52 (m, 1H), 7.34 (d, J=2 Hz, 1H), 6.96 (dd, J=9, 2 Hz, 1H), 4.96 (s, 2H), 3.51 (septet, J=7 Hz, 1H), 1.34 (d, J=7 Hz, 6H). HRMS C₂₉H₂₂Cl₂N₂O₄ m/z 533.10294 (M+H)⁺_{Cal}; 533.10297 (M+H)⁺_{Obs}.

### Example 21

### 2-[6-({[3-(2,6-Dichlorophenyl)-5-(1-methylethyl)-4-isoxazolyl]methyl}oxy)-2-naphthalenyl]-4-pyridinecarboxylic acid

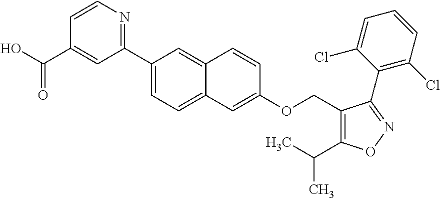

### 21a) Methyl 2-[6-(methyloxy)-2-naphthalenyl]-4-pyridinecarboxylate

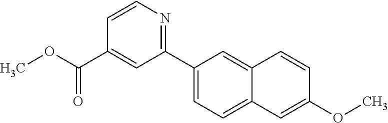

To a slurry of 2-bromo-4-pyridinecarboxylic acid (1.2 g, 5.94 mmol), tetrakis(triphenylphosphine)palladium(0) (0.23 g, 0.198 mmol), ethylene glycol dimethyl ether (27 mL) and 2 N sodium carbonate (24 mL) was added [6-(methyloxy)-2-naphthalenyl]boronic acid (1 g, 4.95 mmol) and the reaction mixture was heated at 80° C. for 24 h. The reaction mixture was cooled to room temperature, then diluted with water, followed by ethyl acetate. The layers were separated and the aqueous layer was acidified to approximately pH 5 (litmus paper) with 1N hydrochloric acid and extracted with ethyl acetate. The ethyl acetate layer was filtered to afford 0.02 g of 2-[6-(methyloxy)-2-naphthalenyl]-4-pyridinecarboxylic acid and the filtrate was washed with brine, dried over magnesium sulfate, filtered, and concentrated to afford 1.3 g of 2-[6-(methyloxy)-2-naphthalenyl]-4-pyridinecarboxylic acid. The materials were combined to afford a total of 1.32 g (96%) of 2-[6-(methyloxy)-2-naphthalenyl]-4-pyridinecarboxylic acid with a small impurity present by ¹H NMR. Thionyl chloride (0.57 mL, 7.80 mmol) was added slowly to a slurry of 2-[6-(methyloxy)-2-naphthalenyl]-4-pyridinecarboxylic acid (1.09 g, 3.90 mmol) in methanol (35 mL) and the reaction mixture was heated at 75° C. for 2 days. The reaction mixture was cooled to room temperature, then concentrated. The crude material was diluted with 5% sodium bicarbonate and ethyl acetate. The mixture was stirred for several minutes, then the layers were separated and the ethyl acetate layer was washed with brine, dried over magnesium sulfate, filtered, and concentrated. The crude material was purified by flash chromatography over silica using a hexanes:ethyl acetate gradient of 0 to 30% ethyl acetate to afford 0.597 g (52%) of methyl 2-[6-(methyloxy)-2-naphthalenyl]-4-pyridinecarboxylate. ¹H NMR (400 MHz, DMSO-d₆): δ 8.88 (d, J=5 Hz, 1H), 8.65 (s, 1H), 8.43 (s, 1H), 8.23 (dd, J=9, 2 Hz, 1H), 8.00 (d, J=9 Hz, 1H), 7.92 (d, J=9 Hz, 1H), 7.77 (d, J=7 Hz, 1H), 7.36 (d, J=2 Hz, 1H), 7.20 (dd, J=9, 3 Hz, 1H), 3.93 (s, 3H), 3.88 (s, 3H).

### 21b) Methyl 2-[6-({[3-(2,6-dichlorophenyl)-5-(1-methylethyl)-4-isoxazolyl]methyl}oxy)-2-naphthalenyl]-4-pyridinecarboxylate

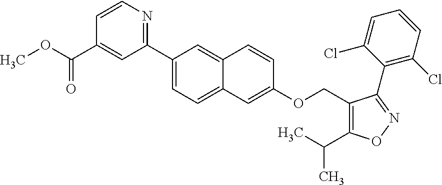

Boron tribromide (1 M in dichloromethane) (8.05 mL, 8.05 mmol) was added slowly to a solution of methyl 2-[6-(methyloxy)-2-naphthalenyl]-4-pyridinecarboxylate (0.59 g, 2.01 mmol) in dichloromethane (29.5 mL) at 0° C. The reaction mixture was stirred for 24 h at room temperature. The reaction mixture was poured into ice-water and stirred for several minutes. Sodium hydroxide (1 N) was added to the aqueous mixture to pH 8 (litmus paper) and the basic mixture was extracted with ethyl acetate. The ethyl acetate layer was separated, washed with brine, dried over magnesium sulfate, filtered, and concentrated to afford 0.5 g of impure methyl 2-(6-hydroxy-2-naphthalenyl)-4-pyridinecarboxylate. A mixture of methyl 2-(6-hydroxy-2-naphthalenyl)-4-pyridinecarboxylate (0.25 g, 0.895 mmol) and cesium carbonate (0.41 g, 1.25 mmol) in N,N-dimethylformamide (3 mL) was heated at 65° C. for 1 h. A solution of 4-(chloromethyl)-3-(2,6-dichlorophenyl)-5-(1-methylethyl)isoxazole (0.273 g, 0.895 mmol) in N,N-dimethylformamide (1 mL) was added to the reaction mixture and heating at 65° C. was continued for 24 h. The reaction mixture was cooled to room temperature and diluted with water, followed by ethyl acetate. The ethyl acetate layer was separated, washed several times with water, followed by brine, dried over magnesium sulfate, filtered, and concentrated. The crude oil was purified by flash chromatography over silica using a hexanes:ethyl acetate gradient of 0 to 30% ethyl acetate to afford 0.13 g (27%) of methyl 2-[6-({[3-(2,6-dichlorophenyl)-5-(1-methylethyl)-4-isoxazolyl]methyl}oxy)-2-naphthalenyl]-4-pyridinecarboxylate. ¹H NMR (400 MHz, DMSO-d₆): δ 8.87 (d, J=5 Hz, 1H), 8.61 (s, 1H), 8.41 (s, 1H), 8.21 (d, J=7 Hz, 1H), 7.93 (d, J=9 Hz, 1H), 7.82 (d, J=9 Hz, 1H), 7.77 (d, J=5 Hz, 1H), 7.61 (m, 2H), 7.52 (m, 1H), 7.32 (s, 1H), 6.94 (dd, J=9, 2 Hz, 1H), 4.95 (s, 2H), 3.93 (s, 3H), 3.50 (septet, J=7 Hz, 1H), 1.34 (d, J=7 Hz, 6H).

### 21c) 2-[6-({[3-(2,6-Dichlorophenyl)-5-(1-methylethyl)-4-isoxazolyl]methyl}oxy)-2-naphthalenyl]-4-pyridinecarboxylic acid

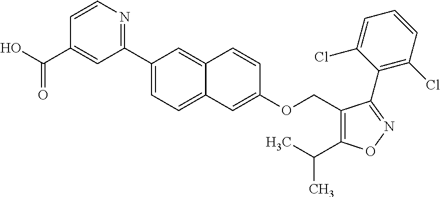

Methyl 2-[6-({[3-(2,6-dichlorophenyl)-5-(1-methylethyl)-4-isoxazolyl]methyl}oxy)-2-naphthalenyl]-4-pyridinecarboxylate (0.13 g, 0.237 mmol) and 1 N sodium hydroxide (0.5 mL) were stirred in tetrahydrofuran (2.5 mL) and methanol (1.23 mL) at room temperature overnight. The reaction mixture was concentrated and the crude material was diluted with 1 N hydrochloric acid to pH 4 (litmus paper). The acidic mixture was extracted with ethyl acetate. The ethyl acetate layer was separated, washed with brine, dried over magnesium sulfate, filtered, and concentrated. The crude material was dissolved in methanol and dichloromethane. A precipitate formed immediately which was filtered, washed with dichloromethane and dried to afford 0.028 (22%) of 2-[6-({[3-(2,6-dichlorophenyl)-5-(1-methylethyl)-4-isoxazolyl]methyl}oxy)-2-naphthalenyl]-4-pyridinecarboxylic acid. ¹H NMR (400 MHz, DMSO-d₆): δ 13.74 (s, 1H), 8.84 (d, J=5 Hz, 1H), 8.60 (s, 1H), 8.40 (s, 1H), 8.21 (dd, J=9, 2 Hz, 1H), 7.92 (d, J=9 Hz, 1H), 7.82 (d, J=9 Hz, 1H), 7.75 (dd, J=5, 1 Hz, 1H), 7.61 (m, 2H), 7.52 (m, 1H), 7.31 (d, J=2 Hz, 1H), 6.93 (dd, J=9, 2 Hz, 1H), 4.95 (s, 2H), 3.50 (septet, J=7 Hz, 1H), 1.34 (d, J=7 Hz, 6H). HRMS C₂₉H₂₂Cl₂N₂O₄ m/z 533.1035 (M+H)⁺_{Cal}; 533.1039 (M+H)⁺_{Obs}.

### Example 22

### 4-[6-({[3-(2,6-Dichlorophenyl)-5-(1-methylethyl)-4-isoxazolyl]methyl}oxy)-2-naphthalenyl]benzoic acid

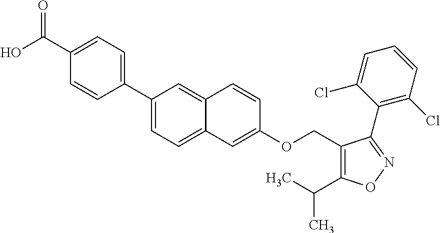

### 22a) 4-{[(6-Bromo-2-naphthalenyl)oxy]methyl}-3-(2,6-dichlorophenyl)-5-(1-methylethyl)isoxazole

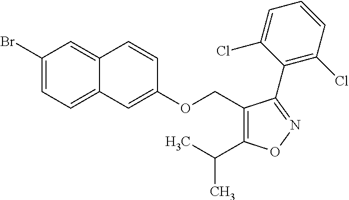

A mixture of 6-bromo-2-naphthalenol (2 g, 8.97 mmol) and cesium carbonate (4.1 g, 12.6 mmol) in N,N-dimethylformamide (20 mL) was heated at 65° C. for 45 min. A solution of 4-(chloromethyl)-3-(2,6-dichlorophenyl)-5-(1-methylethyl)isoxazole (2.73 g, 8.97 mmol) in N,N-dimethylformamide (3 mL) was added to the reaction mixture and heating at 65° C. was continued for 24 h. The reaction mixture was cooled to room temperature and diluted with water, followed by ethyl acetate. The ethyl acetate layer was separated, washed several times with water, followed by brine, dried over magnesium sulfate, filtered, and concentrated. The crude material was purified by flash chromatography over silica using a hexanes:ethyl acetate gradient of 0 to 50% ethyl acetate to afford 2.6 g (59%) of 4-{[(6-bromo-2-naphthalenyl)oxy]methyl}-3-(2,6-dichlorophenyl)-5-(1-methylethyl)isoxazole. ¹H NMR (400 MHz, DMSO-d₆): δ 8.05 (d, J=2 Hz, 1H), 7.73 (d, J=9 Hz, 1H), 7.66 (d, J=9 Hz, 1H), 7.60 (m, 2H), 7.51 (m, 2H), 7.29 (d, J=2 Hz, 1H), 6.93 (dd, J=9, 2 Hz, 1H), 4.91 (s, 2H), 3.48 (septet, J=7 Hz, 1H), 1.32 (d, J=7 Hz, 6H).

### 22b) 4-[6-({[3-(2,6-Dichlorophenyl)-5-(1-methylethyl)-4-isoxazolyl]methyl}oxy)-2-naphthalenyl]benzoic acid

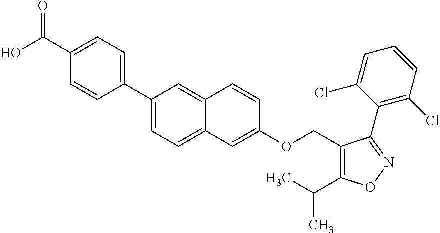

To a slurry of 4-{[(6-bromo-2-naphthalenyl)oxy]methyl}-3-(2,6-dichlorophenyl)-5-(1-methylethyl)isoxazole (0.1 g, 0.204 mmol), tetrakis(triphenylphosphine)palladium(0) (0.009 g, 0.008 mmol), ethylene glycol dimethyl ether (1.1 mL) and 2 N sodium carbonate (1 mL, 2 mmol) was added 4-(dihydroxyboranyl)benzoic acid (0.041 g, 0.244 mmol) and the reaction mixture was heated at 80° C. for 24 h. The reaction mixture was cooled to room temperature and diluted with water, followed by ethyl acetate. Hydrochloric acid (1 N) was added to the mixture until an acidic pH (litmus paper) was obtained, and then the layers were separated. The ethyl acetate layer was washed with brine, dried over magnesium sulfate, filtered, and concentrated. The crude material was purified by flash chromatography over silica using a hexanes:ethyl acetate gradient of 0 to 50% ethyl acetate to afford impure product which was purified again by flash chromatography over silica using a dichloromethane:methanol gradient of 0 to 5% methanol to afford 0.019 g (18%) of 4-[6-({[3-(2,6-dichlorophenyl)-5-(1-methylethyl)-4-isoxazolyl]methyl}oxy)-2-naphthalenyl]benzoic acid. ¹H NMR (400 MHz, DMSO-d₆): δ 12.95 (s, 1H), 8.20 (s, 1H), 8.02 (d, J=8 Hz, 2H), 7.87 (m, 5H), 7.61 (m, 2H), 7.52 (m, 1H), 7.31 (s, 1H), 6.93 (dd, J=9, 2 Hz, 1H), 4.95 (s, 2H), 3.51 (septet, J=7 Hz, 1H), 1.34 (d, J=6 Hz, 6H). HRMS C₃₀H₂₃Cl₂NO₄ m/z 532.1082 (M+H)⁺_{Cal}; 532.1072 (M+H)⁺_{Obs}.

### Example 23

### 3-{4-[6-({[3-(2,6-Dichlorophenyl)-5-(1-methylethyl)-4-isoxazolyl]methyl}oxy)-2-naphthalenyl]phenyl}propanoic acid

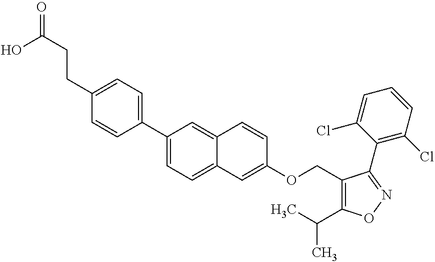

### 23a) 3-{4-[6-({[3-(2,6-Dichlorophenyl)-5-(1-methylethyl)-4-isoxazolyl]methyl}oxy)-2-naphthalenyl]phenyl}propanoic acid

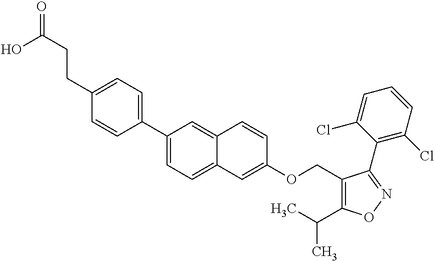

To a slurry of 4-{[(6-bromo-2-naphthalenyl)oxy]methyl}-3-(2,6-dichlorophenyl)-5-(1-methylethyl)isoxazole (prepared according to the general procedure described for Example 22a) (0.15 g, 0.305 mmol), tetrakis(triphenylphosphine)palladium(0) (0.014 g, 0.0122 mmol), ethylene glycol dimethyl ether (1.65 mL) and 2 N sodium carbonate (1.5 mL, 3 mmol) was added 3-[4-(dihydroxyboranyl)phenyl]propanoic acid (0.071 g, 0.366 mmol) and the reaction mixture was heated at 80° C. for 2 h. The reaction mixture was cooled to room temperature and diluted with water, followed by ethyl acetate. The mixture was acidified with 1 N hydrochloric acid to pH approximately 4 (litmus paper) and the layers were separated. The ethyl acetate layer was washed with brine, dried over magnesium sulfate, filtered, and concentrated. The crude material was purified by flash chromatography over silica using a dichloromethane:methanol gradient of 0 to 5% methanol to afford impure product which was purified again by flash chromatography over silica using a hexanes:ethyl acetate gradient of 0 to 50% ethyl acetate to afford 0.047 g (27%) of 3-{4-[6-({[3-(2,6-dichlorophenyl)-5-(1-methylethyl)-4-isoxazolyl]methyl}oxy)-2-naphthalenyl]phenyl}propanoic acid. ¹H NMR (400 MHz, DMSO-d₆): δ 12.12 (s, 1H), 8.05 (s, 1H), 7.77 (m, 3H), 7.65 (d, J=8 Hz, 2H), 7.61 (m, 2H), 7.52 (m, 1H), 7.31 (d, J=8 Hz, 2H), 7.27 (s, 1H), 6.90 (dd, J=9, 2 Hz, 1H), 4.93 (s, 2H), 3.50 (septet, J=7 Hz, 1H), 2.84 (t, J=8 Hz, 2H), 2.55 (t, J=8 Hz, 2H), 1.33 (d, J=7 Hz, 6H). HRMS C₃₂H₂₇Cl₂NO₄ m/z 560.1395 (M+H)⁺_{Cal}; 560.1393 (M+H)⁺_{Obs}.

### Example 24

### 6-[6-({[3-(2,6-Dichlorophenyl)-5-(1-methylethyl)-4-isoxazolyl]methyl}oxy)-2-naphthalenyl]-3-pyridinecarboxylic acid

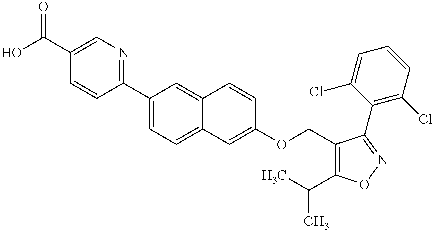

### 24a) 6-[6-(Methyloxy)-2-naphthalenyl]-3-pyridinecarboxylic acid

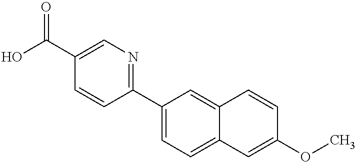

To a slurry of 6-bromo-3-pyridinecarboxylic acid (1.2 g, 5.94 mmol), tetrakis(triphenylphosphine)palladium(0) (0.23 g, 0.198 mmol), ethylene glycol dimethyl ether (27 mL) and 2 N sodium carbonate (24 mL, 48 mmol) was added [6-(methyloxy)-2-naphthalenyl]boronic acid (1 g, 4.95 mmol) and the reaction mixture was heated at 80° C. for 3 h. The reaction mixture was cooled to room temperature and diluted with water, followed by ethyl acetate. The layers were separated and the aqueous layer was acidified with 1N hydrochloric acid to pH 5 (litmus paper) and extracted with ethyl acetate. The organic extracts were combined, washed with brine, dried over magnesium sulfate, filtered, and concentrated to afford 0.49 g (36%) of 6-[6-(methyloxy)-2-naphthalenyl]-3-pyridinecarboxylic acid. ¹H NMR (400 MHz, DMSO-d₆) δ 13.32 (br s, 1H), 9.14 (dd, J=2, 1 Hz, 1H), 8.69 (s, 1H), 8.33 (dd, J=8, 2 Hz, 1H), 8.25 (dd, J=9, 2 Hz, 1H), 8.21 (dd, J=8, 1 Hz, 1H), 7.97 (d, J=9 Hz, 1H), 7.93 (d, J=9 Hz, 1H), 7.37 (d, J=3 Hz, 1H), 7.21 (dd, J=9, 3 Hz, 1H), 3.89 (s, 3H).

### 24b) Methyl 6-[6-(methyloxy)-2-naphthalenyl]-3-pyridinecarboxylate

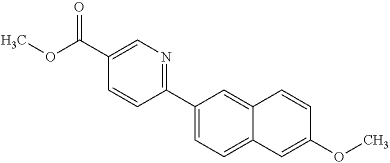

Thionyl chloride (0.26 mL, 3.51 mmol) was added slowly to a slurry of 6-[6-(methyloxy)-2-naphthalenyl]-3-pyridinecarboxylic acid (0.49 g, 1.75 mmol) in methanol (16 mL) and the reaction mixture was heated at 75° C. for 2 weeks. The reaction mixture was cooled to room temperature and concentrated. The crude material was diluted with 5% sodium bicarbonate and ethyl acetate. The mixture was stirred for several minutes and the layers were separated. The ethyl acetate layer was washed with brine, dried over magnesium sulfate, filtered, and concentrated to afford 0.154 g (30%) of methyl 6-[6-(methyloxy)-2-naphthalenyl]-3-pyridinecarboxylate. ¹H NMR (400 MHz, DMSO-d₆): δ 9.16 (d, J=2 Hz, 1H), 8.70 (s, 1H), 8.36 (dd, J=8, 2 Hz, 1H), 8.25 (m, 2H), 7.98 (d, J=9 Hz, 1H), 7.93 (d, J=9 Hz, 1H), 7.37 (d, J=2 Hz, 1H), 7.21 (dd, J=9, 3 Hz, 1H), 3.90 (s, 3H), 3.89 (s, 3H).

### 24c) Methyl 6-[6-({[3-(2,6-dichlorophenyl)-5-(1-methylethyl)-4-isoxazolyl]methyl}oxy)-2-naphthalenyl]-3-pyridinecarboxylate

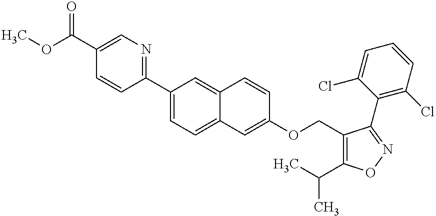

Boron tribromide (1 M in dichloromethane) (2.1 mL, 2.10 mmol) was added slowly to a solution of methyl 6-[6-(methyloxy)-2-naphthalenyl]-3-pyridinecarboxylate (0.154 g, 0.525 mmol) in dichloromethane (8 mL) at 0° C. The reaction mixture was stirred for 2 h at 0° C. The reaction mixture was poured into ice-water and stirred for several minutes. The pH of the aqueous mixture was adjusted to approximately 8 (litmus paper) with sodium hydroxide (1 N) and extracted with ethyl acetate. The ethyl acetate layer was separated, washed with brine, dried over magnesium sulfate, filtered, and concentrated to afford 0.130 g (88%) of impure methyl 6-(6-hydroxy-2-naphthalenyl)-3-pyridinecarboxylate. Methyl 6-(6-hydroxy-2-naphthalenyl)-3-pyridinecarboxylate (0.13 g, 0.465 mmol) and cesium carbonate (0.21 g, 0.652 mmol) in N,N-dimethylformamide (1 mL) were heated at 65° C. for 1 h. A solution of 4-(chloromethyl)-3-(2,6-dichlorophenyl)-5-(1-methylethyl)isoxazole (0.142 g, 0.465 mmol) in N,N-dimethylformamide (1 mL) was added to the reaction mixture and heating at 65° C. was continued for 3 h. The reaction mixture was cooled to room temperature and diluted with water, followed by ethyl acetate. The ethyl acetate layer was separated, washed several times with water, followed by brine, dried over magnesium sulfate, filtered, and concentrated. The crude oil was purified by flash chromatography over silica using a hexanes:ethyl acetate gradient of 0 to 50% ethyl acetate to afford 0.095 g (38%) of methyl 6-[6-({[3-(2,6-dichlorophenyl)-5-(1-methylethyl)-4-isoxazolyl]methyl}oxy)-2-naphthalenyl]-3-pyridinecarboxylate. ¹H NMR (400 MHz, DMSO-d₆): δ 9.15 (s, 1H), 8.66 (s, 1H), 8.35 (d, J=9 Hz, 1H), 8.23 (m, 2H), 7.90 (d, J=9 Hz, 1H), 7.83 (d, J=9 Hz, 1H), 7.61 (m, 2H), 7.52 (m, 1H), 7.33 (s, 1H), 6.94 (dd, J=9, 2 Hz, 1H), 4.96 (s, 2H), 3.89 (s, 3H), 3.50 (septet, J=6 Hz, 1H), 1.34 (d, J=7 Hz, 6H).

### 24d) 6-[6-({[3-(2,6-Dichlorophenyl)-5-(1-methylethyl)-4-isoxazolyl]methyl}oxy)-2-naphthalenyl]-3-pyridinecarboxylic acid

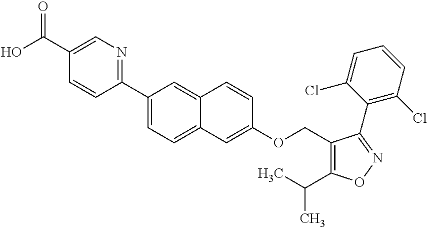

Methyl 6-[6-({[3-(2,6-dichlorophenyl)-5-(1-methylethyl)-4-isoxazolyl]methyl}oxy)-2-naphthalenyl]-3-pyridinecarboxylate (0.095 g, 0.174 mmol) and 1 N sodium hydroxide (0.37 mL) were stirred in tetrahydrofuran (1.8 mL) and methanol (0.9 mL) at room temperature overnight. The reaction mixture was concentrated and diluted with 1 N hydrochloric acid to pH 4 (litmus paper). The acidic aqueous mixture was extracted with ethyl acetate. The organic layer was separated, washed with brine, dried over magnesium sulfate, filtered, and concentrated. The crude material was purified by flash chromatography over silica using a dichloromethane:methanol gradient of 0 to 10% methanol to afford 0.016 g (17%) of 6-[6-({[3-(2,6-dichlorophenyl)-5-(1-methylethyl)-4-isoxazolyl]methyl}oxy)-2-naphthalenyl]-3-pyridinecarboxylic acid. ¹H NMR (400 MHz, DMSO-d₆): δ 13.36 (s, 1H), 9.13 (d, J=2 Hz, 1H), 8.65 (s, 1H), 8.32 (dd, J=9, 2 Hz, 1H), 8.23 (d, J=9 Hz, 1H), 8.19 (d, J=8 Hz, 1H), 7.90 (d, J=9 Hz, 1H), 7.83 (d, J=9 Hz, 1H), 7.61 (m, 2H), 7.52 (m, 1H), 7.32 (d, J=3 Hz, 1H), 6.94 (dd, J=9, 3 Hz, 1H), 4.95 (s, 2H), 3.50 (septet, J=7 Hz, 1H), 1.34 (d, J=7 Hz, 6H). HRMS C₂₉H₂₂Cl₂N₂O₄ m/z 533.1035 (M+H)⁺_{Cal}; 533.1033 (M+H)⁺_{Obs}.

### Example 25

### 5-[6-({[3-(2,6-Dichlorophenyl)-5-(1-methylethyl)-4-isoxazolyl]methyl}oxy)-2-naphthalenyl]-2-thiophenecarboxylic acid

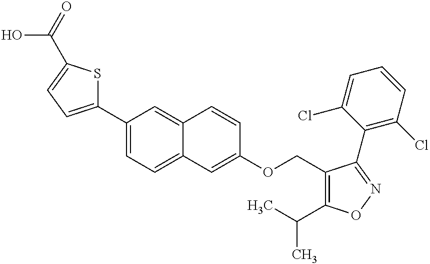

### 25a) 5-[6-({[3-(2,6-Dichlorophenyl)-5-(1-methylethyl)-4-isoxazolyl]methyl}oxy)-2-naphthalenyl]-2-thiophenecarboxylic acid

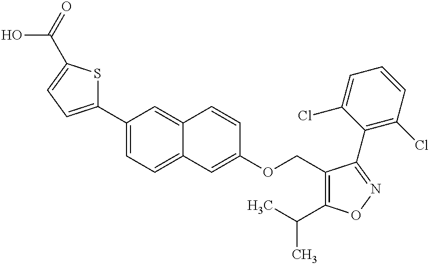

To a slurry of 4-{[(6-bromo-2-naphthalenyl)oxy]methyl}-3-(2,6-dichlorophenyl)-5-(1-methylethyl)isoxazole (prepared according to the general procedure described for Example 22a) (0.2 g, 0.407 mmol), palladium (II) acetate (0.0045 g, 0.0204 mmol), tri-o-toylphosphine (0.0124 g, 0.0407 mmol), N,N-dimethylformamide (26 mL) and 2

N sodium carbonate (0.53 mL. 1.06 mmol) was added 5-(dihydroxyboranyl)-2-thiophenecarboxylic acid (0.105 g, 0.611 mmol) and the reaction mixture was heated at 80° C. for 3 h. The reaction mixture was cooled to room temperature, then filtered through a pad of Celite® and the Celite® pad was washed with ethyl acetate. The filtrate was washed several times with water followed by brine then dried over magnesium sulfate, filtered, and concentrated. The crude material was purified by flash chromatography over silica using a hexanes:acetone gradient of 0 to 50% acetone to afford 0.008 g (3.7%) of 5-[6-({[3-(2,6-dichlorophenyl)-5-(1-methylethyl)-4-isoxazolyl]methyl}oxy)-2-naphthalenyl]-2-thiophenecarboxylic acid. ¹H NMR (400 MHz, DMSO-d₆): δ 13.11 (s, 1H), 8.19 (s, 1H), 7.83 (d, J=9 Hz, 1H), 7.78 (m, 2H), 7.72 (dd, J=4, 2 Hz, 1H), 7.62 (m, 3H), 7.52 (m, 1H), 7.30 (s, 1H), 6.93 (d, J=9 Hz, 1H), 4.93 (m, 2H), 3.50 (septet, J=7 Hz, 1H), 1.33 (d, J=7 Hz, 6H). HRMS C₂₈H₂₁Cl₂NO₄S m/z 538.0647 (M+H)⁺_{Cal}; 538.0646 (M+H)⁺_{Obs}.

### Example 26

### N-{3-[6-({[3-(2,6-Dichlorophenyl)-5-(1-methylethyl)-4-isoxazolyl]methyl}oxy)-2-naphthalenyl]phenyl}-1,1,1-trifluoro-N-[(trifluoromethyl)sulfonyl]methanesulfonamide

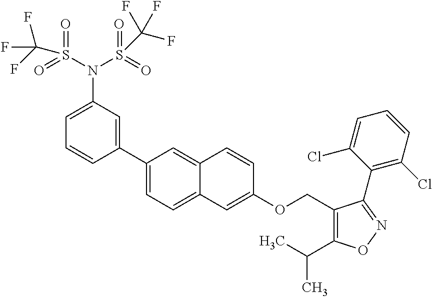

### 26a) 6-(3-Aminophenyl)-2-naphthalenol

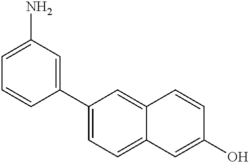

To a slurry of 6-bromo-2-naphthalenol (0.68 g, 3.04 mmol), tetrakis(triphenylphosphine)palladium(0) (0.14 g, 0.122 mmol), ethylene glycol dimethyl ether (17 mL) and 2 N sodium carbonate (15 mL, 30 mmol) was added (3-aminophenyl)boronic acid (0.5 g, 3.65 mmol) and the reaction mixture was heated at 80° C. for 2 h. The reaction was cooled to room temperature and diluted with water, followed by ethyl acetate. The ethyl acetate layer was separated, washed with water, followed by brine, dried over magnesium sulfate, filtered, and concentrated. Dichloromethane was added to the crude product, followed by hot methanol. Precipitate formed immediately and the mixture was cooled to room temperature and filtered. The filtrate was concentrated and the resulting solid was triturated with hot dichloromethane and methanol. The combined solids from the two triturations afforded 0.384 g (54%) of 6-(3-aminophenyl)-2-naphthalenol. ¹H NMR (400 MHz, DMSO-d₆): δ 9.72 (s, 1H), 7.92 (s, 1H), 7.79 (d, J=9 Hz, 1H), 7.70 (d, J=8 Hz, 1H), 7.59 (m, 1H), 7.8 (m, 3H), 6.91 (s, 1H), 6.84 (d, J=8 Hz, 1H), 6.53 (d, J=8 Hz, 1H), 5.13 (s, 2H).

### 26b) 3-[6-({[3-(2,6-Dichlorophenyl)-5-(1-methylethyl)-4-isoxazolyl]methyl}oxy)-2-naphthalenyl]aniline

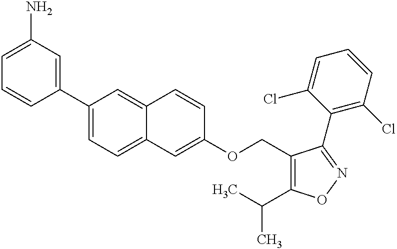

A mixture of 6-(3-aminophenyl)-2-naphthalenol (0.38 g, 1.62 mmol) and cesium carbonate (0.74 g, 2.26 mmol) in N,N-dimethylformamide (5.5 mL) was heated at 65° C. for 1 h. A solution of 4-(chloromethyl)-3-(2,6-dichlorophenyl)-5-(1-methylethyl)isoxazole (0.49 g, 1.62 mmol) in N,N-dimethylformamide (2 mL) was added to the reaction mixture and heating at 65° C. was continued for 3 h. The reaction mixture was cooled to room temperature and diluted with water, followed by ethyl acetate. The ethyl acetate layer was separated, washed several times with water, followed by brine, dried over magnesium sulfate, filtered, and concentrated. The crude oil was purified by flash chromatography over silica using a hexanes:ethyl acetate gradient of 0 to 30% ethyl acetate to afford 0.49 g (60%) of 3-[6-({[3-(2,6-dichlorophenyl)-5-(1-methylethyl)-4-isoxazolyl]methyl}oxy)-2-naphthalenyl]aniline. ¹H NMR (400 MHz, DMSO-d₆): δ 7.94 (s, 1H), 7.78 (d, J=9 Hz, 1H), 7.75 (d, J=9 Hz, 1H), 7.64 (dd, J=9, 1.71 Hz, 1H), 7.61 (m, 2H), 7.52 (m, 1H), 7.26 (d, J=2 Hz, 1H), 7.09 (t, J=8 Hz, 1H), 6.89 (m, 2H), 6.85 (m, 1H), 6.54 (d, J=8 Hz, 1H), 5.14 (s, 2H), 4.93 (s, 2H), 3.49 (septet, J=7 Hz, 1H), 1.33 (d, J=7 Hz, 6H).

### 26c) N-{3-[6-({[3-(2,6-Dichlorophenyl)-5-(1-methylethyl)-4-isoxazolyl]methyl}oxy)-2-naphthalenyl]phenyl}-1,1,1-trifluoro-N-[(trifluoromethyl)sulfonyl]methanesulfonamide

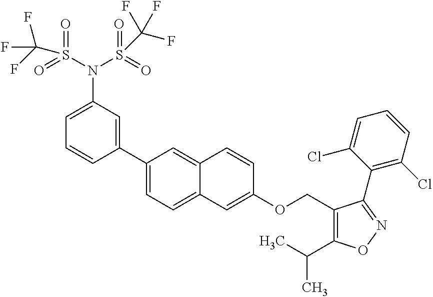

To a solution of 3-[6-({[3-(2,6-dichlorophenyl)-5-(1-methylethyl)-4-isoxazolyl]methyl}oxy)-2-naphthalenyl]aniline (0.1 g, 0.199 mmol) and triethylamine (0.042 mL, 0.298 mmol) in dichloromethane (1.1 mL) at −78° C. was slowly added a solution of trifluoromethane sulfonic anhydride (0.033 mL, 0.199 mmol) in dichloromethane (1 mL). The reaction mixture was stirred at −78° C. for 1 h. Ethyl acetate was added to the reaction mixture, followed by saturated sodium hydrogencarbonate. The ethyl acetate layer was separated, washed with brine, dried over magnesium sulfate, filtered, and concentrated. The crude material was purified by flash chromatography over silica using a hexanes:ethyl acetate gradient of 0 to 30% ethyl acetate to afford impure product which was purified by flash chromatography over silica using a hexanes:dichloromethane gradient of 0 to 30% dichloromethane to afford 0.0845 g (56%) of N-{3-[6-({[3-(2,6-dichlorophenyl)-5-(1-methylethyl)-4-isoxazolyl]methyl}oxy)-2-naphthalenyl]phenyl}-1,1,1-trifluoro-N-[(trifluoromethyl)sulfonyl]methanesulfonamide. ¹H NMR (400 MHz, DMSO-d₆): δ 8.19 (s, 1H), 8.11 (m, 2H), 7.88-7.78 (m, 3H), 7.72 (m, 1H), 7.66 (m, 1H), 7.61 (m, 2H), 7.52 (m, 1H), 7.32 (d, J=2 Hz, 1H), 6.94 (dd, J=9, 2 Hz, 1H), 4.95 (s, 2H), 3.51 (septet, J=7 Hz, 1H), 1.34 (d, J=7 Hz, 6H).

### Example 27

### N-acetyl-N-{3-[6-({[3-(2,6-dichlorophenyl)-5-(1-methylethyl)-4-isoxazolyl]methyl}oxy)-2-naphthalenyl]phenyl}acetamide

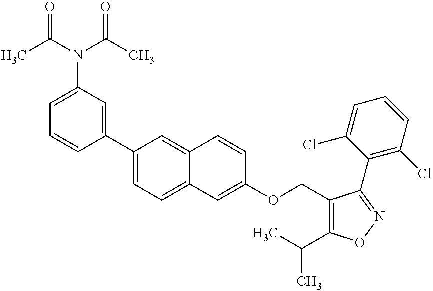

### 27a) N-acetyl-N-{3-[6-({[3-(2,6-dichlorophenyl)-5-(1-methylethyl)-4-isoxazolyl]methyl}oxy)-2-naphthalenyl]phenyl}acetamide

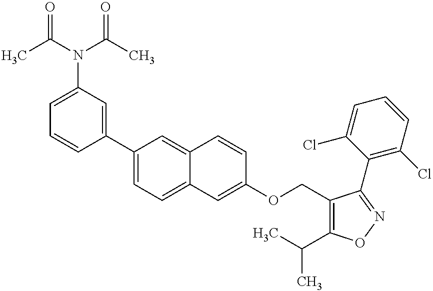

To a solution of 3-[6-({[3-(2,6-dichlorophenyl)-5-(1-methylethyl)-4-isoxazolyl]methyl}oxy)-2-naphthalenyl]aniline (Example 26b) (0.1 g, 0.199 mmol) and N-methylmorpholine (0.044 mL, 0.397 mmol) in dichloromethane (3 mL) at 0° C. was slowly added a solution of acetyl chloride (0.017 mL, 0.238 mmol) in dichloromethane (2 mL). The reaction mixture was stirred at room temperature for 1 h and diluted with dichloromethane, followed by water. The dichloromethane layer was separated, washed with brine, dried over magnesium sulfate, filtered and concentrated. The crude material was purified by flash chromatography over silica using a hexanes:ethyl acetate gradient of 0 to 50% ethyl acetate to afford 0.036 g (31%) of N-acetyl-N-{3-[6-({[3-(2,6-dichlorophenyl)-5-(1-methylethyl)-4-isoxazolyl]methyl}oxy)-2-naphthalenyl]phenyl}acetamide. ¹H NMR (400 MHz, DMSO-d₆): δ 8.16 (s, 1H), 7.82 (m, 4H), 7.73 (m, 1H), 7.61 (m, 2H), 7.54 (m, 2H), 7.29 (d, J=2 Hz, 1H), 7.26 (d, J=7 Hz, 1H), 6.92 (dd, J=9, 3 Hz, 1H), 4.93 (s, 2H), 3.50 (septet, J=7 Hz, 1H), 2.21 (s, 6H), 1.33 (d, J=7 Hz, 6H). HRMS C₃₃H₂₉Cl₂N₂O₄ m/z 587.15007 (M+H)⁺_{Cal}; 587.14989 (M+H)⁺_{Obs}.

### Example 28

### N-{3-[6-({[3-(2,6-dichlorophenyl)-5-(1-methylethyl)-4-isoxazolyl]methyl}oxy)-2-naphthalenyl]phenyl}-1,1,1-trifluoromethanesulfonamide

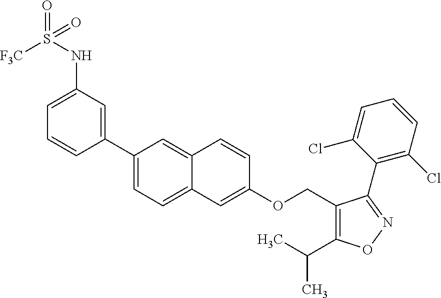

### 28a) N-{3-[6-({[3-(2,6-Dichlorophenyl)-5-(1-methylethyl)-4-isoxazolyl]methyl}oxy)-2-naphthalenyl]phenyl}-1,1,1-trifluoromethanesulfonamide

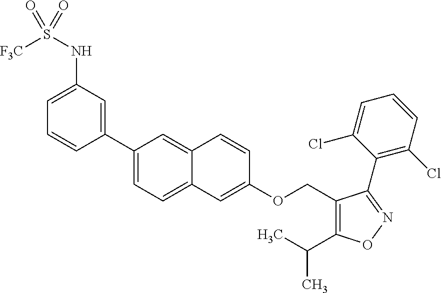

To a solution of 3-[6-({[3-(2,6-dichlorophenyl)-5-(1-methylethyl)-4-isoxazolyl]methyl}oxy)-2-naphthalenyl]aniline (Example 26b) (0.1 g, 0.199 mmol) and triethylamine (0.042 mL, 0.298 mmol) in dichloromethane (1.1 mL) at −78° C. was slowly added a solution of trifluoromethane sulfonic anhydride (0.033 mL, 0.199 mmol) in dichloromethane (1 mL). The reaction mixture was stirred at −78° C. for approximately 20 min, then diluted with water, followed by ethyl acetate. The ethyl acetate layer was separated, washed with brine, dried over magnesium sulfate, filtered, and concentrated. The crude material was purified by flash chromatography over silica using a hexanes:ethyl acetate gradient of 0 to 30% ethyl acetate to afford 0.045 g (36%) of N-{3-[6-({[3-(2,6-dichlorophenyl)-5-(1-methylethyl)-4-isoxazolyl]methyl}oxy)-2-naphthalenyl]phenyl}-1,1,1-trifluoromethanesulfonamide. ¹H NMR (400 MHz, DMSO-d₆): δ 12.03 (br s, 1H), 8.05 (d, J=1 Hz, 1H), 7.82 (m, 2H), 7.69 (dd, J=9, 2 Hz, 1H), 7.65 (d, J=7 Hz, 1H), 7.61 (m, 2H), 7.56 (t, J=2 Hz, 1H), 7.51 (m, 2H), 7.29 (d, J=2 Hz, 1H), 7.23 (d, J=7 Hz, 1H), 6.92 (dd, J=9, 3 Hz, 1H), 4.94 (s, 2H), 3.50 (septet, J=7 Hz, 1H), 1.34 (d, J=7 Hz, 6H). HRMS C₃₀H₂₃Cl₂F₃N₂O₄S m/z 635.0786 (M+H)⁺_{Cal}; 635.0788 (M+H)⁺_{Obs}.

### Example 29

### N-{3-[6-({[3-(2,6-dichlorophenyl)-5-(1-methylethyl)-4-isoxazolyl]methyl}oxy)-2-naphthalenyl]phenyl}acetamide

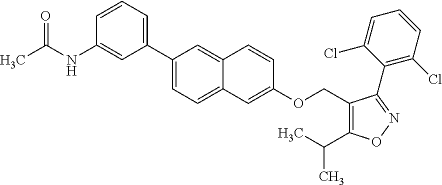

### 29a) N-{3-[6-({[3-(2,6-dichlorophenyl)-5-(1-methylethyl)-4-isoxazolyl]methyl}oxy)-2-naphthalenyl]phenyl}acetamide

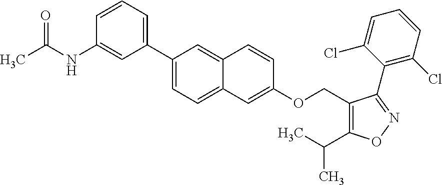

To a slurry of 4-{[(6-bromo-2-naphthalenyl)oxy]methyl}-3-(2,6-dichlorophenyl)-5-(1-methylethyl)isoxazole (example 22a) (0.1 g, 0.204 mmol), tetrakis(triphenylphosphine)palladium(0) (0.009 g, 0.008 mmol), ethylene glycol dimethyl ether (1.1 mL) and 2 N sodium carbonate (1 mL, 2 mmol) was added [3-(acetylamino)phenyl]boronic acid (0.044 g, 0.244 mmol) and the reaction mixture was heated at 80° C. for 1 h. The reaction mixture was cooled to room temperature and diluted with water, followed by ethyl acetate. The layers were separated and the ethyl acetate layer was washed with brine, dried over magnesium sulfate, filtered, and concentrated. The crude material was purified by flash chromatography over silica using a hexanes:ethyl acetate gradient of 0 to 30% ethyl acetate to afford 0.032 g (29%) of N-{3-[6-({[3-(2,6-dichlorophenyl)-5-(1-methylethyl)-4-isoxazolyl]methyl}oxy)-2-naphthalenyl]phenyl}acetamide. ¹H NMR (400 MHz, DMSO-d₆): δ 10.02 (s, 1H), 8.00 (s, 1H), 7.96 (s, 1H), 7.81 (t, J=9 Hz, 2H), 7.68 (dd, J=8, 2 Hz, 1H), 7.61 (m, 2H), 7.53 (m, 2H), 7.38 (m, 2H), 7.28 (d, J=2 Hz, 1H), 6.91 (dd, J=9, 2 Hz, 1H), 4.94 (s, 2H), 3.50 (septet, J=7 Hz, 1H), 2.05 (s, 3H), 1.34 (d, J=7 Hz, 6H). HRMS C₃₁H₂₆Cl₂N₂O₃ m/z 545.1399 (M+H)⁺_{Cal}; 545.1403 (M+H)⁺_{Obs}.

### Example 30

### 3-(2,6-Dichlorophenyl)-5-(1-methylethyl)-4-{[(6-{3-[(trifluoromethyl)oxy]phenyl}-2-naphthalenyl)oxy]methyl}isoxazole

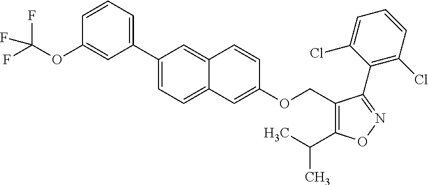

### 30a) 3-(2,6-Dichlorophenyl)-5-(1-methylethyl)-4-{[(6-{3-[(trifluoromethyl)oxy]phenyl}-2-naphthalenyl)oxy]methyl}isoxazole

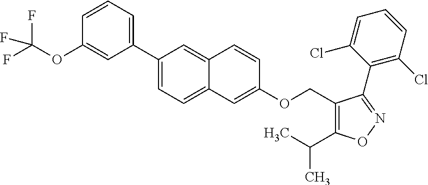

To a slurry of 4-{[(6-bromo-2-naphthalenyl)oxy]methyl}-3-(2,6-dichlorophenyl)-5-(1-methylethyl)isoxazole (example 22a) (0.1 g, 0.204 mmol), tetrakis(triphenylphosphine)palladium(0) (0.009 g, 0.008 mmol), ethylene glycol dimethyl ether (1.1 mL) and 2 N sodium carbonate (1 mL, 2 mmol) was added {3-[(trifluoromethyl)oxy]phenyl}boronic acid (0.05 g, 0.244 mmol) and the reaction mixture was heated at 80° C. for 1 h. The reaction mixture was cooled to room temperature and diluted with water, followed by ethyl acetate. The layers were separated and the ethyl acetate layer was washed with brine, dried over magnesium sulfate, filtered, and concentrated. The crude material was purified by flash chromatography over silica using a hexanes:ethyl acetate gradient of 0 to 50% ethyl acetate to afford 0.0588 g (50%) of 3-(2,6-dichlorophenyl)-5-(1-methylethyl)-4-{[(6-{3-[(trifluoromethyl)oxy]phenyl}-2-naphthalenyl)oxy]methyl} isoxazole. ¹H NMR (400 MHz, DMSO-d₆): δ 8.18 (s, 1H), 7.82 (m, 4H), 7.72 (s, 1H), 7.60 (m, 3H), 7.52 (dd, J=9, 7 Hz, 1H), 7.34 (d, J=8 Hz, 1H), 7.30 (d, J=2 Hz, 1H), 6.93 (dd, J=9, 3 Hz, 1H), 4.94 (s, 2H), 3.50 (septet, J=7 Hz, 1H), 1.34 (d, J=7 Hz, 6H). HRMS C₃₀H₂₂Cl₂F₃NO₃ m/z 572.1007 (M+H)⁺_{Cal}; 572.1012 (M+H)⁺_{Obs}.

### Example 31

### N-{4-[6-({[3-(2,6-dichlorophenyl)-5-(1-methylethyl)-4-isoxazolyl]methyl}oxy)-2-naphthalenyl]phenyl}acetamide

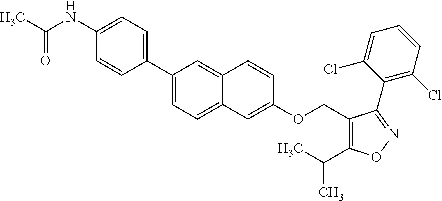

### 31a) N-{4-[6-({[3-(2,6-dichlorophenyl)-5-(1-methylethyl)-4-isoxazolyl]methyl}oxy)-2-naphthalenyl]phenyl}acetamide

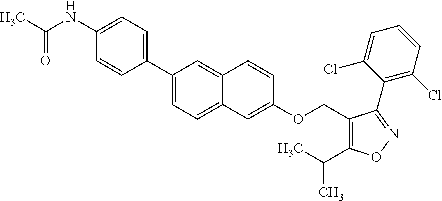

To a slurry of 4-{[(6-bromo-2-naphthalenyl)oxy]methyl}-3-(2,6-dichlorophenyl)-5-(1-methylethyl)isoxazole (example 22a) (0.1 g, 0.204 mmol), tetrakis(triphenylphosphine)palladium(0) (0.009 g, 0.008 mmol), ethylene glycol dimethyl ether (1.1 mL) and 2 N sodium carbonate (1 mL, 2 mmol) was added [4-(acetylamino)phenyl]boronic acid (0.044 g, 0.244 mmol) and the reaction mixture was heated at 80° C. for 1 h. The reaction mixture was cooled to room temperature and diluted with water, followed by ethyl acetate. The layers were separated and the ethyl acetate layer was washed with brine, dried over magnesium sulfate, filtered, and concentrated. The crude material was purified by flash chromatography over silica using a hexanes:ethyl acetate gradient of 0 to 30% ethyl acetate to afford 0.054 g (49%) of N-{4-[6-({[3-(2,6-dichlorophenyl)-5-(1-methylethyl)-4-isoxazolyl]methyl}oxy)-2-naphthalenyl]phenyl}acetamide. ¹H NMR (400 MHz, DMSO-d₆): δ 10.01 (s, 1H), 8.04 (s, 1H), 7.77 (m, 3H), 7.68 (m, 4H), 7.61 (m, 2H), 7.52 (dd, J=9, 7 Hz, 1H), 7.26 (d, J=2 Hz, 1H), 6.89 (dd, J=9, 2 Hz, 1H), 4.93 (s, 2H), 3.50 (septet, J=7 Hz, 1H), 2.04 (s, 3H), 1.33 (d, J=7 Hz, 6H). HRMS C₃₁H₂₆Cl₂N₂O₃ m/z 545.13932 (M+H)⁺_{Cal}; 545.13944 (M+H)⁺_{Obs}.

### Example 32

### N-{4-[6-({[3-(2,6-dichlorophenyl)-5-(1-methylethyl)-4-isoxazolyl]methyl}oxy)-2-naphthalenyl]phenyl}-1,1,1-trifluoromethanesulfonamide

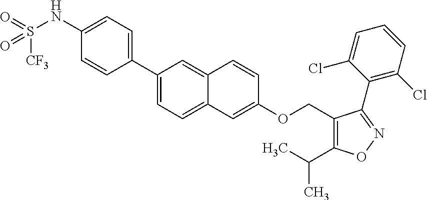

### 32a) 4-[6-({[3-(2,6-Dichlorophenyl)-5-(1-methylethyl)-4-isoxazolyl]methyl}oxy)-2-naphthalenyl]aniline

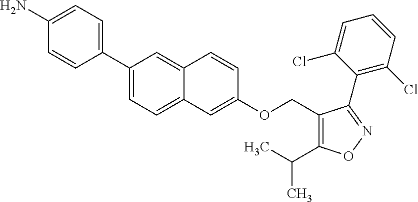

To a slurry of 4-{[(6-bromo-2-naphthalenyl)oxy]methyl}-3-(2,6-dichlorophenyl)-5-(1-methylethyl)isoxazole (example 22a) (0.2 g, 0.407 mmol), tetrakis(triphenylphosphine)palladium(0) (0.0188 g, 0.016 mmol), ethylene glycol dimethyl ether (2.2 mL) and 2 N sodium carbonate (2 mL, 4 mmol) was added 4-(4,4,5,5-tetramethyl-1,3,2-dioxaborolan-2-yl)aniline (0.107 g, 0.489 mmol) and the reaction mixture was heated at 80° C. for 1 h. The reaction mixture was cooled to room temperature and diluted with water, followed by ethyl acetate. The layers were separated and the ethyl acetate layer was washed with brine, dried over magnesium sulfate, filtered, and concentrated. The crude material was purified by flash chromatography over silica using a hexanes:ethyl acetate gradient of 0 to 30% ethyl acetate to afford impure product. This material was dissolved in ethyl acetate and washed several times with water. The layers were separated and the organic layer was dried over magnesium sulfate, filtered, and concentrated to afford 0.090 g (44%) of 4-[6-({[3-(2,6-dichlorophenyl)-5-(1-methylethyl)-4-isoxazolyl]methyl}oxy)-2-naphthalenyl]aniline. ¹H NMR (400 MHz, DMSO-d₆): δ 7.90 (s, 1H), 7.74-7.63 (m, 3H), 7.61 (m, 2H), 7.51 (dd, J=9, 7 Hz, 1H), 7.43 (d, J=9 Hz, 2H), 7.21 (d, J=3 Hz, 1H), 6.85 (dd, J=9, 2 Hz, 1H), 6.63 (d, J=9 Hz, 2H), 5.21 (s, 2H), 4.90 (s, 2H), 3.49 (septet, J=7 Hz, 1H), 1.33 (d, J=7 Hz, 6H).

### 32b) N-{4-[6-({[3-(2,6-dichlorophenyl)-5-(1-methylethyl)-4-isoxazolyl]methyl}oxy)-2-naphthalenyl]phenyl}-1,1,1-trifluoromethanesulfonamide

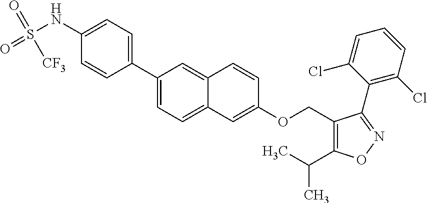

To a solution of 4-[6-({[3-(2,6-dichlorophenyl)-5-(1-methylethyl)-4-isoxazolyl]methyl}oxy)-2-naphthalenyl]aniline (0.09 g, 0.179 mmol) and triethylamine (0.037 mL, 0.268 mmol) in dichloromethane (1 mL) at −78° C. was slowly added a solution of trifluoromethane sulfonic anhydride (0.030 mL, 0.179 mmol) in dichloromethane (1 mL). The reaction mixture was stirred at −78° C. for approximately 15 min. The reaction mixture was diluted with water, followed by ethyl acetate. The ethyl acetate layer was washed with brine, dried over magnesium sulfate, filtered and concentrated. The crude material was purified by flash chromatography over silica using a hexanes:ethyl acetate gradient of 0 to 30% ethyl acetate to afford 0.061 g (54%) of N-{4-[6-({[3-(2,6-dichlorophenyl)-5-(1-methylethyl)-4-isoxazolyl]methyl}oxy)-2-naphthalenyl]phenyl}-1,1,1-trifluoromethanesulfonamide. ¹H NMR (400 MHz, DMSO-d₆): δ 8.08 (s, 1H), 7.78 (m, 5H), 7.61 (m, 2H), 7.52 (m, 1H), 7.33 (d, J=8 Hz, 2H), 7.28 (d, J=2 Hz, 1H), 6.91 (dd, J=9, 3 Hz, 1H), 4.93 (s, 2H), 3.50 (septet, J=7 Hz, 1H), 1.33 (d, J=7 Hz, 6H). HRMS C₃₀H₂₃Cl₂F₃N₂O₄S m/z 635.0786 (M+H)⁺_{Cal}; 635.0803 (M+H)⁺_{Obs}.

### Example 33

### 3-[7-({[3-(2,6-Dichlorophenyl)-5-(1-methylethyl)-4-isoxazolyl]methyl}oxy)-2-naphthalenyl]benzoic acid

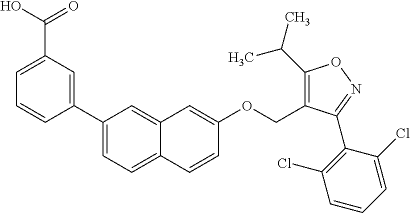

### 33a) 7-(Methyloxy)-2-naphthalenyl trifluoromethanesulfonate

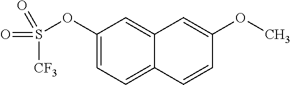

To a solution of 7-(methyloxy)-2-naphthalenol (1.5 g, 8.61 mmol) and pyridine (4.2 mL, 51.7 mmol) in dichloromethane (40 mL) was added trifluoromethane sulfonic anhydride (1.74 mL, 10.3 mmol) at 0° C. The reaction mixture was stirred for 24 h at room temperature, then diluted with water, followed by diethyl ether. The ether layer was washed several times with water, followed by brine, dried over magnesium sulfate, filtered, and concentrated. The crude oil was purified by flash chromatography over silica using a hexanes:ethyl acetate gradient of 0 to 30% ethyl acetate to afford 2.7 g (100%) of 7-(methyloxy)-2-naphthalenyl trifluoromethanesulfonate with a minor impurity. ¹H NMR (400 MHz, DMSO-d₆): δ 8.02 (d, J=9 Hz, 1H), 7.95 (d, J=2 Hz, 1H), 7.92 (d, J=9 Hz, 1H), 7.48 (d, J=3 Hz, 1H), 7.38 (dd, J=9, 3 Hz, 1H), 7.25 (dd, J=9, 3 Hz, 1H), 3.87 (s, 3H).

### 33b) Methyl 3-[7-(methyloxy)-2-naphthalenyl]benzoate

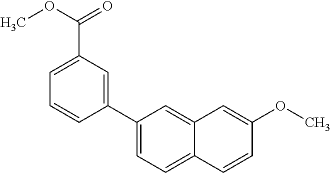

To a slurry of 7-(methyloxy)-2-naphthalenyl trifluoromethanesulfonate (2.7 g, 8.82 mmol), tetrakis(triphenylphosphine)palladium(0) (0.41 g, 0.353 mmol), ethylene glycol dimethyl ether (47.5 mL) and 2 N sodium carbonate (43 mL, 86 mmol) was added {3-[(methyloxy)carbonyl]phenyl}boronic acid (1.9 g, 10.58 mmol) and the reaction mixture was heated at 80° C. for 1 h. The reaction mixture was cooled to room temperature and diluted with water, followed by ethyl acetate. The organic layer was washed with water followed by brine, dried over magnesium sulfate, filtered, and concentrated. The crude material was purified by flash chromatography over silica using a hexanes:ethyl acetate gradient of 0 to 30% ethyl acetate to afford 2.25 g (88%) of methyl 3-[7-(methyloxy)-2-naphthalenyl]benzoate. ¹H NMR (400 MHz, DMSO-d₆): δ 8.31 (t, J=2 Hz, 1H), 8.17 (d, J=2 Hz, 1H), 8.07 (m, 1H), 7.97 (m, 1H), 7.94 (d, J=9 Hz, 1H), 7.85 (d, J=9 Hz, 1H), 7.67 (m, 2H), 7.45 (d, J=2 Hz, 1H), 7.17 (dd, J=9, 3 Hz, 1H), 3.89 (s, 3H), 3.87 (s, 3H)

### 33c) Methyl 3-(7-hydroxy-2-naphthalenyl)benzoate

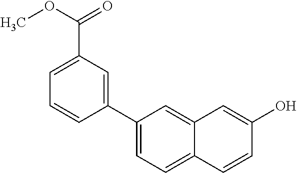

Boron tribromide (1 M in dichloromethane) (31 mL, 31 mmol) was added slowly to a solution of methyl 3-[7-(methyloxy)-2-naphthalenyl]benzoate (2.25 g, 7.70 mmol) in dichloromethane (113 mL) at 0° C. The reaction mixture was stirred for approximately 2 h at 0° C., then poured into ice water and stirred for several minutes. The layers were separated and the aqueous layer was extracted with ethyl acetate and the combined organic layer was washed with brine, dried over magnesium sulfate, filtered and concentrated. The crude material was purified by flash chromatography over silica using a hexanes:ethyl acetate gradient of 0 to 30% ethyl acetate to afford 1.9 g (89%) of methyl 3-(7-hydroxy-2-naphthalenyl)benzoate. ¹H NMR (400 MHz, DMSO-d₆): δ 9.81 (s, 1H), 8.28 (s, 1H), 8.04 (m, 1H), 8.02 (s, 1H), 7.95 (d, J=8 Hz, 1H), 7.86 (d, J=9 Hz, 1H), 7.77 (d, J=9 Hz, 1H), 7.63 (t, J=8 Hz, 1H), 7.58 (d, J=9 Hz, 1H), 7.23 (d, J=2 Hz, 1H), 7.09 (dd, J=9, 2 Hz, 1H), 3.88 (s, 3H).

### 33d) Methyl 3-[7-({[3-(2,6-dichlorophenyl)-5-(1-methylethyl)-4-isoxazolyl]methyl}oxy)-2-naphthalenyl]benzoate

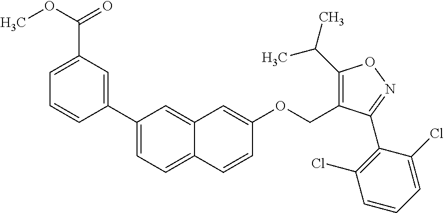

A mixture of methyl 3-(7-hydroxy-2-naphthalenyl)benzoate (0.15 g, 0.54 mmol) and cesium carbonate (0.25 g, 0.755 mmol) in N,N-dimethylformamide (1.5 mL) was heated at 65° C. for 1 h. To the reaction mixture was added a solution of 4-(chloromethyl)-3-(2,6-dichlorophenyl)-5-(1-methylethyl)isoxazole (0.16 g, 0.54 mmol) in N,N-dimethylformamide (1 mL). Heating at 65° C. was continued for 4 h. The reaction mixture was cooled to room temperature and diluted with water, followed by ethyl acetate. The ethyl acetate layer was separated, washed several times with water, followed by brine, dried over magnesium sulfate, filtered, and concentrated. The crude oil was purified by flash chromatography over silica using a hexanes:ethyl acetate gradient of 0 to 30% ethyl acetate to afford 0.145 g (49%) of methyl 3-[7-({[3-(2,6-dichlorophenyl)-5-(1-methylethyl)-4-isoxazolyl]methyl}oxy)-2-naphthalenyl]benzoate. ¹H NMR (400 MHz, DMSO-d₆): δ 8.29 (s, 1H), 8.05 (m, 2H), 7.97 (d, J=8 Hz, 1H), 7.90 (d, J=9 Hz, 1H), 7.78 (d, J=9 Hz, 1H), 7.69 (s, 1H), 7.67 (s, 1H), 7.63 (d, J=1 Hz, 1H), 7.61 (s, 1H), 7.53 (m, 1H), 7.39 (d, J=2 Hz, 1H), 6.92 (dd, J=9, 2 Hz, 1H), 4.93 (s, 2H), 3.89 (s, 3H), 3.50 (septet, J=7 Hz, 1H), 1.34 (d, J=7 Hz, 6H).

### 33e) 3-[7-({[3-(2,6-Dichlorophenyl)-5-(1-methylethyl)-4-isoxazolyl]methyl}oxy)-2-naphthalenyl]benzoic acid

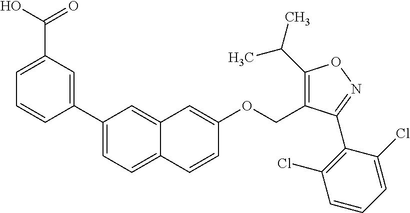

Methyl 3-[7-({[3-(2,6-dichlorophenyl)-5-(1-methylethyl)-4-isoxazolyl]methyl}oxy)-2-naphthalenyl]benzoate (0.145 g, 0.265 mmol) and 1 N sodium hydroxide (0.56 mL) were stirred in tetrahydrofuran (2.8 mL) and methanol (1.4 mL) at room temperature overnight. The reaction mixture was concentrated then diluted with 1 N hydrochloric acid, followed by ethyl acetate. The organic layer was separated, washed with brine, dried over magnesium sulfate, filtered, and concentrated. The crude material was purified by flash chromatography over silica using a dichloromethane:methanol gradient of 0 to 5% methanol to afford 0.074 g (53%) of 3-[7-({[3-(2,6-dichlorophenyl)-5-(1-methylethyl)-4-isoxazolyl]methyl}oxy)-2-naphthalenyl]benzoic acid. ¹H NMR (400 MHz, DMSO-d₆): δ 13.10 (s, 1H), 8.29 (s, 1H), 8.05 (s, 1H), 8.01 (d, J=9 Hz, 1H), 7.94 (d, J=8 Hz, 1H), 7.89 (d, J=9 Hz, 1H), 7.78 (d, J=9 Hz, 1H), 7.68 (d, J=8 Hz, 1H), 7.62 (m, 3H), 7.53 (m, 1H), 7.38 (d, J=2 Hz, 1H), 6.92 (dd, J=9, 2 Hz, 1H), 4.93 (s, 2H), 3.50 (septet, J=7 Hz, 1H), 1.34 (d, J=7 Hz, 6H).

### Example 34

### 2-Chloro-5-[6-({[3-(2,6-dichlorophenyl)-5-(1-methylethyl)-4-isoxazolyl]methyl}oxy)-2-naphthalenyl]benzoic acid

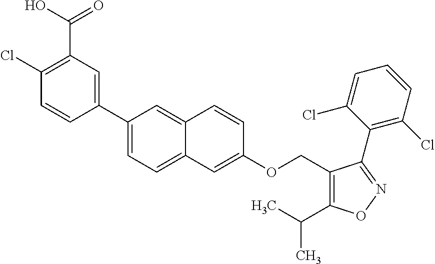

### 34a) Ethyl 2-chloro-5-[6-(methyloxy)-2-naphthalenyl]benzoate

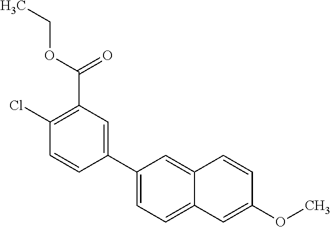

To a slurry of ethyl 5-bromo-2-chlorobenzoate (0.21 mL, 1.24 mmol), tetrakis(triphenylphosphine)palladium(0) (0.057 g, 0.050 mmol), ethylene glycol dimethyl ether (6.6 mL) and 2 N sodium carbonate (6 mL, 12 mmol) was added [6-(methyloxy)-2-naphthalenyl]boronic acid (0.3 g, 1.49 mmol) and the reaction mixture was heated at 80° C. for 1 h. The reaction mixture was cooled to room temperature and diluted with water, followed by ethyl acetate. The organic layer was separated, washed with water, followed by brine, dried over magnesium sulfate, filtered, and concentrated. The crude material was purified by flash chromatography over silica using a hexanes:ethyl acetate gradient of 0 to 30% ethyl acetate to afford 0.43 g (100%) of ethyl 2-chloro-5-[6-(methyloxy)-2-naphthalenyl]benzoate. ¹H NMR (400 MHz, DMSO-d₆): δ 8.21 (s, 1H), 8.12 (d, J=2 Hz, 1H), 7.96 (dd, J=8, 2 Hz, 1H), 7.92 (s, 1H), 7.90 (s, 1H), 7.80 (d, J=9 Hz, 1H), 7.66 (d, J=9 Hz, 1H), 7.35 (d, J=2 Hz, 1H), 7.19 (dd, J=9, 2 Hz, 1H), 4.35 (q, J=7 Hz, 2H), 3.87 (s, 3H), 1.33 (t, J=7 Hz, 3H).

### 34b) Ethyl 2-chloro-5-(6-hydroxy-2-naphthalenyl)benzoate

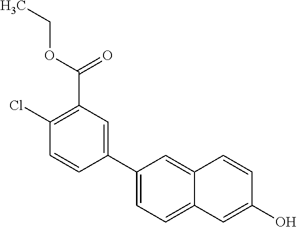

Boron tribromide (1 M in dichloromethane) (4.93 mL, 4.93 mmol) was added slowly to a solution of ethyl 2-chloro-5-[6-(methyloxy)-2-naphthalenyl]benzoate (0.42 g, 1.23 mmol) in dichloromethane (18 mL) at 0° C. The reaction mixture was stirred at 0° C. for 2 h. The reaction was poured into ice water and extracted with ethyl acetate. The organic layer was separated, washed with brine, dried over magnesium sulfate, filtered, and concentrated. The crude material was purified by flash chromatography over silica using a hexanes:ethyl acetate gradient of 0 to 30% ethyl acetate to afford 0.399 g (99%) of ethyl 2-chloro-5-(6-hydroxy-2-naphthalenyl)benzoate. ¹H NMR (400 MHz, DMSO-d₆): δ 9.86 (s, 1H), 8.14 (s, 1H), 8.10 (d, J=2 Hz, 1H), 7.94 (dd, J=8, 2 Hz, 1H), 7.85 (d, J=9 Hz, 1H), 7.78 (m, 1H), 7.72 (m, 1H), 7.65 (d, J=8 Hz, 1H), 7.13 (m, 1H), 7.11 (m, 1H), 4.35 (q, J=7 Hz, 2H), 1.33 (t, J=7 Hz, 3H).

### 34c) Ethyl 2-chloro-5-[6-({[3-(2,6-dichlorophenyl)-5-(1-methylethyl)-4-isoxazolyl]methyl}oxy)-2-naphthalenyl]benzoate

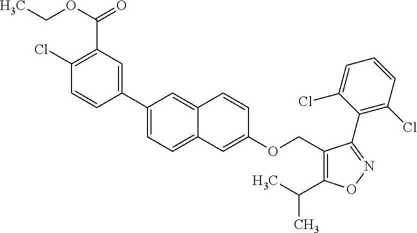

A mixture of ethyl 2-chloro-5-(6-hydroxy-2-naphthalenyl)benzoate (0.15 g, 0.459 mmol) and cesium carbonate (0.209 g, 0.643 mmol) in N,N-dimethylformamide (1.1 mL) was heated at 65° C. for 1 h. To the reaction mixture was added a solution of 4-(chloromethyl)-3-(2,6-dichlorophenyl)-5-(1-methylethyl)isoxazole (0.14 g, 0.459 mmol) in N,N-dimethylformamide (1 mL) and heating was continued at 65° C. for 24 hours. The reaction mixture was cooled to room temperature and diluted with water, followed by ethyl acetate. The ethyl acetate layer was separated, washed several times with water, followed by brine, dried over magnesium sulfate, filtered, and concentrated. The crude oil was purified by flash chromatography over silica using a hexanes:ethyl acetate gradient of 0 to 30% ethyl acetate to afford 0.18 g (66%) of ethyl 2-chloro-5-[6-({[3-(2,6-dichlorophenyl)-5-(1-methylethyl)-4-isoxazolyl]methyl}oxy)-2-naphthalenyl]benzoate. ¹H NMR (400 MHz, DMSO-d₆): δ 8.17 (s, 1H), 8.10 (d, J=2 Hz, 1H), 7.94 (dd, J=8, 2 Hz, 1H), 7.84 (d, J=9 Hz, 1H), 7.79 (m, 2H), 7.66 (d, J=9 Hz, 1H), 7.62 (d, J=1 Hz, 1H), 7.61 (m, 1H), 7.52 (m, 1H), 7.30 (d, J=2 Hz, 1H), 6.93 (dd, J=9, 3 Hz, 1H), 4.94 (s, 2H), 4.35 (q, J=7 Hz, 2H), 3.50 (septet, J=7 Hz, 1H), 1.33 (m, 9H).

### 34d) 2-Chloro-5-[6-({[3-(2,6-dichlorophenyl)-5-(1-methylethyl)-4-isoxazolyl]methyl}oxy)-2-naphthalenyl]benzoic acid

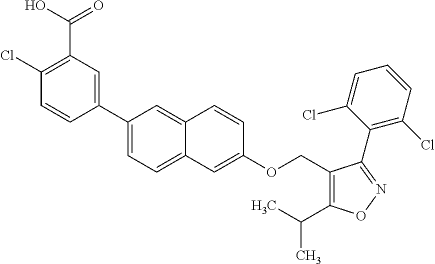

Ethyl 2-chloro-5-[6-({[3-(2,6-dichlorophenyl)-5-(1-methylethyl)-4-isoxazolyl]methyl}oxy)-2-naphthalenyl]benzoate (0.18 g, 0.303 mmol) and 1 N sodium hydroxide (0.64 mL, 0.64 mmol) were stirred in tetrahydrofuran (3 mL) and ethanol (1.6 mL) at room temperature overnight. The reaction mixture was concentrated and diluted with 1 N hydrochloric acid, followed by ethyl acetate. The organic layer was separated, washed with brine, dried over magnesium sulfate, filtered, and concentrated. The crude product was purified by flash chromatography over silica using a dichloromethane:methanol gradient of 0 to 5% methanol to afford 0.124 g (72%) of 2-chloro-5-[6-({[3-(2,6-dichlorophenyl)-5-(1-methylethyl)-4-isoxazolyl]methyl}oxy)-2-naphthalenyl]benzoic acid. ¹H NMR (400 MHz, DMSO-d₆): δ 13.50 (s, 1H), 8.17 (s, 1H), 8.11 (d, J=2 Hz, 1H), 7.91 (dd, J=8, 2 Hz, 1H), 7.84 (d, J=9 Hz, 1H), 7.79 (m, 2H), 7.63 (m, 2H), 7.60 (s, 1H), 7.51 (dd, J=9, 7 Hz, 1H), 7.30 (d, J=2 Hz, 1H), 6.92 (dd, J=9, 3 Hz, 1H), 4.94 (s, 2H), 3.50 (septet, J=7 Hz, 1H), 1.34 (d, J=7 Hz, 6H). HRMS C₃₀H₂₂Cl₃NO₄ m/z 566.0693 (M+H)⁺_{Cal}; 566.0698 (M+H)⁺_{Obs}.

### Example 35

### 5-[6-({[3-(2,6-Dichlorophenyl)-5-(1-methylethyl)-4-isoxazolyl]methyl}oxy)-2-naphthalenyl]-2-fluorobenzoic acid

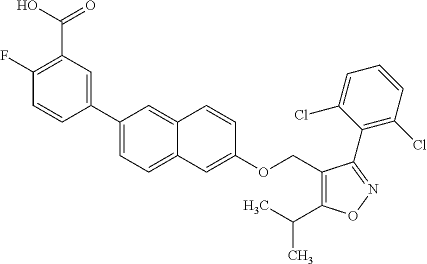

### 35a) 2-Fluoro-5-[6-(methyloxy)-2-naphthalenyl]benzoic acid

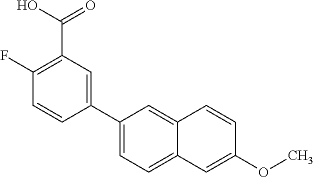

To a slurry of 5-bromo-2-fluorobenzoic acid (0.45 g, 2.06 mmol), tetrakis(triphenylphosphine)palladium(0) (0.095 g, 0.083 mmol), ethylene glycol dimethyl ether (11 mL) and 2 N sodium carbonate (10 mL, 20 mmol) was added [6-(methyloxy)-2-naphthalenyl]boronic acid (0.5 g, 2.48 mmol) and the reaction mixture was heated at 80° C. for 2 h. The reaction was cooled to room temperature and diluted with water, followed by ethyl acetate. The layers were separated and the aqueous layer was acidified to pH 2 (litmus paper) with 1 N hydrochloric acid. The acidic aqueous phase was extracted with ethyl acetate. The organic extracts were combined, washed with water, followed by brine, dried over magnesium sulfate, filtered through a pad of Celite® and concentrated to afford 0.53 g (72%) of 2-fluoro-5-[6-(methyloxy)-2-naphthalenyl]benzoic acid. ¹H NMR (400 MHz, DMSO-d₆): δ 13.38 (s, 1H), 8.18 (dd, J=7, 2 Hz, 1H), 8.16 (d, J=1 Hz, 1H), 8.02 (m, 1H), 7.92 (d, J=7 Hz, 1H), 7.90 (d, J=6 Hz, 1H), 7.78 (dd, J=8, 2 Hz, 1H), 7.42 (dd, J=11, 9 Hz, 1H), 7.34 (d, J=2 Hz, 1H), 7.18 (dd, J=9, 2 Hz, 1H), 3.87 (s, 3H).

### 35b) Methyl 2-fluoro-5-[6-(methyloxy)-2-naphthalenyl]benzoate

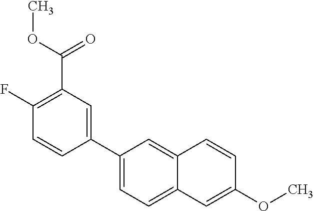

Thionyl chloride (0.26 mL, 3.58 mmol) was added slowly to a slurry of 2-fluoro-5-[6-(methyloxy)-2-naphthalenyl]benzoic acid (0.53 g, 1.79 mmol) in methanol (16 mL) and the reaction mixture was heated at 75° C. overnight. The reaction was cooled to room temperature and concentrated. The crude material was diluted with saturated sodium bicarbonate and extracted with ethyl acetate. The ethyl acetate layer was dried over magnesium sulfate, filtered, and concentrated. The crude material was purified by flash chromatography over silica using a hexanes:ethyl acetate gradient of 0 to 30% ethyl acetate to afford 0.427 g (77%) of methyl 2-fluoro-5-[6-(methyloxy)-2-naphthalenyl]benzoate. ¹H NMR (400 MHz, DMSO-d₆): δ 8.20 (dd, J=7, 2 Hz, 1H), 8.17 (d, J=12 Hz, 1H), 8.07 (m, 1H), 7.92 (d, J=5 Hz, 1H), 7.90 (d, J=4 Hz, 1H), 7.78 (dd, J=8, 2 Hz, 1H), 7.47 (dd, J=11, 9 Hz, 1H), 7.35 (d, J=3 Hz, 1H), 7.19 (dd, J=9, 3 Hz, 1H), 3.88 (s, 3H), 3.87 (s, 3H).

### 35c) Methyl 2-fluoro-5-(6-hydroxy-2-naphthalenyl)benzoate

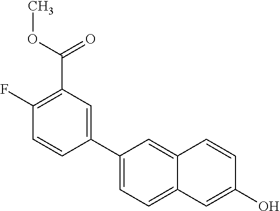

Boron tribromide (1 M in dichloromethane) (5.5 mL, 5.5 mmol) was added slowly to a solution of methyl 2-fluoro-5-[6-(methyloxy)-2-naphthalenyl]benzoate (0.427 g, 1.38 mmol) in dichloromethane (20 mL) at 0° C. The reaction mixture was stirred for 4 h at 0° C. The reaction mixture was poured into ice water and extracted with ethyl acetate. The organic layer was separated, washed with brine, dried over magnesium sulfate, filtered. and concentrated. The crude material was purified by flash chromatography over silica using a hexanes:ethyl acetate gradient of 0 to 30% ethyl acetate to afford 0.304 g (75%) of methyl 2-fluoro-5-(6-hydroxy-2-naphthalenyl)benzoate. ¹H NMR (400 MHz, DMSO-d₆): δ 9.84 (s, 1H), 8.18 (dd, J=7, 3 Hz, 1H), 8.10 (d, J=2 Hz, 1H), 8.04 (m, 1H), 7.85 (d, J=9 Hz, 1H), 7.78 (m, 1H), 7.71 (dd, J=9, 2 Hz, H), 7.46 (dd, J=11, 9 Hz, 1H), 7.13 (m, 1H), 7.10 (m, 1H), 3.88 (s, 3H).

### 35d) Methyl 5-[6-({[3-(2,6-dichlorophenyl)-5-(1-methylethyl)-4-isoxazolyl]methyl}oxy)-2-naphthalenyl]-2-fluorobenzoate

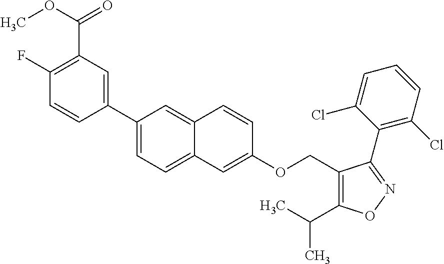

A mixture of methyl 2-fluoro-5-(6-hydroxy-2-naphthalenyl)benzoate (0.15 g, 0.506 mmol) and cesium carbonate (0.23 g, 0.708 mmol) in N,N-dimethylformamide (1.3 mL) was heated at 65° C. for 1 h. To the reaction mixture was added a solution of 4-(chloromethyl)-3-(2,6-dichlorophenyl)-5-(1-methylethyl)isoxazole (0.154 g, 0.506 mmol) in N,N-dimethylformamide (1 mL) and heating was continued at 65° C. for 24 h. The reaction mixture was cooled to room temperature and diluted with water. followed by ethyl acetate. The ethyl acetate layer was separated, washed several times with water. followed by brine, dried over magnesium sulfate, filtered, and concentrated. The crude oil was purified by flash chromatography over silica using a hexanes:ethyl acetate gradient of 0 to 30% ethyl acetate to afford 0.186 g (65%) of methyl 5-[6-({[3-(2,6-dichlorophenyl)-5-(1-methylethyl)-4-isoxazolyl]methyl}oxy)-2-naphthalenyl]-2-fluorobenzoate. ¹H NMR (400 MHz, DMSO-d₆): δ 8.18 (dd, J=7, 2 Hz, 1H), 8.13 (s, 1H), 8.04 (m, 1H), 7.84 (d, J=9 Hz, 1H), 7.79 (m, 2H), 7.61 (m, 2H), 7.52 (m, 1H), 7.46 (m, 1H), 7.30 (d, J=2 Hz, 1H), 6.92 (dd, J=9, 2 Hz, 1H), 4.94 (s, 2H), 3.88 (s, 3H), 3.50 (septet, J=7 Hz, 1H), 1.34 (d, J=7 Hz, 6H).

### 35e) 5-[6-({[3-(2,6-Dichlorophenyl)-5-(1-methylethyl)-4-isoxazolyl]methyl}oxy)-2-naphthalenyl]-2-fluorobenzoic acid

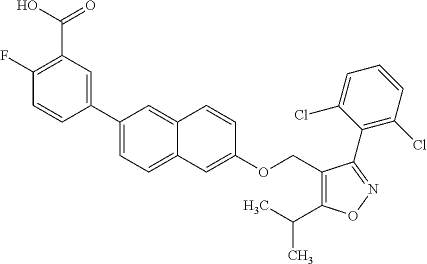

Methyl 5-[6-({[3-(2,6-dichlorophenyl)-5-(1-methylethyl)-4-isoxazolyl]methyl}oxy)-2-naphthalenyl]-2-fluorobenzoate (0.18 g, 0.319 mmol) and 1 N sodium hydroxide (0.68 mL, 0.68 mmol) were stirred in tetrahydrofuran (3.3 mL) and methanol (1.7 mL) overnight at room temperature. The reaction mixture was concentrated and diluted with 1 N hydrochloric acid followed by ethyl acetate. The organic layer was separated, washed with brine, dried over magnesium sulfate, filtered, and concentrated to afford 0.159 g (91%) of 5-[6-({[3-(2,6-dichlorophenyl)-5-(1-methylethyl)-4-isoxazolyl]methyl}oxy)-2-naphthalenyl]-2-fluorobenzoic acid. ¹H NMR (400 MHz, DMSO-d₆): δ 13.39 (s, 1H), 8.16 (dd, J=7, 2 Hz, 1H), 8.12 (s, 1H), 8.00 (m, 1H), 7.84 (d, J=9 Hz, 1H), 7.79 (m, 2H), 7.61 (m, 2H), 7.52 (m, 1H), 7.41 (m 1H), 7.29 (d, J=2 Hz, 1H), 6.92 (dd, J=9, 2 Hz, 1H), 4.94 (s, 2H), 3.50 (septet, J=7 Hz, 1H), 1.34 (d, J=7 Hz, 6H). HRMS C₃₀H₂₂Cl₂FNO₄ m/z 550.0988 (M+H)⁺_{Cal}; 550.0989 (M+H)⁺_{Obs}.

### Example 36

### 3-[6-({[3-(2,6-Dichlorophenyl)-5-(1-methylethyl)-4-isoxazolyl]methyl}oxy)-1-benzothien-2-yl]benzoic acid

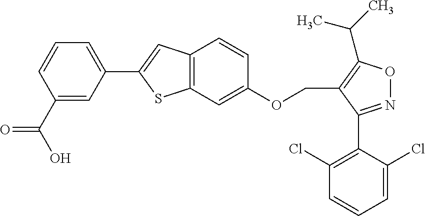

### 36a) 1-{[2,2-Bis(ethyloxy)ethyl]thio}-3-(methyloxy)benzene

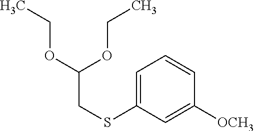

1-{[2,2-Bis(ethyloxy)ethyl]thio}-3-(methyloxy)benzene was prepared according to the general procedure described by S. L. Graham et al. (1989 J. Med. Chem. 32:2548-2554) by employing bromoacetaldehyde diethyl acetal (11 mL, 73.1 mmol), 3-methoxybenzenethiol (10 mL, 80.6 mmol), potassium carbonate (11.2 g, 81 mmol) and acetone (100 mL) to give 18.82 g of 1-{[2,2-bis(ethyloxy)ethyl]thio}-3-(methyloxy)benzene as a yellow liquid. The crude product was used without further purification. ¹H NMR (400 MHz, CDCl₃): 7.18 (t, J=8 Hz, 1H), 6.94 (m, 2H), 6.71 (dd, J=8, 2 Hz, 1H), 4.65 (t, J=6 Hz, 1H), 3.78 (s, 3H), 3.67 (m, 2H), 3.55 (m, 2H), 3.13 (d, J=6 Hz, 2H), 1.20 (t, J=7 Hz, 6H).

### 36b) 6-(Methyloxy)-1-benzothiophene

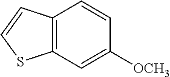

6-(Methyloxy)-1-benzothiophene was prepared according to the general procedure described by S. L. Graham et al. (1989 J. Med. Chem. 32:2548-2554) with modification and purified as described by K. Takeuchi et al. (1999 Bioorg. Med. Chem. Lett. 9:759-764). To a stirred solution of boron trifluoride diethyl etherate (9.7 mL, 76.8 mmol) in dichloromethane (1000 mL) was added, very slowly, dropwise, a solution of 1-{[2,2-bis(ethyloxy)ethyl]thio}-3-(methyloxy)benzene (18.8 g) in dichloromethane (150 mL) at room temperature under a nitrogen atmosphere. The reaction mixture was stirred for 30 min. To the stirred reaction mixture was slowly added a saturated aqueous solution of sodium bicarbonate. The reaction mixture was stirred at room temperature for 3 days. To the reaction mixture was slowly added an additional 500 mL of saturated aqueous sodium bicarbonate and the reaction mixture was stirred for 1 h. The organic phase was separated, dried over magnesium sulfate, filtered, and the filtrate was concentrated to give the crude product as a dark brown-orange liquid. The crude product was partially purified by flash chromatography over silica gel with hexanes:ethyl acetate (95:5) to give 8.3 g of an approximately 3:1 mixture of 6-(methyloxy)-1-benzothiophene and 4-(methyloxy)-1-benzothiophene, respectively. Purification of the 3:1 mixture by flash chromatography over silica gel with a hexanes:ethyl acetate gradient (100:0 to 95:5) failed to purify the desired 6-isomer. Purification of the impure product by flash chromatography over silica gel with hexanes as eluant gave 4.86 g (40% over two steps) of 6-(methyloxy)-1-benzothiophene as a colorless liquid. ¹H NMR (400 MHz, CDCl₃): δ 7.69 (d, J=9 Hz, 1H), 7.35 (d, J=2 Hz, 1H), 7.25 (m, 2H), 7.00 (dd, J=9, 2 Hz, 1H), 3.87 (s, 3H).

36c) [6-(Methyloxy)-1-benzothien-2-yl]boronic acid

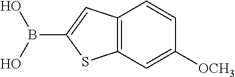

A solution of 6-(methyloxy)-1-benzothiophene (3.5 g, 21.3 mmol) in tetrahydrofuran (30 mL) was cooled between −60° C. and −70° C. in a dry ice/acetone bath and a solution of n-butyl lithium (1.6 M in hexanes) (14.8 mL, 23.7 mmol) was added slowly, dropwise, with stirring under a nitrogen atmosphere. The reaction mixture became a viscous suspension upon addition of the n-butyl lithium. The reaction mixture was manually swirled to facilitate mixing. Once the addition of n-butyl lithium was complete, the reaction mixture was stirred and occasionally swirled between −65° C. and −75° C. for 30 min. To the cold suspension was slowly added triisopropyl borate (5.6 mL, 24.3 mmol). The reaction mixture was manually swirled during addition of the triisopropyl borate, however, toward the end of the addition, the reaction mixture became a very thick mass. The reaction mixture was allowed to warm to 0° C. The reaction mixture was partitioned between ethyl acetate and 1 N hydrochloric acid. The organic phase was separated, dried over magnesium sulfate, filtered, and the filtrate was concentrated to give the crude product as a pale yellow solid. The solid was triturated with hexanes:diethyl ether (1:1) to give 1.92 g (43%) of [6-(methyloxy)-1-benzothien-2-yl]boronic acid as a pale yellow powder. ¹H NMR (400 MHz, DMSO-d₆): δ 7.79 (d, J=9 Hz, 1H), 7.75 (s, 1H), 7.52 (d, J=2 Hz, 1H), 6.97 (dd, J=9, 2 Hz, 1H), 3.81 (s, 3H). ESI-LCMS m/z 207 (M−H)⁻.

### 36d) Ethyl 3-[6-(methyloxy)-1-benzothien-2-yl]benzoate

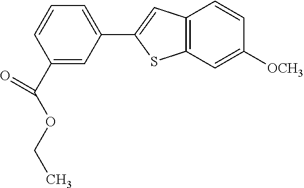

[6-(Methyloxy)-1-benzothien-2-yl]boronic acid (1.2 g, 5.77 mmol), ethyl-3-iodobenzoate (1.1 mL, 6.53 mmol), sodium carbonate (2 M) (6 mL, 12 mmol), tetrakis(triphenylphosphine)palladium(0) (0.241 g, 0.21 mmol), and toluene (30 mL) were combined and the stirred reaction mixture was heated at reflux for 3 h under a nitrogen atmosphere. The reaction mixture was allowed to stand at room temperature overnight. The reaction mixture was partitioned between ethyl acetate and water. The aqueous phase was separated and extracted with ethyl acetate. The organic extracts were combined, dried over magnesium sulfate, filtered, and the filtrate was concentrated to give an oil. The crude product was purified by flash chromatography over silica with a hexanes:ethyl acetate gradient (100:0 to 95:5) to give 0.90 g (50%) of ethyl 3-[6-(methyloxy)-1-benzothien-2-yl]benzoate as a white solid. ¹H NMR (400 MHz, DMSO-d₆): δ 8.19 (s, 1H), 8.00 (d, J=8 Hz, 1H), 7.90 (d, J=8 Hz, 1H), 7.87 (s, 1H), 7.74 (d, J=9 Hz, 1H), 7.60 (t, J=8 Hz, 1H), 7.57 (d, J=2 Hz, 1H), 7.01 (dd, J=9, 2 Hz, 1H), 4.35 (q, J=7 Hz, 2H), 3.82 (s, 3H), 1.33 (t, J=7 Hz, 3H). ESI-LCMS m/z 313 (M+H)⁺.

### 36e) Ethyl 3-(6-hydroxy-1-benzothien-2-yl)benzoate

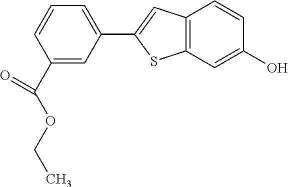

To a stirred ice-water cooled solution of ethyl 3-[6-(methyloxy)-1-benzothien-2-yl]benzoate (0.269 g, 0.86 mmol) in dichloromethane (10 mL) was slowly added a solution of boron tribromide in dichloromethane (1 M) (3.4 mL, 3.4 mmol) under a nitrogen atmosphere. The reaction mixture was stirred with cooling for 2 h. The reaction mixture was poured onto ice and the mixture was stirred at room temperature. The aqueous mixture was extracted with ethyl acetate. The organic phase was separated, dried over magnesium sulfate, filtered, and the filtrate was concentrated to give a pale tan solid. The crude product was purified by flash chromatography over silica with a hexanes:ethyl acetate gradient (100:0 to 60:40) to give 0.198 g (77%) of ethyl 3-(6-hydroxy-1-benzothien-2-yl)benzoate as a white solid. ¹H NMR (400 MHz, DMSO-d₆): δ 9.73 (s, 1H), 8.17 (s, 1H), 7.97 (d, J=8 Hz, 1H), 7.88 (d, J=8 Hz, 1H), 7.81 (s, 1H), 7.65 (d, J=9 Hz, 1H), 7.58 (t, J=8 Hz, 1H), 7.28 (d, J=2 Hz, 1H), 6.87 (dd, J=9, 2 Hz, 1H), 4.34 (q, J=7 Hz, 2H), 1.33 (t, J=7 Hz, 3H).

### 36f) Ethyl 3-[6-({[3-(2,6-dichlorophenyl)-5-(1-methylethyl)-4-isoxazolyl]methyl}oxy)-1-benzothien-2-yl]benzoate

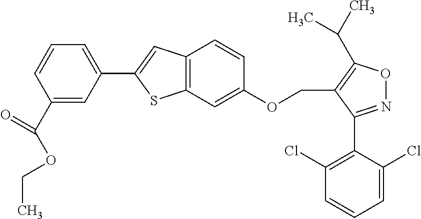

To a stirred ice-water cooled mixture of ethyl 3-(6-hydroxy-1-benzothien-2-yl)benzoate (0.198 g, 0.66 mmol), [3-(2,6-dichlorophenyl)-5-(1-methylethyl)-4-isoxazolyl]methanol (prepared according to the general procedure described in Maloney, P. R., et al., 2000 J. Med. Chem. 43:2971-2974) (0.19 g, 0.66 mmol), triphenylphosphine (0.172 g, 0.66 mmol), and dichloromethane (10 mL) was slowly added, dropwise, a solution of diisopropylazodicarboxylate (0.13 mL, 0.66 mmol) in dichloromethane (0.13 mL) under a nitrogen atmosphere. The reaction mixture was stirred with cooling for 10 min and the ice-water bath was removed. The reaction mixture was stirred overnight at room temperature under a nitrogen atmosphere. The reaction mixture was concentrated and the crude product was purified by flash chromatography over silica with a hexanes:ethyl acetate gradient (100:0 to 75:25) to give 0.251 g of an oil which partially solidified upon standing. The oil was dissolved in dichloromethane and acetonitrile and the solution was concentrated to give 0.246 g of ethyl 3-[6-({[3-(2,6-dichlorophenyl)-5-(1-methylethyl)-4-isoxazolyl]methyl}oxy)-1-benzothien-2-yl]benzoate as a white solid. ¹H NMR (400 MHz, CDCl₃): δ 8.32 (s, 1H), 7.97 (d, J=8 Hz, 1H), 7.81 (d, J=8 Hz, 1H), 7.60 (d, J=9 Hz, 1H), 7.50 (s, 1H), 7.47 (t, J=8 Hz, 1H), 7.41 (d, J=8 Hz, 2H), 7.32 (dd, J=9, 7 Hz, 1H), 7.17 (d, J=2 Hz, 1H), 6.85 (dd, J=9, 2 Hz, 1H), 4.80 (s, 2H), 4.42 (q, J=7 Hz, 2H), 3.35 (septet, J=7H, 1H), 1.43 (d, J=7 Hz, 6H), 1.42 (t, J=7 Hz, 3H). ESI-LCMS m/z 566 (M+H)⁺.

### 36g) 3-[6-({[3-(2,6-Dichlorophenyl)-5-(1-methylethyl)-4-isoxazolyl]methyl}oxy)-1-benzothien-2-yl]benzoic acid

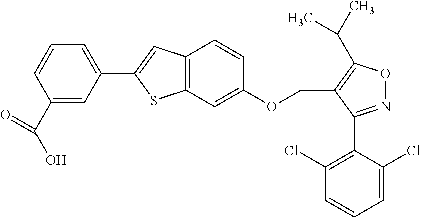

To a stirred solution of ethyl 3-[6-({[3-(2,6-dichlorophenyl)-5-(1-methylethyl)-4-isoxazolyl]methyl}oxy)-1-benzothien-2-yl]benzoate (0.217 g, 0.38 mmol) in tetrahydrofuran (1 mL) was added, dropwise, a solution of lithium hydroxide (1 N) (1 mL, 1 mmol). The reaction mixture was stirred overnight at room temperature under a nitrogen atmosphere. To the reaction mixture was added tetrahydrofuran (1 mL) and stirring was continued for another four days. The reaction mixture was concentrated and the residue was partitioned between ethyl acetate (15 mL), water (5 mL), and saturated sodium hydrogensulfate (0.20 mL). The organic phase was separated, washed with water (3 mL), followed by saturated sodium chloride (3 mL), dried over magnesium sulfate, filtered, and the filtrate was concentrated to give 0.203 g (99%) of 3-[6-({[3-(2,6-dichlorophenyl)-5-(1-methylethyl)-4-isoxazolyl]methyl}oxy)-1-benzothien-2-yl]benzoic acid as an oil which solidified upon standing to give a white solid. ¹H NMR (400 MHz, DMSO-d₆): δ 13.15 (br s, 1H), 8.16 (s, 1H), 7.96 (d, J=8 Hz, 1H), 7.88 (d, J=8 Hz, 1H), 7.83 (s, 1H), 7.50-7.66 (m, 5H), 7.47 (s, 1H), 6.77 (d, J=9 Hz, 1H), 4.88 (s, 2H), 3.46 (septet, J=7 Hz, 1H), 1.32 (d, J=7 Hz, 6H). HRMS C₂₈H₂₂NO₄SCl₂ m/z 538.0647 (M+H)⁺_{Cal}; 538.0657 (M+H)⁺_{Obs}.

### Example 37

### 3-[2-({[3-(2,6-Dichlorophenyl)-5-(1-methylethyl)-4-isoxazolyl]methyl}amino)-1,3-benzothiazol-6-yl]benzoic acid

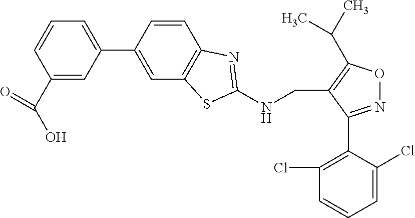

### 37a) Methyl 3-(2-amino-1,3-benzothiazol-6-yl)benzoate

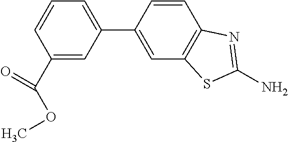

2-Amino-6-bromobenzothiazole (1.75 g, 7.6 mmol), (3-methoxycarbonylphenyl)boronic acid (1.8 g, 10 mmol), sodium carbonate (2 M) (7 mL, 14 mmol), tetrakis(triphenylphosphine)palladium(0) (0.48 g, 0.42 mmol), and 1,2-dimethoxyethane (75 mL) were combined and the stirred reaction mixture was heated at 85° C. for 4 h under a nitrogen atmosphere. The reaction mixture was allowed to stand at room temperature overnight. To the reaction mixture was added tetrakis(triphenylphosphine)palladium(0) (0.10 g, 0.087 mmol) and the reaction mixture was heated at 85° C. for 3 h. The reaction mixture was allowed to stand at room temperature for three days. To the reaction mixture was added tetrakis(triphenylphosphine)palladium(0) (0.146 g, 0.126 mmol) and sodium carbonate (2 M) (20 mL, 40 mmol). The reaction mixture was heated at 85° C. for 3 h under a nitrogen atmosphere. The reaction mixture was allowed to cool at room temperature. The reaction mixture was partitioned between water and ethyl acetate. The aqueous phase was separated and extracted with ethyl acetate. The organic extracts were combined, washed with saturated sodium chloride, dried over magnesium sulfate, filtered, and the filtrate was concentrated to give a red-orange liquid which partially solidified upon standing. Ethyl acetate was added to the crude product and the mixture was heated. The solvent was removed in vacuo. To the crude product was added dichloromethane, methanol, and ethyl acetate. The suspension was filtered to give 0.179 g of methyl 3-(2-amino-1,3-benzothiazol-6-yl)benzoate as an off-white solid. The filtrate was adsorbed onto silica and purified by flash chromatography with hexanes, followed by hexanes:ethyl acetate (1:1) and finally ethyl acetate to give 0.377 g of methyl 3-(2-amino-1,3-benzothiazol-6-yl)benzoate as a tan solid for a total yield of 0.556 g (26%). ¹H NMR (400 MHz, DMSO-d₆): δ 8.17 (s, 1H), 8.03 (d, J=2 Hz, 1H), 7.92 (d, J=8 Hz, 1H), 7.88 (d, J=8 Hz, 1H), 7.52-7.59 (m, 4H), 7.39 (d, J=8 Hz, 1H), 3.86 (s, 3H). ESI-LCMS m/z 285 (M+H)⁺.

### 37b) 3-(2,6-Dichlorophenyl)-5-(1-methylethyl)-4-isoxazolecarbaldehyde

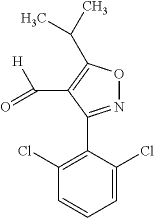

To a stirred turbid mixture of pyridinium chlorochromate (0.363 g, 1.68 mmol) and magnesium sulfate (0.542 g, 4.5 mmol) in dichloromethane (5 mL), was slowly added a solution of [3-(2,6-dichlorophenyl)-5-(1-methylethyl)-4-isoxazolyl]methanol (prepared according to the general procedure described in Maloney, P. R., et al., 2000 J. Med. Chem. 43:2971-2974 (0.224 g, 0.78 mmol) in dichloromethane (5 mL) at room temperature under a nitrogen atmosphere. After 2 h, the reaction mixture was diluted with diethyl ether (10 mL) and filtered through a pad of silica. The filtrate was concentrated to give 0.181 g (82%) of 3-(2,6-dichlorophenyl)-5-(1-methylethyl)-4-isoxazolecarbaldehyde as a pale yellow solid.

¹H NMR (400 MHz, CDCl₃): δ 9.69 (s, 1H), 7.41-7.50 (m, 3H), 3.79 (septet, J=7 Hz, 1H), 1.50 (d, J=7 Hz, 6H). ESI-LCMS m/z 284 (M+H)⁺.

### 37c) Methyl 3-[2-({[3-(2,6-dichlorophenyl)-5-(1-methylethyl)-4-isoxazolyl]methyl}amino)-1,3-benzothiazol-6-yl]benzoate

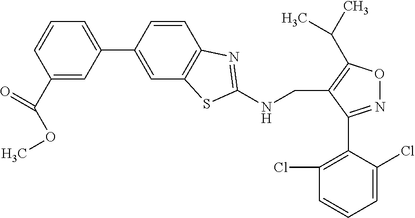

To a stirred mixture of 3-(2,6-dichlorophenyl)-5-(1-methylethyl)-4-isoxazolecarbaldehyde (0.172 g, 0.61 mmol) and methyl 3-(2-amino-1,3-benzothiazol-6-yl)benzoate (0.173 g, 0.61 mmol) in tetrahydrofuran (2 mL) was added dibutyltin dichloride (0.012 g, 0.039 mmol), followed by phenylsilane (0.08 mL, 0.65 mmol) at room temperature under a nitrogen atmosphere. After approximately 15 min, tetrahydrofuran (2 mL) was added to the reaction mixture. The reaction mixture was stirred at room temperature overnight. Thin layer chromatography indicated that only starting material was present. The reaction mixture was heated at 75° C. overnight. The reaction mixture was allowed to cool at room temperature. To the reaction mixture was added dibutyltin dichloride (0.0136 g, 0.045 mmol), followed by phenylsilane (0.08 mL, 0.65 mmol). The stirred reaction mixture was heated at 75° C. overnight under a nitrogen atmosphere. The reaction mixture was allowed to cool at room temperature and adsorbed onto silica. The crude product was purified by flash chromatography over silica with a hexanes:ethyl acetate gradient (100:0 to 75:25) to give 0.016 g (9%) of methyl 3-[2-({[3-(2,6-dichlorophenyl)-5-(1-methylethyl)-4-isoxazolyl]methyl}amino)-1,3-benzothiazol-6-yl]benzoate. ¹H NMR (400 MHz, CDCl₃): δ 8.25 (s, 1H), 8.01 (d, J=8 Hz, 1H), 7.79 (s, 1H), 7.76 (d, J=8 Hz, 1H), 7.55-7.61 (m, 2H), 7.51 (t, J=8 Hz, 1H), 7.39 (m, 2H), 7.30 (m, 1H), 4.36 (s, 2H), 3.95 (s, 3H), 3.42 (septet, J=7 Hz, 1H), 1.46 (d, J=7 Hz, 6H). ESI-LCMS m/z 552 (M+H)⁺.

### 37d) 3-[2-({[3-(2,6-Dichlorophenyl)-5-(1-methylethyl)-4-isoxazolyl]methyl}amino)-1,3-benzothiazol-6-yl]benzoic acid

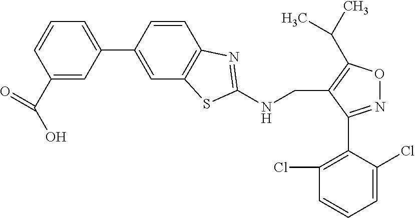

To methyl 3-[2-({[3-(2,6-dichlorophenyl)-5-(1-methylethyl)-4-isoxazolyl]methyl}amino)-1,3-benzothiazol-6-yl]benzoate (0.014 g, 0.025 mmol) was added tetrahydrofuran (0.2 mL), followed by lithium hydroxide (1 M) (0.10 mL, 0.10 mmol). The pale yellow solution was stirred overnight at room temperature. ES-LCMS analysis of the reaction mixture indicated that the reaction was not complete. To the reaction mixture was added tetrahydrofuran (0.10 mL). The reaction mixture was stirred at room temperature for 8 h. To the reaction mixture was added tetrahydrofuran (0.10 mL). The reaction mixture was stirred overnight at room temperature. The reaction mixture was concentrated and the crude product was partitioned between ethyl acetate (15 mL) and water (5 mL) and saturated sodium hydrogensulfate (0.20 mL). The organic phase was separated, washed with water (3 mL), followed by saturated sodium chloride (3 mL), dried over magnesium sulfate, filtered, and the filtrate was concentrated to give a white solid. The crude product was purified by flash chromatography over silica with ethyl acetate as eluant to give 2 mg of the desired product as a white solid. Methanol was added to the silica column and additional compound quickly eluted to give another 10 mg of the desired product as a white solid. The two crops were independently dissolved in dichloromethane with the aid of minimal methanol to fully dissolve the solids. The solution of the second crop was filtered to remove trace silica, if present. The filtrate was combined with the solution of the first crop and the solvent was removed in vacuo to give 0.008 g (59%) of 3-[2-({[3-(2,6-dichlorophenyl)-5-(1-methylethyl)-4-isoxazolyl]methyl}amino)-1,3-benzothiazol-6-yl]benzoic acid as a white solid. ¹H NMR (400 MHz, DMSO-d₆): δ 8.18 (t, J=6 Hz, 1H), 8.13 (s, 1H), 7.92 (s, 1H), 7.82 (d, J=8 Hz, 1H), 7.66 (br s, 1H), 7.53 (m, 2H), 7.38-7.48 (m, 3H), 7.27 (d, J=8 Hz, 1H), 4.27 (d, J=6 Hz, 2H), 3.59 (septet, J=7 Hz, 1H), 1.36 (d, J=7 Hz, 6H). HRMS C₂₇H₂₂N₃O₃SCl₂ m/z 538.0759 (M+H)⁺_{Cal}; 538.0761 (M+H)⁺_{Obs}.

### Example 38

### 3-[6-({[3-(2,6-Dichlorophenyl)-5-(1-methylethyl)-4-isoxazolyl]methyl}oxy)-2-naphthalenyl]benzoic acid

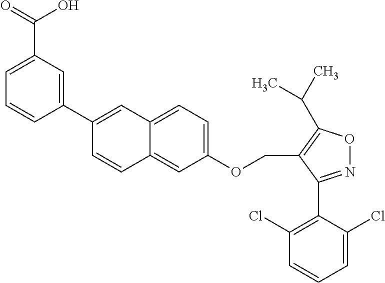

### 38a) Methyl 3-[6-(methyloxy)-2-naphthalenyl]benzoate

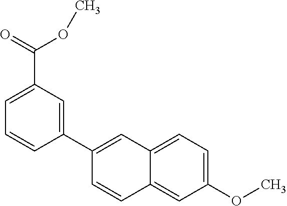

2-Bromo-6-methoxynaphthalene (0.824 g, 3.48 mmol), (3-methoxycarbonylphenyl)boronic acid (0.57 g, 3.17 mmol), tetrakistriphenylphosphine palladium(0) (0.217 g, 0.188 mmol), sodium carbonate (2 M) (6.4 mL, 12.8 mmol), and toluene (20 mL) were combined in a round bottom flask and the stirred reaction mixture was heated at reflux for 3 h under a nitrogen atmosphere. The reaction mixture was allowed to stand at room temperature overnight. To the reaction mixture was added (3-methoxycarbonylphenyl)boronic acid (0.496 g, 2.7 mmol) and the reaction mixture was heated at reflux for 2 h under a nitrogen atmosphere. The reaction mixture was allowed to cool at room temperature and partitioned between water and ethyl acetate. The organic phase was separated, dried over magnesium sulfate, filtered, and the filtrate was concentrated to give the crude product. The crude product was purified by flash chromatography over silica with a hexanes:ethyl acetate gradient (100:0 to 80:20) to give 0.335 g (36%) of methyl 3-[6-(methyloxy)-2-naphthalenyl]benzoate as a white solid. ¹H NMR (400 MHz, CDCl₃): δ 8.38 (s, 1H), 8.05 (m, 2H), 7.89 (d, J=8 Hz, 1H), 7.82 (t, J=8 Hz, 2H), 7.73 (dd, J=8, 2 Hz, 1H), 7.54 (t, J=8 Hz, 1H), 7.19 (m, 2H), 3.96 (s, 3H), 3.94 (s, 3H). ESI-LCMS m/z 293 (M+H)⁺.

### 38b) Methyl 3-(6-hydroxy-2-naphthalenyl)benzoate

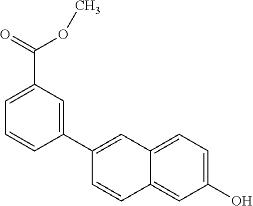

To a stirred ice-water cooled solution of methyl 3-[6-(methyloxy)-2-naphthalenyl]benzoate (0.224 g, 0.766 mmol) in dichloromethane (10 mL) was slowly added, dropwise, boron tribromide (1 M in dichloromethane) (3.2 mL, 3.2 mmol) under a nitrogen atmosphere. After 90 min, the reaction mixture was poured onto ice and the mixture was partitioned between water and ethyl acetate. The organic phase was separated, dried over magnesium sulfate, filtered, and the filtrate was concentrated to give a gold-yellow oil which solidified upon standing. The crude product was purified by flash chromatography over silica with a hexanes:ethyl acetate gradient (100:0 to 75:25) to give 0.12 g (56%) of methyl 3-(6-hydroxy-2-naphthalenyl)benzoate as a white solid. ¹H NMR (400 MHz, CDCl₃): δ 8.38 (s, 1H), 8.02 (m, 2H), 7.88 (d, J=8 Hz, 1H), 7.82 (d, J=9 Hz, 1H), 7.77 (d, J=9 Hz, 1H), 7.72 (dd, J=9, 2 Hz, 1H), 7.54 (t, J=8 Hz, 1H), 7.18 (d, J=2 Hz, 1H), 7.14 (dd, J=9, 3 Hz, 1H), 4.95 (br s, 1H), 3.96 (s, 3H). ESI-LCMS m/z 277 (M−H)⁻.

### 38c) Methyl 3-[6-({[3-(2,6-dichlorophenyl)-5-(1-methylethyl)-4-isoxazolyl]methyl}oxy)-2-naphthalenyl]benzoate

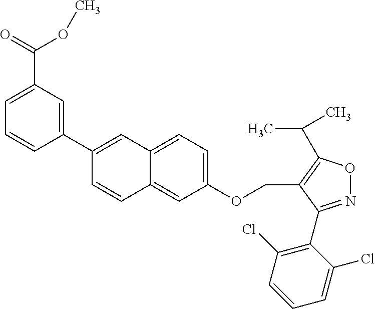

To a stirred ice-water cooled turbid mixture of methyl 3-(6-hydroxy-2-naphthalenyl)benzoate (0.12 g, 0.43 mmol), [3-(2,6-dichlorophenyl)-5-(1-methylethyl)-4-isoxazolyl]methanol (prepared according to the general procedure described in Maloney, P. R., et al., 2000 J. Med. Chem. 43:2971-2974) (0.13 g, 0.45 mmol), and triphenylphosphine (0.12 g, 0.46 mmol) in dichloromethane (10 mL) was slowly added, dropwise, a solution of diisopropyl azodicarboxylate (0.085 mL, 0.43 mmol) in dichloromethane (0.2 mL) under a nitrogen atmosphere. The reaction mixture was stirred with cooling for 10 min and the ice-water bath was removed. The reaction mixture was stirred at room temperature overnight under a nitrogen atmosphere. The reaction mixture was concentrated and the crude product was partially purified by flash chromatography over silica with a hexanes:ethyl acetate gradient (100:0 to 75:25) to give 0.013 g of methyl 3-[6-({[3-(2,6-dichlorophenyl)-5-(1-methylethyl)-4-isoxazolyl]methyl}oxy)-2-naphthalenyl]benzoate as a white amorphous solid as well as 0.247 g of impure product. The impure product was purified by flash chromatography over silica with dichloromethane as eluant to give 0.126 g (total yield, 0.139 g (59%)) of methyl 3-[6-({[3-(2,6-dichlorophenyl)-5-(1-methylethyl)-4-isoxazolyl]methyl}oxy)-2-naphthalenyl]benzoate as a viscous colorless oil. ¹H NMR (400 MHz, CDCl₃): δ 8.36 (s, 1H), 8.02 (d, J=8 Hz, 1H), 7.98 (s, 1H), 7.87 (d, J=8 Hz, 1H), 7.70-7.76 (m, 3H), 7.53 (t, J=8 Hz, 1H), 7.40 (d, J=8 Hz, 2H), 7.31 (dd, J=9, 7 Hz, 1H), 7.05 (m, 1H), 7.04 (dd, J=9, 2 Hz, 1H), 4.86 (s, 2H), 3.96 (s, 3H), 3.39 (septet, J=7 Hz, 1H), 1.44 (d, J=7 Hz, 6H). ESI-LCMS m/z 546 (M+H)⁺ and 568 (M+Na)⁺.

### 38d) 3-[6-({[3-(2,6-Dichlorophenyl)-5-(1-methylethyl)-4-isoxazolyl]methyl}oxy)-2-naphthalenyl]benzoic acid

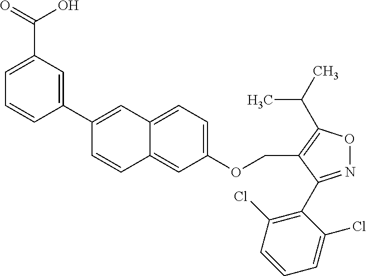

To a stirred solution of methyl 3-[6-({[3-(2,6-dichlorophenyl)-5-(1-methylethyl)-4-isoxazolyl]methyl}oxy)-2-naphthalenyl]benzoate (0.113 g, 0.21 mmol) in tetrahydrofuran (1.6 mL) was slowly added, dropwise, a solution of lithium hydroxide (1 N) (0.75 mL, 0.75 mmol) at room temperature under a nitrogen atmosphere. The reaction mixture was stirred overnight at room temperature. The reaction mixture was partially concentrated in vacuo and the residue was partitioned between water (5 mL), ethyl acetate (15 mL), and saturated sodium hydrogensulfate (0.2 mL). The organic phase was separated, washed with water (3 mL), followed by saturated sodium chloride (3 mL), dried over magnesium sulfate, filtered, and the filtrate was concentrated to give a white amorphous solid. To the solid was added acetonitrile (˜2 mL). A white solid was filtered and dried under vacuum at ˜75° C. to give 0.067 g (60%) of 3-[6-({[3-(2,6-dichlorophenyl)-5-(1-methylethyl)-4-isoxazolyl]methyl}oxy)-2-naphthalenyl]benzoic acid. ¹H NMR (400 MHz, DMSO-d₆): δ 13.03 (br s, 1H), 8.27 (s, 1H), 8.15 (s, 1H), 8.00 (d, J=8 Hz, 1H), 7.92 (d, J=8 Hz, 1H), 7.86 (d, J=9 Hz, 1H), 7.81 (m, 2H), 7.61 (m, 3H), 7.52 (dd, J=9, 7 Hz, 1H), 7.30 (d, J=2 Hz, 1H), 6.93 (dd, J=9, 2 Hz, 1H), 4.94 (s, 2H), 3.50 (septet, J=7 Hz, 1H), 1.34 (d, J=7 Hz, 6H). HRMS C₃₀H₂₄Cl₂NO₄ m/z 532.1082 (M+H)⁺_{Cal}; 532.1088 (M+H)⁺_{Obs}.

### Example 39

### 3-(2-{2-[3-(2,6-dichlorophenyl)-5-(1-methylethyl)-4-isoxazolyl]ethyl}-1,3-benzoxazol-5-yl)benzoic acid

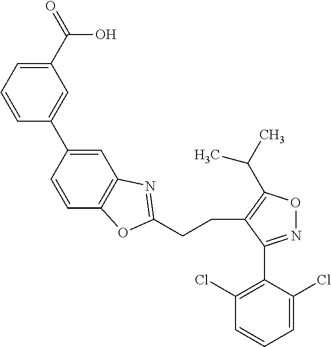

### 39a) 3-(2,6-Dichlorophenyl)-5-(1-methylethyl)-4-isoxazolecarbaldehyde

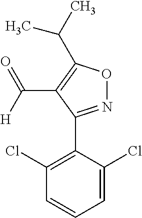

To a stirred suspension of pyridinium chlorochromate (1.28 g, 5.94 mmol) and magnesium sulfate (2.0 g, 16.6 mmol) in dichloromethane (20 mL) was slowly added, dropwise, over a 30-min period a solution of [3-(2,6-dichlorophenyl)-5-(1-methylethyl)-4-isoxazolyl] (prepared according to the general procedure described in Maloney, P. R., et al., 2000 J. Med. Chem. 43:2971-2974) (0.766 g, 2.68 mmol) at room temperature under a nitrogen atmosphere. The reaction mixture was stirred for 75 min. The reaction mixture was diluted with diethyl ether (30 mL) and filtered through a pad of silica. The pad of silica was washed with diethyl ether and the filtrate was concentrated to give 0.692 g (91%) of 3-(2,6-dichlorophenyl)-5-(1-methylethyl)-4-isoxazolecarbaldehyde as a pale green oil which solidified to give a pale green solid. ¹H NMR (400 MHz, CDCl₃): δ 9.66 (s, 1H), 7.45 (m, 2H), 7.39 (dd, J=10, 7 Hz, 1H), 3.76 (septet, J=7 Hz, 1H), 1.47 (d, J=7 Hz, 6H).

### 39b) 3-[3-(2,6-Dichlorophenyl)-5-(1-methylethyl)-4-isoxazolyl]propanoic acid

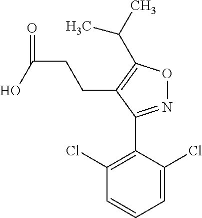

3-(2,6-Dichlorophenyl)-5-(1-methylethyl)-4-isoxazolecarbaldehyde (0.293 g, 1.03 mmol), triethylamine formate buffer (0.77 mL) (prepared by slowly adding formic acid (0.67 mL) to stirred triethylamine (1.0 mL)), 2,2-dimethyl-1,3-dioxane-4,6-dione (0.156 g, 1.08 mmol), and N,N-dimethylformamide (0.77 mL) were combined in a round bottom flask and the stirred solution was heated between 95° C.-100° C. for 5 h under a nitrogen atmosphere. The reaction mixture was allowed to stand overnight at room temperature. Water was added to the reaction mixture and the pH was adjusted to approximately 1 (litmus paper) with 1 N hydrochloric acid. The acidic aqueous mixture was extracted with dichloromethane. The organic extract was dried over magnesium sulfate, filtered, and the filtrate was concentrated to give 0.81 g of a gold-yellow liquid. The crude product was combined with 0.070 g of crude product prepared similarly in a previous reaction and purified by flash chromatography over silica with a dichloromethane:methanol gradient (100:0 to 98:2) to give 0.301 g (79% for the two reactions) of 3-[3-(2,6-dichlorophenyl)-5-(1-methylethyl)-4-isoxazolyl]propanoic acid as an off-white solid. ¹H NMR (400 MHz, CDCl₃): δ 7.42 (m, 2H), 7.34 (dd, J=9, 7 Hz, 1H), 3.24 (septet, J=7 Hz, 1H), 2.58 (t, J=8 Hz, 2H), 2.34 (t, J=8 Hz, 2H), 1.38 (d, J=7 Hz, 6H). ESI-LCMS m/z 326 (M−H)⁻.

### 39c) Methyl 4′-hydroxy-3′-nitro-3-biphenylcarboxylate

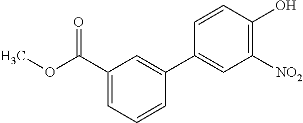

4-Bromo-2-nitrophenol (0.624 g, 2.86 mmol), (3-methoxycarbonylphenyl)boronic acid (0.632 g, 3.51 mmol), sodium carbonate (2 M) (2 mL, 4 mmol), tetrakis(triphenylphosphine)palladium(0) (0.198 g, 0.17 mmol), and 1,2-dimethoxyethane (20 mL) were combined and the stirred reaction mixture was heated between 85° C.-90° C. under a nitrogen atmosphere. After 1 h, sodium carbonate (2 M) (6 mL, 12 mmol) was added to the reaction mixture and heating was continued for 3 h. The oil bath was removed and the reaction mixture was allowed to stand at room temperature overnight. The reaction mixture was partitioned between water and ethyl acetate. The organic phase was separated, dried over magnesium sulfate, filtered, and the filtrate was concentrated to give the crude product. The crude product was partially purified by flash chromatography over silica with a hexanes:ethyl acetate gradient (100:0 to 70:30) to give a yellow oil which partially solidified upon standing. The impure product was dissolved in diethyl ether and the solution was washed with sodium hydroxide (1 N). The layers were separated and the pH of the basic aqueous phase was adjusted to approximately 1 (litmus paper) with 1 N hydrochloric acid. The acidic aqueous phase was extracted with diethyl ether. The organic extract was dried over magnesium sulfate, filtered, and the filtrate was concentrated to give 0.116 g of a mixture of methyl 4′-hydroxy-3′-nitro-3-biphenylcarboxylate and 4′-hydroxy-3′-nitro-3-biphenylcarboxylic acid in a ratio of ˜1:9 as determined by ES-LCMS (ES-LCMS m/z 272 (M−H)⁻ and 258 (M−H)⁻ for the carboxylic ester and carboxylic acid, respectively). To the 1:9 mixture of methyl 4′-hydroxy-3′-nitro-3-biphenylcarboxylate and 4′-hydroxy-3′-nitro-3-biphenylcarboxylic (0.114 g) was added methanol (15 mL) and concentrated sulfuric acid (5 drops). The stirred reaction mixture was heated at reflux under a nitrogen atmosphere for 3.75 h. The reaction mixture was allowed to cool at room temperature. The reaction mixture was partitioned between water and ethyl acetate. The organic phase was separated, washed with water, dried over magnesium sulfate, filtered, and the filtrate was concentrated to give 0.113 g (14%) of methyl 4′-hydroxy-3′-nitro-3-biphenylcarboxylate as a yellow solid. ¹H NMR (400 MHz, CDCl₃): δ 10.61 (s, 1H), 8.36 (d, J=2 Hz, 1H), 8.23 (s, 1H), 8.05 (d, J=8 Hz, 1H), 7.87 (dd, J=9, 2 Hz, 1H), 7.75 (d, J=8 Hz, 1H), 7.54 (t, J=8 Hz, 1H), 7.27 (d, J=9 Hz, 1H), 3.96 (s, 3H). ESI-LCMS m/z 272 (M−H)⁻.

### 39d) Methyl 3′-amino-4′-hydroxy-3-biphenylcarboxylate

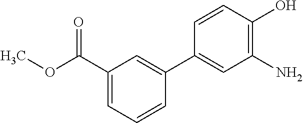

To a suspension of methyl 4′-hydroxy-3′-nitro-3-biphenylcarboxylate (0.11 g, 0.40 mmol) in ethanol (10 mL) was added 10% palladium on carbon (Degussa type; 50% water by weight) (0.023 g). The flask was evacuated and filled with nitrogen (three times), evacuated, then filled with hydrogen via a balloon. The reaction mixture was stirred overnight at room temperature under a hydrogen atmosphere. The reaction mixture was filtered through a pad of Celite® and the pad was washed with ethanol. The filtrate was concentrated to give 0.10 g of methyl 3′-amino-4′-hydroxy-3-biphenylcarboxylate as a beige solid. The compound was used directly without further purification. ¹H NMR (400 MHz, DMSO-d₆): δ 9.36 (br s, 1H), 8.05 (s, 1H), 7.80 (dd, J=10, 7 Hz, 2H), 7.52 (t, J=8 Hz, 1H), 6.98 (d, J=2 Hz, 1H), 6.79 (dd, J=8, 2 Hz, 1H), 6.74 (d, J=8 Hz, 1H), 5.17 (br s, 1H), 4.33 (br s, ˜0.5H), 3.86 (s, 3H). ESI-LCMS m/z 244 (M+H)⁺.

### 39e) 3-(2-{2-[3-(2,6-dichlorophenyl)-5-(1-methylethyl)-4-isoxazolyl]ethyl}-1,3-benzoxazol-5-yl)benzoic acid

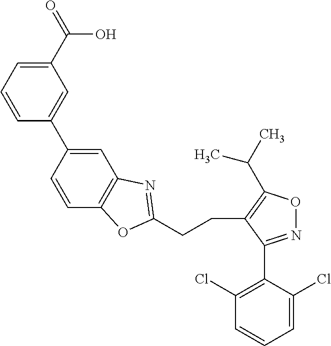

A mixture of 3-[3-(2,6-dichlorophenyl)-5-(1-methylethyl)-4-isoxazolyl]propanoic acid (from Example 39 (b)) (0.135 g, 0.41 mmol), triethylamine (0.06 mL, 0.43 mmol), and dichloromethane (5 mL) was cooled to between −5° C. and −15° C. (bath temperature) using a dry ice/acetone bath. To the cold mixture was slowly added, dropwise, isobutylchloroformate (0.055 mL, 0.42 mmol) with stirring under a nitrogen atmosphere. The reaction mixture was stirred between −5° C. and −15° C. (bath temperature) for 30 min. To the cold reaction mixture was added, portionwise, a slightly turbid solution of methyl 3′-amino-4′-hydroxy-3-biphenylcarboxylate (0.10 g, 0.41 mmol) in dichloromethane (5 mL) via an addition funnel. The addition funnel was rinsed with dichloromethane (1 mL) into the cold reaction mixture. The reaction mixture was allowed to slowly warm to room temperature overnight under a nitrogen atmosphere. The reaction mixture was partitioned between dichloromethane and water. The organic phase was separated and filtered to give 0.055 g of the intermediate amide (i.e. methyl 3′-({3-[3-(2,6-dichlorophenyl)-5-(1-methylethyl)-4-isoxazolyl]propanoyl}amino)-4′-hydroxy-3-biphenylcarboxylate) as a white solid. The filtrate was washed with saturated sodium chloride, dried over magnesium sulfate, filtered, and the filtrate was separated into two equal volumes. The two solutions were independently concentrated to give two batches (0.068 and 0.069 g) of crude methyl 3′-({3-[3-(2,6-dichlorophenyl)-5-(1-methylethyl)-4-isoxazolyl]propanoyl}amino)-4′-hydroxy-3-biphenylcarboxylate as a green solid. To crude methyl 3′-({3-[3-(2,6-dichlorophenyl)-5-(1-methylethyl)-4-isoxazolyl]propanoyl}amino)-4′-hydroxy-3-biphenylcarboxylate (0.069 g) was added propionic acid (0.5 mL). The reaction mixture was heated between 135° C.-150° C. with stirring under a nitrogen atmosphere for 2.5 h. Analysis of the reaction mixture by electrospray LCMS indicated that the intermediate amide cyclized to methyl 3-(2-{2-[3-(2,6-dichlorophenyl)-5-(1-methylethyl)-4-isoxazolyl]ethyl}-1,3-benzoxazol-5-yl)benzoate. The reaction mixture was allowed to stand at room temperature. To the second batch of crude methyl 3′-({3-[3-(2,6-dichlorophenyl)-5-(1-methylethyl)-4-isoxazolyl]propanoyl}amino)-4′-hydroxy-3-biphenylcarboxylate (0.068 g) was added propionic acid (0.5 mL). The reaction mixture was heated between 135° C.-150° C. for 2.5 h. The oil bath was removed and the reaction mixture was allowed to stand overnight at room temperature. The two reaction mixtures which contained methyl 3-(2-{2-[3-(2,6-dichlorophenyl)-5-(1-methylethyl)-4-isoxazolyl]ethyl}-1,3-benzoxazol-5-yl)benzoate were combined and partitioned between saturated sodium bicarbonate and ethyl acetate. The organic phase was separated, dried over magnesium sulfate, filtered, and the filtrate was concentrated to give an orange oil. The oil was partially purified by flash chromatography over silica with hexanes:ethyl acetate (2:1) followed by a second column with dichloromethane:methanol (99:1) to give impure methyl 3-(2-{2-[3-(2,6-dichlorophenyl)-5-(1-methylethyl)-4-isoxazolyl]ethyl}-1,3-benzoxazol-5-yl)benzoate. The impure ester (0.026 g) was dissolved in tetrahydrofuran (0.40 mL) and 1 N lithium hydroxide (0.2 mL) was added. The reaction mixture was stirred at room temperature overnight. The reaction mixture was partitioned between water (10 mL), ethyl acetate (30 mL) and saturated sodium hydrogensulfate (0.4 mL). The organic phase was separated, dried over magnesium sulfate, filtered, and the filtrate was concentrated to give crude 3-(2-{2-[3-(2,6-dichlorophenyl)-5-(1-methylethyl)-4-isoxazolyl]ethyl}-1,3-benzoxazol-5-yl)benzoic acid as an oil (0.029 g). An attempt to purify approximately 10% of the crude product by reverse phase preparative HPLC using an acetonitrile:water gradient (50:50 to 100:0) with 0.05% trifluoroacetic acid as a modifier had failed. The remaining 90% of crude 3-(2-{2-[3-(2,6-dichlorophenyl)-5-(1-methylethyl)-4-isoxazolyl]ethyl}-1,3-benzoxazol-5-yl)benzoic acid was applied to a flash silica column and eluted with a dichloromethane:methanol gradient (99:1 to 97:3) to give impure product. The impure product was purified by reverse phase preparative HPLC with an acetonitrile:water gradient (30:70 to 70:30) using 0.1% formic acid as a modifier to give 3.3 mg of 3-(2-{2-[3-(2,6-dichlorophenyl)-5-(1-methylethyl)-4-isoxazolyl]ethyl}-1,3-benzoxazol-5-yl)benzoic acid as an off-white solid. ¹H NMR (400 MHz, CDCl₃): δ 8.33 (m, 1H), 8.08 (d, J=8 Hz, 1H), 7.85 (m, 1H), 7.83 (d, J=8 Hz, 1H), 7.56 (m, 2H), 7.50 (d, J=8 Hz, 1H), 7.42 (m, 2H), 7.34 (m, 1H) 3.27 (septet, J=7 Hz, 1H), 2.88-3.00 (m, 4H), 1.37 (d, J=7 Hz, 6H). AP-LCMS m/z 521 (M+H)⁺.

### Example 40

### 3-{[6-({[3-(2,6-Dichlorophenyl)-5-(1-methylethyl)-4-isoxazolyl]methyl}oxy)-1-naphthalenyl]amino}benzoic acid

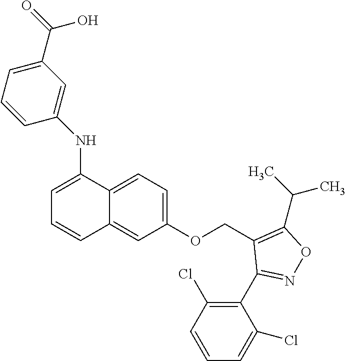

### 40a) 6-(Methyloxy)-1-naphthalenyl trifluoromethanesulfonate

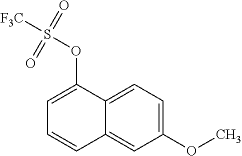

To an ice-water cooled solution of 6-(methyloxy)-1-naphthalenol (0.167 g, 0.96 mmol) in dichloromethane (5 mL) was slowly added pyridine (0.47 mL, 5.8 mmol). The solution was allowed to stir for several minutes before trifluoromethanesulfonic anhydride (0.2 mL, 1.2 mmol) was slowly added with stirring under a nitrogen atmosphere. The reaction mixture was stirred in the ice-water bath for 2.5 h. The reaction mixture was partitioned between diethyl ether and 1 N hydrochloric acid. The organic phase was separated, washed with saturated sodium chloride, dried over magnesium sulfate, filtered, and the filtrate was concentrated to give a dark brown liquid. The crude product was purified by flash chromatography over silica with a hexanes:ethyl acetate gradient (100:0 to 90:10) to give 0.238 g (81%) of 6-(methyloxy)-1-naphthalenyl trifluoromethanesulfonate as a colorless liquid. ¹H NMR (400 MHz, CDCl₃): δ 7.97 (d, J=9 Hz, 1H), 7.74 (d, J=8 Hz, 1H), 7.43 (t, J=8 Hz, 1H), 7.29 (m, 2H), 7.18 (d, J=2 Hz, 1H), 3.94 (s, 3H). ES-LCMS m/z 305 (M−H)⁻.

### 40b) Ethyl 3-{[6-(methyloxy)-1-naphthalenyl]amino}benzoate

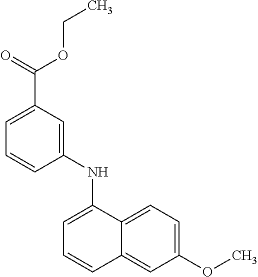

6-(Methyloxy)-1-naphthalenyl trifluoromethanesulfonate (0.050 g, 0.16 mmol), tris(dibenzylideneacetone)dipalladium(0) (0.0067 g, 0.007 mmol), rac-2,2′-bis(diphenylphosphino)-1,1′-binaphthyl (0.006 g, 0.0096 mmol), cesium carbonate (0.088 g, 0.27 mmol), ethyl-3-aminobenzoate (0.035 mL, 0.24 mmol), and toluene (2 mL) were combined and the stirred reaction mixture was heated at reflux for 21 h under a nitrogen atmosphere. The oil bath was removed and the reaction mixture was allowed to stand at room temperature. This reaction was repeated wherein 6-(methyloxy)-1-naphthalenyl trifluoromethanesulfonate (0.168 g, 0.55 mmol), tris(dibenzylideneacetone)dipalladium(0) (0.022 g, 0.024 mmol), rac-2,2′-bis(diphenylphosphino)-1,1′-binaphthyl (0.022 g, 0.035 mmol), cesium carbonate (0.294 g, 0.90 mmol), ethyl-3-aminobenzoate (0.12 mL, 0.80 mmol), and toluene (7 mL) were combined and heated at reflux for 20 h. The two reaction mixtures were combined and partitioned between ethyl acetate and 1 N hydrochloric acid (25 mL). The organic phase was separated, washed with saturated sodium chloride, dried over magnesium sulfate, filtered, and the filtrate was concentrated to give the crude product. The crude product was purified by flash chromatography over silica with a hexanes:ethyl acetate gradient (100:0 to 80:20) to give 0.16 g (70% for the two reactions) of ethyl 3-{[6-(methyloxy)-1-naphthalenyl]amino}benzoate as a yellow oil. ¹H NMR (400 MHz, CDCl₃): δ 7.90 (d, J=9 Hz, 1H), 7.64 (d, J=2 Hz, 1H), 7.54 (d, J=8 Hz, 1H), 7.51 (d, J=8 Hz, 1H), 7.37 (t, J=8 Hz, 1H), 7.20-7.28 (m, 2H), 7.17 (d, J=3 Hz, 1H), 7.13 (dd, J=9, 3 Hz, 1H), 7.08 (dd, J=8, 2 Hz, 1H), 4.35 (q, J=7 Hz, 2H), 3.93 (s, 3H), 1.36 (t, J=7 Hz, 3H). ES-LCMS m/z 322 (M+H)⁺.

### 40c) Ethyl 3-[(6-hydroxy-1-naphthalenyl)amino]benzoate

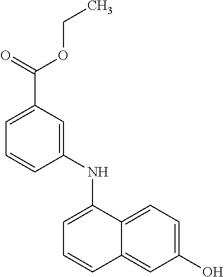

To a stirred ice-water cooled solution of ethyl 3-{[6-(methyloxy)-1-naphthalenyl]amino}benzoate (0.16 g, 0.50 mmol) in dichloromethane (10 mL) was slowly added, dropwise, boron tribromide (1 M in dichloromethane) (2 mL, 2 mmol) under a nitrogen atmosphere. After 3.5 h, boron tribromide (1 M in dichloromethane) (0.76 mL 0.76 mmol) was slowly added to the reaction mixture. The reaction mixture was stirred for 1 h with cooling. The ice-water bath was removed, and the reaction mixture was allowed to stir at room temperature for approximately 1 h. The reaction mixture was poured onto ice and the mixture was partitioned between water and dichloromethane. The organic phase was separated and the aqueous phase was extracted with ethyl acetate. The organic extracts were combined, dried over magnesium sulfate, filtered, and the filtrate was concentrated to give a dark orange oil. The crude product was purified by flash chromatography over silica with hexanes to give ethyl 3-[(6-hydroxy-1-naphthalenyl)amino]benzoate as a cloudy yellow oil. [Note: The product eluted from the column very quickly, possibly because the crude product was applied to a silica pre-column as a solution in ethyl acetate, dichloromethane, and methanol.] The product was dissolved in ethyl acetate and the solution was dried over magnesium sulfate, filtered, and the filtrate was concentrated to give 0.053 g of ethyl 3-[(6-hydroxy-1-naphthalenyl)amino]benzoate as a cloudy yellow oil. ¹H NMR indicates that an impurity is present. The material was used without further purification. ¹H NMR (400 MHz, CDCl₃): δ 7.92 (d, J=9 Hz, 1H), 7.63 (m, 1H), 7.55 (d, J=8 Hz, 1H), 7.45 (d, J=8 Hz, 1H), 7.36 (t, J=8 Hz, 1H), 7.27 (m, 1H), 7.21 (d, J=7 Hz, 1H), 7.18 (d, J=3 Hz, 1H), 7.08 (dd, J=9, 3 Hz, 2H), 4.35 (q, J=7 Hz, 2H), 1.36 (t, J=7 Hz, 3H). ES-LCMS m/z 306 (M−H)⁻; 308 (M+H)⁺.

### 40d) Ethyl 3-{[6-({[3-(2,6-dichlorophenyl)-5-(1-methylethyl)-4-isoxazolyl]methyl}oxy)-1-naphthalenyl]amino}benzoate

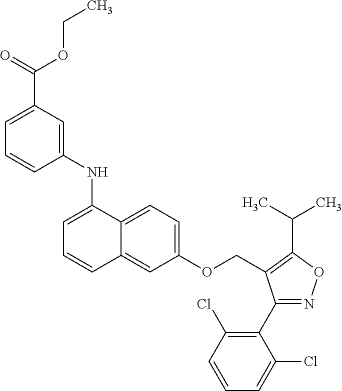

To an stirred ice-water cooled solution of ethyl 3-[(6-hydroxy-1-naphthalenyl)amino]benzoate (0.053 g, 0.17 mmol), [3-(2,6-dichlorophenyl)-5-(1-methylethyl)-4-isoxazolyl]methanol (prepared according to the general procedure described in Maloney, P. R., et al., 2000 J. Med. Chem. 43:2971-2974) (0.058 g, 0.20 mmol), and triphenylphosphine (0.053 g, 0.20 mmol) in dichloromethane (6 mL) was added, dropwise, a solution of diisopropyl azodicarboxylate (0.04 mL, 0.20 mmol) in dichloromethane (0.05 mL). The syringe and glass vial which contained the diisopropyl azodicarboxylate were rinsed with dichloromethane (0.1 mL) and the solution was added to the reaction mixture. After 12 min, the ice-water bath was removed and the yellow solution was allowed to stir at room temperature overnight under a nitrogen atmosphere. The reaction mixture was concentrated and the crude product was purified by flash chromatography over silica with a hexanes:ethyl acetate gradient (100:0 to 80:20) to give 0.037 g (37%) of ethyl 3-{[6-({[3-(2,6-dichlorophenyl)-5-(1-methylethyl)-4-isoxazolyl]methyl}oxy)-1-naphthalenyl]amino}benzoate as a yellow oil. ¹H NMR indicates that an impurity is present. The material was used without further purification. ¹H NMR (400 MHz, CDCl₃): δ 7.84 (d, J=9 Hz, 1H), 7.61 (m, 1H), 7.54 (d, J=8 Hz, 1H), 7.20-7.43 (m, 7H), 7.05 (m, 2H), 6.96 (dd, J=9, 3 Hz, 1H), 4.85 (s, 2H), 4.34 (q, J=7 Hz, 2H), 3.37 (septet, J=7 Hz, 1H), 1.44 (d, J=7 Hz, 6H), 1.36 (t, J=7 Hz, 3H). ES-LCMS m/z 575 (M+H)⁺.

### 40e) 3-{[6-({[3-(2,6-Dichlorophenyl)-5-(1-methylethyl)-4-isoxazolyl]methyl}oxy)-1-naphthalenyl]amino}benzoic acid

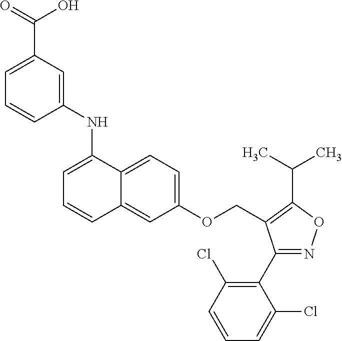

To a stirred solution of ethyl 3-{[6-({[3-(2,6-dichlorophenyl)-5-(1-methylethyl)-4-isoxazolyl]methyl}oxy)-1-naphthalenyl]amino}benzoate (0.037 g, 0.064 mmol) in tetrahydrofuran (0.80 mL) was added a solution of lithium hydroxide (1 N) (0.25 mL, 0.25 mmol). The reaction mixture was stirred at room temperature for 18 h. The reaction mixture was then heated at 60° C. for 3 h. To the reaction mixture was added tetrahydrofuran (0.5 mL) and heating was continued for another 4 h. The reaction mixture was allowed to stand at room temperature overnight. To the reaction mixture was added tetrahydrofuran (0.5 mL). The reaction mixture was heated at 60° C. for 8 h. The reaction mixture was allowed to stand at room temperature for approximately 1.5 days. The reaction mixture was then heated at 60° C. ES-LCMS analysis of the reaction mixture indicated that the reaction was approximately 85% complete after 15 h of heating at 60° C. To the reaction mixture was added tetrahydrofuran (0.2 mL) and lithium hydroxide (1 N) (0.05 mL, 0.05 mmol). The reaction mixture was heated at 60° C. for 7 h and allowed to stand at room temperature overnight. To the reaction mixture was added a solution of lithium hydroxide (1 N) (0.1 mL) and the reaction mixture was heated at reflux for 10 h. The reaction mixture was allowed to stand at room temperature overnight. The reaction mixture was partitioned between ethyl acetate (20 mL), water (5 mL), and saturated sodium hydrogensulfate (0.2 mL). The organic phase was separated, washed with water (3 mL), followed by saturated sodium chloride (4 mL), dried over magnesium sulfate, filtered, and the filtrate concentrated to give the crude product as an oil. The crude product was purified by flash chromatography over silica with hexanes:ethyl acetate (1:1), followed by hexanes:ethyl acetate (1:2) and finally ethyl acetate to give 0.013 g of 3-{[6-({[3-(2,6-dichlorophenyl)-5-(1-methylethyl)-4-isoxazolyl]methyl}oxy)-1-naphthalenyl]amino}benzoic acid. Additional product was eluted from the column with ethyl acetate:methanol (95:5). The two batches of product were combined and dried under high vacuum at 75° C. to give 0.019 g (54%) of 3-{[6-({[3-(2,6-dichlorophenyl)-5-(1-methylethyl)-4-isoxazolyl]methyl}oxy)-1-naphthalenyl]amino}benzoic acid as a tan amorphous solid. [Note: Additional product was eluted from the column with ethyl acetate:methanol (9:1), followed by methanol. The fractions which contained product were combined and concentrated. The residue was dissolved in dichloromethane and the solution was filtered. The filtrate was concentrated to give another 0.016 g of 3-{[6-({[3-(2,6-dichlorophenyl)-5-(1-methylethyl)-4-isoxazolyl]methyl}oxy)-1-naphthalenyl]amino}benzoic acid (as determined by TLC only).] ¹H NMR (400 MHz, CDCl₃): δ 7.83 (d, J=9 Hz, 1H), 7.60 (m, 1H), 7.57 (d, J=8 Hz, 1H), 7.44 (d, J=8 Hz, 1H), 7.34-7.40 (m 3H), 7.21-7.31 (m, 3H), 7.09 (dd, J=8, 2 Hz, 1H), 7.06 (d, J=3 Hz, 1H), 6.97 (dd, J=3, 9 Hz, 1H), 4.86 (s, 2H), 3.37 (septet, J=7 Hz, 1H), 1.43 (d, J=7 Hz, 6H). HRMS C₃₀H₂₅Cl₂N₂O₄ m/z 547.1191 (M+H)⁺^{Cal}; 547.1182 (M+H)⁺_{Obs}.

### Example 41

### 3-[(2-{2-[3-(2,6-Dichlorophenyl)-5-(1-methylethyl)-4-isoxazolyl]ethyl}-1,3-benzoxazol-7-yl)amino]benzoic acid

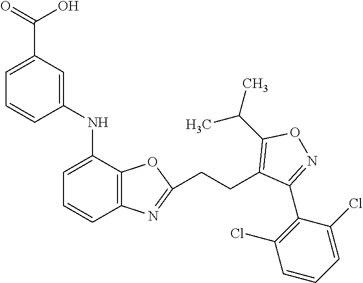

### 41a) 1-Bromo-3-nitro-2-[(phenylmethyl)oxy]benzene

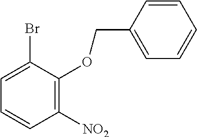

2-Bromo-6-nitrophenol (1.59 g, 7.29 mmol), benzyl bromide (0.87 mL, 7.32 mmol), potassium carbonate (2.5 g, 18.1 mmol), and acetonitrile (15 mL) were combined in a round bottom flask and the mixture was heated at 70° C. with stirring under a nitrogen atmosphere for 3 h. The reaction mixture was allowed to stand at room temperature overnight. The reaction mixture was partitioned between ethyl acetate and water. The organic phase was separated, washed with saturated sodium chloride, dried over magnesium sulfate, filtered, and the filtrate was concentrated to give 2.12 g (94%) of 1-bromo-3-nitro-2-[(phenylmethyl)oxy]benzene as a yellow liquid which solidified upon standing to a yellow solid. ¹H NMR (400 MHz, CDCl₃): δ 7.83 (dd, J=8, 2 Hz, 1H), 7.78 (dd, J=8, 2 Hz, 1H), 7.55 (m, 2H), 7.35-7.43 (m, 3H), 7.15 (t, J=8 Hz, 1H), 5.19 (s, 2H).

### 41b) Ethyl 3-({3-nitro-2-[(phenylmethyl)oxy]phenyl}amino)benzoate

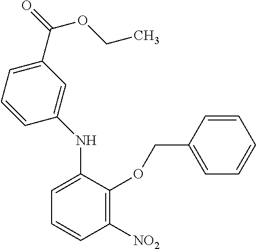

1-Bromo-3-nitro-2-[(phenylmethyl)oxy]benzene (0.81 g, 2.63 mmol), tris(dibenzylideneacetone)dipalladium(0) (0.098 g, 0.107 mmol), rac-2,2′-bis(diphenylphosphino)-1,1′-binaphthyl (0.096 g, 0.154 mmol), cesium carbonate (1.33 g, 4.08 mmol), ethyl-3-aminobenzoate (0.59 mL, 3.95 mmol), and toluene (35 mL) were combined and the stirred reaction mixture was heated at 100° C. for 6 h.

The reaction mixture was allowed to stand at room temperature overnight. The reaction mixture was heated at 100° C. for 22.5 h. The reaction mixture was allowed to stand at room temperature. The reaction mixture was partitioned between ethyl acetate and water. The organic phase was separated, dried over magnesium sulfate, filtered, and the filtrate was concentrated to give a dark brown liquid. The crude product was purified by flash chromatography over silica with a hexanes:ethyl acetate gradient (100:0 to 80:20) to give 0.61 g of ethyl 3-({3-nitro-2-[(phenylmethyl)oxy]phenyl}amino)benzoate as a viscous orange oil. ¹H NMR indicates the product is ˜85 mol % pure. The product was used directly without further purification. ¹H NMR (400 MHz, DMSO-d₆): δ 8.37 (s, 1H), 7.61 (s, 1H), 7.46 (m, 2H), 7.38 (m, 2H), 7.19-7.28 (m, 7H), 4.94 (s, 2H), 4.27 (q, J=7 Hz, 2H), 1.28 (t, J=7 Hz, 3H). AP-LCMS m/z 415 (M+Na)⁺.

### 41c) Ethyl 3-[(3-amino-2-hydroxyphenyl)amino]benzoate

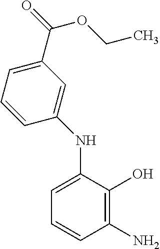

To a solution of ethyl 3-({3-nitro-2-[(phenylmethyl)oxy]phenyl}amino)benzoate (0.61 g) in ethanol (10 mL) in a round bottom flask, was added 10% palladium on carbon (Degussa Type; ˜50% water by weight) (0.068 g). The round bottom flask was evacuated and filled with nitrogen several times. The flask was evacuated and filled with hydrogen using a balloon. The reaction mixture was stirred under a hydrogen atmosphere overnight at room temperature. After 28 h, the reaction mixture was filtered through a pad of Celite® and the pad was washed with ethanol. The filtrate was filtered through a second pad of Celite® and the pad was washed with ethanol. The filtrate was concentrated (water bath temperature ˜40° C.) to give 0.32 g of crude ethyl 3-[(3-amino-2-hydroxyphenyl)amino]benzoate as a dark brown oil. ¹H NMR and AP-LCMS indicated the product contained ethanol and one or more impurities. The product was used directly without further purification. ¹H NMR (400 MHz, DMSO-d₆): δ 7.39 (m, 2H), 7.21 (m, 2H), 7.00 (m, 1H), 6.53 (t, J=8 Hz, 1H), 6.36 (d, J=8 Hz, 2H), 4.23 (q, J=7 Hz, 2H), 1.25 (t, J=7 Hz, 3H). AP-LCMS m/z 273 (M+H)⁺.

### 41d) Ethyl 3-{[3-({3-[3-(2,6-dichlorophenyl)-5-(1-methylethyl)-4-isoxazolyl]propanoyl}amino)-2-hydroxyphenyl]amino}benzoate

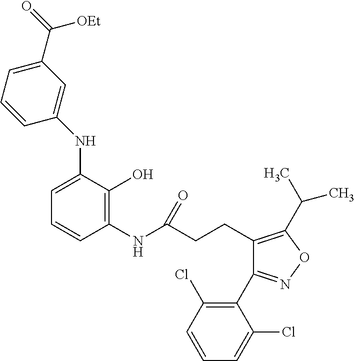

Ethyl 3-[(3-amino-2-hydroxyphenyl)amino]benzoate (0.16 g) (Impure) 1,3-dicyclohexylcarbodiimide (0.114 g, 0.55 mmol), 1-hydroxybenzotriazole hydrate (0.070 g, 0.52 mmol), 3-[3-(2,6-dichlorophenyl)-5-(1-methylethyl)-4-isoxazolyl]propanoic acid (from Example 39(b)) (0.133 g, 0.405 mmol), and acetonitrile (14 mL) were combined in a round bottom flask and the reaction mixture was stirred for 20 h at room temperature under a nitrogen atmosphere. The reaction mixture was concentrated and the crude product was partitioned between water and ethyl acetate. The organic phase was separated and washed with saturated sodium chloride. The water and saturated sodium chloride washes were combined and extracted with ethyl acetate. The organic extracts were combined, dried over magnesium sulfate, filtered, and the filtrate was concentrated to give a dark brown oil. The crude product was partially purified by flash chromatography over silica with a hexanes:ethyl acetate gradient (100:0 to 60:40) to give 0.090 g of ethyl 3-{[3-({3-[3-(2,6-dichlorophenyl)-5-(1-methylethyl)-4-isoxazolyl]propanoyl}amino)-2-hydroxyphenyl]amino}benzoate as a brown-orange oil. ¹H NMR and ES-LCMS indicated the product contained solvent and an impurity. The compound was used directly without further purification. ¹H NMR (400 MHz, CDCl₃): δ 8.92 (s, 1H), 7.76 (s, 1H), 7.59 (d, J=8 Hz, 1H), 7.27-7.48 (m, 6H), 7.11 (d, J=7 Hz, 1H), 6.75 (t, J=8 Hz, 1H), 6.42 (d, J=7 Hz, 1H), 4.35 (q, J=7 Hz, 2H), 3.24 (septet, J=7 Hz, 1H), 2.75 (t, J=7 Hz, 2H), 2.38 (t, J=7 Hz, 2H), 1.37 (t, J=7 Hz, 3H), 1.31 (d, J=7 Hz, 6H).

### 41e) Ethyl 3-[(2-{2-[3-(2,6-dichlorophenyl)-5-(1-methylethyl)-4-isoxazolyl]ethyl}-1,3-benzoxazol-7-yl)amino]benzoate

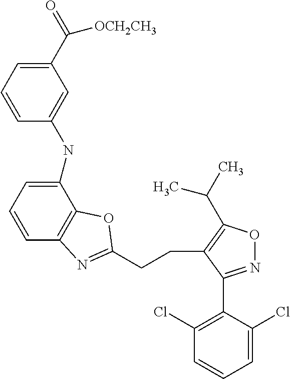

Ethyl 3-{[3-({3-[3-(2,6-dichlorophenyl)-5-(1-methylethyl)-4-isoxazolyl]propanoyl}amino)-2-hydroxyphenyl]amino}benzoate (0.0035 g) and propionic acid (0.10 mL) were combined and the stirred reaction mixture was heated for 1.5 h between 130° C.-150° C. under a nitrogen atmosphere. ES-LCMS indicated that the desired product was formed. To the reaction mixture was added ethyl 3-{[3-({3-[3-(2,6-dichlorophenyl)-5-(1-methylethyl)-4-isoxazolyl]propanoyl}amino)-2-hydroxyphenyl]amino}benzoate (0.086 g) and propionic acid (1 mL). The reaction mixture was heated at 135° C. under a nitrogen atmosphere for 2.5 h. The reaction mixture was allowed to cool at room temperature and carefully partitioned between saturated sodium bicarbonate and ethyl acetate. (Caution, significant carbon dioxide is released!) The organic phase was separated, washed with water, dried over magnesium sulfate, filtered, and the filtrate was concentrated to give a dark brown-orange oil. The crude product was purified by flash chromatography over silica with a hexanes:ethyl acetate gradient (100:0 to 80:20) to give 0.055 g (7.4% from 1-bromo-3-nitro-2-[(phenylmethyl)oxy]benzene) of ethyl 3-[(2-{2-[3-(2,6-dichlorophenyl)-5-(1-methylethyl)-4-isoxazolyl]ethyl}-1,3-benzoxazol-7-yl)amino]benzoate as an amorphous solid. ¹H NMR (400 MHz, CDCl₃): δ 7.74 (m, 1H), 7.65 (d, J=8 Hz, 1H), 7.39 (m, 2H), 7.34 (d, J=8 Hz, 1H), 7.31-7.23 (m, 3H), 7.19 (t, J=8 Hz, 1H), 7.14 (dd, J=8, 1 Hz, 1H), 5.92 (br s, 1H), 4.37 (q, J=7 Hz, 2H), 3.25 (septet, J=7 Hz, 1H), 2.85-2.94 (m, 4H), 1.38 (t, J=7 Hz, 3H), 1.36 (d, J=7 Hz, 6H).

### 41f) 3-[(2-{2-[3-(2,6-Dichlorophenyl)-5-(1-methylethyl)-4-isoxazolyl]ethyl}-1,3-benzoxazol-7-yl)-amino]benzoic acid

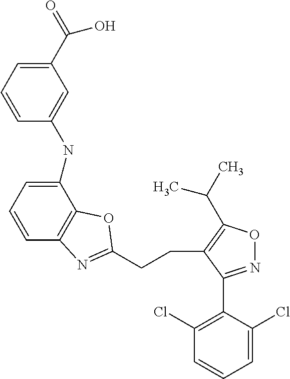

To a stirred solution of ethyl 3-[(2-{2-[3-(2,6-dichlorophenyl)-5-(1-methylethyl)-4-isoxazolyl]ethyl}-1,3-benzoxazol-7-yl)amino]benzoate (0.055 g, 0.097 mmol) in tetrahydrofuran (0.40 mL) was added lithium hydroxide (1 N) (0.40 mL, 0.40 mmol) at room temperature. The reaction mixture was stirred for 1 h. To the reaction mixture was added tetrahydrofuran (0.40 mL). The reaction mixture was stirred at room temperature overnight. ES-LCMS analysis of the reaction mixture indicated that the reaction was approximately 15% complete. An aliquot of the reaction mixture (0.05 mL) was transferred to a glass pressure tube and the aliquot was diluted with tetrahydrofuran (0.05 mL). The diluted aliquot was heated at 60° C. in a sealed pressure tube for 5 h. ES-LCMS analysis of the heated aliquot indicated that the reaction was approximately 70% complete. The diluted aliquot was combined with the original reaction mixture and tetrahydrofuran (1 mL) was added. The stirred reaction mixture was heated overnight at 60° C. under a nitrogen atmosphere. The reaction mixture was partitioned between water (5 mL), ethyl acetate (20 mL), and saturated sodium hydrogensulfate (0.2 mL). The organic phase was separated, washed with water (3 mL), followed by saturated sodium chloride (3 mL), dried over magnesium sulfate, filtered, and the filtrate was concentrated to give an oil. The crude product was purified by flash chromatography over silica with a dichloromethane:methanol gradient (100:0 to 98:2) to give 0.023 g of 3-[(2-{2-[3-(2,6-dichlorophenyl)-5-(1-methylethyl)-4-isoxazolyl]ethyl}-1,3-benzoxazol-7-yl)amino]benzoic acid as an off-white amorphous solid and 0.0067 g of a second crop of 3-[(2-{2-[3-(2,6-dichlorophenyl)-5-(1-methylethyl)-4-isoxazolyl]ethyl}-1,3-benzoxazol-7-yl)amino]benzoic acid as an white amorphous solid for a total yield of 0.0297 g (57%). ¹H NMR (400 MHz, DMSO-d₆): δ 12.76 (br s, 1H), 8.56 (s, 1H), 7.61 (d, J=2 Hz, 1H), 7.59 (s, 1H), 7.54 (d, J=8 Hz, 1H), 7.52 (m, 1H), 7.40 (d, J=8 Hz, 1H), 7.29 (t, J=8 Hz, 1H), 7.20 (m, 2H), 7.12 (dd, J=8, 2 Hz, 1H), 7.08 (dd, J=6, 3 Hz, 1H), 3.30 (m, 1H), 2.82 (t, J=7 Hz, 2H), 2.75 (t, J=7 Hz, 2H), 1.20 (d, J=7 Hz, 6H). HRMS C₂₈H₂₄Cl₂N₃O₄ m/z 536.1144 (M+H)⁺_{Cal}; 536.1146 (M+H)⁺_{Obs}.

### Example 42

### 3-{[5-({[3-(2,6-Dichlorophenyl)-5-(1-methylethyl)-4-isoxazolyl]methyl}oxy)-1H-benzimidazol-1-yl]methyl}benzoic acid

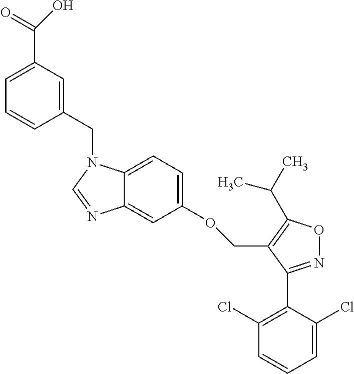

### 42a) Methyl 3-({[4-(methyloxy)-2-nitrophenyl]amino}methyl)benzoate

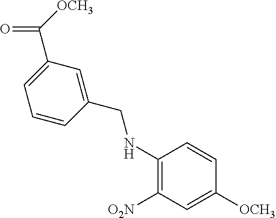

To a stirred suspension of potassium carbonate (2.4 g, 17.4 mmol) and 4-methoxy-2-nitroaniline (2.33 g, 13.9 mmol) in N,N-dimethylformamide (50 mL) at 110° C. was slowly added a solution of methyl-(3-bromomethyl)benzoate (3.85 g, 16.8 mmol) in N,N-dimethylformamide (20 mL) under a nitrogen atmosphere. The reaction mixture was heated at 110° C. for 4 h then allowed to cool at room temperature. The reaction mixture was partitioned between water and dichloromethane. The organic phase was separated, washed with water, followed by saturated sodium chloride, dried over magnesium sulfate, filtered, and the filtrate was concentrated to give crude methyl 3-({[4-(methyloxy)-2-nitrophenyl]amino}methyl)benzoate as a red-orange liquid. In an attempt to reduce the nitro group, the crude methyl 3-({[4-(methyloxy)-2-nitrophenyl]amino}methyl)benzoate was dissolved in ethanol (75 mL) and the solution was added to 10% palladium on carbon (Degussa Type; ˜50% water by weight) (1.3 g). The flask was evacuated, then filled with nitrogen (3 times). The flask was evacuated and filled with hydrogen using a balloon. The reaction mixture was stirred at room temperature for 2.5 h. ES-LCMS analysis of the reaction mixture indicated that significant debenzylation occurred to give 4-(methyloxy)-2-nitroaniline. The reaction mixture was filtered through a pad of Celite®. The pad of Celite® was washed with ethanol, followed by water, and finally ethyl acetate. The ethyl acetate filtrate was washed with water, dried over magnesium sulfate, filtered, and the filtrate was concentrated to give 0.91 g (21%) of methyl 3-({[4-(methyloxy)-2-nitrophenyl]amino}methyl)benzoate as a red-orange solid. ¹H NMR (400 MHz, CDCl₃): δ 8.37 (m, 1H), 8.01 (s, 1H), 7.97 (d, J=8 Hz, 1H), 7.65 (d, J=3 Hz, 1H), 7.53 (d, J=8 Hz, 1H), 7.43 (t, J=8 Hz, 1H), 7.07 (dd, J=9, 3 Hz, 1H), 6.72 (d, J=9 Hz, 1H), 4.59 (d, J=6 Hz, 2H), 3.91 (s, 3H), 3.78 (s, 3H). ES-LCMS m/z 317 (M+H)⁺.

### 42b) Methyl 3-({[2-amino-4-(methyloxy)phenyl]amino}methyl)benzoate

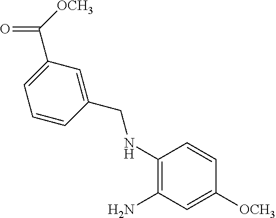

Methyl 3-({[4-(methyloxy)-2-nitrophenyl]amino}methyl)benzoate (0.79 g, 2.5 mmol), tin(II) chloride dihydrate (2.6 g, 11.5 mmol), and ethanol (30 mL) were combined and the stirred reaction mixture was heated at reflux under a nitrogen atmosphere for 3.5 h. The reaction mixture was allowed to cool at room temperature and cooled in an ice-water bath. To the cold reaction mixture was slowly added saturated sodium bicarbonate (60 mL) with stirring. The reaction mixture was transferred to a separatory funnel and extracted with ethyl acetate. The organic phase was separated and washed with saturated sodium chloride. The aqueous phase was combined with the saturated sodium chloride wash and the mixture was extracted with ethyl acetate. The organic extracts were combined, dried over magnesium sulfate, filtered, and the filtrate was concentrated to give 0.659 g (92%) of methyl 3-({[2-amino-4-(methyloxy)phenyl]amino}methyl)benzoate as a viscous orange oil. ¹H NMR (400 MHz, CDCl₃): δ 8.06 (s, 1H), 7.94 (d, J=8 Hz, 1H), 7.59 (d, J=8 Hz, 1H), 7.40 (t, J=8 Hz, 1H), 6.60 (d, J=9 Hz, 1H), 6.36 (d, J=3 Hz, 1H), 6.28 (dd, J=9, 3 Hz, 1H), 4.29 (s, 2H), 3.91 (s, 3H), 3.72 (s, 3H). ES-LCMS m/z 287 (M+H)⁺.

### 42c) Methyl 3-{[5-(methyloxy)-1H-benzimidazol-1-yl]methyl}benzoate

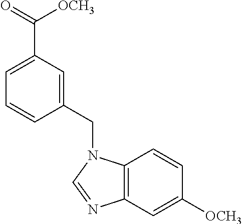

Methyl 3-({[2-amino-4-(methyloxy)phenyl]amino}methyl)benzoate (0.64 g, 2.24 mmol) was dissolved in formic acid (96%) (12 mL) and the solution was stirred overnight at room temperature under a nitrogen atmosphere. The formic acid was removed in vacuo at room temperature. The crude product was partitioned between ethyl acetate and saturated sodium bicarbonate (Caution, gas evolution!). The organic phase was separated, dried over magnesium sulfate, filtered, and the filtrate was concentrated to give 0.572 g (86%) of methyl 3-{[5-(methyloxy)-1H-benzimidazol-1-yl]methyl}benzoate as a red-orange oil. ¹H NMR (400 MHz, CDCl₃): δ 7.99 (d, J=8 Hz, 1H), 7.93 (s, 2H), 7.40 (t, J=8 Hz, 1H), 7.30 (m 2H), 7.10 (d, J=9 Hz, 1H), 7.89 (dd, J=9, 2 Hz, 1H), 5.37 (s, 2H), 3.90 (s, 3H), 3.86 (s, 3H). ES-LCMS m/z 297 (M+H)⁺.

### 42d) Methyl 3-[(5-hydroxy-1H-benzimidazol-1-yl)methyl]benzoate

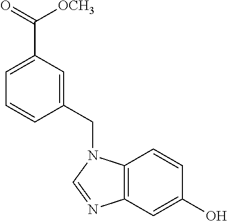

To an ice-water cooled solution of methyl 3-{[5-(methyloxy)-1H-benzimidazol-1-yl]methyl}benzoate (0.618 g, 2.1 mmol, from multiple batches) in dichloromethane (20 mL) was slowly added, dropwise, boron tribromide (1 M in dichloromethane) (8.4 mL, 8.4 mmol) with stirring under a nitrogen atmosphere. The reaction mixture was stirred with cooling for 1.25 h. The reaction mixture was poured into ice-water and the round bottom flask was rinsed with dichloromethane. The dichloromethane rinse was poured into the ice-water. A dark purple solid remained in the round bottom flask in spite of numerous rinses with dichloromethane. ES-LCMS analysis of the purple solid indicated that it was a mixture of product and 3-[(5-hydroxy-1H-benzimidazol-1-yl)methyl]benzoic acid. To the dark purple solid was added methanol (25 mL) and sulfuric acid (5 drops). The stirred solution was heated overnight at reflux under a nitrogen atmosphere. The reaction mixture was concentrated and the dark purple residue was partitioned between saturated sodium bicarbonate and ethyl acetate. The organic phase was separated, washed with saturated sodium chloride, dried over magnesium sulfate, filtered, and the filtrate was concentrated to give the crude product. The crude product was purified by flash chromatography over silica with dichloromethane, followed by dichloromethane:methanol (96:4) to give 0.25 g (42%) of methyl 3-[(5-hydroxy-1H-benzimidazol-1-yl)methyl]benzoate as a pale yellow solid. ¹H NMR (400 MHz, DMSO-d₆): δ 9.01 (s, 1H), 8.26 (s, 1H), 7.84 (m, 2H), 7.53 (d, J=8 Hz, 1H), 7.47 (t, J=8 Hz, 1H), 7.21 (d, J=9 Hz, 1H), 6.93 (d, J=2 Hz, 1H), 6.66 (dd, J=9, 2 Hz, 1H), 5.48 (s, 2H), 3.80 (s, 3H). ES-LCMS m/z 283 (M+H)⁺.

### 42e) 3-{[5-({[3-(2,6-Dichlorophenyl)-5-(1-methylethyl)-4-isoxazolyl]methyl}oxy)-1H-benzimidazol-1-yl]methyl}benzoic acid

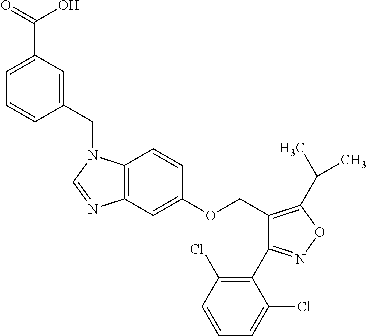

To a stirred ice-water cooled mixture of methyl 3-[(5-hydroxy-1H-benzimidazol-1-yl)methyl]benzoate (0.13 g, 0.46 mmol), triphenylhosphine (0.134 g, 0.51 mmol) and [3-(2,6-dichlorophenyl)-5-(1-methylethyl)-4-isoxazolyl]methanol (prepared by the general procedure described in Maloney, P. R., et al., 2000 J. Med. Chem. 43:2971-2974) (0.145 g, 0.51 mmol) in dichloromethane (10 mL) was slowly added, dropwise, a solution of diisopropyl azodicarboxylate (0.10 mL, 0.51 mmol) in dichloromethane (0.2 mL) under a nitrogen atmosphere. The reaction mixture was stirred in the ice-water bath for 5 min and the ice-water bath was removed. The reaction mixture was stirred overnight at room temperature. The reaction mixture was adsorbed onto silica and the product was partially purified by flash chromatography over silica with a dichloromethane:methanolic ammonia (2 M) gradient (100:0 to 97.5:2.5) to give 0.28 g of a mixture of methyl 3-{[5-({[3-(2,6-dichlorophenyl)-5-(1-methylethyl)-4-isoxazolyl]methyl}oxy)-1H-benzimidazol-1-yl]methyl}benzoate and triphenylphosphine oxide. To a stirred solution of impure methyl 3-{[5-({[3-(2,6-dichlorophenyl)-5-(1-methylethyl)-4-isoxazolyl]methyl}oxy)-1H-benzimidazol-1-yl]methyl}benzoate (0.28 g) in 1,4-dioxane (2.5 mL) in a round bottom flask was added, dropwise, lithium hydroxide (1 N) (0.75 mL, 0.75 mmol) at room temperature. The reaction mixture was stirred overnight at room temperature and concentrated. Water (5 mL) was added to the residue and the aqueous solution was washed twice with diethyl ether. The reaction flask was rinsed with water (1 mL) and the solution was added to the washed aqueous phase. The aqueous solution was washed with diethyl ether. To the washed aqueous solution was added saturated sodium hydrogensulfate (0.2 mL), followed by ethyl acetate (20 mL). The mixture was transferred to a reparatory funnel and the organic phase was separated. The organic phase was washed with water (3 mL), followed by saturated sodium chloride (3 mL). A solid precipitated from the organic phase. The organic phase was filtered to give a solid which was washed with water and dried to give 0.027 g of 3-{[5-({[3-(2,6-dichlorophenyl)-5-(1-methylethyl)-4-isoxazolyl]methyl}oxy)-1H-benzimidazol-1-yl]methyl}benzoic acid as an off-white solid. The organic filtrate was dried over magnesium sulfate, filtered, concentrated, and dried to give 0.035 g of 3-{[5-({[3-(2,6-dichlorophenyl)-5-(1-methylethyl)-4-isoxazolyl]methyl}oxy)-1H-benzimidazol-1-yl]methyl}benzoic acid as an off-white solid. The total yield was 0.062 g (25%).

[Note: Approximately 0.006 g of additional product was recovered when the flask was rinsed with dichloromethane and methanol after the solid was transferred to a vial.] ¹H NMR (400 MHz, DMSO-d₆): δ 12.97 (br s, 1H), 8.32 (s, 1H), 7.81 (d, J=7 Hz, 1H), 7.78 (s, 1H), 7.56 (m, 2H), 7.48 (m, 2H), 7.43 (t, J=8 Hz, 1H), 7.27 (d, J=9 Hz, 1H), 7.09 (d, J=2 Hz, 1H), 6.58 (dd, J=9, 2 Hz, 1H), 5.48 (s, 2H), 4.78 (s, 2H), 3.38 (septet, J=7 Hz, 1H), 1.26 (d, J=7 Hz, 6H). ES-LCMS m/z 534 (M−H)⁻.

### Example 43

### 3-{[5-({[3-(2,6-Dichlorophenyl)-5-(1-methylethyl)-4-isoxazolyl]methyl}oxy)-1H-indol-1-yl]sulfonyl}benzoic acid

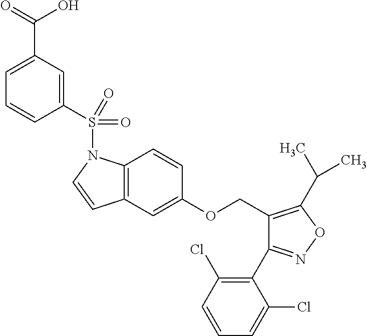

### 43a) Methyl 3-(chlorosulfonyl)benzoate

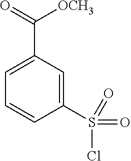

3-(Chlorosulfonyl)benzoic acid (2.13 g, 9.65 mmol), thionyl chloride (8 mL, 110 mmol) and dichloroethane (8 mL) were combined in a round bottom flask and the reaction mixture was heated at reflux for 1 h under a nitrogen atmosphere. The oil bath was removed and the reaction mixture was allowed to cool at room temperature. The reaction mixture was concentrated, and the resulting brown-orange liquid was diluted with toluene. The toluene was removed in vacuo to give crude 3-(chlorosulfonyl)benzoyl chloride as a brown-orange liquid. The crude 3-(chlorosulfonyl)benzoyl chloride was cooled in ice-water bath and cold methanol (16 mL) was added. The cold reaction mixture was stirred for 10 min. The ice-water bath was removed and the reaction mixture was stirred at room temperature for 15 min. To the reaction mixture was added ice-cold water (16 mL). The resulting suspension was filtered to give a pale tan solid. The solid was washed with ice-cold water and dried under vacuum overnight to give 1.03 g of methyl 3-(chlorosulfonyl)benzoate as a pale tan solid. The aqueous filtrate was extracted with dichloromethane. The organic extract was washed with saturated sodium chloride, dried over magnesium sulfate, filtered, and the filtrate was concentrated to give 0.833 g of methyl 3-(chlorosulfonyl)benzoate as an oil which solidified upon standing to give a pale tan solid for a total yield of 1.86 g (82%) of methyl 3-(chlorosulfonyl)benzoate. ¹H NMR (400 MHz, CDCl₃): δ 8.69 (m, 1H), 8.40 (d, J=8 Hz, 1H), 8.22 (d, J=8 Hz, 1H), 7.73 (t, J=8 Hz, 1H), 3.99 (s, 3H).

### 43b) Methyl 3-({5-[(phenylmethyl)oxy]-1H-indol-1-yl}sulfonyl)benzoate

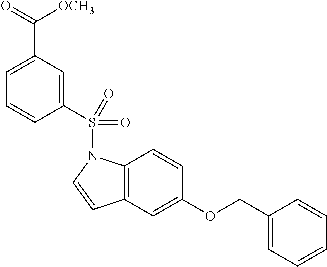

To a 3-neck round bottom flask was added sodium hydride (60% dispersion in oil) (0.143 g, 3.58 mmol). The sodium hydride was washed with hexanes and the solid was cooled in an ice-water bath. To the washed sodium hydride was slowly added a solution of 5-benzyloxyindole (0.515 g, 2.31 mmol) in N,N-dimethylformamide (5 mL) with stirring under a nitrogen atmosphere. The reaction mixture was stirred with cooling for 10 min. To the cold reaction mixture was added a solution of methyl 3-(chlorosulfonyl)benzoate (0.762 g, 3.2 mmol) in N,N-dimethylformamide (5 mL). The ice-water bath was removed and the reaction mixture was stirred overnight at room temperature. The reaction mixture was partitioned between water and ethyl acetate. The organic phase was separated, dried over magnesium sulfate, filtered, and the filtrate was concentrated to give a brown-orange liquid. The crude product was purified by flash chromatography over silica with a hexanes:ethyl acetate gradient (100:0 to 60:40) to give 0.352 g (36%) of methyl 3-({5-[(phenylmethyl)oxy]-1H-indol-1-yl}sulfonyl)benzoate as a colorless oil.

¹H NMR (400 MHz, CDCl₃): δ 8.51 (m, 1H), 8.18 (d, J=8 Hz, 1H), 8.00 (d, J=8 Hz, 1H), 7.89 (d, J=9 Hz, 1H), 7.52 (m, 2H), 7.30-7.43 (m, 5H), 7.03 (m, 2H), 6.60 (d, J=4 Hz, 1H), 5.05 (s, 2H), 3.93 (s, 3H). ES-LCMS m/z 444 (M+Na)⁺.

### 43c) Methyl 3-[(5-hydroxy-1H-indol-1-yl)sulfonyl]benzoate

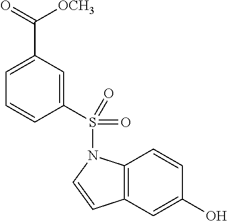

To an dry ice/acetone cooled, stirred solution of methyl 3-({5-[(phenylmethyl)oxy]-1H-indol-1-yl}sulfonyl)benzoate (0.16 g, 0.38 mmol) in dichloromethane (8 mL) between −60° C. and −65° C. was slowly added, dropwise, a solution of boron tribromide (1 M in dichloromethane) (2 mL, 2 mmol). The reaction mixture was stirred under a nitrogen atmosphere between −55° C. and −65° C. for 0.5 h. The dry ice/acetone bath was replaced with an ice-water bath and the reaction mixture was stirred for 2 h. The reaction mixture was poured onto ice and the quenched reaction mixture was transferred to a reparatory funnel. The aqueous mixture was extracted with dichloromethane. The organic phase was separated, washed with saturated sodium chloride, dried over magnesium sulfate, filtered, and the filtrate was concentrated to give an oil. The crude product was purified by flash chromatography over silica with a hexanes:ethyl acetate gradient (100:0 to 60:40) to give 0.082 g (65%) of methyl 3-[(5-hydroxy-1H-indol-1-yl)sulfonyl]benzoate as oil. ¹H NMR (400 MHz, CDCl₃): δ 8.50 (s, 1H), 8.18 (d, J=8 Hz, 1H), 7.99 (d, J=8 Hz, 1H), 7.86 (d, J=9 Hz, 1H), 7.51 (m, 2H), 6.92 (d, J=2 Hz, 1H), 6.85 (dd, J=9, 2 Hz, 1H), 6.57 (d, J=4 Hz, 1H), 4.62 (br s, 1H), 3.92 (s, 3H). ES-LCMS m/z 332 (M+H)⁺.

### 43d) Methyl 3-{[5-({[3-(2,6-dichlorophenyl)-5-(1-methylethyl)-4-isoxazolyl]methyl}oxy)-1H-indol-1-yl]sulfonyl}benzoate

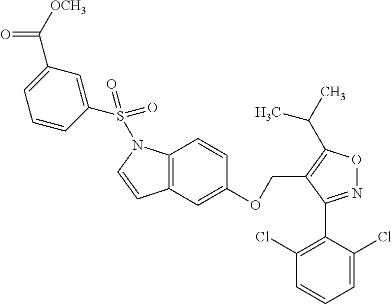

To a stirred mixture of methyl 3-[(5-hydroxy-1H-indol-1-yl)sulfonyl]benzoate (0.08 g, 0.24 mmol), [3-(2,6-dichlorophenyl)-5-(1-methylethyl)-4-isoxazolyl]methanol (prepared by the general procedure described in Maloney, P. R., et al., 2000 J. Med. Chem. 43:2971-2974) (0.075 g, 0.26 mmol), triphenylphosphine (0.067 g, 0.255 mmol), and dichloromethane (8 mL) was slowly added, dropwise, diisopropyl azodicarboxylate (0.05 mL, 0.25 mmol) at room temperature under a nitrogen atmosphere. The reaction mixture was stirred overnight at room temperature. The reaction mixture was concentrated and the crude product was purified by flash chromatography over silica with hexanes:dichloromethane (1:4) to give 0.095 g (66%) of methyl 3-{[5-({[3-(2,6-dichlorophenyl)-5-(1-methylethyl)-4-isoxazolyl]methyl}oxy)-1H-indol-1-yl]sulfonyl}benzoate as an amorphous solid. ¹H NMR (400 MHz, CDCl₃): δ 8.48 (s, 1H), 8.17 (d, J=8 Hz, 1H), 7.98 (d, J=8 Hz, 1H), 7.81 (d, J=9 Hz, 1H), 7.50 (m, 2H), 7.35 (m, 2H), 7.29 (m, 1H), 6.83 (d, J=2 Hz, 1H), 6.78 (dd, J=9, 2 Hz, 1H), 6.53 (d, J=4 Hz, 1H), 4.71 (s, 2H), 3.92 (s, 3H), 3.28 (septet, J=7 Hz, 1H), 1.36 (d, J=7 Hz, 6H). ES-LCMS m/z 599 (M+H)⁺.

### 43e) 3-{[5-({[3-(2,6-Dichlorophenyl)-5-(1-methylethyl)-4-isoxazolyl]methyl}oxy)-1H-indol-1-yl]sulfonyl}benzoic acid

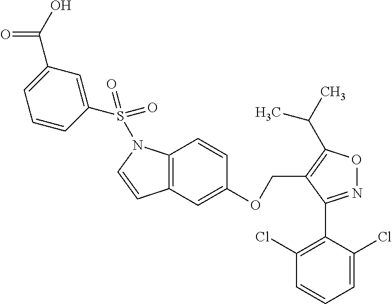

Methyl 3-{[5-({[3-(2,6-dichlorophenyl)-5-(1-methylethyl)-4-isoxazolyl]methyl}oxy)-1H-indol-1-yl]sulfonyl}benzoate (0.086 g, 0.14 mmol), lithium hydroxide (1 N) (0.20 mL, 0.20 mmol), and 1,4-dioxane (2 mL) were combined and the reaction mixture was stirred at room temperature for 3.5 h. To the reaction mixture was added lithium hydroxide (1 N) (0.05 mL, 0.05 mmol) and stirring was continued for 3 h. To the reaction mixture was added water (3 mL), followed by saturated sodium hydrogensulfate (0.10 mL) and ethyl acetate (10 mL). The mixture was transferred to a reparatory funnel and the layers were separated. The organic phase was washed with water (2 mL), followed by saturated sodium chloride (2 mL), dried over magnesium sulfate, filtered, and the filtrate was concentrated to give an oil. The crude product was purified by flash chromatography over silica with a dichloromethane:methanol gradient (100:0 to 95:5) to give 0.02 g of 3-{[5-({[3-(2,6-dichlorophenyl)-5-(1-methylethyl)-4-isoxazolyl]methyl}oxy)-1H-indol-1-yl]sulfonyl}benzoic acid as an amorphous solid. An impure fraction from the flash column was concentrated and the residue was purified by reverse phase preparative HPLC with an acetonitrile:water gradient (50:50 to 100:0) using 0.05% trifluoroacetic acid as a modifier. The HPLC fractions which contained product were combined, frozen, and placed in a lypholizer. The solid partially melted during lyophilization, therefore, the frozen material was allowed to melt and the resulting solution was concentrated in vacuo. The product obtained from the preparative HPLC purification was dissolved in dichloromethane and the solution was concentrated to give another 0.029 g of 3-{[5-({[3-(2,6-dichlorophenyl)-5-(1-methylethyl)-4-isoxazolyl]methyl}oxy)-1H-indol-1-yl]sulfonyl}benzoic acid as a white amorphous solid for a total yield of 0.049 g (60%). ¹H NMR (400 MHz, CDCl₃): δ 8.53 (d, J=2 Hz, 1H), 8.22 (d, J=8 Hz, 1H), 8.04 (d, J=8 Hz, 1H), 7.82 (d, J=9 Hz, 1H), 7.55 (t, J=8 Hz, 1H), 7.52 (d, J=4 Hz, 1H), 7.36 (m, 2H), 7.28 (dd, J=9, 7 Hz, 1H), 6.84 (d, J=2 Hz, 1H), 6.79 (dd, J=9, 3 Hz, 1H), 6.55 (d, J=4 Hz, 1H), 4.72 (s, 2H), 3.29 (septet, J=7 Hz, 1H), 1.37 (d, J=7 Hz, 6H). HRMS C₂₈H₂₃Cl₂N₂O₆S m/z 585.0654 (M+H)⁺_{Cal}; 585.0658 (M+H)⁺^{Obs}.

### Example 44

### 3-[5-({[3-(2,6-Dichlorophenyl)-5-(1-methylethyl)-4-isoxazolyl]methyl}oxy)-1-oxo-1,3-dihydro-2H-isoindol-2-yl]benzoic acid

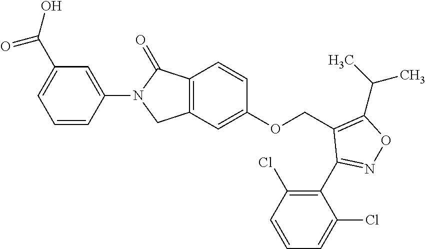

### 44a) Methyl 2-methyl-4-(methyloxy)benzoate

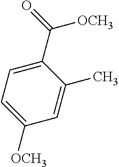

To a stirred mixture of 4-methoxy-2-methyl benzoic acid (1.0 g, 6 mmol) and methanol (50 mL) was added thionyl chloride (1.3 mL, 17.8 mmol), dropwise, at room temperature under a nitrogen atmosphere. The reaction mixture was heated at reflux for 3 h. The reaction mixture was allowed to cool at room temperature and concentrated to give a pale yellow liquid. The crude product was purified by flash chromatography over silica with hexanes:ethyl acetate (9:1) to give 1.11 g (100%) of methyl 2-methyl-4-(methyloxy)benzoate as a colorless liquid. ¹H NMR (400 MHz, DMSO-d₆): δ 7.81 (d, J=9 Hz, 1H), 6.86 (d, J=3 Hz, 1H), 6.83 (dd, J=9, 3 Hz, 1H), 3.78 (s, 3H), 3.75 (s, 3H), 2.49 (s, 3H). ES-LCMS m/z 181 (M+H)⁺.

### 44b) Methyl 2-(bromomethyl)-4-(methyloxy)benzoate

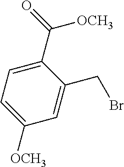

Methyl 2-methyl-4-(methyloxy)benzoate (1.1 g, 6.1 mmol), N-bromosuccinimide (1.2 g, 6.74 mmol), benzoyl peroxide (0.073 g, 0.30 mmol), and carbon tetrachloride (40 mL) were combined and the stirred reaction mixture was heated at reflux for 24 h under a nitrogen atmosphere. The reaction mixture was allowed to cool at room temperature and filtered through a pad of Celite®. The pad was washed with ethyl acetate. The filtrate was concentrated to give the crude product which was purified by flash chromatography over silica with a hexanes: dichloromethane gradient (100:0 to 50:50) to give 0.95 g (60%) of methyl 2-(bromomethyl)-4-(methyloxy)benzoate as an oil which solidified to a white solid. ¹H NMR (400 MHz, CDCl₃): δ 7.98 (d, J=9 Hz, 1H), 6.96 (d, J=3 Hz, 1H), 6.85 (dd, J=9, 3 Hz, 1H), 4.96 (s, 2H), 3.90 (s, 3H), 3.86 (s, 3H).

### 44c) Methyl 3-(5-hydroxy-1-oxo-1,3-dihydro-2H-isoindol-2-yl)benzoate

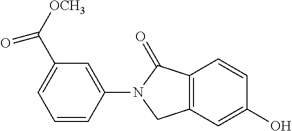

Methyl 2-(bromomethyl)-4-(methyloxy)benzoate (0.876 g, 3.38 mmol), methyl-3-aminobenzoate (0.52 g, 3.43 mmol), triethylamine (1 mL, 7.17 mmol), and N,N-dimethylformamide (10 mL) were combined in a sealed glass tube and the reaction mixture was heated in a microwave at 150° C. with stirring for 20 min. The reaction mixture was allowed to cool at room temperature and concentrated to give an oil. The crude material was purified by flash chromatography over silica with a hexanes:ethyl acetate gradient (100:0 to 50:50) to give 0.692 g of methyl 4-(methyloxy)-2-[({3-[(methyloxy)carbonyl]phenyl}amino)methyl]benzoate. This non-cyclized intermediate was further purified by reverse phase preparative HPLC using an acetonitrile:water gradient (50:50 to 100:0) with 0.05% trifluoroacetic acid as a modifier. The fractions containing methyl 4-(methyloxy)-2-[({3-[(methyloxy)carbonyl]phenyl}amino)methyl]benzoate were combined and concentrated in vacuo. Partial cyclization occurred during concentration to give a mixture of methyl 4-(methyloxy)-2-[({3-[(methyloxy)carbonyl]phenyl}amino)methyl]benzoate and methyl 3-[5-(methyloxy)-1-oxo-1,3-dihydro-2H-isoindol-2-yl]benzoate. The mixture was dissolved in a solution of 0.05% trifluoroacetic acid in acetonitrile and the solvent was removed in vacuo. The mixture was repeatedly dissolved in 0.05% trifluoroacetic acid in acetonitrile and subsequently concentrated until there was approximately 85-90% conversion to methyl 3-[5-(methyloxy)-1-oxo-1,3-dihydro-2H-isoindol-2-yl]benzoate. The impure isoindolinone was dissolved in dichloromethane and the solution was dried over magnesium sulfate, filtered, and the filtrate was concentrated to give 0.359 g of an approximately 85:15 mixture of methyl 3-[5-(methyloxy)-1-oxo-1,3-dihydro-2H-isoindol-2-yl]benzoate and methyl 4-(methyloxy)-2-[({3-[(methyloxy)carbonyl]phenyl}amino)methyl]benzoate, respectively. To 0.342 g of this mixture was added dichloromethane (10 mL) and the solution was cooled in an ice-water bath. To the cold solution was slowly added boron tribromide (1 M in dichloromethane) (5 mL, 5 mmol) with stirring under a nitrogen atmosphere. The reaction mixture was stirred for 1 h and the ice-water bath was removed. The reaction mixture was stirred for 5.5 h at room temperature. To the reaction mixture was slowly added boron tribromide (1 M in dichloromethane) (2 mL, 2 mmol) with stirring at room temperature under a nitrogen atmosphere. The reaction mixture was allowed to stir at room temperature overnight. ES-LCMS analysis of the reaction mixture indicated that it is a mixture of methyl 3-(5-hydroxy-1-oxo-1,3-dihydro-2H-isoindol-2-yl)benzoate and 3-(5-hydroxy-1-oxo-1,3-dihydro-2H-isoindol-2-yl)benzoic acid in a ratio of approximately 1:1. The reaction mixture was poured into ice-water and the aqueous mixture was filtered to give a solid. The flasks were rinsed with methanol and the methanolic solutions were combined with the filtered solid. The solvent was removed in vacuo and methanol was added to the solid. The solvent was removed in vacuo and toluene was added to the solid. Toluene was added and evaporated twice more to give 0.23 g of a mixture of methyl 3-(5-hydroxy-1-oxo-1,3-dihydro-2H-isoindol-2-yl)benzoate and 3-(5-hydroxy-1-oxo-1,3-dihydro-2H-isoindol-2-yl)benzoic acid as a pale tan solid. To the mixture of methyl 3-(5-hydroxy-1-oxo-1,3-dihydro-2H-isoindol-2-yl)benzoate and 3-(5-hydroxy-1-oxo-1,3-dihydro-2H-isoindol-2-yl)benzoic acid (0.23 g) was added methanol (15 mL). To the suspension was slowly added thionyl chloride (0.25 mL). The stirred reaction mixture was heated at reflux for 3 h under a nitrogen atmosphere. The reaction mixture was allowed to cool at room temperature and concentrated to give a solid. Toluene was added to the solid and the solvent was removed in vacuo to give 0.23 g (24% from methyl 2-(bromomethyl)-4-(methyloxy)benzoate) of methyl 3-(5-hydroxy-1-oxo-1,3-dihydro-2H-isoindol-2-yl)benzoate as a pale yellow solid. ¹H NMR (400 MHz, DMSO-d₆): δ 10.34 (s, 1H), 8.50 (s, 1H), 8.10 (dd, J=8, 2 Hz, 1H), 7.70 (d, J=8 Hz, 1H), 7.59 (d, J=8 Hz, 1H), 7.55 (t, J=8 Hz, 1H), 6.97 (s, 1H), 6.89 (dd, J=8, 2 Hz, 1H), 4.94 (s, 2H), 3.86 (s, 3H). ES-LCMS m/z 284 (M+H)⁺.

### 44d) Methyl 3-[5-({[3-(2,6-dichlorophenyl)-5-(1-methylethyl)-4-isoxazolyl]methyl}oxy)-1-oxo-1,3-dihydro-2H-isoindol-2-yl]benzoate

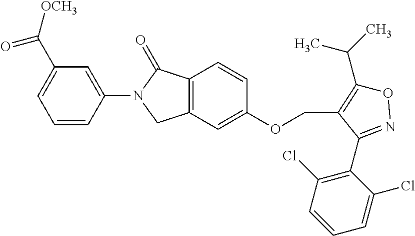

To a stirred suspension of methyl 3-(5-hydroxy-1-oxo-1,3-dihydro-2H-isoindol-2-yl)benzoate (0.102 g, 0.36 mmol), [3-(2,6-dichlorophenyl)-5-(1-methylethyl)-4-isoxazolyl]methanol (prepared according to the general procedure described in Maloney, P. R., et al., 2000 J. Med. Chem. 43:2971-2974) (0.102 g, 0.36 mmol), triphenylphosphine (0.092 g, 0.35 mmol), and dichloromethane (10 mL) was slowly added diisopropyl azodicarboxylate (0.07 mL, 0.36 mmol) at room temperature under a nitrogen atmosphere. After 21 h, the turbid reaction mixture was filtered and the filtrate was adsorbed onto silica. The crude product was purified by flash chromatography over silica with a hexanes:ethyl acetate gradient (100:0 to 70:30) to give an oil. The product was dissolved in dichloromethane and the solution was concentrated. The residue was once again dissolved in dichloromethane and the solution was concentrated to give 0.074 g (37%) of methyl 3-[5-({[3-(2,6-dichlorophenyl)-5-(1-methylethyl)-4-isoxazolyl]methyl}oxy)-1-oxo-1,3-dihydro-2H-isoindol-2-yl]benzoate as a cloudy oil. ¹H NMR (400 MHz, CDCl₃): δ 8.37 (m, 1H), 8.20 (s, 1H), 7.83 (d, J=8 Hz, 1H), 7.78 (m, 1H), 7.50 (m, 1H), 7.41 (d, J=8 Hz, 2H), 7.33 (m, 1H), 6.85-6.93 (m, 2H), 4.83 (s, 2H), 4.81 (s, 2H), 3.94 (s, 3H), 3.35 (septet, J=7 Hz, 1H), 1.44 (d, J=7 Hz, 6H). ES-LCMS m/z 551 (M+H)⁺.

### 44e) 3-[5-({[3-(2,6-dichlorophenyl)-5-(1-methylethyl)-4-isoxazolyl]methyl}oxy)-1-oxo-1,3-dihydro-2H-isoindol-2-yl]benzoic acid

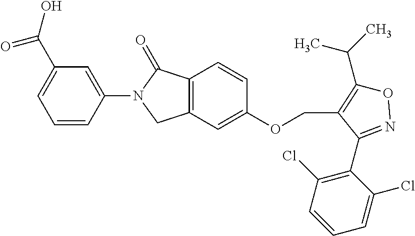

To a stirred solution of methyl 3-[5-({[3-(2,6-dichlorophenyl)-5-(1-methylethyl)-4-isoxazolyl]methyl}oxy)-1-oxo-1,3-dihydro-2H-isoindol-2-yl]benzoate (0.074 g, 0.13 mmol) in 1,4-dioxane (5 mL) was added lithium hydroxide (1 N) (0.28 mL, 0.28 mmol) at room temperature under a nitrogen atmosphere. After 17 h, lithium hydroxide (1 N) (0.2 mL, 0.2 mmol) was added to the reaction mixture and stirring was continued at room temperature for 27 h. To the reaction mixture was added lithium hydroxide (1 N) (0.1 mL, 0.1 mmol) and stirring was continued for 6 h. The reaction mixture was concentrated and the crude product was partitioned between water (8 mL), ethyl acetate (8 mL) and saturated sodium hydrogensulfate (0.4 mL). The organic phase was separated, washed with water (4 mL), followed by saturated sodium chloride (4 mL), dried over magnesium sulfate, filtered, and the filtrate was concentrated to give 0.05 g of 3-[5-({[3-(2,6-dichlorophenyl)-5-(1-methylethyl)-4-isoxazolyl]methyl}oxy)-1-oxo-1,3-dihydro-2H-isoindol-2-yl]benzoic acid a white solid. ¹H NMR indicated that a small aliphatic impurity was present. A portion of the product (30 mg) was purified further by preparative reverse phase HPLC with an acetonitrile:water gradient (50:50 to 100:0) using 0.05% trifluoroacetic acid as a modifier to give 4.6 mg of an analytical sample. ¹H NMR (400 MHz, DMSO-d₆): δ 13.06 (br s, 1H), 8.45 (s, 1H), 8.07 (dm, J=8 Hz, 1H), 7.69 (d, J=8 Hz, 1H), 7.62 (m, 3H), 7.54 (d, J=7 Hz, 1H), 7.52 (d, J=7 Hz, 1H), 7.08 (d, J=2 Hz, 1H), 6.90 (dd, J=8, 2 Hz, 1H), 4.94 (s, 2H), 4.93 (s, 2H), 3.47 (septet, J=7 Hz, 1H), 1.33 (d, J=7 Hz, 6H). ES-LCMS m/z 537 (M+H)⁺.

### Example 45

### 3-[6-({[3-(2,6-Dichlorophenyl)-5-(1-methylethyl)-4-isoxazolyl]methyl}oxy)-3-oxo-3,4-dihydro-2(1H)-isoquinolinyl]benzoic acid

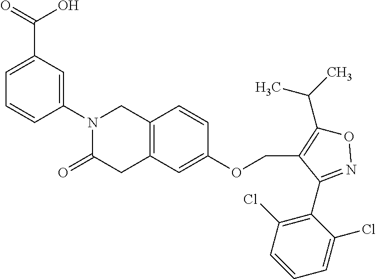

### 45a) 7-(Methyloxy)-1,4-dihydro-3H-2-benzopyran-3-one

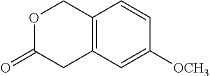

This compound was prepared according to the general procedure described by R. J. Spangler et al. (1997 J. Org. Chem. 42:2989-2996) with modification. 3-Methoxyphenyl acetic acid (3.05 g, 18.4 mmol), formaldehyde (37% aqueous) (4.5 mL, 60 mmol), hydrochloric acid (12 N) (1 mL), and glacial acetic acid (12 mL) were combined and the solution was stirred for five days at room temperature under a nitrogen atmosphere. The reaction mixture was poured into water (80 mL) and the aqueous mixture was extracted with chloroform (3×30 mL). The organic extracts were combined, washed carefully with 5% sodium bicarbonate (venting frequently to release carbon dioxide) followed by saturated sodium chloride, dried over magnesium sulfate, filtered, and the filtrate was concentrated to give a pale yellow oil. The crude product was purified by flush chromatography over silica with hexanes:ethyl acetate (2:1) to give 1.27 g (38%) of 7-(methyloxy)-1,4-dihydro-3H-2-benzopyran-3-one as a colorless oil which solidified upon standing to a white solid. ¹H NMR (400 MHz, CDCl₃): δ 7.15 (d, J=8 Hz, 1H), 6.82 (dd, J=8, 3 Hz, 1H), 6.77 (d, J=2 Hz, 1H), 5.25 (s, 2H), 3.81 (s, 3H), 3.67 (s, 2H).

### 45b) Methyl [2-(bromomethyl)-5-(methyloxy)phenyl]acetate

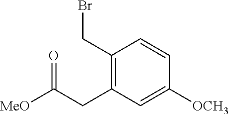

To a stirred solution of 7-(methyloxy)-1,4-dihydro-3H-2-benzopyran-3-one (1.26 g, 7.07 mmol), methanol (0.9 mL, 22.2 mmol), and toluene (50 mL) was slowly added dropwise thionyl bromide (0.70 mL, 9.1 mmol) at room temperature under a nitrogen atmosphere. The temperature of the reaction mixture was maintained below 30° C. during the addition of thionyl bromide. The reaction mixture was allowed to stir at room temperature for 4 h. The reaction mixture was carefully poured into an excess of 20% sodium bicarbonate (carbon dioxide is evolved!) and the mixture was stirred for 10 min. The quenched reaction mixture was transferred to a separatory funnel and the layers were separated. The aqueous phase was extracted with dichloromethane. The organic extracts were independently washed with water. The washed organic extracts were combined, dried over magnesium sulfate, filtered, and the filtrate was concentrated to give a liquid. The product was dissolved in dichloromethane and the solution was concentrated to give 1.73 g (90%) of methyl [2-(bromomethyl)-5-(methyloxy)phenyl]acetate. ¹H NMR (400 MHz, CDCl₃): δ 7.28 (d, J=8 Hz, 1H), 6.78 (m, 2H), 4.57 (s, 2H), 3.80 (s, 3H), 3.77 (s, 2H), 3.70 (s, 3H).

### 45c) Methyl 3-[6-({[3-(2,6-dichlorophenyl)-5-(1-methylethyl)-4-isoxazolyl]methyl}oxy)-3-oxo-3,4-dihydro-2(1H)-isoquinolinyl]benzoate

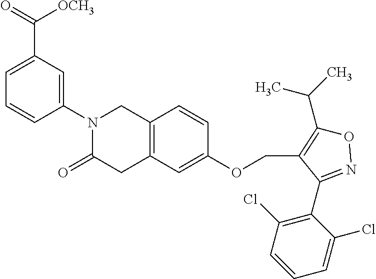

Methyl [2-(bromomethyl)-5-(methyloxy)phenyl]acetate (0.84 g, 3.08 mmol), methyl-3-aminobenzoate (0.553 g, 3.66 mmol), triethylamine (0.90 mL, 6.5 mmol), and toluene (25 mL) were combined in a round bottom flask and the stirred reaction mixture was heated at 90° C. for 48 h under a nitrogen atmosphere. The reaction mixture was filtered and the filtrate was concentrated to give an oil (1.48 g). To the oil was added toluene (30 mL) and triethylamine (0.9 mL). The solution was heated at 90° C. with stirring under a nitrogen atmosphere for 48 h. The reaction mixture was concentrated to give an oil. The oil was dissolved in toluene and the solvent was removed in vacuo to give an oil. The oil was once again dissolved in toluene and the solvent was removed in vacuo to give an oil (1.25 g). LCMS analysis indicated that the oil was predominantly the non-cyclized intermediate methyl 3-[({4-(methyloxy)-2-[2-(methyloxy)-2-oxoethyl]phenyl}methyl)amino]benzoate. To methyl 3-[({4-(methyloxy)-2-[2-(methyloxy)-2-oxoethyl]phenyl}methyl)amino]benzoate (1.18 g) was added p-toluenesulfonic acid monohydrate (0.16 g), and toluene (50 mL). The stirred reaction mixture was heated at 100° C. for 16 h under a nitrogen atmosphere.

The reaction mixture was partitioned between ethyl acetate and saturated sodium bicarbonate. The layers were separated and the organic phase was washed with water, followed by saturated sodium chloride, dried over magnesium sulfate, filtered, and the filtrate was concentrated to give 1.14 g of crude methyl 3-[6-(methyloxy)-3-oxo-3,4-dihydro-2(1H)-isoquinolinyl]benzoate as an orange oil. To an ice-water cooled solution of crude methyl 3-[6-(methyloxy)-3-oxo-3,4-dihydro-2(1H)-isoquinolinyl]benzoate (1.14 g) in dichloromethane (75 mL) was slowly added boron tribromide (1 M in dichloromethane) (15 mL, 15 mmol) with stirring under a nitrogen atmosphere. The ice-water bath was removed and the reaction mixture was allowed to stir at room temperature for 3.5 h. The reaction mixture was poured into ice-water and the mixture was partitioned between dichloromethane and water. The organic phase was separated, washed with saturated sodium chloride, dried over magnesium sulfate, filtered, and the filtrate was concentrated to give 0.528 g of crude methyl 3-(6-hydroxy-3-oxo-3,4-dihydro-2(1H)-isoquinolinyl)benzoate as an olive-green amorphous solid. Methyl 3-(6-hydroxy-3-oxo-3,4-dihydro-2(1H)-isoquinolinyl)benzoate (crude) (0.147 g), [3-(2,6-dichlorophenyl)-5-(1-methylethyl)-4-isoxazolyl]methanol (prepared according to the general procedure described in Maloney, P. R., et al., 2000 J. Med. Chem. 43:2971-2974) (0.133 g, 0.46 mmol), triphenylphosphine (0.124 g, 0.47 mmol), diisopropyl azodicarboxylate (0.095 mL, 0.48 mmol), and toluene (3 mL) were combined and the reaction mixture was heated at 80° C. in a microwave for 1500 seconds. The reaction mixture was adsorbed onto silica and purified by flash chromatography over silica with a hexanes:ethyl acetate gradient (100:0 to 0:100) to give 0.041 g (8.5% from methyl [2-(bromomethyl)-5-(methyloxy)phenyl]acetate) of methyl 3-[6-({[3-(2,6-dichlorophenyl)-5-(1-methylethyl)-4-isoxazolyl]methyl}oxy)-3-oxo-3,4-dihydro-2(1H)-isoquinolinyl]benzoate as an amorphous solid. ¹H NMR (400 MHz, CDCl₃): δ 7.94 (m, 2H), 7.55 (d, J=8 Hz, 1H), 7.49 (m, 1H), 7.42 (m, 2H), 7.33 (dd, J=9, 7 Hz, 1H), 7.05 (d, J=8 Hz, 1H), 6.68 (dd, J=8, 2 Hz, 1H), 6.62 (m, 1H), 4.78 (s, 2H), 4.75 (s, 2H), 3.91 (s, 3H), 3.69 (s, 2H), 3.34 (septet, J=7 Hz, 1H), 1.43 (d, J=7 Hz, 6H). ES-LCMS m/z 565 (M+H)⁺.

### 45d) 3-[6-({[3-(2,6-Dichlorophenyl)-5-(1-methylethyl)-4-isoxazolyl]methyl}oxy)-3-oxo-3,4-dihydro-2(1H)-isoquinolinyl]benzoic acid

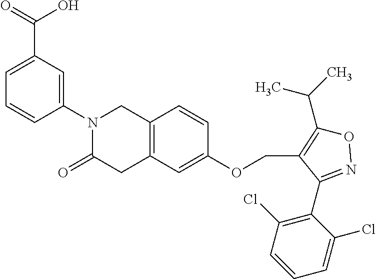

Methyl 3-[6-({[3-(2,6-dichlorophenyl)-5-(1-methylethyl)-4-isoxazolyl]methyl}oxy)-3-oxo-3,4-dihydro-2(1H)-isoquinolinyl]benzoate (0.041 g, 0.073 mmol), lithium hydroxide (1 N) (0.15 mL, 0.15 mmol), tetrahydrofuran (2 mL), and methanol (1 mL) were combined and the stirred reaction mixture was heated at 100° C. in the microwave for 500 seconds. The reaction mixture was concentrated and the crude product was partitioned between ethyl acetate (10 mL), water (4 mL), and saturated sodium hydrogensulfate (0.1 mL). The organic phase was separated, washed with water (4 mL). followed by saturated sodium chloride (4 mL), dried over magnesium sulfate, filtered, and the filtrate concentrated to give an oil. The crude product was applied to a silica column and eluted by flash chromatography with dichloromethane:methanol (95:5) to give the impure product. The impure product was once again applied to a silica column and eluted by flash chromatography with a dichloromethane:methanol gradient (100:0 to 95:5), followed by methanol to give the impure product. The impure product was finally purified by reverse phase preparative HPLC with an acetonitrile:water gradient (50:50 to 100:0) with 0.05% trifluoroacetic acid as a modifier to give 0.0024 g (6%) of 3-[6-({[3-(2,6-dichlorophenyl)-5-(1-methylethyl)-4-isoxazolyl]methyl}oxy)-3-oxo-3,4-dihydro-2(1H)-isoquinolinyl]benzoic acid as a pale yellow amorphous solid. ¹H NMR (400 MHz, DMSO-d₆): δ 13.08 (br s, 1H), 7.85 (m, 1H), 7.79 (d, J=8 Hz, 1H), 7.62 (m, 2H), 7.49-7.57 (m, 3H), 7.15 (d, J=8 Hz, 1H), 6.75 (d, J=2 Hz, 1H), 6.63 (dd, J=8, 2 Hz, 1H), 4.79 (s, 4H), 3.62 (s, 2H), 3.43 (septet, J=7 Hz, 1H), 1.32 (d, J=7 Hz, 6H). HRMS C₂₉H₂₅Cl₂N₂O₅ m/z 551.1135 (M+H)⁺_{Cal}; 551.1138 (M+H)⁺_{Obs}.

### Example 46

### 5-({[3-(2,6-Dichlorophenyl)-5-(1-methylethyl)-4-isoxazolyl]methyl}oxy)-1-{[4-(1H-tetrazol-5-yl)phenyl]methyl}-1H-indole

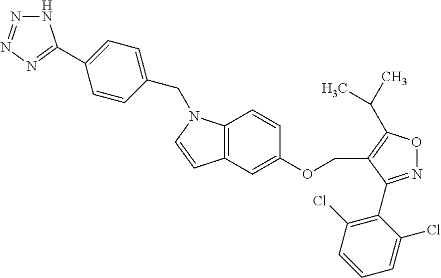

### 46a) 5-({[3-(2,6-Dichlorophenyl)-5-(1-methylethyl)-4-isoxazolyl]methyl}oxy)-1H-indole

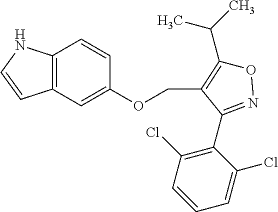

To a stirred mixture of 5-hydroxyindole (0.142 g, 1.07 mmol), [3-(2,6-dichlorophenyl)-5-(1-methylethyl)-4-isoxazolyl]methanol (prepared according to the general procedure described in Maloney, P. R., et al., 2000 J. Med. Chem. 43:2971-2974) (0.30 g, 1.05 mmol), triphenylphosphine (PS-polymer bound; 1 mmol/g) (1.2 g, 1.2 mmol), and dichloromethane (20 mL) was added, dropwise, diisopropyl azodicarboxylate (0.23 mL, 1.17 mmol) at room temperature under a nitrogen atmosphere. The reaction mixture was stirred for 2 days and allowed to stand at room temperature for 8 days. The reaction mixture was filtered and the resin was washed with dichloromethane. The filtrate was concentrated to give a gold-yellow oil. The crude product was purified by flash chromatography over silica with a hexanes:dichloromethane gradient (100:0 to 50:50) to give 0.128 g (30%) of 5-({[3-(2,6-dichlorophenyl)-5-(1-methylethyl)-4-isoxazolyl]methyl}oxy)-1H-indole as a white amorphous solid. ¹H NMR (400 MHz, CDCl₃): δ 8.03 (br s, 1H), 7.39 (m, 2H), 7.30 (dd, J=9, 7 Hz, 1H), 7.22 (d, J=9 Hz, 1H), 7.17 (br t, J=3 Hz, 1H), 6.98 (d, J=2 Hz, 1H), 6.71 (dd, J=9, 2 Hz, 1H), 6.42 (m, 1H), 4.75 (s, 2H), 3.34 (septet, J=7 Hz, 1H), 1.40 (d, J=7 Hz, 6H). ES-LCMS m/z 401 (M+H)⁺.

### 46b) 4-{[5-({[3-(2,6-Dichlorophenyl)-5-(1-methylethyl)-4-isoxazolyl]methyl}oxy)-1H-indol-1-yl]methyl}benzonitrile

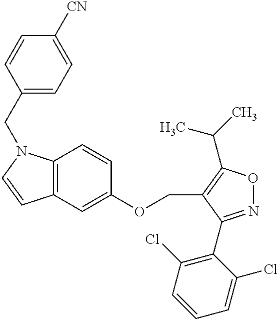

Sodium hydride (60% dispersion in oil) (0.016 g, 0.4 mmol) was washed with hexanes. To the washed sodium hydride was added N,N-dimethylformamide (1 mL). To the stirred sodium hydride suspension was slowly added, dropwise, a solution of 5-({[3-(2,6-dichlorophenyl)-5-(1-methylethyl)-4-isoxazolyl]methyl}oxy)-1H-indole (0.124 g, 0.31 mmol) in N,N-dimethylformamide (1.5 mL) at room temperature under a nitrogen atmosphere. The reaction mixture was stirred for several minutes and a solution of α-bromo-p-tolunitrile (0.073 g, 0.37 mmol) in N,N-dimethylformamide (1 mL) was added. The reaction mixture was stirred at room temperature under a nitrogen atmosphere for 4 days. The reaction mixture was concentrated to give an oil.

The crude product was purified by flash chromatography over silica with a hexanes:ethyl acetate gradient (100:0 to 75:25) to give a colorless oil. The oil was dissolved in toluene and the solution was concentrated in vacuo to give an oil. The oil was dissolved in toluene and the solution was concentrated to give 0.12 g (75%) of 4-{[5-({[3-(2,6-dichlorophenyl)-5-(1-methylethyl)-4-isoxazolyl]methyl}oxy)-1H-indol-1-yl]methyl}benzonitrile. ¹H NMR (400 MHz, CDCl₃): δ 7.56 (d, J=8 Hz, 2H), 7.38 (m, 2H), 7.30 (dd, J=9, 7 Hz, 1H), 7.09 (d, J=8 Hz, 2H), 7.08 (d, J=3 Hz, 1H), 7.00 (d, J=2 Hz, 1H), 6.96 (d, J=9 Hz, 1H), 6.68 (dd, J=9, 2 Hz, 1H), 6.45 (d, J=2 Hz, 1H), 5.32 (s, 2H), 4.74 (s, 2H), 3.31 (septet, J=7 Hz, 1H), 1.38 (d, J=7 Hz, 6H). ES-LCMS m/z 516 (M+H)⁺.

### 46c) 5-({[3-(2,6-Dichlorophenyl)-5-(1-methylethyl)-4-isoxazolyl]methyl}oxy)-1-{[4-(1H-tetrazol-5-yl)phenyl]methyl}-1H-indole

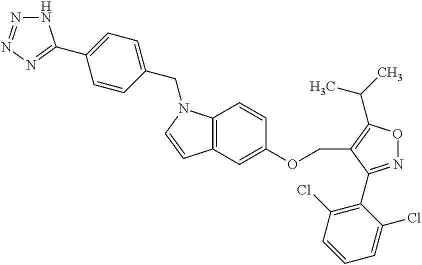

4-{[5-({[3-(2,6-Dichlorophenyl)-5-(1-methylethyl)-4-isoxazolyl]methyl}oxy)-1H-indol-1-yl]methyl}benzonitrile (0.113 g, 0.22 mmol), 1-methyl-2-pyrrolidinone (2 mL), sodium azide (0.03 g, 0.46 mmol), and triethylamine hydrochloride (0.042 g, 0.31 mmol) were combined and the stirred reaction mixture was heated at 150° C. under a nitrogen atmosphere for 3 h. (Note: The reaction was conducted behind a blast shield.) The reaction mixture was allowed to stand overnight at room temperature under a nitrogen atmosphere. The reaction mixture was diluted with water (10 mL) and the pH of the aqueous reaction mixture was adjusted to approximately 1 (litmus paper) with 1 N hydrochloric acid. The acidic aqueous phase was extracted twice with ethyl acetate. The organic extracts were combined, washed with saturated sodium chloride, dried over magnesium sulfate, filtered, and the filtrate was concentrated to give a brown liquid. The crude product was applied to a silica column and eluted by flash chromatography with a dichloromethane:methanol gradient (100:0 to 90:10) to give the impure product as a brown oil. The impure product was purified by reverse phase preparative HPLC with an acetonitrile:water gradient (50:50 to 100:0) using 0.05% trifluoroacetic acid as a modifier to give after drying 0.0345 g (28%) of 5-({[3-(2,6-dichlorophenyl)-5-(1-methylethyl)-4-isoxazolyl]methyl}oxy)-1-{[4-(1H-tetrazol-5-yl)phenyl]methyl}-1H-indole as an off-white amorphous solid. ¹H NMR (400 MHz, DMSO-d₆): δ 7.92 (d, J=8 Hz, 2H), 7.59 (m, 2H), 7.51 (dd, J=9, 7 Hz, 1H), 7.46 (d, J=3 Hz, 1H), 7.30 (d, J=8 Hz, 2H), 7.22 (d, J=9 Hz, 1H), 6.96 (d, J=2 Hz, 1H), 6.51 (dd, J=9, 2 Hz, 1H), 6.34 (d, J=3 Hz, 1H), 5.43 (s, 2H), 4.74 (s, 2H), 3.35 (septet, J=7 Hz, 1H), 1.26 (d, J=7 Hz, 6H). ES-LCMS m/z 557 (M−H)⁻.

### Example 47

### 2-{[5-({[3-(2,6-Dichlorophenyl)-5-(1-methylethyl)-4-isoxazolyl]methyl}oxy)-1H-indol-1-yl]methyl}-1,3-oxazole-4-carboxylic acid

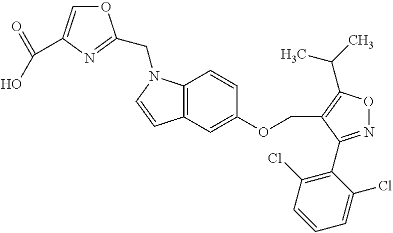

### 47a) Methyl 2-(dichloromethyl)-4,5-dihydro-1,3-oxazole-4-carboxylate

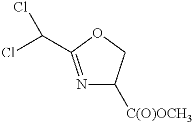

This compound was prepared according to the general procedure described by S. A. Hermitage, et al., 2001 Org. Proc. Res. Devel. 5:37-44. To methanol (10 mL) was added sodium methoxide (25% by weight in methanol) (1.15 mL, 5 mmol). The sodium methoxide solution was cooled in an acetone/ice bath to −15° C. (bath temperature) and dichloroacetonitrile (4 mL, 50 mmol) was added slowly, dropwise, over a 30 min period with stirring under a nitrogen atmosphere. The temperature of the reaction mixture was maintained below −3° C. during the addition of dichloroacetonitrile. The reaction mixture was stirred with cooling for 20 min. To the cold reaction mixture was added DL-serine methyl ester hydrochloride (8.9 g, 57 mmol) via a powder addition funnel, followed by methanol (8 mL). The stirred reaction mixture was allowed to slowly warm to room temperature overnight. To the reaction mixture was added water (16 mL) and dichloromethane (30 mL). The mixture was transferred to a separatory funnel and the layers were separated. The aqueous phase was extracted with dichloromethane (16 mL). The organic extracts were combined, dried over magnesium sulfate, filtered, and the filtrate was concentrated to give 8.58 g (81% crude from dichloroacetonitrile) of methyl 2-(dichloromethyl)-4,5-dihydro-1,3-oxazole-4-carboxylate. ¹H NMR (400 MHz, CDCl₃): δ 6.28 (s, 1H), 4.92-4.87 (m, 1H), 4.77-4.72 (m, 1H), 4.68-4.64 (m, 1H), 3.82 (s, 3H).

### 47b) Methyl 2-(chloromethyl)-1,3-oxazole-4-carboxylate

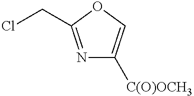

This compound was prepared according to the general procedure described by S. A. Hermitage, et al., (2001 Org. Proc. Res. Devel. 5:37-44) with modification. To an ice-water cooled solution of methyl 2-(dichloromethyl)-4,5-dihydro-1,3-oxazole-4-carboxylate (8.54 g, 40.3 mmol) in methanol (8 mL) was slowly added, dropwise, a solution of sodium methoxide (25% by weight in methanol) (9.2 mL, 40.2 mmol) with stirring under a nitrogen atmosphere. Approximately 5 min after the addition of the sodium methoxide solution had begun, the addition was stopped and the ice-water bath was replaced with an acetone/ice bath. The addition of the sodium methoxide solution was resumed. The temperature of the reaction mixture was maintained below 10° C. during the addition of the sodium methoxide solution. Once addition of the sodium methoxide solution was complete, the acetone/ice bath was replaced with an ice-water bath and the reaction mixture was allowed to slowly warm to room temperature overnight with stirring under a nitrogen atmosphere. The reaction mixture was partitioned between dichloromethane (25 mL) and water (15 mL). The layers were separated, and the aqueous phase was extracted with dichloromethane (15 mL). The organic extracts were combined, washed with saturated sodium chloride, dried over magnesium sulfate, filtered, and the filtrate was concentrated to give 7.01 g of a crude brown-orange oil. The crude material (7.0 g) was dissolved in toluene (16 mL), and camphorsulfonic acid (1.25 g, 5.4 mmol) was added at room temperature. The stirred reaction mixture was heated at 70° C. for 1 h under a nitrogen atmosphere. The reaction mixture was allowed to stand at room temperature overnight. The reaction mixture was washed with potassium carbonate (10% w/v) (10 mL). The layers were separated and the organic phase was washed with water (15 mL). The layers were separated and the aqueous washes were combined and extracted with toluene (20 mL). The organic extracts were combined, dried over magnesium sulfate, filtered, and the filtrate was concentrated to give the crude product as a brown oil. The crude product was purified by flash chromatography over silica with a hexanes:ethyl acetate gradient (100:0 to 70:30) to give 2.44 g (28% from dichloroacetonitrile) of methyl 2-(chloromethyl)-1,3-oxazole-4-carboxylate as a white solid. ¹H NMR (400 MHz, CDCl₃): δ 8.25 (s, 1H), 4.63 (s, 2H), 3.92 (s, 3H). ES-LCMS m/z 176 (M+H)⁺.

### 47c) Methyl 2-{[5-({[3-(2,6-dichlorophenyl)-5-(1-methylethyl)-4-isoxazolyl]methyl}oxy)-1H-indol-1-yl]methyl}-1,3-oxazole-4-carboxylate

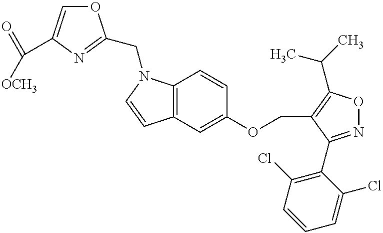

Sodium hydride (60% dispersion in oil) (0.083 g, 2.08 mmol) was washed with hexanes. To the washed sodium hydride was added N,N-dimethylformamide (2 mL). To the stirred sodium hydride suspension was slowly added a solution of 5-({[3-(2,6-dichlorophenyl)-5-(1-methylethyl)-4-isoxazolyl]methyl}oxy)-1H-indole (prepared according to the general procedure described for Example 46a (0.705 g, 1.76 mmol) in N,N-dimethylformamide (4 mL) at room temperature under a nitrogen atmosphere.

The reaction mixture was stirred for 10 min and a solution of methyl 2-(chloromethyl)-1,3-oxazole-4-carboxylate (0.31 g, 1.77 mmol) in N,N-dimethylformamide (3 mL), was added slowly over 5 min. The reaction mixture was stirred at room temperature under a nitrogen atmosphere for 21 h. To the reaction mixture was added water and the mixture was partitioned between ethyl acetate and water. The phases were separated and the aqueous phase was extracted with ethyl acetate. The organic extracts were combined, washed with water, followed by saturated sodium chloride, dried over magnesium sulfate, filtered, and the filtrate was concentrated to give a gold-yellow oil. The oil was purified by flash chromatography over silica with a hexanes:ethyl acetate gradient (100:0 to 50:50) to give 0.166 g of methyl 2-{[5-({[3-(2,6-dichlorophenyl)-5-(1-methylethyl)-4-isoxazolyl]methyl}oxy)-1H-indol-1-yl]methyl}-1,3-oxazole-4-carboxylate as a white amorphous solid as well as 0.122 g of indole starting material. ¹H NMR indicates that a small impurity was present. The material was used without further purification. ¹H NMR (400 MHz, CDCl₃): δ 8.09 (s, 1H), 7.38 (m, 2H), 7.29 (m, 2H), 7.15 (d, J=3 Hz, 1H), 6.95 (d, J=2 Hz, 1H), 6.73 (dd, J=9, 2 Hz, 1H), 6.41 (d, J=3 Hz, 1H), 5.36 (s, 2H), 4.72 (s, 2H), 3.90 (s, 3H), 3.32 (septet, J=7 Hz, 1H), 1.38 (d, J=7 Hz, 6H). ES-LCMS m/z 540 (M+H)⁺.

[Note: The aqueous phase of the reaction work-up noted above contained 2-{[5-({[3-(2,6-dichlorophenyl)-5-(1-methylethyl)-4-isoxazolyl]methyl}oxy)-1H-indol-1-yl]methyl}-1,3-oxazole-4-carboxylic acid according to ES-LCMS. The pH of the aqueous phase was adjusted to approximately 2-3 (litmus paper) with 10% citric acid and the acidic aqueous phase was extracted with ethyl acetate (2 times). The organic extracts were combined, washed with water, followed by saturated sodium chloride, dried over magnesium sulfate, filtered, and the filtrate was concentrated to give an oil.

The oil was partially purified by flash chromatography over silica with dichloromethane and methanol to give approximately 0.08 g of impure 2-{[5-({[3-(2,6-dichlorophenyl)-5-(1-methylethyl)-4-isoxazolyl]methyl}oxy)-1H-indol-1-yl]methyl}-1,3-oxazole-4-carboxylic.]

### 47d) 2-{[5-({[3-(2,6-Dichlorophenyl)-5-(1-methylethyl)-4-isoxazolyl]methyl}oxy)-1H-indol-1-yl]methyl}-1,3-oxazole-4-carboxylic acid

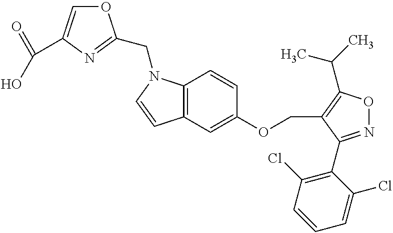

To a stirred solution of methyl 2-{[5-({[3-(2,6-dichlorophenyl)-5-(1-methylethyl)-4-isoxazolyl]methyl}oxy)-1H-indol-1-yl]methyl}-1,3-oxazole-4-carboxylate (0.166 g, 0.31 mmol) in tetrahydrofuran (8 mL) and methanol (4 mL) was added sodium hydroxide (1 N) (0.65 mL, 0.65 mmol). The reaction mixture was stirred at room temperature under a nitrogen atmosphere for 19 h. The tetrahydrofuran and methanol were removed in vacuo and the aqueous mixture was diluted with water (5 mL). The pH of the aqueous mixture was adjusted to approximately 3 (litmus paper) with 10% citric acid. The acidic aqueous mixture was extracted twice with ethyl acetate. The organic extracts were combined, washed with water, followed by saturated sodium chloride, dried over magnesium sulfate, filtered, and the filtrate was concentrated to give 0.158 g (98%) of 2-{[5-({[3-(2,6-dichlorophenyl)-5-(1-methylethyl)-4-isoxazolyl]methyl}oxy)-1H-indol-1-yl]methyl}-1,3-oxazole-4-carboxylic acid as a white amorphous solid. ¹H NMR (400 MHz, DMSO-d₆): δ 13.08 (br s, 1H), 8.63 (s, 1H), 7.60 (m, 2H), 7.51 (dd, J=9, 7 Hz, 1H), 7.37 (d, J=2 Hz, 1H), 7.27 (d, J=9 Hz, 1H), 6.94 (d, J=3 Hz, 1H), 6.55 (dd, J=9, 3 Hz, 1H), 6.32 (d, J=3 Hz, 1H), 5.55 (s, 2H), 4.74 (s, 2H), 3.38 (septet, J=7 Hz, 1H), 1.27 (d, J=7 Hz, 6H). HRMS C₂₆H₂₂Cl₂N₃O₅ m/z 526.0937 (M+H)⁺_{Cal}; 526.0930 (M+H)⁺_{Obs}.

### Example 48

### 5-{[5-({[3-(2,6-Dichlorophenyl)-5-(1-methylethyl)-4-isoxazolyl]methyl}oxy)-1H-indol-1-yl]methyl}-2-methylbenzoic acid

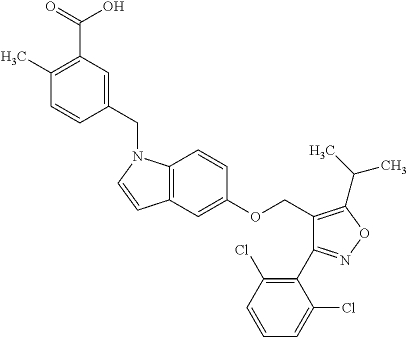

### 48a) 5-(Bromomethyl)-2-methylbenzoic acid

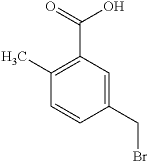

This compound was prepared according to the method described by Paul Soumendu et al. (2003 Eur. J. Org. Chem. 128-137) with modification. To a mixture of paraformaldehyde (1.2 g, 40 mmol), o-toluic acid (2.0 g, 14.7 mmol), and phosphoric acid (85%) (0.29 mL) was added hydrogen bromide (33% in acetic acid) (7 mL). The stirred reaction mixture was heated overnight at 115° C. under a nitrogen atmosphere. The oil bath was removed and the reaction mixture was allowed to stand at room temperature under a nitrogen atmosphere for 5 days. The reaction mixture was poured into ice-water and the mixture was filtered to give an off-white solid. The crude product was purified by flash chromatography over silica with a hexanes:ethyl acetate gradient (100:0 to 50:50) to give 1.23 g of 5-(bromomethyl)-2-methylbenzoic acid as a white solid. ¹H NMR indicates that the product is approximately 87 mol % pure and was taken on without further purification. ¹H NMR (400 MHz, CDCl₃): δ 8.08 (d, J=2 Hz, 1H), 7.49 (dd, J=8, 2 Hz, 1H), 7.28 (d, J=8 Hz, 1H), 4.50 (s, 2H), 2.64 (s, 3H). ES-LCMS m/z 227 (M−H)⁻.

### 48b) 1,1-Dimethylethyl 5-(bromomethyl)-2-methylbenzoate

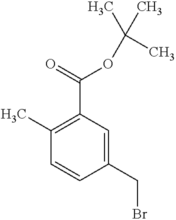

This compound was prepared according to the method described by Paul Soumendu et al. (2003 Eur. J. Org. Chem. 128-137) with modification. To a stirred solution of 5-(bromomethyl)-2-methylbenzoic acid (1.16 g) in cyclohexane (40 mL) and dichloromethane (40 mL) was added a solution of tert-butyl trichloroacetimidate (1.7 mL, 0.5 mmol) in cyclohexane (5 mL) at room temperature under a nitrogen atmosphere. To the stirred reaction mixture was slowly added, dropwise, boron trifluoride diethyl etherate (0.15 mL, 1.18 mmol). After 2 h, thin layer chromatography indicated that the reaction was not complete. The reaction mixture was stirred for an additional 45 min. To the reaction mixture was added a solution of tert-butyl trichloroacetimidate (0.80 mL, 4.5 mmol) in cyclohexane (2 mL), followed by the dropwise addition of boron trifluoride diethyl etherate (0.05 mL, 0.40 mmol). The reaction mixture was stirred for 1.5 h. The reaction mixture was washed with 5% sodium bicarbonate (100 mL) with the aid of dichloromethane. The organic phase was separated, washed with saturated sodium chloride, dried over magnesium sulfate, filtered, and the filtrate was concentrated to give a white solid. The crude product was purified by flash chromatography over silica with a hexanes:ethyl acetate gradient (100:0 to 90:10) to give 0.527 g of 1,1-dimethylethyl 5-(bromomethyl)-2-methylbenzoate as a clear colorless oil. ¹H NMR indicates the product is approximately 80 mol % pure. The impure product was purified by flash chromatography over silica with a hexanes:ethyl acetate gradient (100:0 to 98:2) to give 0.485 g of 1,1-dimethylethyl 5-(bromomethyl)-2-methylbenzoate as a clear colorless oil. ¹H NMR indicates that the product was approximately 81 mol % pure. The product was taken on without further purification. ¹H NMR (400 MHz, CDCl₃): δ 7.82 (d, J=2 Hz, 1H), 7.39 (dd, J=8, 2 Hz 1H), 7.19 (d, J=8 Hz, 1H), 4.48 (s, 2H), 2.55 (s, 3H), 1.59 (s, 9H).

### 48c) 1,1-Dimethylethyl 5-[(5-hydroxy-1H-indol-1-yl)methyl]-2-methylbenzoate

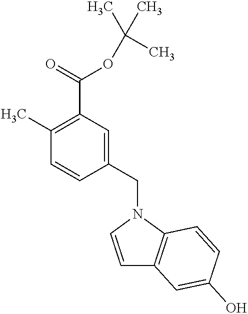

Sodium hydride (60% oil dispersion) (0.078 g, 1.95 mmol) was washed with hexanes and N,N-dimethylformamide (4 mL) was added. To the stirred suspension was added slowly a solution of 5-benzyloxyindole (0.398 g, 1.78 mmol) in N,N-dimethylformamide (4 mL) at room temperature under a nitrogen atmosphere. To the stirred reaction mixture was added a solution of 1,1-dimethylethyl 5-(bromomethyl)-2-methylbenzoate (˜81 mol %) (0.477 g) in N,N-dimethylformamide (2 mL). The reaction mixture was allowed to stir overnight at room temperature under a nitrogen atmosphere. The reaction mixture was partitioned between water and ethyl acetate. The organic phase was separated, washed with saturated sodium chloride, dried over magnesium sulfate, filtered, and the filtrate was concentrated to give an orange oil. The crude product was purified by flash chromatography over silica with a hexanes:ethyl acetate gradient (100:0 to 90:10) to give 0.57 of 1,1-dimethylethyl 2-methyl-5-({5-[(phenylmethyl)oxy]-1H-indol-1-yl}methyl)benzoate as a clear colorless oil. ¹H NMR indicates that the 1,1-dimethylethyl 2-methyl-5-({5-[(phenylmethyl)oxy]-1H-indol-1-yl}methyl)benzoate is approximately 82 mol % pure. A solution of 1,1-dimethylethyl 2-methyl-5-({5-[(phenylmethyl)oxy]-1H-indol-1-yl}methyl)benzoate (˜82 mol %) (0.57 g) in ethyl acetate (20 mL) and ethanol (20 mL) was combined with 10% palladium on carbon (Degussa Type; 50% water by weight) (0.12 g). The flask containing the reaction mixture was evacuated and filled with nitrogen twice before evacuating and filling with hydrogen using a balloon. The reaction mixture was stirred for 24 h at room temperature under a hydrogen atmosphere. The reaction mixture was filtered through a pad of Celite® and the pad was washed with ethyl acetate. The filtrate was concentrated to give an oil. The oil was purified by reverse phase preparative HPLC using an acetonitrile:water gradient (30:70 to 100:0) and 0.05% trifluoroacetic acid as a modifier. Each of the six HPLC runs yielded one major UV-peak. The UV-peak of the first HPLC run was collected over three fractions. The three fractions from the first HPLC run were independently concentrated, partitioned between ethyl acetate and water, and the organic phases were dried over magnesium sulfate, filtered, and the filtrates were concentrated to give three oils. ¹H NMR analysis of the three oils indicated that all three fractions corresponding to the UV-peak of the first HPLC run contained impure product. The fractions of the remaining five HPLC runs were combined, concentrated, and partitioned between ethyl acetate and water. The organic phase was separated, dried over magnesium sulfate, filtered, and the filtrate was concentrated to give a total of 0.193 g of the crude product as an oil. The oil was purified on a Chiralpak AS-H chiral column using carbon dioxide:methanol (92:8) at 3000 psi and 40° C. at 2 mL/min flow rate to give 0.09 g (1.8% from o-toluic acid) of 1,1-dimethylethyl 5-[(5-hydroxy-1H-indol-1-yl)methyl]-2-methylbenzoate as an oil. ¹H NMR (400 MHz, CDCl₃): δ 7.67 (d, J=2 Hz, 1H), 7.12 (d, J=2 Hz, 1H), 7.09 (m, 2H), 7.04 (d, J=3 Hz, 1H), 6.97 (dd, J=8, 2 Hz, 1H), 6.74 (dd, J=9, 3 Hz, 1H), 6.41 (d, J=3 Hz, 1H), 5.25 (s, 2H), 4.44 (br s, 1H), 2.51 (s, 3H), 1.56 (s, 9H).

### 48d) 5-{[5-({[3-(2,6-Dichlorophenyl)-5-(1-methylethyl)-4-isoxazolyl]methyl}oxy)-1H-indol-1-yl]methyl}-2-methylbenzoic acid

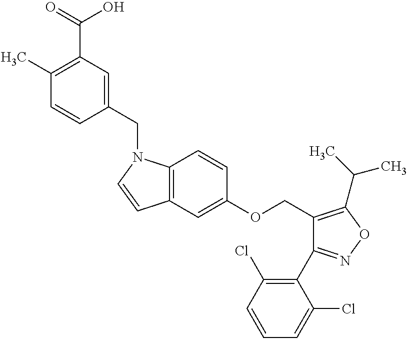

To a stirred mixture of 1,1-dimethylethyl 5-[(5-hydroxy-1H-indol-1-yl)methyl]-2-methylbenzoate (0.09 g, 0.27 mmol), [3-(2,6-dichlorophenyl)-5-(1-methylethyl)-4-isoxazolyl]methanol (prepared according to the general procedure described in Maloney, P. R., et al., 2000 J. Med. Chem. 43:2971-2974) (0.078 g, 0.27 mmol), triphenylphosphine (polystyrene-bound; 2.1 mmol/g) (0.181 g, 0.38 mmol), and dichloromethane (6 mL) was slowly added diisopropyl azodicarboxylate (0.075 mL, 0.38 mmol) at room temperature under a nitrogen atmosphere. The reaction mixture was stirred at room temperature for 3 days. The reaction mixture was filtered and the resin was washed with dichloromethane. The filtrate was concentrated to give a gold-yellow oil. The oil was purified by flash chromatography over silica with a hexanes:ethyl acetate gradient (100:0 to 75:25) to give an impure gold-yellow oil. The impure oil was purified by a second flash column over silica with a hexanes:ethyl acetate gradient (100:0 to 80:20) to give 0.155 g of impure 1,1-dimethylethyl 5-{[5-({[3-(2,6-dichlorophenyl)-5-(1-methylethyl)-4-isoxazolyl]methyl}oxy)-1H-indol-1-yl]methyl}-2-methylbenzoate as an oil. To a stirred ice-water cooled solution of the crude 1,1-dimethylethyl 5-{[5-({[3-(2,6-dichlorophenyl)-5-(1-methylethyl)-4-isoxazolyl]methyl}oxy)-1H-indol-1-yl]methyl}-2-methylbenzoate (0.155 g) in dichloromethane (2 mL) was added trifluoroacetic acid (1 mL). The reaction mixture was stirred at 0° C. for 1 h. AP-LCMS indicated that the reaction was incomplete. The reaction mixture was allowed to continue stirring as the ice-water bath was allowed to slowly warm at room temperature for 2 h. The reaction mixture was concentrated and the residue was dissolved in toluene. The toluene was removed in vacuo to give a brown oil. The oil was purified by flash chromatography over silica with a hexanes:ethyl acetate gradient (75:25 to 50:50) to give impure product. The impure product was purified by flash chromatography over silica with methylene chloride:methanol (100:0 to 99:1 to 98:2) to give impure product. The impure product was purified by reverse phase preparative HPLC with an acetonitrile:water gradient (50:50 to 100:0) and 0.05% trifluoroacetic acid as a modifier to give 0.0031 g (2.1% from 1,1-dimethylethyl 5-[(5-hydroxy-1H-indol-1-yl)methyl]-2-methylbenzoate) of 5-{[5-({[3-(2,6-dichlorophenyl)-5-(1-methylethyl)-4-isoxazolyl]methyl}oxy)-1H-indol-1-yl]methyl}-2-methylbenzoic acid as a white solid.

¹H NMR (400 MHz, DMSO-d₆): δ 12.81 (br s, 1H), 7.59 (m, 3H), 7.51 (dd, J=9, 7 Hz, 1H), 7.42 (d, J=3 Hz, 1H), 7.20 (d, J=9 Hz, 1H), 7.18 (s, 2H), 6.93 (d, J=2 Hz, 1H), 6.50 (dd, J=9, 2 Hz, 1H), 6.30 (d, J=3 Hz, 1H), 5.33 (s, 2H), 4.73 (s, 2H), 3.36 (septet, J=7 Hz, 1H), 2.42 (s, 3H), 1.25 (d, J=7 Hz, 6H). ES-LCMS m/z 549 (M+H)⁺.

### Example 49

### 6-{[5-({[3-(2,6-Dichlorophenyl)-5-(1-methylethyl)-4-isoxazolyl]methyl}oxy)-1H-indol-1-yl]methyl}-2-pyridinecarboxylic acid

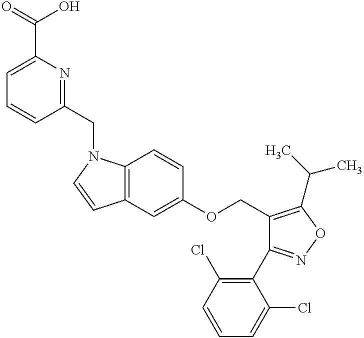

### 49a) Methyl 6-{[5-({[3-(2,6-dichlorophenyl)-5-(1-methylethyl)-4-isoxazolyl]methyl}oxy)-1H-indol-1-yl]methyl}-2-pyridinecarboxylate

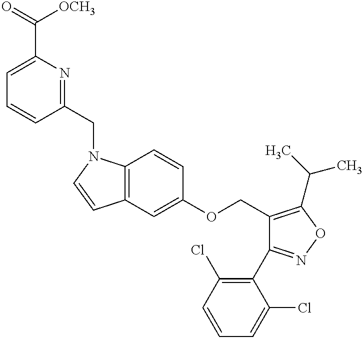

Sodium hydride (60% dispersion in oil) (0.027 g, 0.675 mmol) was washed with hexanes and N,N-dimethylformamide (1 mL) was added. To the stirred sodium hydride suspension was slowly added a solution of 5-({[3-(2,6-dichlorophenyl)-5-(1-methylethyl)-4-isoxazolyl]methyl}oxy)-1H-indole (prepared according to the general procedure described for Example 46a) (0.224 g, 0.56 mmol) in N,N-dimethylformamide (3 mL) at room temperature under a nitrogen atmosphere. The reaction mixture was stirred for 5 min. A solution of methyl 6-(bromomethyl)-2-pyridinecarboxylate (0.131 g, 0.57 mmol) in N,N-dimethylformamide (1 mL) was slowly added to the reaction mixture. The reaction mixture was stirred for 3.5 h at room temperature under a nitrogen atmosphere. The reaction mixture was partitioned between ethyl acetate and water. The layers were separated and the aqueous phase was extracted with ethyl acetate. The organic extracts were combined, washed with water followed by saturated sodium chloride, dried over magnesium sulfate, filtered, and the filtrate was concentrated to give an oil. The crude product was purified by flash chromatography over silica with a hexanes:ethyl acetate gradient (100:0 to 50:50) to give 0.10 g (32%) of methyl 6-{[5-({[3-(2,6-dichlorophenyl)-5-(1-methylethyl)-4-isoxazolyl]methyl}oxy)-1H-indol-1-yl]methyl}-2-pyridinecarboxylate as a white amorphous solid. ¹H NMR (400 MHz, DMSO-d₆): δ 7.90 (d, J=8 Hz, 1H), 7.84 (t, J=8 Hz, 1H), 7.59 (m, 2H), 7.51 (dd, J=9, 7 Hz, 1H), 7.43 (d, J=3 Hz, 1H), 7.17 (d, J=9 Hz, 1H), 6.96 (d, J=2 Hz, 1H), 6.84 (d, J=8 Hz, 1H), 6.50 (dd, J=9, 2 Hz, 1H), 6.36 (d, J=3 Hz, 1H), 5.50 (s, 2H), 4.74 (s, 2H), 3.87 (s, 3H), 3.37 (septet, J=7 Hz, 1H), 1.26 (d, J=7 Hz, 6H). ES-LCMS m/z 550 (M+H)⁺.

### 49b) 6-{[5-({[3-(2,6-Dichlorophenyl)-5-(1-methylethyl)-4-isoxazolyl]methyl}oxy)-1H-indol-1-yl]methyl}-2-pyridinecarboxylic acid

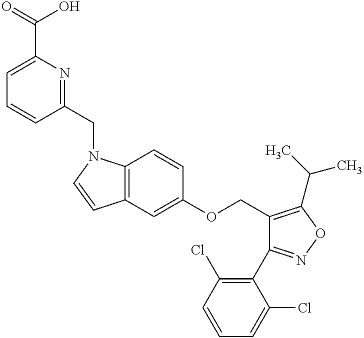

To a stirred solution of methyl 6-{[5-({[3-(2,6-dichlorophenyl)-5-(1-methylethyl)-4-isoxazolyl]methyl}oxy)-1H-indol-1-yl]methyl}-2-pyridinecarboxylate (0.10 g, 0.18 mmol) in tetrahydrofuran (4 mL) and methanol (2 mL) was added sodium hydroxide (1 N) (0.36 mL, 0.36 mmol) at room temperature under a nitrogen atmosphere. The reaction mixture was stirred for 19 h. The methanol and tetrahydrofuran were removed in vacuo and water (3 mL) was added to the aqueous mixture. The pH of the aqueous mixture was adjusted to approximately 3 (litmus paper) with 10% citric acid. The acidic aqueous mixture was extracted with ethyl acetate. The layers were separated and the aqueous phase was extracted with ethyl acetate. The organic extracts were combined, washed with water (5 mL), followed by saturated sodium chloride, dried over magnesium sulfate, filtered, and the filtrate concentrated to give an amorphous solid. The solid was dried under high vacuum at 50° C. to give 0.092 g (95%) of 6-{[5-({[3-(2,6-dichlorophenyl)-5-(1-methylethyl)-4-isoxazolyl]methyl}oxy)-1H-indol-1-yl]methyl}-2-pyridinecarboxylic acid as a pale yellow amorphous solid. ¹H NMR (400 MHz, DMSO-d₆): δ 13.22 (br s, 1H), 7.87 (d, J=8 Hz, 1H), 7.81 (t, J=8 Hz, 1H), 7.59 (m, 2H), 7.51 (dd, J=9, 7 Hz, 1H), 7.45 (d, J=3 Hz, 1H), 7.20 (d, J=9 Hz, 1H), 6.96 (d, J=2 Hz, 1H), 6.84 (d, J=8 Hz, 1H), 6.50 (dd, 9, 2 Hz, 1H), 6.35 (d, J=3 Hz, 1H), 5.49 (s, 2H), 4.74 (s, 2H), 3.37 (septet, J=7 Hz, 1H), 1.26 (d, J=7 Hz, 6H). HRMS C₂₈H₂₄Cl₂N₃O₄ m/z 536.1144 (M+H)⁺_{Cal}; 536.1134 (M+H)⁺_{Obs}.

### Example 50

### 3-{[5-({[3-(2,6-Dichlorophenyl)-5-(1-methylethyl)-4-isoxazolyl]methyl}oxy)-1H-indol-3-yl]methyl}benzoic acid

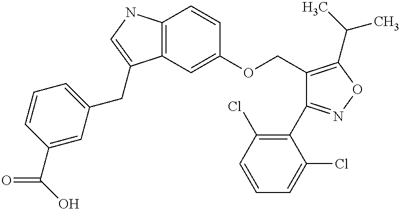

### 50a) Methyl 3-{[5-({[3-(2,6-dichlorophenyl)-5-(1-methylethyl)-4-isoxazolyl]methyl}oxy)-1H-indol-3-yl]methyl}benzoate

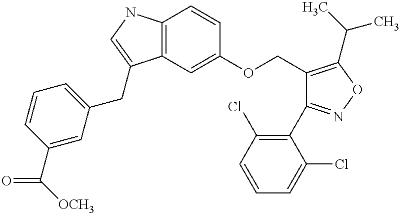

To a stirred mixture of 5-({[3-(2,6-dichlorophenyl)-5-(1-methylethyl)-4-isoxazolyl]methyl}oxy)-1H-indole (prepared according to the general procedure described for Example 46a) (0.504 g, 1.26 mmol), zinc (II) trifluoromethanesulfonate (0.277 g, 0.76 mmol), tetrabutylammonium iodide (0.239 g, 0.65 mmol), and toluene (5 mL), was added N,N-diisopropylethylamine (0.24 mL, 1.38 mmol) at room temperature under a nitrogen atmosphere. The reaction mixture was stirred for approximately 20 min and methyl-3-bromomethylbenzoate (0.148 g, 0.65 mmol) was added to the reaction mixture. The reaction mixture was allowed to stir at room temperature for 23 h. To the reaction mixture were added saturated ammonium chloride, water, and ethyl acetate. The layers were separated and the organic phase was washed with water, followed by saturated sodium chloride, dried over magnesium sulfate, filtered, and the filtrate was concentrated to give an oil. The crude product was purified by flash chromatography over silica with a hexanes:ethyl acetate gradient (100:0 to 50:50) to give 0.212 g (59%) of methyl 3-{[5-({[3-(2,6-dichlorophenyl)-5-(1-methylethyl)-4-isoxazolyl]methyl}oxy)-1H-indol-3-yl]methyl}benzoate as a viscous oil. ¹H NMR (400 MHz, CDCl₃): δ 7.96 (s, 1H), 7.87 (m, 2H), 7.42 (d, J=8 Hz, 1H), 7.37-7.27 (m, 4H), 7.18 (d, J=9 Hz, 1H), 6.87 (d, J=2 Hz, 1H), 6.80 (d, J=2 Hz, 1H), 6.69 (dd, J=9, 2 Hz, 1H), 4.69 (s, 2H), 4.06 (s, 2H), 3.89 (s, 3H), 3.27 (septet, J=7 Hz, 1H), 1.36 (d, J=7 Hz, 6H). ES-LCMS m/z 549 (M+H)⁺.

### 50b) 3-{[5-({[3-(2,6-Dichlorophenyl)-5-(1-methylethyl)-4-isoxazolyl]methyl}oxy)-1H-indol-3-yl]methyl}benzoic acid

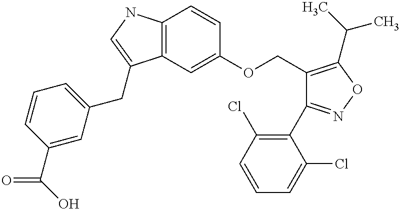

To a stirred solution of methyl 3-{[5-({[3-(2,6-dichlorophenyl)-5-(1-methylethyl)-4-isoxazolyl]methyl}oxy)-1H-indol-3-yl]methyl}benzoate (0.21 g, 0.38 mmol) in tetrahydrofuran (6 mL) and methanol (3 mL) was added sodium hydroxide (1 N) (0.90 mL, 0.90 mmol) at room temperature under a nitrogen atmosphere. The reaction mixture was stirred for 25 h. To the reaction mixture was added sodium hydroxide (1 N) (0.5 mL, 0.5 mmol). The reaction mixture was stirred for 31 h at room temperature under a nitrogen atmosphere. The tetrahydrofuran and methanol were removed from the reaction mixture in vacuo and to the resulting aqueous mixture was added water (5 mL). The pH of the aqueous mixture was adjusted to approximately 2 (litmus paper) with 10% citric acid. The acidic aqueous phase was extracted with ethyl acetate (10 mL). The layers were separated and the aqueous phase was extracted with ethyl acetate. The organic extracts were combined, washed with water (5 mL), followed by saturated sodium chloride (5 mL), dried over magnesium sulfate, filtered, and the filtrate concentrated to give an amorphous solid. The crude product was purified by flash chromatography over silica with a dichloromethane:methanol gradient (100:0 to 98:2) to give 0.136 g (67%) of 3-{[5-({[3-(2,6-dichlorophenyl)-5-(1-methylethyl)-4-isoxazolyl]methyl}oxy)-1H-indol-3-yl]methyl}benzoic acid after drying as an off-white amorphous solid. ¹H NMR (400 MHz, DMSO-d₆): δ 12.83 (s, 1H), 10.70 (s, 1H), 7.79 (s, 1H), 7.72 (d, J=8 Hz, 1H), 7.57 (m, 2H), 7.49 (m, 2H), 7.36 (t, J=8 Hz, 1H), 7.12 (d, J=9 Hz, 1H), 7.10 (d, J=2 Hz, 1H), 6.75 (d, J=2 Hz, 1H), 6.49 (dd, J=9, 2 Hz, 1H), 4.68 (s, 2H), 3.99 (s, 2H), 3.33 (septet, J=7 Hz, 1H), 1.22 (d, J=7 Hz, 6H). HRMS C₂₉H₂₅Cl₂N₂O₄ m/z 535.1191 (M+H)⁺_{Cal}; 535.1190 (M+H)⁺_{Obs}.

### Example 51

### 2-{[5-({[3-(2,6-Dichlorophenyl)-5-(1-methylethyl)-4-isoxazolyl]methyl}oxy)-1H-indol-1-yl]methyl}-1,3-thiazole-4-carboxylic acid

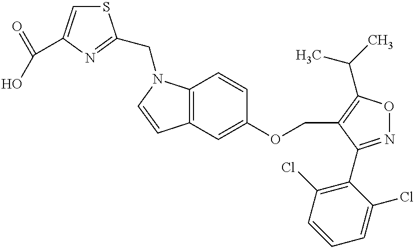

### 51a) Methyl 2-(dichloromethyl)-4,5-dihydro-1,3-thiazole-4-carboxylate

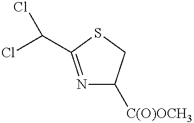

This compound was prepared according to the general procedure described by S. A. Hermitage, et al., (2001 Org. Proc. Res. Devel. 5:37-44). To methanol (10 mL) was added sodium methoxide (25% by weight in methanol) (1.15 mL, 5 mmol). The sodium methoxide solution was cooled to −15° C. (bath temperature) in an acetone/ice bath and dichloroacetonitrile (4 mL, 50 mmol) was added slowly, dropwise, over a 35 min period with stirring under a nitrogen atmosphere. The reaction mixture was stirred with cooling for 20 min. To the cold reaction mixture was added L-cysteine methyl ester hydrochloride (9.8 g, 57 mmol) via a powder addition funnel, followed by methanol (8 mL). The stirred reaction mixture was allowed to slowly warm to room temperature overnight. To the reaction mixture was added water (17 mL) and dichloromethane (32 mL). The mixture was transferred to a separatory funnel and the layers were separated. The aqueous phase was extracted with dichloromethane (30 mL). The organic extracts were combined, dried over magnesium sulfate, filtered, and the filtrate was concentrated to give 9.4 g (82% crude from dichloroacetonitrile) of methyl 2-(dichloromethyl)-4,5-dihydro-1,3-thiazole-4-carboxylate as an oil. ¹H NMR (400 MHz, CDCl₃): δ 6.49 (s, 1H), 5.18 (m, 1H), 3.83 (s, 3H), 3.64-3.77 (m, 2H).

### 51b) Methyl 2-(chloromethyl)-1,3-thiazole-4-carboxylate

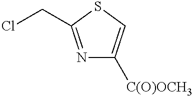

This compound was prepared according to the general procedure described by S. A. Hermitage, et al., (2001 Org. Proc. Res. Devel. 5:37-44) with modification. To an acetone/ice cooled solution of methyl 2-(dichloromethyl)-4,5-dihydro-1,3-thiazole-4-carboxylate (9.4 g) in methanol (10 mL) between −10° C. and −15° C. was slowly added dropwise over 45 min a solution of sodium methoxide (25% wt/wt in methanol) (9.4 mL, 41 mmol) with stirring under a nitrogen atmosphere. The acetone/ice bath was removed and the reaction mixture was allowed to stir at room temperature for 2 h. To the reaction mixture was added dichloromethane (30 mL) and water (17 mL). The mixture was transferred to a reparatory funnel and the layers were separated. The aqueous phase was extracted with dichloromethane (17 mL). The organic extracts were combined and the solution was concentrated to give a brown oil which quickly solidified to a brown solid. The crude product was purified by flash chromatography over silica with hexanes:ethyl acetate gradient (75:25 to 50:50) to give 5.9 g (62% from dichloroacetonitrile) of methyl 2-(chloromethyl)-1,3-thiazole-4-carboxylate as a pale yellow solid. ¹H NMR (400 MHz, CDCl₃): δ 8.22 (s, 1H), 4.88 (s, 2H), 3.95 (s, 3H).

### 51c) Methyl 2-{[5-({[3-(2,6-dichlorophenyl)-5-(1-methylethyl)-4-isoxazolyl]methyl}oxy)-1H-indol-1-yl]methyl}-1,3-thiazole-4-carboxylate

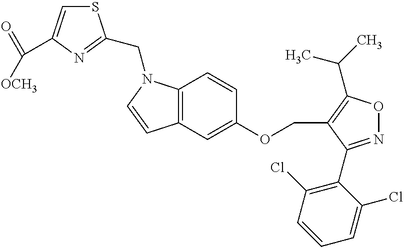

Sodium hydride (60% dispersion in oil) (0.089 g, 2.2 mmol) was washed with hexanes and N,N-dimethylformamide (2 mL) was added. To the stirred suspension of sodium hydride was slowly added, dropwise, a solution of 5-({[3-(2,6-dichlorophenyl)-5-(1-methylethyl)-4-isoxazolyl]methyl}oxy)-1H-indole (prepared according to the general procedure described for Example 46a) (0.7 g, 1.7 mmol) in N,N-dimethylformamide (4 mL) at room temperature under a nitrogen atmosphere. The addition funnel was rinsed with N,N-dimethylformamide (1 mL) and the solution was added to the reaction mixture. The reaction mixture was stirred for 10 min. To the reaction mixture was slowly added dropwise, a solution of methyl 2-(chloromethyl)-1,3-thiazole-4-carboxylate (0.35 g, 1.8 mmol) in N,N-dimethylformamide (3 mL). The reaction mixture was stirred overnight at room temperature under a nitrogen atmosphere. The reaction mixture was quenched with water and the aqueous mixture was partitioned between water and ethyl acetate. The organic phase was separated and the aqueous phase was extracted with ethyl acetate. The second extraction with ethyl acetate resulted in an emulsion. The emulsion was allowed to stand at room temperature over 3 days. The layers were separated, and the organic extracts were combined, dried over magnesium sulfate, filtered, and the filtrate was concentrated to give a brown-orange liquid. The crude product was purified by flash chromatography over silica with a hexanes:ethyl acetate gradient (100:0 to 50:50) to give 0.491 g of an orange liquid. ¹H NMR indicated that the product contained N,N-dimethylformamide and dichloromethane. The product was approximately 64% methyl 2-{[5-({[3-(2,6-dichlorophenyl)-5-(1-methylethyl)-4-isoxazolyl]methyl}oxy)-1H-indol-1-yl]methyl}-1,3-thiazole-4-carboxylate by weight and was taken on without further purification. ¹H NMR (400 MHz, CDCl₃): δ 8.04 (s, 1H), 7.38 (m, 2H), 7.30 (dd, J=9, 7 Hz, 1H), 7.14 (d, J=3 Hz, 1H), 7.11 (d, J=9 Hz, 1H), 6.98 (d, J=2 Hz, 1H), 6.71 (dd, J=9, 2 Hz, 1H), 6.46 (d, J=3 Hz, 1H), 5.59 (s, 2H), 4.74 (s, 2H), 3.96 (s, 3H), 3.31 (septet, J=7 Hz, 1H), 1.38 (d, J=7 Hz, 6H). ES-LCMS, m/z 556 (M+H)⁺.

### 51d) 2-{[5-({[3-(2,6-Dichlorophenyl)-5-(1-methylethyl)-4-isoxazolyl]methyl}oxy)-1H-indol-1-yl]methyl}-1,3-thiazole-4-carboxylic acid

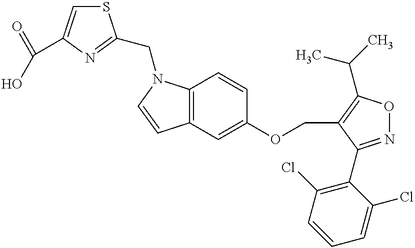

To a stirred solution of methyl 2-{[5-({[3-(2,6-dichlorophenyl)-5-(1-methylethyl)-4-isoxazolyl]methyl}oxy)-1H-indol-1-yl]methyl}-1,3-thiazole-4-carboxylate (64%) (0.49 g, 0.56 mmol) in tetrahydrofuran (10 mL) and methanol (5 mL) was added sodium hydroxide (1 N) (2.2 mL, 2.2 mmol). The reaction mixture was stirred at room temperature under a nitrogen atmosphere for 17 h. The tetrahydrofuran and methanol were removed in vacuo and water (5 mL) was added to the aqueous residue. The pH of the aqueous mixture was adjusted to approximately 2-3 (litmus paper) with 10% citric acid. The acidic aqueous mixture was extracted with ethyl acetate (10 mL). The layers were separated and the organic phase was washed with water (5 mL), followed by saturated sodium chloride (6 mL), dried over magnesium sulfate, filtered, and the filtrate was concentrated and dried to give 0.233 g (76%) of 2-{[5-({[3-(2,6-dichlorophenyl)-5-(1-methylethyl)-4-isoxazolyl]methyl}oxy)-1H-indol-1-yl]methyl}-1,3-thiazole-4-carboxylic acid as a pale yellow amorphous solid. ¹H NMR (400 MHz, DMSO-d₆): δ 13.02 (s, 1H), 8.27 (s, 1H), 7.60 (m, 2H), 7.51 (dd, J=9, 7 Hz, 1H), 7.44 (d, J=2 Hz, 1H), 7.28 (d, J=9 Hz, 1H), 6.96 (d, J=2 Hz, 1H), 6.55 (dd, J=9, 2 Hz, 1H), 6.36 (d, J=2 Hz, 1H), 5.71 (d, 2H), 4.75 (s, 2H), 3.38 (septet, J=7 Hz, 1H), 1.26 (d, J=7 Hz, 6H). HRMS C₂₆H₂₂Cl₂N₃O₄S m/z 542.0708 (M+H)⁺_{Cal}; 542.0703 (M+H)⁺_{Obs}.

### Example 52

### 3-{[6-({[3-(2,6-Dichlorophenyl)-5-(1-methylethyl)-4-isoxazolyl]methyl}oxy)-1H-indol-3-yl]methyl}benzoic acid

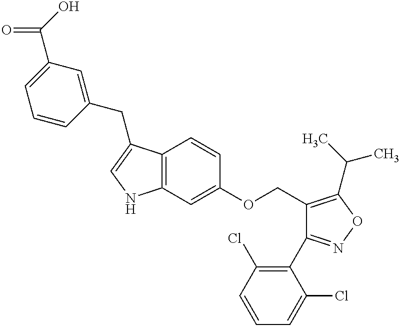

### 52a) Methyl 3-({6-[(phenylmethyl)oxy]-1H-indol-3-yl}methyl)benzoate

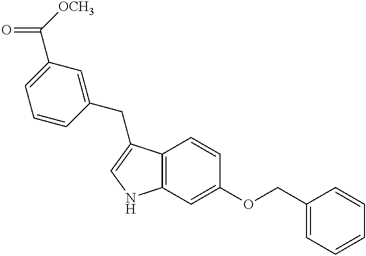

To a stirred mixture of 6-benzyloxyindole (0.435 g, 1.95 mmol), zinc(II) trifluoromethanesulfonate (0.448 g, 1.23 mmol), tetrabutylammonium iodide (0.362 g, 0.98 mmol), and toluene (8 mL) was added, dropwise, N,N-diisopropylethylamine (0.36 mL, 2.07 mmol). The reaction mixture was stirred at room temperature under a nitrogen atmosphere for 20 min. To the reaction mixture was added methyl-3-bromomethyl benzoate (0.229 g, 1 mmol) and the reaction mixture was stirred at room temperature overnight. To the reaction mixture was added water, ethyl acetate, and saturated ammonium chloride. The layers were separated and the organic phase was washed with water, followed by saturated sodium chloride, dried over magnesium sulfate, filtered, and the filtrate was concentrated to give an orange oil. The crude product was purified by flash chromatography over silica with a hexanes:ethyl acetate gradient (100:0 to 60:40) to give 0.195 g (53%) of methyl 3-({6-[(phenylmethyl)oxy]-1H-indol-3-yl}methyl)benzoate. ¹H NMR (400 MHz, DMSO-d₆): δ 10.66 (s, 1H), 7.81 (s, 1H), 7.73 (d, J=8 Hz, 1H), 7.55 (d, J=8 Hz, 1H), 7.43-7.33 (m, 5H), 7.28 (m, 1H), 7.21 (d, J=9 Hz, 1H), 7.03 (d, J=2 Hz, 1H), 6.88 (d, J=2 Hz, 1H), 6.64 (dd, J=9, 2 Hz, 1H), 5.06 (s, 2H), 4.03 (s, 2H), 3.78 (s, 3H).

### 52b) Methyl 3-[(6-hydroxy-1H-indol-3-yl)methyl]benzoate

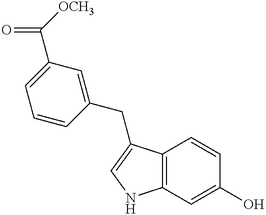

A solution of methyl 3-({6-[(phenylmethyl)oxy]-1H-indol-3-yl}methyl)benzoate (0.18 g, 0.49 mmol) in ethyl acetate (8 mL) and ethanol (4 mL) was added to 10% palladium on carbon (Degussa Type; 50% water by wt.) (0.046 g). The parr bottle was evacuated and filled with nitrogen twice, then evacuated and filled with hydrogen to 50 psi. The reaction mixture was shaken in the parr bottle under hydrogen for 24 h. The reaction mixture was filtered through a pad of Celite® and the pad was washed with ethyl acetate followed by ethanol. The filtrate was concentrated and the crude product was dissolved in ethyl acetate. The solution was filtered through a pad of Celite® and the pad was washed with ethyl acetate. The filtrate was concentrated to give an olive-green oil. The crude product was stored in the freezer for 2 days. The crude product was dissolved in dichloromethane and the solution was concentrated. The crude product was dissolved in dichloromethane and the solution was concentrated to give 0.144 g (104%) of crude methyl 3-[(6-hydroxy-1H-indol-3-yl)methyl]benzoate as an olive-green amorphous solid. The product was used without further purification. ¹H NMR (400 MHz, DMSO-d₆): δ 10.44 (s, 1H), 8.82 (s, 1H), 7.80 (s, 1H), 7.72 (d, J=8 Hz, 1H), 7.54 (d, J=8 Hz, 1H), 7.38 (t, J=8 Hz, 1H), 7.09 (d, J=8 Hz, 1H), 6.92 (m, 1H), 6.66 (d, J=2 Hz, 1H), 6.42 (dd, J=8, 2 Hz, 1H), 4.00 (s, 2H), 3.78 (s, 3H). ES-LCMS m/z 282 (M+H)⁺.

### 52c) 3-{[6-({[3-(2,6-Dichlorophenyl)-5-(1-methylethyl)-4-isoxazolyl]methyl}oxy)-1H-indol-3-yl]methyl}benzoic acid

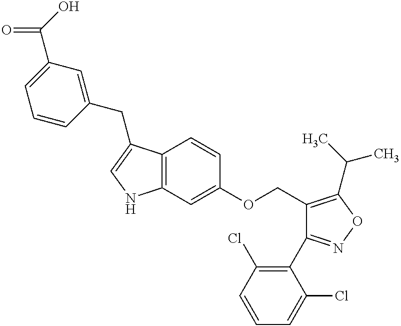

To a stirred mixture of methyl 3-[(6-hydroxy-1H-indol-3-yl)methyl]benzoate (0.14 g, 0.5 mmol), [3-(2,6-dichlorophenyl)-5-(1-methylethyl)-4-isoxazolyl]methanol (prepared according to the general procedure described in Maloney, P. R., et al., 2000 J. Med. Chem. 43:2971-2974) (0.154 g, 0.54 mmol), triphenylphosphine (polystyrene resin bound; 3 mmol/g) (0.173 g, 0.52 mmol), and dichloromethane (10 mL) was added, dropwise, diisopropyl azodicarboxylate (0.10 mL, 0.51 mmol), followed by dichloromethane (2 mL). The reaction mixture was allowed to stir overnight at room temperature under a nitrogen atmosphere. The reaction mixture was filtered and the resin was washed with dichloromethane. The filtrate was concentrated to give a yellow-green oil. The crude product was partially purified by flash chromatography over silica with a hexanes:ethyl acetate gradient (100:0 to 50:50) to give 0.183 g of crude methyl 3-{[6-({[3-(2,6-dichlorophenyl)-5-(1-methylethyl)-4-isoxazolyl]methyl}oxy)-1H-indol-3-yl]methyl}benzoate as a yellow oil. The product was used without further purification. To a stirred solution of crude methyl 3-{[6-({[3-(2,6-dichlorophenyl)-5-(1-methylethyl)-4-isoxazolyl]methyl}oxy)-1H-indol-3-yl]methyl}benzoate (0.18 g) in tetrahydrofuran (6 mL) and methanol (3 mL) was added 1N sodium hydroxide (1.3 mL, 1.3 mmol). The reaction mixture was stirred overnight at room temperature under a nitrogen atmosphere. The tetrahydrofuran and methanol were removed in vacuo and water (5 mL) was added to the aqueous residue. The pH of the aqueous mixture was adjusted to approximately 2 (litmus paper) with 10% citric acid. The acidic aqueous mixture was extracted with ethyl acetate. The organic phase was separated, washed with water (5 mL), followed by saturated sodium chloride, dried over magnesium sulfate, filtered, and the filtrate concentrated to give a yellow oil. The crude product was purified by flash chromatography over silica with a hexanes:ethyl acetate gradient (100:0 to 50:50), followed by reverse phase preparative HPLC with an acetonitrile:water gradient (50:50 to 100:0) using 0.05% trifluoroacetic acid as a modifier to give 0.027 g (10% from methyl 3-({6-[(phenylmethyl)oxy]-1H-indol-3-yl}methyl)benzoate) of 3-{[6-({[3-(2,6-dichlorophenyl)-5-(1-methylethyl)-4-isoxazolyl]methyl}oxy)-1H-indol-3-yl]methyl}benzoic acid as a white solid. ¹H NMR (400 MHz, DMSO-d₆): δ 12.82 (br s, 1H), 10.61 (br s, 1H), 7.76 (s, 1H), 7.70 (d, J=8 Hz, 1H), 7.57 (m, 2H), 7.49 (m, 2H), 7.34 (t, J=8 Hz, 1H), 7.13 (d, J=9 Hz, 1H), 7.02 (m, 1H), 6.73 (d, J=2 Hz, 1H), 6.36 (dd, J=9, 2 Hz, 1H), 4.73 (s, 2H), 4.00 (s, 2H), 3.38 (septet, J=7 Hz, 1H), 1.27 (d, J=7 Hz, 6H). HRMS C₂₉H₂₅Cl₂N₂O₄ m/z 535.1191 (M+H)⁺_{Cal}; 535.1195 (M+H)⁺_{Obs}.

### Example 53

### (3R)-1-{[5-({[3-(2,6-Dichlorophenyl)-5-(1-methylethyl)-4-isoxazolyl]methyl}oxy)-1H-indol-1-yl]carbonyl}-3-pyrrolidinecarboxylic acid

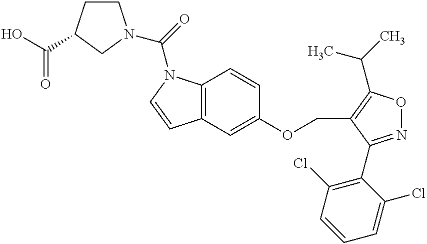

### 53a) Methyl (3R)-3-pyrrolidinecarboxylate hydrochloride

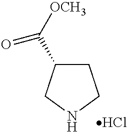

To a stirred solution of (3R)-1-{[(1,1-dimethylethyl)oxy]carbonyl}-3-pyrrolidinecarboxylic (1.03 g, 4.8 mmol) in methanol (25 mL) was added, dropwise, thionyl chloride (1 mL, 13.7 mmol) at room temperature under a nitrogen atmosphere. The reaction mixture was refluxed for 2 h and allowed to cool at room temperature. The reaction mixture was concentrated and the crude product was dissolved in dichloromethane. The solution was concentrated to give 0.792 g (100%) of methyl (3R)-3-pyrrolidinecarboxylate hydrochloride as an off-white solid. ¹H NMR (400 MHz, CDCl₃): δ 9.93 (m, 2H), 3.74 (s, 3H), 3.58 (br s, 2H), 3.42 (br s, 2H), 3.26 (m, 1H), 2.36-2.25 (m, 2H).

### 53b) Methyl (3R)-1-(1H-imidazol-1-ylcarbonyl)-3-pyrrolidinecarboxylate

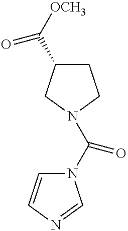

To a stirred turbid mixture of methyl (3R)-3-pyrrolidinecarboxylate hydrochloride (0.78 g, 4.71 mmol) and 1,1′-carbonyldiimidazole (0.83 g, 5.1 mmol) in dichloromethane (15 mL) was added triethylamine (1.4 mL, 10 mmol), dropwise, at room temperature under a nitrogen atmosphere. The reaction mixture was stirred at room temperature for 19 h. The reaction mixture was washed with water (2×10 mL). The organic phase was dried over magnesium sulfate, filtered, and the filtrate was concentrated to give a pale yellow oil which solidified upon standing to give 0.89 g (85%) of methyl (3R)-1-(1H-imidazol-1-ylcarbonyl)-3-pyrrolidinecarboxylate as an off-white solid. ¹H NMR (400 MHz, CDCl₃): δ 8.43 (s, 1H), 7.42 (s, 1H), 7.20 (s, 1H), 3.94-3.85 (m, 2H), 3.81-3.70 (m, 5H), 3.21 (m, 1H), 2.30 (m, 2H).

### 53c) Methyl (3R)-1-{[5-({[3-(2,6-dichlorophenyl)-5-(1-methylethyl)-4-isoxazolyl]methyl}oxy)-1H-indol-1-yl]carbonyl}-3-pyrrolidinecarboxylate

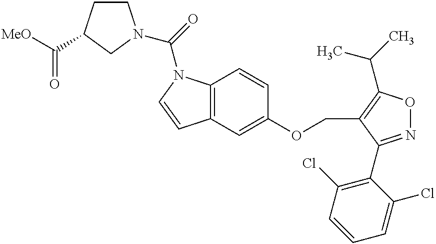

To a stirred suspension of sodium hydride (60% oil dispersion) (0.084 g, 2.1 mmol) in N,N-dimethylformamide (4 mL) was slowly added a solution of 5-({[3-(2,6-dichlorophenyl)-5-(1-methylethyl)-4-isoxazolyl]methyl}oxy)-1H-indole (Example 46a) (0.647 g, 1.61 mmol) in N,N-dimethylformamide (4 mL) at room temperature under a nitrogen atmosphere. The reaction mixture was stirred at room temperature for 20 min, then cooled in an ice-water bath. To the stirred ice-water cooled reaction mixture was slowly added a solution of methyl (3R)-1-(1H-imidazol-1-ylcarbonyl)-3-pyrrolidinecarboxylate (0.416 g, 1.86 mmol) in N,N-dimethylformamide (4 mL). The reaction mixture was stirred with cooling for 10 min. The ice-water bath was removed and the reaction mixture was stirred for 2.5 h. The reaction mixture was partitioned between water and ethyl acetate. The organic phase was separated, washed with water, followed by saturated sodium chloride, dried over magnesium sulfate, filtered, and the filtrate was concentrated to give an orange oil. The crude product was purified by flash chromatography over silica with a hexanes:ethyl acetate gradient (100:0 to 50:50) to give 0.484 g (54%) of methyl (3R)-1-{[5-({[3-(2,6-dichlorophenyl)-5-(1-methylethyl)-4-isoxazolyl]methyl}oxy)-1H-indol-1-yl]carbonyl}-3-pyrrolidinecarboxylate as an amorphous solid. ¹H NMR indicates a small impurity is present. The material was used without further purification. ¹H NMR (400 MHz, CDCl₃): δ 7.66 (d, J=9 Hz, 1H), 7.39 (m, 2H), 7.32 (m, 2H), 6.91 (d, J=3 Hz, 1H), 6.77 (dd, J=9, 2 Hz, 1H), 6.45 (d, J=3 Hz, 1H), 4.75 (s, 2H), 3.85 (m, 2H), 3.76-3.63 (m, 2H), 3.73 (s, 3H), 3.34 (septet, J=7 Hz, 1H), 3.14 (quin, J=7 Hz, 1H), 2.23 (q, J=7 Hz, 2H), 1.40 (d, J=7 Hz, 6H). ES-LCMS m/z 556 (M+H)⁺.

### 53d) (3R)-1-{[5-({[3-(2,6-dichlorophenyl)-5-(1-methylethyl)-4-isoxazolyl]methyl}oxy)-1H-indol-1-yl]carbonyl}-3-pyrrolidinecarboxylic acid

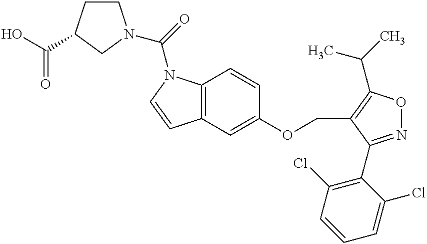

To a stirred solution of methyl (3R)-1-{[5-({[3-(2,6-dichlorophenyl)-5-(1-methylethyl)-4-isoxazolyl]methyl}oxy)-1H-indol-1-yl]carbonyl}-3-pyrrolidinecarboxylate (0.46 g, 0.827 mmol) in tetrahydrofuran (20 mL) and methanol (10 mL) was added sodium hydroxide (1 N) (0.92 mL, 0.92 mmol) at room temperature. The reaction mixture was stirred for 18 h at room temperature under a nitrogen atmosphere. The reaction mixture was concentrated and water (5 mL) was added to the residue. The pH of the aqueous mixture was adjusted to approximately 3 (litmus paper) with 10% citric acid. The acidic aqueous mixture was extracted with ethyl acetate. The organic extract was washed with water (10 mL), followed by saturated sodium chloride, dried over magnesium sulfate, filtered, and the filtrate concentrated to give a white amorphous solid. The crude product was purified by flash chromatography over silica with a dichloromethane:methanol gradient (100:0 to 98:2) to give 0.283 g (63%) of (3R)-1-{[5-({[3-(2,6-dichlorophenyl)-5-(1-methylethyl)-4-isoxazolyl]methyl}oxy)-1H-indol-1-yl]carbonyl}-3-pyrrolidinecarboxylic acid as a white amorphous solid. ¹H NMR (400 MHz, DMSO-d₆): δ 12.56 (br s, 1H), 7.60 (m, 3H), 7.52 (m, 2H), 6.98 (d, J=2 Hz, 1H), 6.62 (dd, J=9, 2 Hz, 1H), 6.47 (d, J=3 Hz, 1H), 4.79 (s, 2H), 3.66 (d, J=7 Hz, 2H), 3.54 (t, J=7 Hz, 2H), 3.42 (septet, J=7 Hz, 1H), 3.09 (quin, J=7 Hz, 1H), 2.17-1.99 (m, 2H), 1.29 (d, J=7 Hz, 6H). HRMS C₂₇H₂₆Cl₂N₃O₅ m/z 542.12440 (M+H)⁺_{Cal}; 542.12443 (M+H)⁺_{Obs}.

### Example 54

### 3-[5-({[3-(2,6-Dichlorophenyl)-5-(1-methylethyl)-4-isoxazolyl]methyl}oxy)-1-benzothien-2-yl]benzoic acid

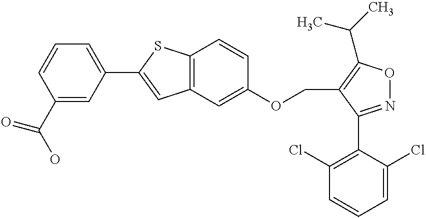

### 54a) 1-{[2,2-Bis(methyloxy)ethyl]thio}-4-bromobenzene

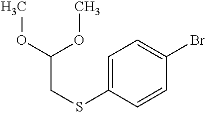

This compound was prepared according to the method described by T. Tsuri et al. (2003 J. Med. Chem. 46:2446-2455) with modification. To an ice-water cooled, stirred solution of sodium methoxide (25% wt/wt in methanol) (8 mL, 35 mmol) and methanol (16 mL) was added, portionwise, 4-bromothiophenol (6.0 g, 31.7 mmol) under a nitrogen atmosphere. To the cold reaction mixture was added, dropwise, bromoacetaldehyde dimethylacetal (4.2 mL, 35.5 mmol). The ice-water bath was removed and the reaction mixture was heated at reflux for 3.5 h. The oil bath was removed and the reaction mixture was allowed to cool at room temperature. The reaction mixture was concentrated and the residue was diluted with cold water. The aqueous mixture was extracted with diethyl ether. The organic extract was washed with water, followed by saturated sodium chloride, dried over magnesium sulfate, filtered, and the filtrate was concentrated to give a gold-yellow oil. The crude product was purified by vacuum distillation and collected, 3.6 g of 1-{[2,2-bis(methyloxy)ethyl]thio}-4-bromobenzene, between 92-94° C. at 0.2 mm as a pale yellow liquid. The orange liquid remaining in the distillation flask was determined by ¹H NMR to be 1-{[2,2-bis(methyloxy)ethyl]thio}-4-bromobenzene and provided another 2.5 g of the desired product for a total yield of 6.1 g (69%) of 1-{[2,2-bis(methyloxy)ethyl]thio}-4-bromobenzene. ¹H NMR (400 MHz, CDCl₃): δ 7.39 (d, J=9 Hz, 2H), 7.24 (d, J=9 Hz, 2H), 4.50 (t, J=6 Hz, 1H), 3.35 (s, 6H), 3.08 (d, J=Hz, 2H).

### 54b) 5-Bromo-1-benzothiophene

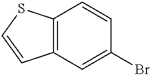

This compound was prepared according to the method described by T. Tsuri et al. (2003 J. Med. Chem. 46:2446-2455) with modification. Chlorobenzene (38 mL) and polyphosphoric acid (11.4 g) were combined and heated at reflux with stirring under a nitrogen atmosphere. To the reaction mixture was added, dropwise, a solution of 1-{[2,2-bis(methyloxy)ethyl]thio}-4-bromobenzene (6.1 g, 22 mmol,) in chlorobenzene (12 mL). The reaction mixture was heated at reflux overnight. The oil bath was removed and the reaction mixture was allowed to cool at room temperature. The supernatant was decanted and the remaining residue was washed twice with toluene.

The decanted solutions were combined and concentrated to give a dark brown-orange liquid. The crude product was purified by flash chromatography over silica with a hexanes:ethyl acetate gradient (100:0 to 98:2) to give 2.54 g of 5-bromo-1-benzothiophene. ¹H NMR indicates that a small impurity was present. An additional 0.50 g of 5-bromo-1-benzothiophene which lacked the aforementioned impurity was also obtained to give a total yield of 3.0 g (64%) of 5-bromo-1-benzothiophene. The two batches of 5-bromo-1-benzothiophene were combined and used without further purification. ¹H NMR (400 MHz, CDCl₃): δ 7.96 (s, 1H), 7.73 (d, J=9 Hz, 1H), 7.47 (d, J=5 Hz, 1H), 7.43 (d, J=9 Hz, 1H), 7.27 (d, J=5 Hz, 1H).

### 54c) 5-(Methyloxy)-1-benzothiophene

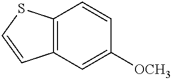

To 5-bromo-1-benzothiophene (3.0 g, 14.1 mmol) was added methanol (32 mL) followed by sodium methoxide (25% wt/wt in methanol) (32 mL, 140 mmol), and copper(I) bromide (0.201 g, 1.4 mmol). The stirred reaction mixture was heated at reflux under a nitrogen atmosphere for 1.5 h. The reaction mixture was allowed to cool at room temperature and copper powder (0.087 g, 1.37 mmol) was added. The reaction mixture was heated at reflux for 18 h and stirred at room temperature for 6 h. Approximately 1 mL of the reaction mixture was filtered and the filtrate was concentrated. The residue was partitioned between water and diethyl ether. The organic phase was separated, dried over magnesium sulfate, filtered, and the filtrate was concentrated to give an oil. ¹H NMR analysis of the oil indicated that the reaction was approximately 15% complete. The reaction mixture was heated at reflux for 5 days. The reaction mixture was allowed to cool at room temperature and concentrated. To the crude product was added ice-water, followed by diethyl ether. The organic phase was separated, washed with water, followed by saturated sodium chloride, dried over magnesium sulfate, filtered, and the filtrate was concentrated to give an orange liquid which quickly solidified to give a tan solid. The crude product was purified by flash chromatography over silica with a hexanes:ethyl acetate gradient (100:0 to 96:4) to give an oil which solidified upon standing. The oil was dissolved in dichloromethane and the solution was concentrated to give 1.48 (64%) of 5-(methyloxy)-1-benzothiophene as an oil which solidified to a white solid. ¹H NMR (400 MHz, CDCl₃): δ 7.73 (d, J=9 Hz, 1H), 7.44 (d, J=5 Hz, 1H), 7.27 (m, 2H), 7.00 (dd, J=9, 2 Hz, 1H), 3.87 (s, 3H).

### 54d) Ethyl 3-[5-(methyloxy)-1-benzothien-2-yl]benzoate

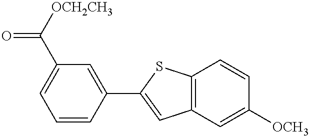

To a stirred dry ice/acetone cooled solution of 5-(methyloxy)-1-benzothiophene (1.48 g, 9 mmol) in tetrahydrofuran (50 mL) was slowly added n-butyl lithium (2.5M in hexanes) (4.0 mL, 10.4 mmol) under a nitrogen atmosphere. The solution was allowed to stir with cooling for 15 min. Triisopropyl borate (2.4 mL, 10.4 mmol) was added dropwise to the reaction mixture with stirring. The dry ice/acetone bath was removed and the reaction mixture was allowed to warm at room temperature for 1 h. The reaction mixture was partitioned between 1 N hydrochloric acid and ethyl acetate. The organic phase was separated, washed with water, dried over magnesium sulfate, filtered, and the filtrate was concentrated to give a pale tan solid. The solid was triturated with diethyl ether:hexane (1:1) to give 1.12 g of crude [5-(methyloxy)-1-benzothien-2-yl]boronic acid [ES-LCMS m/z 207 (M−H)⁻] as a pale gray solid. The material was used without further purification. Ethyl-3-iodobenzoate (1.3 mL, 7.7 mmol), [5-(methyloxy)-1-benzothien-2-yl]boronic acid (crude) (1.1 g), tetrakistriphenylphosphinepalladium(0) (0.246 g, 0.21 mmol), sodium carbonate (2 M) (6 mL, 12 mmol), and toluene (25 mL) were combined and the reaction mixture was heated at reflux under a nitrogen atmosphere for 3.5 h. The oil bath was removed and the reaction mixture was allowed to stand at room temperature overnight. The reaction mixture was heated at reflux for 3 h. The reaction mixture was allowed to cool at room temperature and partitioned between ethyl acetate and water. The organic phase was separated, dried over magnesium sulfate, filtered, and the filtrate was concentrated to give a liquid. The crude product was purified by flash chromatography over silica with a hexanes:ethyl acetate gradient (100:0 to 90:10) to give 0.21 g (7.4% from 5-(methyloxy)-1-benzothiophene) of ethyl 3-[5-(methyloxy)-1-benzothien-2-yl]benzoate as a gold-yellow oil. ¹H NMR (400 MHz, CDCl₃): δ 8.37 (s, 1H), 8.00 (d, J=8 Hz, 1H), 7.86 (d, J=8 Hz, 1H), 6.69 (d, J=9 Hz, 1H), 7.55 (s, 1H), 7.49 (t, J=8 Hz, 1H), 7.25 (s, 1H, overlapping CDCl₃), 6.99 (dd, J=9, 2 Hz, 1H), 4.42 (q, J=7 Hz, 2H), 3.88 (s, 3H), 1.43 (t, J=7 Hz, 3H).

### 54e) Ethyl 3-(5-hydroxy-1-benzothien-2-yl)benzoate

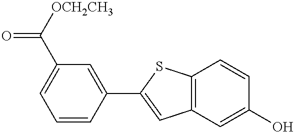

To an ice-water cooled solution of ethyl 3-[5-(methyloxy)-1-benzothien-2-yl]benzoate (0.21 g, 0.67 mmol) in dichloromethane (10 mL) was slowly added dropwise boron tribromide (1 M in dichloromethane) (2.8 mL, 2.8 mmol) with stirring under a nitrogen atmosphere. The reaction mixture was stirred with cooling for 2 h. The reaction mixture was poured into ice-water and the aqueous mixture was extracted with ethyl acetate. The organic phase was separated, dried over magnesium sulfate, filtered, and the filtrate was concentrated to give a tan solid. The crude product was purified by flash chromatography over silica with a hexanes:ethyl acetate gradient (100:0 to 90:10) to give 0.064 g (32%) of ethyl 3-(5-hydroxy-1-benzothien-2-yl)benzoate as a white solid. ¹H NMR (400 MHz, DMSO-d6): δ 9.50 (s, 1H), 8.21 (s, 1H), 8.01 (d, J=8 Hz, 1H), 7.92 (d, J=8 Hz, 1H), 7.82 (s, 1H), 7.74 (d, J=9 Hz, 1H), 7.61 (t, J=8 Hz, 1H), 7.19 (d, J=2 Hz, 1H), 6.87 (dd, J=9, 2 Hz, 1H), 4.35 (q, J=7 Hz, 2H), 1.34 (t, J=7 Hz, 3H). ES-LCMS m/z 297 (M−H)⁻.

### 54f) Ethyl 3-[5-({[3-(2,6-dichlorophenyl)-5-(1-methylethyl)-4-isoxazolyl]methyl}oxy)-1-benzothien-2-yl]benzoate

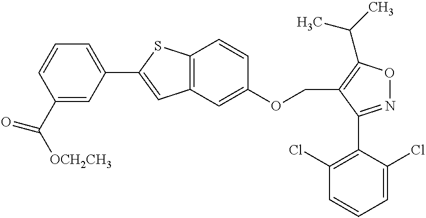

To a stirred mixture of ethyl 3-(5-hydroxy-1-benzothien-2-yl)benzoate (0.063 g, 0.21 mmol), [3-(2,6-dichlorophenyl)-5-(1-methylethyl)-4-isoxazolyl]methanol (prepared according to the general procedure described in Maloney, P. R., et al., 2000 J. Med. Chem. 43:2971-2974) (0.063 g, 0.22 mmol), triphenylphosphine (polystyrene resin bound; 3 mmol/g) (0.083 g, 0.25 mmol), and dichloromethane (10 mL) was added, dropwise, diisopropyl azodicarboxylate (0.050 mL, 0.25 mmol) at room temperature under a nitrogen atmosphere. After 14 h, dichloromethane (8 mL) was added to the reaction mixture and stirring was continued at room temperature for another 27 h. The reaction mixture was filtered and the resin was washed with dichloromethane. The filtrate was concentrated to give a gold-yellow oil. The crude product was purified by flash chromatography over silica with a hexanes:ethyl acetate gradient (100:0 to 80:20) to give 0.065 g of ethyl 3-[5-({[3-(2,6-dichlorophenyl)-5-(1-methylethyl)-4-isoxazolyl]methyl}oxy)-1-benzothien-2-yl]benzoate. ¹H NMR indicates that the product contains an impurity. The material was taken on without further purification. ¹H NMR (400 MHz, CDCl₃): δ 8.34 (s, 1H), 8.00 (d, J=8 Hz, 1H), 7.83 (d, J=8 Hz, 1H), 7.63 (d, J=9 Hz, 1H), 7.49 (m, 2H), 7.40 (m, 2H), 7.31 (dd, J=9, 7 Hz, 1H), 7.12 (d, J=8 Hz, 1H), 6.83 (dd, J=9, 2 Hz, 1H), 4.79 (s, 2H), 4.42 (q, J=7 Hz, 2H), 3.35 (septet, J=7 Hz, 1H), 1.42 (t, J=7 Hz, 3H), 1.42 (d, J=7 Hz, 6H). ES-LCMS m/z 566 (M+H)⁺.

### 54g) 3-[5-({[3-(2,6-Dichlorophenyl)-5-(1-methylethyl)-4-isoxazolyl]methyl}oxy)-1-benzothien-2-yl]benzoic acid

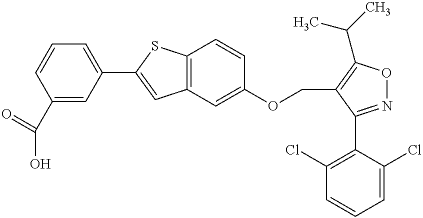

To a stirred solution of ethyl 3-[5-({[3-(2,6-dichlorophenyl)-5-(1-methylethyl)-4-isoxazolyl]methyl}oxy)-1-benzothien-2-yl]benzoate (0.065 g) in tetrahydrofuran (3 mL) and methanol (1.5 mL) was added sodium hydroxide (1 N) (0.13 mL 0.13 mmol). The reaction mixture was stirred for 8 h at room temperature under a nitrogen atmosphere. To the reaction mixture was added sodium hydroxide (1 N) (0.13 mL, 0.13 mmol). The reaction mixture was stirred at room temperature for 20 h. The reaction mixture was concentrated and water (5 mL) was added to the residue. The pH of the aqueous mixture was adjusted to approximately 4 (litmus paper) with 10% citric acid. The acidic aqueous phase was extracted with ethyl acetate. The organic phase was separated, dried over magnesium sulfate, filtered, and the filtrate was concentrated to give the crude product as an oil. The crude product was purified by flash chromatography over silica with a dichloromethane:methanol gradient (100:0 to 95:5) to give 0.051 g of impure product. A portion of this material (0.045 g) was purified by reverse phase preparative HPLC with an acetonitrile:water gradient (30:70 to 100:0) with 0.05% trifluoroacetic acid as a modifier to give 0.032 g (32% from ethyl 3-(5-hydroxy-1-benzothien-2-yl)benzoate) of 3-[5-({[3-(2,6-dichlorophenyl)-5-(1-methylethyl)-4-isoxazolyl]methyl}oxy)-1-benzothien-2-yl]benzoic acid as a white solid. ¹H NMR (400 MHz, DMSO-d₆): δ 13.25 (br s, 1H), 8.19 (s, 1H), 7.99 (d, J=8 Hz, 1H), 7.92 (d, J=8 Hz, 1H), 7.78 (m, 2H), 7.60 (m, 3H), 7.52 (dd, J=9, 7 Hz, 1H), 7.27 (d, J=2 Hz, 1H), 6.79 (dd, J=9, 2 Hz, 1H), 4.87 (s, 2H), 3.46 (septet, J=7 Hz, 1H), 1.32 (d, J=7 Hz, 6H). HRMS C₂₈H₂₂Cl₂NO₄S m/z 538.06411 (M+H)⁺_{Cal}; 538.06418 (M+H)⁺_{Obs}.

### Example 55

### (3S)-1-{[5-({[3-(2,6-Dichlorophenyl)-5-(1-methylethyl)-4-isoxazolyl]methyl}oxy)-1H-indol-1-yl]carbonyl}-3-pyrrolidinecarboxylic acid

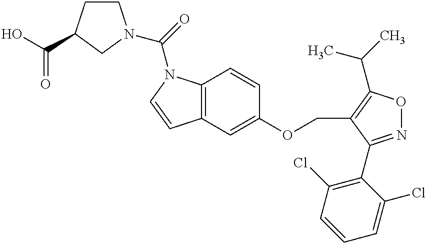

### 55a) Methyl (3S)-3-pyrrolidinecarboxylate hydrochloride

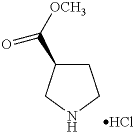

To a stirred solution of (3S)-1-{[(1,1-dimethylethyl)oxy]carbonyl}-3-pyrrolidinecarboxylic acid (1.0 g, 4.6 mmol) in methanol (25 mL) was added, dropwise, thionyl chloride (1.63 g, 13.7 mmol) at room temperature under a nitrogen atmosphere. The reaction mixture was heated at reflux for 2 h and allowed to cool at room temperature. The reaction mixture was concentrated to give 0.766 g (100%) of methyl (3S)-3-pyrrolidinecarboxylate hydrochloride as an oil which solidified upon standing. ¹H NMR (400 MHz, CDCl₃): δ 9.91 (m, 2H), 3.76 (s, 3H), 3.59 (br s, 2H), 3.49 (br s, 2H), 3.28 (br s, 1H), 2.33 (br s, 2H).

### 55b) Methyl (3S)-1-(1H-imidazol-1-ylcarbonyl)-3-pyrrolidinecarboxylate

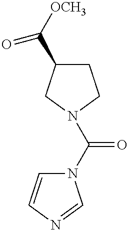

To a stirred mixture of methyl (3S)-3-pyrrolidinecarboxylate hydrochloride (0.765 g, 4.6 mmol), 1,1′-carbonyldiimidazole (0.83 g, 5.1 mmol), and dichloromethane (15 mL) was added triethylamine (1.4 mL, 10 mmol) at room temperature under a nitrogen atmosphere. The reaction mixture was stirred at room temperature for 23 h. The reaction mixture was washed with water (2×10 mL) and the organic phase was dried over magnesium sulfate, filtered, and the filtrate was concentrated to give 0.75 g (73%) of methyl (3S)-1-(1H-imidazol-1-ylcarbonyl)-3-pyrrolidinecarboxylate as an oil which solidified upon standing to give an off-white solid. ¹H NMR (400 MHz, CDCl₃): δ 8.00 (s, 1H), 7.33 (s, 1H), 7.08 (s, 1H), 3.92-3.82 (m, 2H), 3.79-3.65 (m, 2H), 3.74 (s, 3H), 3.17 (quin, J=7 Hz, 1H), 2.27 (q, J=7 Hz, 2H).

### 55c) Methyl (3S)-1-{[5-({[3-(2,6-dichlorophenyl)-5-(1-methylethyl)-4-isoxazolyl]methyl}oxy)-1H-indol-1-yl]carbonyl}-3-pyrrolidinecarboxylate

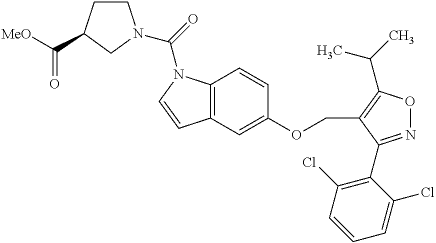

To a stirred suspension of sodium hydride (60% oil dispersion) (0.038 g, 0.95 mmol) in N,N-dimethylformamide (2 mL) was added dropwise a solution of 5-({[3-(2,6-dichlorophenyl)-5-(1-methylethyl)-4-isoxazolyl]methyl}oxy)-1H-indole (prepared according to the general procedure described for Example 46a) (0.326 g, 0.81 mmol) in N,N-dimethylformamide (2 mL) at room temperature under a nitrogen atmosphere. The reaction mixture was stirred at room temperature for 10 min, then cooled in an ice-water bath for 10 min. To the cooled reaction mixture was added, dropwise, a solution of methyl (3S)-1-(1H-imidazol-1-ylcarbonyl)-3-pyrrolidinecarboxylate (0.198 g, 0.89 mmol) in N,N-dimethylformamide (2 mL). The ice-water bath was removed and the reaction mixture was stirred overnight at room temperature. The reaction mixture was partitioned between water and ethyl acetate. The layers were separated and the organic phase was washed with water, followed by saturated sodium chloride, dried over magnesium sulfate, filtered, and the filtrate was concentrated to give the crude product. The crude product was purified by flash chromatography over silica with a hexanes:ethyl acetate gradient (100:0 to 50:50) to give an oil. The oil was dissolved in dichloromethane and the solution was concentrated. The product was dissolved in dichloromethane and the solution was concentrated to give 0.086 g (23% based on recovered starting material) of methyl (3S)-1-{[5-({[3-(2,6-dichlorophenyl)-5-(1-methylethyl)-4-isoxazolyl]methyl}oxy)-1H-indol-1-yl]carbonyl}-3-pyrrolidinecarboxylate. ¹H NMR (400 MHz, CDCl₃): δ 7.65 (d, J=9 Hz, 1H), 7.39 (m, 2H), 7.30 (m, 2H), 6.91 (d, J=2 Hz, 1H), 6.77 (dd, J=9, 2 Hz, 1H), 6.45 (d, J=3 Hz, 1H), 4.75 (s, 2H), 3.88-3.79 (m, 2H), 3.76-3.62 (m, 2H), 3.72 (s, 3H), 3.34 (septet, J=7 Hz, 1H), 3.13 (quin, J=7 Hz, 1H), 2.23 (q, J=7 Hz, 2H), 1.40 (d, J=7 Hz, 6H). ES-LCMS m/z 556 (M+H)⁺.

### 55d) (3S)-1-{[5-({[3-(2,6-dichlorophenyl)-5-(1-methylethyl)-4-isoxazolyl]methyl}oxy)-1H-indol-1-yl]carbonyl}-3-pyrrolidinecarboxylic acid

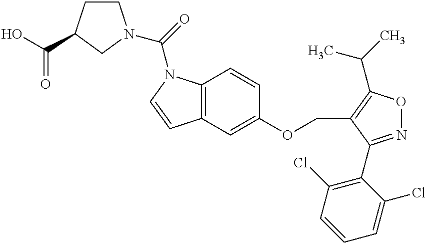

To a solution of methyl (3S)-1-{[5-({[3-(2,6-dichlorophenyl)-5-(1-methylethyl)-4-isoxazolyl]methyl}oxy)-1H-indol-1-yl]carbonyl}-3-pyrrolidinecarboxylate (0.086 g, 0.15 mmol) in tetrahydrofuran (4 mL) and methanol (2 mL) was added sodium hydroxide (1 N) (0.17 mL, 0.17 mmol) at room temperature. The reaction mixture was stirred for 18 h at room temperature under a nitrogen atmosphere. The reaction mixture was concentrated and water (5 mL) was added to the residue. The pH of the aqueous solution was adjusted to approximately 3 (litmus paper) with 10% citric acid. The acidic aqueous phase was extracted with ethyl acetate (10 mL). The organic extract was washed with water (5 mL), followed by saturated sodium chloride, dried over magnesium sulfate, filtered, and the filtrate concentrated to give an oil. The oil was dissolved in dichloromethane and the solution was concentrated to give the desired product. ¹H NMR indicated that the product contained an impurity. The impure product was purified by flash chromatography over silica with a dichloromethane:methanol gradient (100:0 to 98:2) to give 0.063 g (78%) of (3S)-1-{[5-({[3-(2,6-dichlorophenyl)-5-(1-methylethyl)-4-isoxazolyl]methyl}oxy)-1H-indol-1-yl]carbonyl}-3-pyrrolidinecarboxylic acid after drying as a white amorphous solid. ¹H NMR (400 MHz, DMSO-d₆): δ 12.56 (br s, 1H), 7.60 (m, 3H), 7.52 (m, 2H), 6.98 (d, J=2 Hz, 1H), 6.62 (dd, J=9, 2 Hz, 1H), 6.47 (d, J=4 Hz, 1H), 4.79 (s, 2H), 3.66 (d, J=7 Hz, 2H), 3.54 (t, J=7 Hz, 2H), 3.42 (septet, J=7 Hz, 1H), 3.09 (quin, J=7

Hz, 1H), 2.17-1.99 (m, 2H), 1.29 (d, J=7 Hz, 6H). HRMS C₂₇H₂₆Cl₂N₃O₅ m/z 542.12440 (M+H)⁺_{Cal}; 542.12439 (M+H)⁺_{Obs}.

### Example 56

### 3-[5-({[3-(2,6-Dichlorophenyl)-5-(1-methylethyl)-4-isoxazolyl]methyl}oxy)-1H-indol-2-yl]benzoic acid

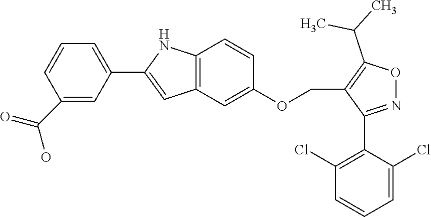

### 56a) 1,1-Dimethylethyl 5-[(phenylmethyl)oxy]-1H-indole-1-carboxylate

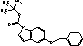

To a stirred solution of 5-benzyloxyindole (5.3 g, 23.7 mmol) and 4-dimethylaminopyridine (0.055 g, 0.45 mmol) in dichloromethane (30 mL) was added, portionwise, di-tert-butyl-dicarbonate (5.65 g, 25.9 mmol). The final portion of di-tert-butyl-dicarbonate was added to the reaction mixture with the aid of dichloromethane. The reaction mixture was stirred overnight at room temperature under a nitrogen atmosphere. The reaction mixture was washed with 1 N hydrochloric acid (30 mL). The layers were separated and the organic phase was washed with saturated sodium chloride, dried over magnesium sulfate, filtered, and the filtrate was concentrated to give an oil. In an attempt to induce crystallization, hexanes were added to the crude product, however, the product failed to crystallize. The hexanes were removed in vacuo and the crude product was purified by flash chromatography over silica with a hexanes:ethyl acetate gradient (100:0 to 50:50) to give 1.76 g of 1,1-dimethylethyl 5-[(phenylmethyl)oxy]-1H-indole-1-carboxylate as well as 4.9 g of a batch of 1,1-dimethylethyl 5-[(phenylmethyl)oxy]-1H-indole-1-carboxylate which contained <5% of di-tert-butyl-dicarbonate as an impurity for a total yield of 6.66 g (87%). ¹H NMR (400 MHz, DMSO-d₆): δ 7.90 (d, J=9 Hz, 1H), 7.61 (d, J=4 Hz, 1H), 7.44 (m, 2H), 7.37 (t, J=7 Hz, 2H), 7.29 (m, 1H), 7.20 (d, J=3 Hz, 1H), 6.99 (dd, J=9, 2 Hz, 1H), 6.60 (d, J=4 Hz, 1H), 5.1 (s, 2H), 1.59 (s, 9H). ES-LCMS m/z 324 (M+H)⁺.

### 56b) {1-{[(1,1-Dimethylethyl)oxy]carbonyl}-5-[(phenylmethyl)oxy]-1H-indol-2-yl}boronic acid

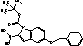

To a solution of 1,1-dimethylethyl 5-[(phenylmethyl)oxy]-1H-indole-1-carboxylate (1.76 g, 4.8 mmol) in tetrahydrofuran (10 mL) was added triisopropyl borate (2.2 mL, 9.5 mmol). The solution was stirred in an ice-water bath under a nitrogen atmosphere and lithium diisopropylamide (2 M in heptane/tetrahydrofuran/ethylbenzene) (4 mL, 8 mmol) was added, portionwise, over 20 min. The reaction mixture was swirled to facilitate mixing, then stirred with cooling for 45 m. Lithium diisopropylamide (2 M in heptane/tetrahydrofuran/ethylbenzene) (0.8 mL, 1.6 mmol) was added to the reaction mixture over a 5-min period. The cold reaction mixture was swirled to facilitate mixing, then stirred for 75 min. Hydrochloric acid (1 N) (50 mL) was added to the reaction mixture and the aqueous mixture was extracted with ethyl acetate. The organic extract was washed with water followed by saturated sodium chloride, dried over magnesium sulfate, filtered, and the filtrate was concentrated to give 2.03 (100%) of {1-{[(1,1-dimethylethyl)oxy]carbonyl}-5-[(phenylmethyl)oxy]-1H-indol-2-yl}boronic acid as a tan solid. ¹H NMR (400 MHz, DMSO-d₆): δ 8.15 (s, 2H), 7.93 (d, J=9 Hz, 1H), 7.44 (d, 2H), 7.37 (m, 2H), 7.30 (m, 1H), 7.15 (d, J=2 Hz, 1H), 6.94 (dd, J=9, 2 Hz, 1H), 6.51 (s, 1H), 5.09 (s, 2H), 1.56 (s, 9H).

### 56c) 1,1-Dimethylethyl 2-{3-[(ethyloxy)carbonyl]phenyl}-5-[(phenylmethyl)oxy]-1H-indole-1-carboxylate

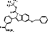

To a solution of {1-{[(1,1-dimethylethyl)oxy]carbonyl}-5-[(phenylmethyl)oxy]-1H-indol-2-yl}boronic acid (1.13 g, 3.08 mmol), ethyl-3-iodobenzoate (0.34 mL, 2 mmol), and tetrakis(triphenylphosphine)palladium(0) (0.126 g, 0.11 mmol), in 1,2-dimethoxyethane (35 mL) was added sodium carbonate (2 M) (4 mL, 8 mmol). The stirred reaction mixture was heated at reflux for 4 h under a nitrogen atmosphere. The reaction mixture was allowed to cool at room temperature and partitioned between water and ethyl acetate. The organic phase was separated, dried over magnesium sulfate, filtered, and the filtrate was concentrated to give an oil. The crude product was purified by flash chromatography over silica with a hexanes:ethyl acetate gradient (100:0 to 70:30) to give an oil. The oil was dissolved in dichloromethane and the solution was concentrated to give the product. The product was dissolved in ethyl acetate and one-half of the solution was concentrated to give 0.42 g of 1,1-dimethylethyl 2-{3-[(ethyloxy)carbonyl]phenyl}-5-[(phenylmethyl)oxy]-1H-indole-1-carboxylate for a total yield of 0.84 g (89%). ¹H NMR (400 MHz, DMSO-d₆): δ 7.99 (d, J=9 Hz, 1H), 7.95 (m, 2H), 7.75 (d, J=8 Hz, 1H), 7.58 (t, J=8 Hz, 1H), 7.45 (m, 2H), 7.37 (m, 2H), 7.31 (m, 1H), 7.21 (d, J=3 Hz, 1H), 7.03 (dd, J=9, 3 Hz, 1H), 6.72 (s, 1H), 5.13 (s, 2H), 4.32 (q, J=7 Hz, 2H), 1.30 (t, J=7 Hz, 3H), 1.23 (s, 9H). ES-LCMS m/z 494 (M+Na)⁺.

### 56d) 1,1-Dimethylethyl 2-{3-[(ethyloxy)carbonyl]phenyl}-5-hydroxy-1H-indole-1-carboxylate

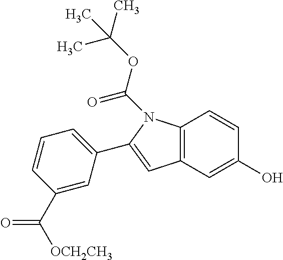

To a solution of 1,1-dimethylethyl 2-{3-[(ethyloxy)carbonyl]phenyl}-5-[(phenylmethyl)oxy]-1H-indole-1-carboxylate (0.42 g, 0.89 mmol) in ethyl acetate (20 mL) and ethanol (10 mL) was added 10% palladium on carbon (Degussa Type; 50% water by wt.) (0.103 g). The flask was evacuated and filled with nitrogen three times, evacuated, and filled with hydrogen using a balloon. The reaction mixture was stirred for 4 h at room temperature under a hydrogen atmosphere. The reaction mixture was filtered through a pad of Celite® and the pad was washed twice with ethyl acetate. The filtrate was allowed to stand at room temperature overnight. The filtrate was concentrated to give an amorphous off-white solid. The crude product was purified by flash chromatography over silica with a hexanes:ethyl acetate gradient (100:0 to 60:40) to give 0.25 g (74%) of 1,1-dimethylethyl 2-{3-[(ethyloxy)carbonyl]phenyl}-5-hydroxy-1H-indole-1-carboxylate. ¹H NMR (400 MHz, DMSO-d₆): δ 9.23 (s, 1H), 7.95-7.87 (m, 3H), 7.73 (d, J=8 Hz, 1H), 7.57 (t, J=8 Hz, 1H), 6.91 (d, J=2 Hz, 1H), 6.79 (dd, J=9, 2 Hz, 1H), 6.66 (s, 1H), 4.32 (q, J=7 Hz, 2H), 1.30 (t, J=7 Hz, 3H), 1.22 (s, 9H). ES-LCMS m/z 404 (M+Na)⁺.

### 56e) 1,1-Dimethylethyl 5-({[3-(2,6-dichlorophenyl)-5-(1-methylethyl)-4-isoxazolyl]methyl}oxy)-2-{3-[(ethyloxy)carbonyl]phenyl}-1H-indole-1-carboxylate

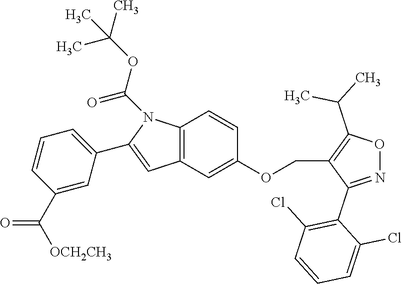

To a stirred solution of 1,1-dimethylethyl 2-{3-[(ethyloxy)carbonyl]phenyl}-5-hydroxy-1H-indole-1-carboxylate (0.25 g, 0.655 mmol), [3-(2,6-dichlorophenyl)-5-(1-methylethyl)-4-isoxazolyl]methanol (prepared according to the general procedure described in Maloney, P. R., et al., 2000 J. Med. Chem. 43:2971-2974) (0.197 g, 0.688 mmol), and triphenylphosphine (0.184 g, 0.702 mmol) in toluene (20 mL) was added, dropwise, diisopropyl azodicarboxylate (0.14 mL, 0.69 mmol) at room temperature. The reaction mixture was stirred for 19 h at room temperature under a nitrogen atmosphere. The reaction mixture was concentrated and the crude product was purified by flash chromatography over silica with a hexanes:ethyl acetate gradient (100:0 to 60:40) to give the product as an oil. The product was dissolved in dichloromethane and the solution was concentrated in vacuo. To the product was added dichloromethane and the solution was concentrated to give 0.186 g (44%) of 1,1-dimethylethyl 5-({[3-(2,6-dichlorophenyl)-5-(1-methylethyl)-4-isoxazolyl]methyl}oxy)-2-{3-[(ethyloxy)carbonyl]phenyl}-1H-indole-1-carboxylate as a white amorphous solid. ¹H NMR (400 MHz, DMSO-d₆): δ 7.95 (d, J=8 Hz, 1H), 7.89 (m, 2H), 7.72 (d, J=8 Hz, 1H), 7.62-7.50 (m, 4H), 7.01 (d, J=2 Hz, 1H), 6.74 (dd, J=9, 2 Hz, 1H), 6.66 (s, 1H), 4.83 (s, 2H), 4.32 (q, J=7 Hz, 2H), 3.44 (septet, J=7 Hz, 1H), 1.30 (m, 9H), 1.21 (s, 9H). ES-LCMS m/z 649 (M+H)⁺.

### 56f) 3-[5-({[3-(2,6-Dichlorophenyl)-5-(1-methylethyl)-4-isoxazolyl]methyl}oxy)-1H-indol-2-yl]benzoic acid

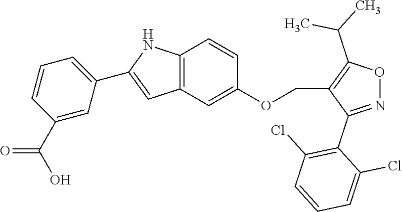

To a stirred solution of 1,1-dimethylethyl 5-({[3-(2,6-dichlorophenyl)-5-(1-methylethyl)-4-isoxazolyl]methyl}oxy)-2-{3-[(ethyloxy)carbonyl]phenyl}-1H-indole-1-carboxylate (0.162 g, 0.25 mmol) in 1,4-dioxane (2 mL) and ethanol (2 mL) was added sodium hydroxide (1 N) (3.2 mL, 3.2 mmol). The turbid reaction mixture was heated at 60° C. under a nitrogen atmosphere for 5 h. The reaction mixture was allowed to stand overnight at room temperature. The reaction mixture was partially concentrated and water (5 mL) was added to the residue. The pH of the aqueous phase was adjusted to approximately 2 (litmus paper) with 10% citric acid. The acidic aqueous mixture was extracted with ethyl acetate. The organic phase was separated, dried over magnesium sulfate, filtered, and the filtrate was concentrated to give an oil. The crude product was purified by flash chromatography over silica with a hexanes:ethyl acetate gradient (100:0 to 0:100) to give 0.0229 g (18%) of 3-[5-({[3-(2,6-dichlorophenyl)-5-(1-methylethyl)-4-isoxazolyl]methyl}oxy)-1H-indol-2-yl]benzoic acid as a pale yellow solid. ¹H NMR (400 MHz, DMSO-d₆): δ 13.10 (br s, 1H), 11.53 (br s, 1H), 8.34 (s, 1H), 8.03 (d, J=8 Hz, 1H), 7.83 (d, J=8 Hz, 1H), 7.62 (m, 2H), 7.53 (m, 2H), 7.19 (d, J=9 Hz, 1H), 6.92 (d, J=2 Hz, 1H), 6.80 (d, J=2 Hz, 1H), 6.52 (dd, J=9, 2 Hz, 1H), 4.77 (s, 2H), 3.41 (septet, J=7 Hz, 1H), 1.29 (d, J=7 Hz, 6H). HRMS C₂₈H₂₃Cl₂N₂O₄ m/z 521.10294 (M+H)⁺_{Cal}; 521.10292 (M+H)⁺_{Obs}.

### Example 57

### 3-[5-({[3-(2,6-Dichlorophenyl)-5-(1-methylethyl)-4-isoxazolyl]methyl}oxy)-2,3-dihydro-1H-inden-2-yl]benzoic acid

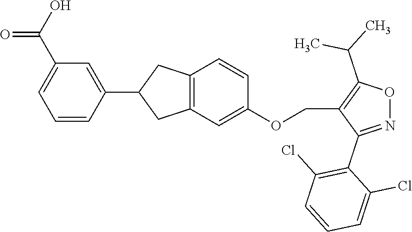

### 57a) 2-Bromo-6-(methyloxy)-2,3-dihydro-1H-inden-1-one

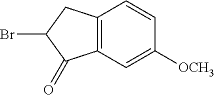

To a stirred suspension of copper(II) bromide (16.6 g, 74.3 mmol) in ethyl acetate (50 mL) at reflux was added, dropwise, over a period of 25 min a solution of 6-methoxy-1-indanone (6.2 g, 38.2 mmol) in chloroform (50 mL) under a nitrogen atmosphere. The reaction mixture was heated at reflux for 1 h. The oil bath was removed and the reaction mixture was allowed to stand overnight at room temperature. The reaction mixture was filtered and the off-white solid was washed with chloroform. The filtrate was concentrated to give the crude product as a turbid liquid. Ethyl acetate was added to the crude product and the cloudy solution was applied to a silica column. The crude product was purified by flush chromatography with hexanes:ethyl acetate (9:1) to give the desired product. The product was dissolved in ethanol and the solution was concentrated to give 8.6 g (93%) of 2-bromo-6-(methyloxy)-2,3-dihydro-1H-inden-1-one as a pale tan solid. ¹H NMR indicated the product contained 6-methoxy-1-indanone (approximately 5% by weight). The product was used without further purification. ¹H NMR (400 MHz, CDCl₃): δ 7.33 (m, 1H), 7.25 (m, 2H), 4.66 (dd, J=7, 3 Hz, 1H), 3.84 (s, 3H), 3.76 (dd, J=18, 7 Hz, 1H), 3.34 (dd, J=18, 3 Hz, 1H). AP-LCMS m/z 241 (M+H)⁺.

### 57b) 2-Bromo-6-(methyloxy)-2,3-dihydro-1H-inden-1-ol

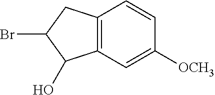

To a stirred, turbid mixture of 2-bromo-6-(methyloxy)-2,3-dihydro-1H-inden-1-one (8.54 g, 35.4 mmol) in ethanol (50 mL) was added sodium borohydride (0.77 g, 20.4 mmol), portionwise, over a period of 20 min at room temperature. The reaction mixture was stirred at room temperature for 15 min. The reaction mixture was poured into water and the aqueous mixture was extracted with chloroform. The organic phase was separated, dried over magnesium sulfate, filtered, and the filtrate was concentrated to give a pale yellow solid. The crude product was purified by flush chromatography over silica with dichloromethane to give 5.14 g (60%) of 2-bromo-6-(methyloxy)-2,3-dihydro-1H-inden-1-ol as a yellow-tan solid. ¹H NMR (400 MHz, CDCl₃): δ 7.15 (d, J=8 Hz, 1H), 6.99 (d, J=2 Hz, 1H), 6.84 (dd, J=8, 2 Hz, 1H), 4.93 (s, 2H), 3.81 (s, 3H), 3.40-3.26 (m, 2H), 2.42 (br s, 1H).

### 57c) 2-Bromo-5-(methyloxy)-1H-indene

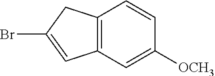

2-Bromo-6-(methyloxy)-2,3-dihydro-1H-inden-1-ol (5.1 g, 21 mmol), p-toluenesulfonic acid monohydrate (0.65 g, 3.4 mmol), and toluene (150 mL) were combined and the stirred reaction mixture was heated at reflux under a nitrogen atmosphere. A Dean-Stark trap was used to remove water from the reaction mixture. After 2 h, the oil bath was removed, and the reaction mixture was allowed to stand at room temperature for three days. The reaction mixture was partitioned between toluene and saturated potassium carbonate. The organic phase was separated and washed with water. The phases did not readily separate from one another upon washing with water. To the mixture was added ethyl acetate and saturated sodium chloride. The organic phase was separated, dried over magnesium sulfate, filtered, and the filtrate was concentrated to give a brown liquid which partially solidified upon standing. The crude product was partially purified by flash chromatography over silica with a hexanes:ethyl acetate gradient (100:0 to 80:20) to give 3.34 g of 2-bromo-5-(methyloxy)-1H-indene as a white solid. ¹H NMR indicates that numerous minor impurities are present. The impure product was used without further purification. ¹H NMR (400 MHz, CDCl₃): δ 7.24 (m, 1H), 6.87 (s, 1H), 6.86 (d, J=2 Hz, 1H), 6.72 (dd, J=8, 2 Hz, 1H), 3.81 (s, 3H), 3.54 (s, 2H).

### 57d) Methyl 3-[5-(methyloxy)-2,3-dihydro-1H-inden-2-yl]benzoate

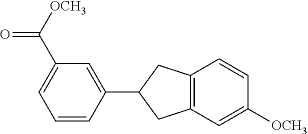

2-Bromo-5-(methyloxy)-1H-indene (impure) (2.14 g), 3-methoxycarbonylphenyl)boronic acid (2.2 g, 12.2 mmol), sodium carbonate (2 M) (22 mL, 44 mmol), tetrakistriphenylphosphinepalladium(0) (0.54 g, 0.47 mmol), and 1,2-dimethoxyethane (75 mL) were combined and the stirred reaction mixture was heated at reflux for 2 h under a nitrogen atmosphere. The oil bath was removed and the reaction mixture was allowed to stand overnight at room temperature. The reaction mixture was partitioned between water and ethyl acetate. The organic phase was separated, dried over magnesium sulfate, filtered, and the filtrate was concentrated to give a dark brown liquid. The crude product was purified by flash chromatography over silica with a hexanes:ethyl acetate gradient (100:0 to 60:40) to give 1.2 g of a viscous gold-yellow oil. ¹H NMR analysis of the oil indicated that it was a mixture of methyl 3-[5-(methyloxy)-1H-inden-2-yl]benzoate and methyl 3-[6-(methyloxy)-1H-inden-2-yl]benzoate as well as an impurity. To a solution of methyl 3-[5-(methyloxy)-1H-inden-2-yl]benzoate and methyl 3-[6-(methyloxy)-1H-inden-2-yl]benzoate (0.87 g) in ethyl acetate (30 mL) and ethanol (15 mL) was added 10% palladium on carbon (Degussa; 50% water by wt) (0.12 g). The flask was evacuated and filled with nitrogen (3 times), evacuated, and filled with hydrogen using a balloon. The reaction mixture was stirred overnight under a hydrogen atmosphere at room temperature. The reaction mixture was filtered through a pad of Celite®. The pad of Celite® was washed with ethyl acetate (2 times) followed by ethanol. The filtrate was concentrated to give a yellow oil. The crude product was purified by flash chromatography over silica with a hexanes:ethyl acetate gradient (100:0 to 80:20) to give 0.57 g (21% from 2-bromo-6-(methyloxy)-2,3-dihydro-1H-inden-1-ol) of methyl 3-[5-(methyloxy)-2,3-dihydro-1H-inden-2-yl]benzoate as an oil. ¹H NMR (400 MHz, CDCl₃): δ 7.99 (s, 1H), 7.88 (d, J=8 Hz, 1H), 7.48 (d, J=8 Hz, 1H), 7.36 (t, J=Hz, 1H), 7.13 (d, J=8 Hz, 1H), 6.80 (s, 1H), 6.74 (dd, J=8, 2 Hz, 1H), 3.91 (s, 3H), 3.80 (s, 3H), 3.74 (quin, J=9 Hz, 1H), 3.36-3.27 (m, 2H), 3.09-2.98 (m, 2H). ES-LCMS m/z 283 (M+H)⁺.

### 57e) Methyl 3-(5-hydroxy-2,3-dihydro-1H-inden-2-yl)benzoate

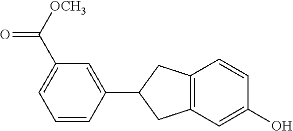

To a stirred ice-water cooled solution of methyl 3-[5-(methyloxy)-2,3-dihydro-1H-inden-2-yl]benzoate (0.57 g, 2 mmol) in dichloromethane (20 mL) was added dropwise boron tribromide (1 M in dichloromethane) (5 mL, 5 mmol) under a nitrogen atmosphere. The reaction mixture was stirred with cooling for 2.75 h. The reaction mixture was poured into ice-water and the aqueous mixture was extracted with dichloromethane. The organic extract was separated, washed with water followed by saturated sodium chloride, dried over magnesium sulfate, filtered, and the filtrate was allowed to stand at room temperature overnight. The filtrate was concentrated to give an oil. The crude product was partitioned between dichloromethane and saturated sodium bicarbonate. The organic phase was separated, washed with saturated sodium chloride, dried over magnesium sulfate, filtered, and the filtrate was concentrated to give an oil. The crude product was purified by flash chromatography over silica with a hexanes:ethyl acetate gradient (100:0 to 50:50) to give 0.10 g (18%) of methyl 3-(5-hydroxy-2,3-dihydro-1H-inden-2-yl)benzoate as a white solid. ¹H NMR (400 MHz, CDCl₃): δ 7.99 (s, 1H), 7.89 (d, J=8 Hz, 1H), 7.48 (d, J=8 Hz, 1H), 7.37 (t, J=8 Hz, 1H), 7.09 (d, J=8 Hz, 1H), 6.73 (s, 1H), 6.66 (dd, J=8, 2 Hz, 1H), 4.56 (br s, 1H), 3.91 (s, 3H), 3.74 (quin, J=9 Hz, 1H), 3.34-3.26 (m, 2H), 3.07-2.97 (m, 2H). ES-LCMS m/z 269 (M+H)⁺.

### 57f) Methyl 3-[5-({[3-(2,6-dichlorophenyl)-5-(1-methylethyl)-4-isoxazolyl]methyl}oxy)-2,3-dihydro-1H-inden-2-yl]benzoate

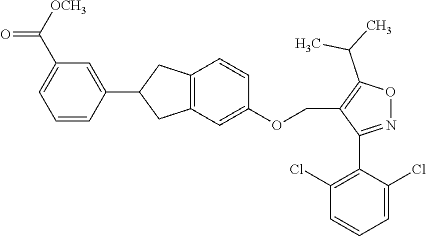

Methyl 3-(5-hydroxy-2,3-dihydro-1H-inden-2-yl)benzoate (0.10 g, 0.36 mmol), cesium carbonate (0.28 g, 0.86 mmol), and N,N-dimethylformamide (6 mL) were combined and the reaction mixture was heated at 65° C. for 2 h with stirring under a nitrogen atmosphere. The oil bath was removed and the reaction mixture was allowed to cool at room temperature. To the reaction mixture was added a solution of 4-(chloromethyl)-3-(2,6-dichlorophenyl)-5-(1-methylethyl)isoxazole (0.112 g, 0.37 mmol) in N,N-dimethylformamide (3 mL). The stirred reaction mixture was heated at 65° C. for 19 h under a nitrogen atmosphere. The reaction mixture was partitioned between water and ethyl acetate. The organic phase was separated, washed with water followed by saturated sodium chloride, dried over magnesium sulfate, filtered, and the filtrate was concentrated to give an oil. The crude product was purified by flash chromatography over silica with a hexanes:ethyl acetate gradient (100:0 to 60:40) to give 0.105 g (55%) of methyl 3-[5-({[3-(2,6-dichlorophenyl)-5-(1-methylethyl)-4-isoxazolyl]methyl}oxy)-2,3-dihydro-1H-inden-2-yl]benzoate as a viscous oil. ¹H NMR (400 MHz, CDCl₃): δ 7.96 (s, 1H), 7.88 (d, J=8 Hz, 1H), 7.45 (d, J=8 Hz, 1H), 7.41-7.29 (m, 4H), 7.06 (d, J=8 Hz, 1H), 6.65 (s, 1H), 6.61 (dd, J=8, 2 Hz, 1H), 4.70 (s, 2H), 3.90 (s, 3H), 3.70 (quin, J=9 Hz, 1H), 3.35-3.24 (m, 3H), 3.03-2.95 (m, 2H), 1.41 (d, J=7 Hz, 6H). ES-LCMS m/z 558 (M+Na)⁺.

### 57g) 3-[5-({[3-(2,6-Dichlorophenyl)-5-(1-methylethyl)-4-isoxazolyl]methyl}oxy)-2,3-dihydro-1H-inden-2-yl]benzoic acid

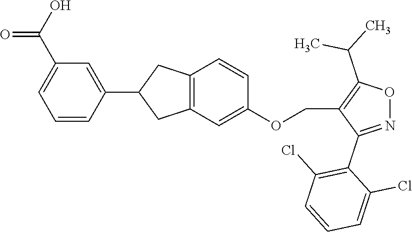

To a stirred solution of methyl 3-[5-({[3-(2,6-dichlorophenyl)-5-(1-methylethyl)-4-isoxazolyl]methyl}oxy)-2,3-dihydro-1H-inden-2-yl]benzoate (0.10 g, 0.19 mmol) in tetrahydrofuran (6 mL) and methanol (3 mL) was added sodium hydroxide (1 N) (2.2 mL, 2.2 mmol). The stirred reaction mixture was heated at 65° C. for 3 h under a nitrogen atmosphere. The reaction mixture was allowed to stand overnight at room temperature. The reaction mixture was partially concentrated to remove the tetrahydrofuran and methanol. Water was added to the residue and the pH of the aqueous mixture was adjusted to approximately 3 (litmus paper) with 10% citric acid. The acidic aqueous phase was extracted with ethyl acetate. The organic extract was separated, washed with water followed by saturated sodium chloride, dried over magnesium sulfate, filtered, and the filtrate was concentrated to give an oil. The product was dissolved in dichloromethane and the solution was concentrated. The off-white amorphous solid was dried to give 0.082 g (85%) of 3-[5-({[3-(2,6-dichlorophenyl)-5-(1-methylethyl)-4-isoxazolyl]methyl}oxy)-2,3-dihydro-1H-inden-2-yl]benzoic acid. ¹H NMR (400 MHz, DMSO-d₆): δ 12.79 (br s, 1H), 7.82 (s, 1H), 7.75 (d, J=8 Hz, 1H), 7.61 (m, 2H), 7.52 (m, 2H), 7.40 (t, J=8 Hz, 1H), 7.03 (d, J=8 Hz, 1H), 6.66 (s, 1H), 6.54 (dd, J=8, 2 Hz, 1H), 4.75 (s, 2H), 3.67 (quin, J=8 Hz, 1H), 3.43-3.38 (m, 1H), 3.23-3.16 (m, 2H), 2.90-2.81 (m, 2H), 1.30 (d, J=7 Hz, 6H). ES-LCMS m/z 522 (M+H)⁺.

### Example 58

### 3-[6-({[3-(2,6-Dichlorophenyl)-5-(1-methylethyl)-4-isoxazolyl]methyl}oxy)-1H-inden-2-yl]benzoic acid and 3-[5-({[3-(2,6-dichlorophenyl)-5-(1-methylethyl)-4-isoxazolyl]methyl}oxy)-1H-inden-2-yl]benzoic acid

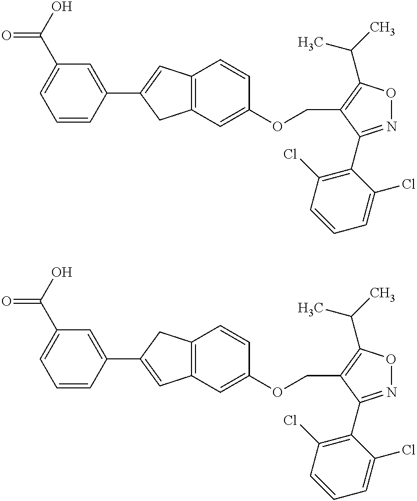

### 58a) 6-Hydroxy-2,3-dihydro-1H-inden-1-one

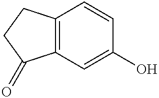

To a stirred suspension of aluminum chloride (5.3 g, 39.7 mmol) in toluene (75 mL) was slowly added 6-methoxy-1-indanone (2.5 g, 15.4 mmol) at room temperature under a nitrogen atmosphere. The residual 6-methoxy-1-indanone which remained in the powder addition funnel was rinsed into the reaction mixture with toluene (25 mL). The reaction mixture was heated at reflux for 1 h. The reaction mixture was allowed to cool at room temperature and slowly poured into ice-water. The mixture was transferred to a separatory funnel with the aid of ethyl acetate. The layers were separated and the organic phase was washed with water (2 times) followed by saturated sodium chloride, dried over magnesium sulfate, filtered, and the filtrate was concentrated to give 1.5 g (66%) of 6-hydroxy-2,3-dihydro-1H-inden-1-one as a pale tan solid. ¹H NMR (400 MHz, DMSO-d₆): δ 9.72 (s, 1H), 7.36 (d, J=8 Hz, 1H), 7.07 (dd, J=8, 3 Hz, 1H), 6.90 (d, J=3 Hz, 1H), 2.94 (m, 2H), 2.58 (m, 2H). ES-LCMS m/z 149 (M+H)⁺.

### 58b) 6-({[3-(2,6-Dichlorophenyl)-5-(1-methylethyl)-4-isoxazolyl]methyl}oxy)-2,3-dihydro-1H-inden-1-one

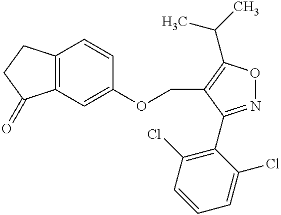

To a stirred ice-water cooled suspension of 6-hydroxy-2,3-dihydro-1H-inden-1-one (1.46 g, 9.85 mmol), triphenylphosphine (polystyrene resin bound; 3 mmol/g) (3.4 g, 10.2 mmol), and [3-(2,6-dichlorophenyl)-5-(1-methylethyl)-4-isoxazolyl]methanol (prepared according to the general procedure described in Maloney, P. R., et al., 2000 J. Med. Chem. 43:2971-2974) (2.95 g, 10.3 mmol) in dichloromethane (50 mL) was slowly added a solution of diisopropyl azodicarboxylate (2.1 mL, 10.7 mmol) in dichloromethane (20 mL). The ice-water bath was removed and the reaction mixture was allowed to stir at room temperature overnight. The reaction mixture was filtered and the resin was washed with dichloromethane. The filtrate was concentrated to give an orange oil. The crude product was purified by flash chromatography over silica with a hexanes:ethyl acetate gradient (100:0 to 0:100) to give impure product. The impure product was purified by flash chromatography over silica with a hexanes:ethyl acetate gradient (100:0 to 40:60) to give 2.7 g (66%) of 6-({[3-(2,6-dichlorophenyl)-5-(1-methylethyl)-4-isoxazolyl]methyl}oxy)-2,3-dihydro-1H-inden-1-one as viscous oil which solidified to a white solid. ¹H NMR (400 MHz, CDCl₃): δ 7.39 (m, 2H), 7.32-7.28 (m, 2H), 7.08 (d, J=2 Hz, 1H), 7.02 (dd, J=8, 2 Hz, 1H), 4.73 (s, 2H), 3.33 (septet, J=7 Hz, 1H), 3.04 (m, 2H), 2.69 (m, 2H), 1.42 (d, J=7 Hz, 6H). ES-LCMS m/z 416 (M+H)⁺.

### 58c) 2-Bromo-6-({[3-(2,6-dichlorophenyl)-5-(1-methylethyl)-4-isoxazolyl]methyl}oxy)-2,3-dihydro-1H-inden-1-one

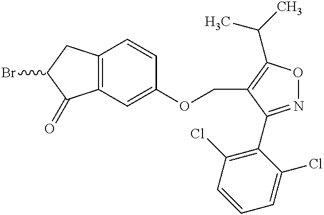

To a stirred suspension of copper (II) bromide (1.87 g, 8.4 mmol) in ethyl acetate (10 mL) at reflux was added dropwise a solution of 6-({[3-(2,6-dichlorophenyl)-5-(1-methylethyl)-4-isoxazolyl]methyl}oxy)-2,3-dihydro-1H-inden-1-one (90%) (1.5 g, 3.2 mmol) in chloroform (10 mL). The reaction mixture was heated at reflux for 1 h. The reaction mixture was allowed to cool at room temperature, filtered, and the filtered solid was washed with dichloromethane. The filtrate was concentrated to give a dark green oil. The crude product was purified by flash chromatography over silica with a hexanes:ethyl acetate gradient (100:0 to 60:40) to give 1.1 g (69%) of 2-bromo-6-({[3-(2,6-dichlorophenyl)-5-(1-methylethyl)-4-isoxazolyl]methyl}oxy)-2,3-dihydro-1H-inden-1-one as a viscous colorless oil. The product was stored in the freezer under a nitrogen atmosphere. ¹H NMR (400 MHz, CDCl₃): δ 7.39 (m, 2H), 7.30 (m, 2H), 7.13 (d, J=2 Hz, 1H), 7.09 (dd, J=8, 3 Hz, 1H), 4.75 (s, 2H), 4.63 (dd, J=7, 3 Hz, 1H), 3.73 (dd, J=18, 7 Hz, 1H), 3.32 (m, 2H), 1.43 (d, J=7 Hz, 6H). ES-LCMS m/z 496 (M+H)⁺.

### 58d) 2-Bromo-6-({[3-(2,6-dichlorophenyl)-5-(1-methylethyl)-4-isoxazolyl]methyl}oxy)-2,3-dihydro-1H-inden-1-ol

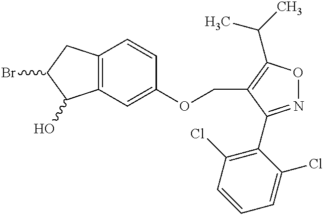

Sodium borohydride (0.046 g, 1.2 mmol) was added portion-wise over ten minutes to a stirred mixture of 2-bromo-6-({[3-(2,6-dichlorophenyl)-5-(1-methylethyl)-4-isoxazolyl]methyl}oxy)-2,3-dihydro-1H-inden-1-one (1.1 g, 2.22 mmol) in ethanol (10 mL) at room temperature. The reaction mixture was stirred at room temperature for 30 min. The reaction mixture was quenched with water and the aqueous mixture was extracted with dichloromethane. The layers were separated and the aqueous phase was extracted a second time with dichloromethane. The second dichloromethane extract did not contain significant product according to thin layer chromatography (hexanes:ethyl acetate (2:1)). The first dichloromethane extract was washed with saturated sodium chloride, dried over magnesium sulfate, filtered, and the filtrate was concentrated to give an oil (0.274 g). The aqueous phase noted above was extracted with ethyl acetate. The ethyl acetate extract was combined with the second dichloromethane extract noted earlier and the solution was washed with saturated sodium chloride, dried over magnesium sulfate, and filtered. The filtrate was combined with the oil obtained from the first dichloromethane extract noted earlier and the solution was concentrated to give 1.07 g (97%) of 2-bromo-6-({[3-(2,6-dichlorophenyl)-5-(1-methylethyl)-4-isoxazolyl]methyl}oxy)-2,3-dihydro-1H-inden-1-ol as a viscous oil. The product was stored in the freezer under a nitrogen atmosphere. ¹H NMR (400 MHz, CDCl₃): δ 7.40 (m, 2H), 7.31 (m, 1H), 7.08 (d, J=8 Hz, 1H), 6.86 (d, J=2 Hz, 1H), 6.71 (dd, J=8, 2 Hz, 1H), 4.88 (m, 2H), 4.71 (s, 2H), 3.36-3.23 (m, 3H), 2.38 (d, J=9 Hz, 1H), 1.41 (d, J=7 Hz, 6H). ES-LCMS m/z 498 (M+H)⁺.

### 58e) 4-{[(2-Bromo-1H-inden-5-yl)oxy]methyl}-3-(2,6-dichlorophenyl)-5-(1-methylethyl)isoxazole

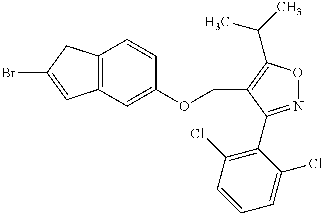

2-Bromo-6-({[3-(2,6-dichlorophenyl)-5-(1-methylethyl)-4-isoxazolyl]methyl}oxy)-2,3-dihydro-1H-inden-1-ol (1.1 g, 2.2 mmol), p-toluenesulfonic acid monohydrate (0.020 g, 0.11 mmol), and toluene (30 mL) were combined and the stirred reaction mixture was heated at reflux for 2 h under a nitrogen atmosphere. Water was removed from the reaction mixture with a Dean-Stark trap during the 2 h reflux period. Toluene was periodically added to the reaction mixture to replace the solvent that was drained from the Dean-Stark trap. The reaction mixture was allowed to cool at room temperature. The reaction mixture was concentrated and the crude product was purified by flash chromatography over silica with a hexanes:ethyl acetate gradient (100:0 to 90:10) to give an oil. The oil was dissolved in dichloromethane and the solution was concentrated to give 0.27 g (26%) of 4-{[(2-bromo-1H-inden-5-yl)oxy]methyl}-3-(2,6-dichlorophenyl)-5-(1-methylethyl)isoxazole as a colorless oil. ¹H NMR indicated that the product contained an impurity. The material was used without further purification. [Note: The column was subsequently flushed with ethyl acetate. Methanol and dichloromethane were added to the turbid ethyl acetate eluant to give a mixture of a red-brown precipitate and the clear yellow solution. The solution was decanted away from the precipitate and concentrated to give 0.68 g of impure 2-bromo-6-({[3-(2,6-dichlorophenyl)-5-(1-methylethyl)-4-isoxazolyl]methyl}oxy)-2,3-dihydro-1H-inden-1-ol as an oil.] ¹H NMR (400 MHz, CDCl₃): δ 7.39 (m, 2H), 7.31 (dd, J=9, 7 Hz, 1H), 7.17 (d, J=8 Hz, 1H), 6.80 (s, 1H), 6.71 (d, J=2 Hz, 1H), 6.58 (dd, J=8, 2 Hz, 1H), 4.72 (s, 2H), 3.50 (s, 2H), 3.32 (septet, J=7 Hz, 1H), 1.40 (d, J=7 Hz, 6H).

### 58f) 3-[6-({[3-(2,6-Dichlorophenyl)-5-(1-methylethyl)-4-isoxazolyl]methyl}oxy)-1H-inden-2-yl]benzoic acid and 3-[5-({[3-(2,6-dichlorophenyl)-5-(1-methylethyl)-4-isoxazolyl]methyl}oxy)-1H-inden-2-yl]benzoic acid

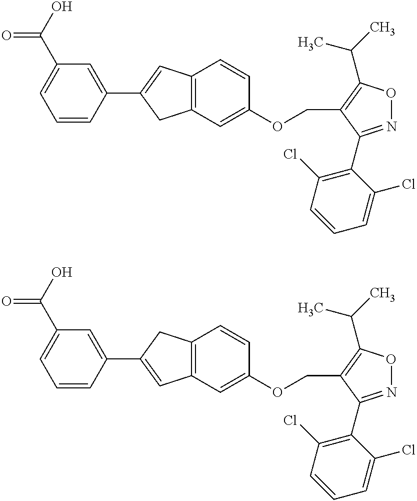

4-{[(2-Bromo-1H-inden-5-yl)oxy]methyl}-3-(2,6-dichlorophenyl)-5-(1-methylethyl)isoxazole (0.27 g, 0.56 mmol), 3-methoxycarbonylphenyl)boronic acid (0.163 g, 0.906 mmol), sodium carbonate (2 M) (1.2 mL, 2.4 mmol), tetrakistriphenylphosphinepalladium(0) (0.034 g, 0.029 mmol), and 1,2-dimethoxyethane (12 mL) were combined and the stirred reaction mixture was heated at reflux for 2 h under a nitrogen atmosphere. The reaction mixture was allowed to cool at room temperature. The reaction mixture was partitioned between water and ethyl acetate. The organic phase was separated, dried over magnesium sulfate, filtered, and the filtrate was concentrated to give an oil. The crude product was purified by flash chromatography over silica with a hexanes:ethyl acetate gradient (100:0 to 80:20) to give 0.163 g of an impure mixture of methyl 3-[6-({[3-(2,6-dichlorophenyl)-5-(1-methylethyl)-4-isoxazolyl]methyl}oxy)-1H-inden-2-yl]benzoate and methyl 3-[5-({[3-(2,6-dichlorophenyl)-5-(1-methylethyl)-4-isoxazolyl]methyl}oxy)-1H-inden-2-yl]benzoate as a viscous yellow oil. The crude mixture of esters was used without further purification. A mixture of methyl 3-[6-({[3-(2,6-dichlorophenyl)-5-(1-methylethyl)-4-isoxazolyl]methyl}oxy)-1H-inden-2-yl]benzoate and methyl 3-[5-({[3-(2,6-dichlorophenyl)-5-(1-methylethyl)-4-isoxazolyl]methyl}oxy)-1H-inden-2-yl]benzoate (0.16 g) was dissolved in tetrahydrofuran (4 mL) and methanol (2 mL). To the stirred solution was added sodium hydroxide (1 N) (0.3 mL, 0.3 mmol) at room temperature under a nitrogen atmosphere. The reaction mixture was stirred overnight at room temperature. Sodium hydroxide (1 N) (0.6 mL, 0.6 mmol) was added to the reaction mixture. The reaction mixture was stirred overnight at room temperature. The reaction mixture was concentrated and water (5 mL) was added to the residue. The pH of the aqueous mixture was adjusted to approximately 3 (litmus paper) with 10% citric acid. The acidic aqueous phase was extracted with ethyl acetate. The organic phase was separated, washed with water, followed by saturated sodium chloride, dried over magnesium sulfate, filtered, and the filtrate was concentrated to give an oil. The crude product was purified by flash chromatography over silica with a hexanes:ethyl acetate gradient (100:0 to 50:50) to give the impure product as a pale tan solid (0.024 g). Additional impure product (0.022 g) was obtained from the bump trap of the rotary evaporator with the aid of dichloromethane and methanol. The impure product (0.046 g) was purified by flash chromatography over silica with dichloromethane:methanol gradient beginning with 100% dichloromethane. The product eluted almost immediately from the column. The solvent was removed in vacuo and the product was dried to give 0.0225 g (7.7% from 4-{[(2-bromo-1H-inden-5-yl)oxy]methyl}-3-(2,6-dichlorophenyl)-5-(1-methylethyl)isoxazole) of an approximately 1:1 mixture of 3-[6-({[3-(2,6-dichlorophenyl)-5-(1-methylethyl)-4-isoxazolyl]methyl}oxy)-1H-inden-2-yl]benzoic acid and 3-[5-({[3-(2,6-dichlorophenyl)-5-(1-methylethyl)-4-isoxazolyl]methyl}oxy)-1H-inden-2-yl]benzoic acid as a pale tan solid. HRMS C₂₉H₂₄Cl₂NO₄ m/z 520.1082 (M+H)⁺_{Cal}; 520.1077 (M+H)⁺_{Obs}.

3-[5-({[3-(2,6-dichlorophenyl)-5-(1-methylethyl)-4-isoxazolyl]methyl}oxy)-1H-inden-2-yl]benzoic. ¹H NMR (400 MHz, DMSO-d₆): δ 13.04 (br s, 1H), 8.14 (s, 1H), 7.91 (d, J=8 Hz, 1H), 7.82 (d, J=8 Hz, 1H), 7.62 (m, 2H), 7.55-7.46 (m, 2H), 7.34 (s, 1H), 7.27 (d, J=8 Hz, 1H), 6.85 (d, J=2 Hz, 1H), 6.57 (dd, J=8, 2 Hz, 1H), 4.80 (s, 2H), 3.76 (s, 2H), 3.43 (septet, J=7 Hz, 1H), 1.31 (d, J=7 Hz, 6H).

3-[6-({[3-(2,6-dichlorophenyl)-5-(1-methylethyl)-4-isoxazolyl]methyl}oxy)-1H-inden-2-yl]benzoic. ¹H NMR (400 MHz, DMSO-d₆): δ 13.04 (br s, 1H), 8.11 (s, 1H), 7.88 (d, J=8 Hz, 1H), 7.79 (d, J=8 Hz, 1H), 7.62 (m, 2H), 7.55-7.46 (m, 2H), 7.36 (s, 1H), 7.23 (d, J=8 Hz, 1H), 6.93 (s, 1H), 6.68 (dd, J=8, 2 Hz, 1H), 4.81 (s, 2H), 3.76 (s, 2H), 3.43 (septet, J=7 Hz, 1H), 1.31 (d, J=7 Hz, 6H).

### Example 59

### 3-{[5-({[3-(2,6-Dichlorophenyl)-5-(1-methylethyl)-4-isoxazolyl]methyl}oxy)-1H-indol-1-yl]methyl}benzoic acid

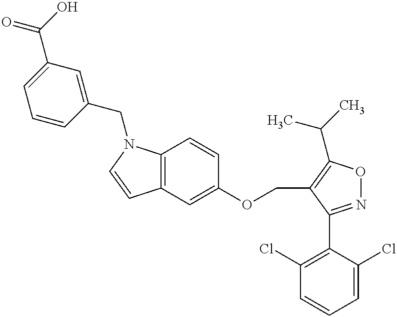

### 59a) Methyl 3-({5-[(phenylmethyl)oxy]-1H-indol-1-yl}methyl)benzoate

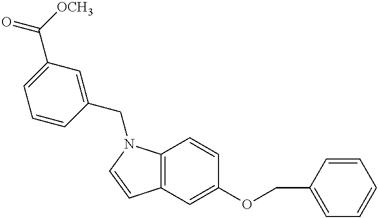

To a 3-neck round bottom flask equipped with a magnetic stirring bar and two nitrogen inlets was added sodium hydride (60% dispersion in oil) (5.1 g, 0.128 mol). The sodium hydride was washed with hexanes and the flask was equipped with an addition funnel. The addition funnel was charged with N,N-dimethylformamide (175 mL) and the solvent was added to the sodium hydride. The flask was equipped with a thermometer and the addition funnel was charged with a solution of 5-benzyloxyindole (25.3 g, 0.113 mol) in N,N-dimethylformamide (350 mL). The 5-benzyloxyindole solution was slowly added dropwise to the stirred sodium hydride suspension at room temperature under a nitrogen atmosphere over a period of 2 h. Gas evolution was observed during the addition of the 5-benzyloxyindole solution. The dark brown reaction mixture was stirred for 10 minutes. To the reaction mixture was added dropwise a solution of methyl-3-bromomethyl benzoate (28.5 g, 0.124 mol) in N,N-dimethylformamide (200 mL) at room temperature under a nitrogen atmosphere over a period of 1 h. The reaction mixture warmed slightly to 28° C. upon addition of the methyl-3-bromomethyl benzoate solution. The reaction mixture was stirred overnight. Water (50 mL) was added to the stirred reaction mixture very slowly dropwise before adding another 50 mL of water more rapidly. No discernible gas evolution was evident upon addition of water, however, the reaction mixture warmed slightly to 28° C. The quenched reaction mixture was transferred to a separatory funnel which contained ethyl acetate (1200 mL) and water (1100 mL). The mixture was stirred with the aid of a spatula. The aqueous phase was separated and the organic phase was washed with water (2×500 mL) followed by brine (500 mL). The organic phase was dried over magnesium sulfate, filtered, and the filtrate was concentrated to give 45.3 g of the crude product as an oil. Approximately one-half of the crude product was purified by flash chromatography over silica with a hexanes:ethyl acetate gradient (100:0 to 70:30) to give 6.9 g of methyl 3-({5-[(phenylmethyl)oxy]-1H-indol-1-yl}methyl)benzoate as a yellow oil. The remaining crude product was purified by flash chromatography over silica with a hexanes:ethyl acetate gradient (100:0 to 70:30) to give another 5.4 g of methyl 3-({5-[(phenylmethyl)oxy]-1H-indol-1-yl}methyl)benzoate as a yellow oil for a total yield of 12.3 g (29%). ¹H NMR (400 MHz; CDCl₃): δ 7.93 (d, J=8 Hz, 1H), 7.91 (s, 1H), 7.47 (d, J=7 Hz, 2H), 7.36 (m, 4H), 7.20 (m, 2H), 7.13 (m, 2H), 6.91 (dd, J=9, 2 Hz, 1H), 6.48 (d, J=3 Hz, 1H), 5.32 (s, 2H), 5.09 (s, 2H), 3.90 (s, 3H). ES-LCMS m/z 372 (M+H)⁺.

### 59b) Methyl 3-[(5-hydroxy-1H-indol-1-yl)methyl]benzoate

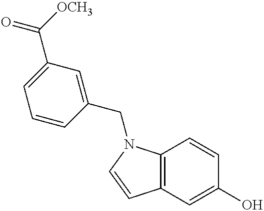

To a suspension of 10% palladium on carbon (Degussa Type; 50% water by wt.) (1.46 g) in ethanol (50 mL) was added a solution of methyl 3-({5-[(phenylmethyl)oxy]-1H-indol-1-yl}methyl)benzoate (6.9 g, 18.6 mmol) in ethyl acetate (100 mL). The round bottom flask was evacuated and filled with nitrogen twice, evacuated, and filled with hydrogen using a balloon. The reaction mixture was stirred overnight at room temperature under hydrogen. After 18 h, the reaction mixture was filtered though a pad of Celite®. The pad was washed with ethyl acetate and the filtrate was concentrated to give the crude product. The crude product was purified by flash chromatography over silica with a dichloromethane:methanol gradient (100:0 to 98:2) to give 3.4 g (66%) of methyl 3-[(5-hydroxy-1H-indol-1-yl)methyl]benzoate as a colorless oil. ¹H NMR (400 MHz; CDCl₃): δ 7.93 (d, J=8 Hz, 1H), 7.90 (s, 1H), 7.34 (t, J=8 Hz, 1H), 7.19 (d, J=8 Hz, 1H), 7.11 (d, J=3 Hz, 1H), 7.08 (d, J=9 Hz, 1H), 7.04 (d, J=2 Hz, 1H), 6.73 (dd, J=9, 3 Hz, 1H), 6.43 (d, J=3 Hz, 1H), 5.30 (s, 2H), 4.46 (br s, 1H), 3.89 (s, 3H). ES-LCMS m/z 282 (M+H)⁺.

### 59c) Methyl 3-{[5-({[3-(2,6-dichlorophenyl)-5-(1-methylethyl)-4-isoxazolyl]methyl}oxy)-1H-indol-1-yl]methyl}benzoate

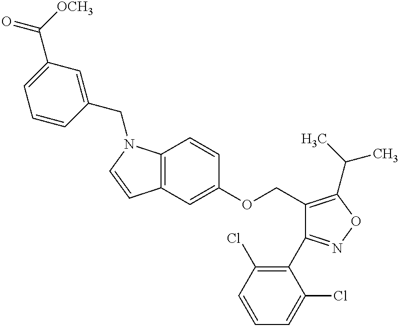

To a solution of methyl 3-[(5-hydroxy-1H-indol-1-yl)methyl]benzoate (3.26 g, 11.6 mmol) in N,N-dimethylformamide (26 mL) was added cesium carbonate (5.85 g, 18 mmol). The suspension was heated at 65° C. with stirring under nitrogen for 1 h. The reaction mixture was allowed to stand at room temperature overnight. The reaction mixture was heated at 65° C. and a solution of 4-(chloromethyl)-3-(2,6-dichlorophenyl)-5-(1-methylethyl)isoxazole (4.0 g, 13.1 mmol) in N,N-dimethylformamide (20 mL) was added dropwise with stirring under nitrogen. The reaction mixture was heated overnight at 65° C. After 16 h, the reaction mixture was allowed to cool at room temperature and partitioned between ethyl acetate and water. The organic phase was separated, washed with water, followed by brine, dried over magnesium sulfate, filtered, and the filtrate was concentrated to give the crude product. The crude product was purified by flash chromatography over silica with a hexanes:ethyl acetate gradient (100:0 to 60:40) to give 4.51 g (71%) of methyl 3-{[5-({[3-(2,6-dichlorophenyl)-5-(1-methylethyl)-4-isoxazolyl]methyl}oxy)-1H-indol-1-yl]methyl}benzoate as a yellow oil. ¹H NMR (400 MHz; CDCl₃): δ 7.92 (d, J=8 Hz, 1H), 7.87 (s, 1H), 7.26-7.38 (m, 4H), 7.16 (d, J=8 Hz, 1H), 7.09 (d, J=2 Hz, 1H), 7.04 (d, J=9 Hz, 1H), 6.98 (d, J=2 Hz, 1H), 6.66 (dd, J=9, 2 Hz, 1H), 6.41 (d, J=3 Hz, 1H), 5.29 (s, 2H), 4.73 (s, 2H), 3.88 (s, 3H), 3.31 (septet, J=7 Hz, 1H), 1.38 (d, J=7 Hz, 6H). ES-LCMS m/z 549 (M+H)⁺.

### 59d) 3-{[5-({[3-(2,6-Dichlorophenyl)-5-(1-methylethyl)-4-isoxazolyl]methyl}oxy)-1H-indol-1-yl]methyl}benzoic acid

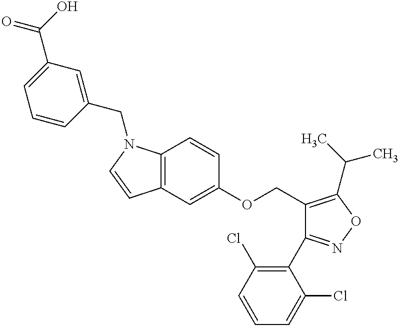

To a solution of methyl 3-{[5-({[3-(2,6-dichlorophenyl)-5-(1-methylethyl)-4-isoxazolyl]methyl}oxy)-1H-indol-1-yl]methyl}benzoate (4.3 g, 7.8 mmol) in tetrahydrofuran (100 mL) and methanol (50 mL) was added sodium hydroxide (1N) (16 mL, 16 mmol). The reaction mixture was heated at 65° C. for 1 h with stirring under nitrogen. The reaction mixture was partially concentrated, and water (100 mL) was added to the aqueous residue. The pH of the aqueous mixture was adjusted to approximately 2 (litmus paper) with 1N hydrochloric acid (approximately 12 mL). To the acidic aqueous mixture was added ethyl acetate followed by 1N hydrochloric acid (approximately 4 mL). The organic phase was separated, washed with water (100 mL), followed by brine, dried over magnesium sulfate, filtered, and the filtrate was concentrated to give the crude product. The crude product was purified by flash chromatography over silica with a hexanes:ethyl acetate gradient (75:25 to 60:40) to give 3.1 g (74%) of 3-{[5-({[3-(2,6-dichlorophenyl)-5-(1-methylethyl)-4-isoxazolyl]methyl}oxy)-1H-indol-1-yl]methyl}benzoic acid as a white amorphous solid.

¹H NMR (400 MHz, DMSO-d₆): δ 12.91 (br s, 1H), 7.77 (d, J=7 Hz, 1H), 7.68 (s, 1H), 7.58 (m, 2H), 7.50 (dd, J=9, 7 Hz, 1H), 7.42 (d, J=3 Hz, 1H), 7.39 (t, J=8 Hz, 1H), 7.35 (d, J=8 Hz, 1H), 7.21 (d, J=9 Hz, 1H), 6.94 (d, J=2 Hz, 1H), 6.50 (dd, J=9, 2 Hz, 1H), 6.31 (d, J=3 Hz, 1H), 5.40 (s, 2H), 4.73 (s, 2H), 3.36 (septet, J=7 Hz, 1H), 1.25 (d, J=7 Hz, 6H). ES-LCMS m/z 535 (M+H)⁺.

### Example 60

### 3-[6-({[3-(2,6-Dichlorophenyl)-5-(1-methylethyl)-4-isoxazolyl]methyl}oxy)-1-oxo-3,4-dihydro-2(1H)-isoquinolinyl]benzoic acid

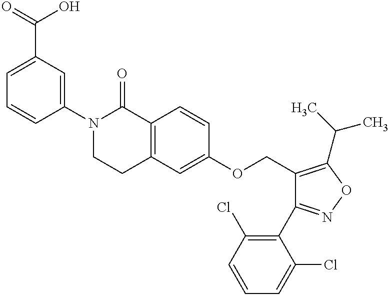

### 60a) Ethyl {2-[3-(methyloxy)phenyl]ethyl}carbamate

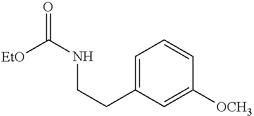

To an ice-water cooled, stirred solution of 3-methoxyphenethylamine (2.4 mL, 16.5 mmol) and triethylamine (2.6 mL, 18.7 mmol) in dichloromethane (50 mL) was slowly added ethyl chloroformate (1.8 mL, 18.8 mmol) under nitrogen. The reaction mixture was stirred for 1.5 h with cooling. The reaction mixture was washed with water, followed by 1N hydrochloric acid, and finally brine. The organic phase was separated, dried over magnesium sulfate, filtered, and the filtrate was concentrated to give 3.37 g of ethyl {2-[3-(methyloxy)phenyl]ethyl}carbamate as a yellow oil. The crude product was used without further purification. ¹H NMR (400 MHz, CDCl₃): δ 7.22 (t, J=8 Hz, 1H), 6.77 (m, 2H), 6.73 (s, 1H), 4.65 (br s, 1H), 4.10 (q, J=7 Hz, 2H), 3.79 (s, 3H), 3.43 (m, 2H), 2.78 (t, J=7 Hz, 2H), 1.22 (t, J=7 Hz, 3H).

### 60b) 6-(Methyloxy)-3,4-dihydro-1(2H)-isoquinolinone

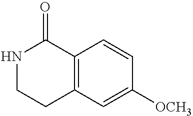

Ethyl {2-[3-(methyloxy)phenyl]ethyl}carbamate (3.36 g) and polyphosphoric acid (12.67 g) were combined and the reaction mixture was heated for 2 h at 120° C. under nitrogen. The oil bath was removed and the reaction mixture was allowed to cool at room temperature. Water was added to the reaction mixture and the aqueous solution was extracted twice with ethyl acetate. The organic extracts were combined, washed with brine, dried over magnesium sulfate, filtered, and the filtrate was concentrated to give the crude product as a sticky tan solid. The crude product was purified by flash chromatography over silica with a hexanes:ethyl acetate gradient (100:0 to 0:100) to give 1.49 g (51% from 3-methoxyphenethylamine) of 6-(methyloxy)-3,4-dihydro-1(2H)-isoquinolinone as a white solid. ¹H NMR (400 MHz, CDCl₃): δ 8.01 (d, J=9 Hz, 1H), 6.87 (dd, J=9, 3 Hz, 1H), 6.71 (d, J=2 Hz, 1H), 6.26 (br s, 1H), 3.85 (s, 3H), 3.57 (t, J=7 Hz, 2H), 2.98 (t, J=7 Hz, 2H).

### 60c) Ethyl 3-[6-(methyloxy)-1-oxo-3,4-dihydro-2(1H)-isoquinolinyl]benzoate

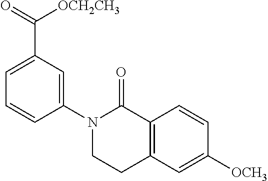

6-(Methyloxy)-3,4-dihydro-1(2H)-isoquinolinone (0.319 g, 1.8 mmol), ethyl-3-iodobenzoate (0.62 mL, 3.68 mmol), copper (I) iodide (0.044 g, 0.23 mmol), potassium carbonate (0.247 g, 1.8 mmol) and N,N-dimethylformamide (4 mL) were combined and the stirred reaction mixture was heated at 150° C. under nitrogen for 28 h. The reaction mixture was partitioned between water and ethyl acetate. The layers were separated and the aqueous phase was extracted with ethyl acetate. The organic extracts were combined, dried over magnesium sulfate, filtered, and the filtrate was concentrated to give a gold-yellow liquid. The crude product was purified by flash chromatography over silica with a hexanes:ethyl acetate gradient (100:0 to 50:50) to give 0.32 g (55%) of ethyl 3-[6-(methyloxy)-1-oxo-3,4-dihydro-2(1H)-isoquinolinyl]benzoate as a clear colorless oil. ¹H NMR (400 MHz; CDCl₃): δ 8.10 (d, J=9 Hz, 1H), 8.00 (s, 1H), 7.91 (d, J=8 Hz, 1H), 7.63 (d, J=8 Hz, 1H), 7.46 (t, J=8 Hz, 1H), 6.89 (dd, J=9, 2 Hz, 1H), 6.73 (d, J=2 Hz, 1H), 4.38 (q, J=7 Hz, 2H), 4.01 (t, J=6 Hz, 2H), 3.87 (s, 3H), 3.12 (t, J=6 Hz, 2H), 1.38 (t, J=7 Hz, 3H). ES-LCMS m/z 326 (M+H)⁺.

### 60d) 3-(6-Hydroxy-1-oxo-3,4-dihydro-2(1H)-isoquinolinyl)benzoic acid

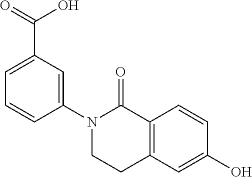

To an stirred ice-water cooled solution of ethyl 3-[6-(methyloxy)-1-oxo-3,4-dihydro-2(1H)-isoquinolinyl]benzoate (0.315 g, 0.97 mmol) in dichloromethane (10 mL) was slowly added boron tribromide (1M in dichloromethane) (6 mL, 6 mmol) under nitrogen.

The ice-water bath was removed and the reaction mixture was stirred at room temperature under nitrogen. After 4 h, ES-LCMS analysis indicated that the reaction mixture contained both methyl 3-(6-hydroxy-1-oxo-3,4-dihydro-2(1H)-isoquinolinyl)benzoate and 3-(6-hydroxy-1-oxo-3,4-dihydro-2(1H)-isoquinolinyl)benzoic acid. The reaction mixture was poured into ice-water and the mixture was extracted with dichloromethane. The layers were separated and the aqueous phase was extracted with dichloromethane. The organic extracts were combined, washed with brine, dried over magnesium sulfate, filtered, and the filtrate was allowed to stand at room temperature overnight. The filtrate was concentrated to give 0.058 g of a residue. ES-LCMS analysis of the residue showed numerous UV-peaks including those peaks corresponding to methyl 3-(6-hydroxy-1-oxo-3,4-dihydro-2(1H)-isoquinolinyl)benzoate and 3-(6-hydroxy-1-oxo-3,4-dihydro-2(1H)-isoquinolinyl)benzoic acid. Both the aqueous phase and the brine from the aforementioned workup were sequentially filtered through a single sintered-glass funnel to give a white solid. The filtered solid was washed with water and dissolved in methanol. The methanolic solution was filtered and the filtrate was concentrated to give 0.186 g (68%) of 3-(6-hydroxy-1-oxo-3,4-dihydro-2(1H)-isoquinolinyl)benzoic acid as a pale tan solid. ES-LCMS m/z 284 (M+H)⁺.

### 60e) Methyl 3-(6-hydroxy-1-oxo-3,4-dihydro-2(1H)-isoquinolinyl)benzoate

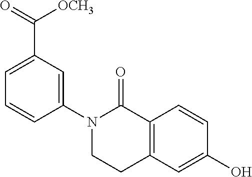

To a stirred suspension of 3-(6-hydroxy-1-oxo-3,4-dihydro-2(1H)-isoquinolinyl)benzoic acid (0.186 g, 0.66 mmol) in methanol (10 mL) was slowly added dropwise thionyl chloride (0.14 mL, 1.92 mmol) at room temperature under nitrogen. The reaction mixture was heated at reflux for 2.5 h. The reaction mixture was concentrated and toluene was added to the residue. The solvent was removed in vacuo and toluene was again added to the residue. The solvent was removed in vacuo to give the crude product as an oil which partially solidified upon standing. The crude product was purified by flash chromatography over silica with a hexanes:ethyl acetate (50:50 to 0:100) gradient to give 0.17 g (87%) of methyl 3-(6-hydroxy-1-oxo-3,4-dihydro-2(1H)-isoquinolinyl)benzoate as a white solid. ¹H NMR (400 MHz, CDCl₃): δ 8.05 (d, J=8 Hz, 1H), 8.01 (s, 1H), 7.91 (d, J=8 Hz, 1H), 7.64 (d, J=8 Hz, 1H), 7.47 (t, J=8 Hz, 1H), 6.80 (d, J=8 Hz, 1H), 6.69 (s, 1H), 4.00 (t, J=6 Hz, 2H), 3.91 (s, 3H), 3.10 (t, J=6 Hz, 2H). ES-LCMS m/z 298 (M+H)⁺.

### 60f) Methyl 3-[6-({[3-(2,6-dichlorophenyl)-5-(1-methylethyl)-4-isoxazolyl]methyl}oxy)-1-oxo-3,4-dihydro-2(1H)-isoquinolinyl]benzoate

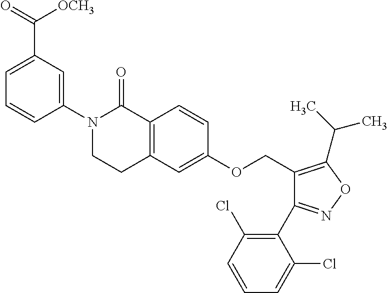

To a stirred mixture of methyl 3-(6-hydroxy-1-oxo-3,4-dihydro-2(1H)-isoquinolinyl)benzoate (0.17 g, 0.57 mmol), [3-(2,6-dichlorophenyl)-5-(1-methylethyl)-4-isoxazolyl]methanol (prepared by the general procedure described in Maloney, P. R., et al., 2000 J. Med. Chem. 43:2971-2974) (0.196 g, 0.68 mmol), triphenylphosphine (polystyrene bound, 2.1 mmol/g) (0.324 g, 0.68 mmol), and dichloromethane (8 mL) was slowly added diisopropyl azodicarboxylate (0.14 mL, 0.71 mmol) at room temperature under nitrogen. The reaction mixture was stirred for 4 days and filtered. The resin was washed with dichloromethane and the filtrate was concentrated to give a yellow oil. The crude product was purified by flash chromatography over silica with a hexanes:ethyl acetate gradient (100:0 to 50:50) to give the product as an oil. The product was dissolved in dichloromethane and concentrated three times to give 0.209 g (65%) of methyl 3-[6-({[3-(2,6-dichlorophenyl)-5-(1-methylethyl)-4-isoxazolyl]methyl}oxy)-1-oxo-3,4-dihydro-2(1H)-isoquinolinyl]benzoate as a white amorphous solid. ¹H NMR (400 MHz, CDCl₃): δ 8.02 (d, J=9 Hz, 1H), 7.99 (m, 1H), 7.90 (d, J=8 Hz, 1H), 7.62 (d, J=9 Hz, 1H), 7.46 (t, J=8 Hz, 1H), 7.41 (m, 2H), 7.33 (dd, J=9, 7 Hz, 1H), 6.75 (dd, J=9, 3 Hz, 1H), 6.59 (d, J=2 Hz, 1H), 4.79 (s, 2H), 3.97 (t, J=6 Hz, 2H), 3.91 (s, 3H), 3.34 (septet, J=7 Hz, 1H), 3.06 (t, J=6 Hz, 2H), 1.43 (d, J=7 Hz, 6H). ES-LCMS m/z 565 (M+H)⁺.

### 60g) 3-[6-({[3-(2,6-Dichlorophenyl)-5-(1-methylethyl)-4-isoxazolyl]methyl}oxy)-1-oxo-3,4-dihydro-2(1H)-isoquinolinyl]benzoic acid

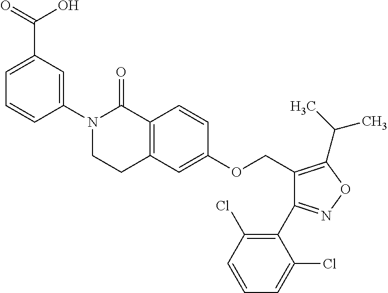

To a solution of methyl 3-[6-({[3-(2,6-dichlorophenyl)-5-(1-methylethyl)-4-isoxazolyl]methyl}oxy)-1-oxo-3,4-dihydro-2(1H)-isoquinolinyl]benzoate (0.204 g, 0.36 mmol) in tetrahydrofuran (8 mL) was added methanol (4 mL) and lithium hydroxide (1N) (0.74 mL, 0.74 mmol). The reaction mixture was heated at 100° C. in a microwave for 500 seconds. The reaction mixture was concentrated and the crude product was partitioned between ethyl acetate (50 mL), water (20 mL) and saturated sodium hydrogensulfate (0.5 mL). The organic phase was separated, washed with water (20 mL), followed by brine (20 mL), dried over magnesium sulfate, filtered, and the filtrate was concentrated. During concentration of the filtrate on the rotary evaporator, a portion of the solution in the round bottom flask bumped into the bump trap. The solutions in the bump trap and round bottom flask were concentrated independently. The solution in the round bottom flask was concentrated and the product was dissolved in methanol. The methanolic solution was concentrated, and the product was dried under high vacuum at 60° C. to give 0.071 of 3-[6-({[3-(2,6-dichlorophenyl)-5-(1-methylethyl)-4-isoxazolyl]methyl}oxy)-1-oxo-3,4-dihydro-2(1H)-isoquinolinyl]benzoic acid as a white amorphous solid. The solution in the bump trap was concentrated to give a viscous oil. The viscous oil was dissolved in tetrahydrofuran and concentrated twice to give 0.106 g of 3-[6-({[3-(2,6-dichlorophenyl)-5-(1-methylethyl)-4-isoxazolyl]methyl}oxy)-1-oxo-3,4-dihydro-2(1H)-isoquinolinyl]benzoic acid as a white amorphous solid for a total yield of 0.177 g (89%). ¹H NMR (400 MHz, DMSO-d₆): δ 13.09 (br s, 1H), 7.88 (m, 1H), 7.77 (m, 2H), 7.62 (m, 2H), 7.57 (m, 1H), 7.53 (dd, J=9, 7 Hz, 1H), 7.47 (t, J=8 Hz, 1H), 6.77 (m, 1H), 6.74 (dd, J=9, 2 Hz, 1H), 4.88 (s, 2H), 3.91 (t, J=6 Hz, 2H), 3.46 (septet, J=7 Hz, 1H), 3.01 (t, J=6 Hz, 2H), 1.33 (d, J=7 Hz, 6H). HRMS C₂₉H₂₅Cl₂N₂O₅ m/z 551.11350 (M+H)⁺_{Cal;} 551.11348 (M+H)⁺_{Obs}.

### Biological Example 61

### FXR Cofactor Binding Assay

Determination of a ligand mediated cofactor peptide interaction to quantify ligand binding to the nuclear receptor Farnesoid X Receptor (FXR). The method measures the ability of putative ligands to modulate the interaction between the purified bacterial expressed FXRα ligand binding domain (LBD) and a synthetic biotinylated peptide based on residues 676-700 of steroid receptor coactivator-1 (SRC-1) (LXXLL-containing domain-2 where L is the amino acid leucine and X indicates any other amino acid (LCD2), 676-700). The sequence of the SRC-1 peptide used is as published in Iannone, M. A., et al., 2001 Cytometry 44:326-337 where the N-terminus was biotinylated (B) and the C-terminus was amidated. Detection of the associated complex was measured by time resolved fluorescence (TRF). The purified LBD of FXR was labeled with biotin, then mixed with stoichiometric amounts of allophycocyanin (APC) labeled streptavidin (Molecular Probes). The biotinylated peptide was then mixed with a ½ stoichiometric amount of europium labeled streptavidin (Wallac Inc). Each was then blocked with a 5 fold excess of biotin and allowed to equilibrate for 15 min. Equimolar amounts of receptor and peptide were mixed together and were allowed to equilibrate for at least 30 min prior to the addition to either a variable or constant concentrations of the sample for which the affinity is to be determined. After equilibration, the time-resolved fluorescent signal was quantitated using a fluorescent plate reader. The affinity of the test compound was estimated from a plot of fluorescence versus concentration of test compound added.

A basal level of FXR: peptide formation is observed in the absence of added ligand. Ligands that promote the complex formation induce a concentration-dependent increase in time-resolved fluorescent signal. Compounds which bind equally well to both monomeric FXR and to the FXR: peptide complex would be expected to give no change in signal, whereas ligands which bind preferentially to the monomeric receptor would be expected to induce a concentration-dependent decrease in the observed signal.

### Methods & Materials

### Advance Preparation: Human Farnesoid X Receptor α Ligand Binding Domain

Human FXRα Ligand Binding Domain (FXRα LBD) was expressed in E. coli strain BL21 (DE3) as an amino-terminal polyhistidine tagged fusion protein. Expression was under the control of an isopropyl-β-D-thiogalactopyranoside (IPTG) inducible T7 promoter. DNA encoding this recombinant protein is subcloned into the pRSET-A expression vector (Invitrogen). The coding sequence of Human FXRα LBD was derived from Genbank accession number U 68233 (amino acids 237 to 472).

Ten-liter fermentation batches were grown in Rich PO₄ media with 0.1 mg/mL Ampicillin at 25° C. for 12 hours, cooled to 9° C. and held at that temperature for 36 hours to a density of OD₆₀₀=14. At this cell density, 0.25 mM IPTG is added and induction proceeded for 24 hours at 9° C., to a final OD₆₀₀=16. Cells are harvested by centrifugation (20 minutes, 3500×gravity, 4° C.), and concentrated cell slurries were stored in phosphate buffered saline (PBS) at −8° C.

### Purification of Receptor Ligand Binding Domain

Routinely, 30-40 g cell paste (equivalent to 2-3 liters of the fermentation batch) was resuspended in 200-250 mL Tris buffered saline (TBS), pH 7.2 (25 mM Tris-hydroxymethylamino methane (Tris), 150 mM sodium chloride). Cells were lysed by passing 3 times through a French Press and cell debris was removed by centrifugation (30 minutes, 20,000×gravity, 4° C.). The cleared supernatant was filtered through course pre-filters, and TBS, pH 7.2, 500 mM imidazole was added to obtain a final imidazole concentration of 50 mM. This lysate was loaded onto a column (6×8 cm) packed with Sepharose [Ni⁺⁺ charged] Chelation resin (Pharmacia) and pre-equilibrated with TBS pH 7.2/50 mM imidazole. After washing to baseline absorbance with equilibration buffer, the column was washed with one column volume of TBS pH 7.2 containing 90 mM imidazole. FXRα LBD was eluted directly with 365 mM imidazole. Column fractions were pooled and dialyzed against TBS, pH 7.2, containing 0.5 mM EDTA and 5 mM DTT. The dialyzed protein sample was concentrated using Centri-prep 10 K (Amicon) and subjected to size exclusion, using a column (3×90 cm) packed with Sepharose S-75 resin (Pharmacia) pre-equilibrated with TBS, pH 7.2, containing 0.5 mM ethylene diamine tetraacetic acid (EDTA) and 5 mM dithiothreitol (DTT).

### Biotinylation of FXR

Purified FXRα LBD was desalted/buffer exchanged using PD-10 gel filtration columns into PBS [100 mM Na₂PO₄, pH 7.2, 150 mM NaCl]. FXRα LBD was diluted to approximately 60 μM in PBS and five-fold molar excess of NHS-LC-Biotin (Pierce) is added in a minimal volume of PBS. This solution was incubated with gentle mixing for 30 minutes at room temperature. The biotinylation modification reaction was stopped by the addition of 2000× molar excess of Tris-HCl, pH 8. The modified FXRα LBD was dialyzed against 4 buffer changes, each of at least 50 volumes, PBS containing 5 mM DTT, 2 mM EDTA and 2% sucrose. The biotinylated FXRα LBD was then subjected to mass spectrometric analysis to reveal the extent of modification by the biotinylation reagent. In general, approximately 95% of the protein had at least a single site of biotinylation; and the overall extent of biotinylation followed a normal distribution of multiple sites, ranging from zero to four.

### Preparation of Streptavidin-(Europium Chelate)-SRC1:Streptavdin-(APC)-FXR Complex

Biotinylated SRC-1 (LCD2, 676-700) peptide and a ½ stoichiometric amount of streptavidin-conjugated europium chelate was incubated in assay buffer containing 10 mM DTT for at least 30 minutes. A second solution of stoichiometric amounts of biotinylated FXR and streptavidin-conjugated APC was incubated in assay buffer containing 10 mM DTT for at least 30 minutes. Each solution was then blocked with a 5 fold molar excess of biotin and allowed to equilibrate for at least 30 min. The labeled receptor and cofactor were mixed and again allowed to equilibrate for at least 30 min, added to the compound plate, utilizing e.g., a Titertek Multidrop 384.

Materials:

Assay Buffer: 50 mM 3-(N-morpholino)propanesulfonic acid (MOPS) pH 7.5, 50 mM NaF, 50 μM 3-[(3-cholamidopropyl)-demethylammonio]-1-propanesulfonate (CHAPS), 0.1 mg/ml Fraction 5 fatty acid free bovine serum albumin (BSA), 1 mM ethylenediaminetetraacetic acid (EDTA). Solid DTT is added to the assay buffer to a final concentration of 10 mM just before use in the assay. BSA, fatty acid free

DTT

NaF

Europium labeled Streptavidin: (Wallac CR28-100)

384 well Plates

Methods:

Experimental Details:

Test compounds and controls were serial diluted in DMSO and 0.1 μL at the desired concentration were added to a 384 well plate.

To each well to be assayed a previously prepared solution of FXR-APC and Europium labeled SRC1 was added to 0.1 μL of test compound and controls for a final assay volume of 10 μL.

The plates were incubated for at least 1 hour at room temperature and the fluorescent signal determined in a Fluorescence Reader in a time resolved mode utilizing e.g., a Wallac Viewlux Imager or Wallac Victor Multilabel counter.

Data Reduction:

For each concentration of test compound, the results of each test well was expressed as % of control, C, calculated according to eq. 1.

$$C = 100 * \frac{{F}_{sample} - {F}_{basal}}{{F}_{std} - {F}_{basal}} \qquad ( 1 )$$

where Fₛₐₘₚₗₑ is the signal observed in a particular sample well, F_{std} is the signal observed in the presence of control agonist and F_{basal} is the count rate observed in the presence of no ligand. The values used for F_{std} and F_{basal} are averages of the corresponding control wells included on every plate. The results are reported in Table 1 below. In Table 1, + indicates a pEC₅₀ of 5-5.99; ++ indicates a pEC₅₀ 6-6.99 and +++ indicates a pEC₅₀ greater than 7.

TABLE 1

| Example | Activity (pEC₅₀) |
|---|---|
| 1 | ++ |
| 2 | + |
| 3 | ++ |
| 4 | + |
| 5 | ++ |
| 6 | + |
| 7 | ++ |
| 8 | ++ |
| 9 | ++ |
| 10 | ++ |
| 11 | ++ |
| 12 | ++ |
| 13 | ++ |
| 14 | ++ |
| 15 | ++ |
| 16 | ++ |
| 17 | ++ |
| 18 | ++ |
| 19 | ++ |
| 20 | ++ |
| 21 | ++ |
| 22 | ++ |
| 23 | ++ |
| 24 | ++ |
| 25 | ++ |
| 26 | ++ |
| 27 | ++ |
| 28 | ++ |
| 29 | ++ |
| 30 | ++ |
| 31 | ++ |
| 32 | ++ |
| 33 | ++ |
| 34 | ++ |
| 35 | ++ |
| 36 | ++ |
| 37 | ++ |
| 38 | ++ |
| 39 | + |
| 40 | ++ |
| 41 | + |
| 42 | + |
| 43 | ++ |
| 44 | ++ |
| 45 | + |
| 46 | +++ |
| 47 | ++ |
| 48 | ++ |
| 49 | ++ |
| 50 | + |
| 51 | ++ |
| 52 | ++ |
| 53 | ++ |
| 54 | +++ |
| 55 | ++ |
| 56 | ++ |
| 57 | ++ |
| 58 | ++ |
| 59 | ++ |
| 60 | ++ |

1. A compound of formula (I): 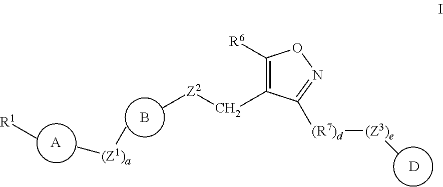
wherein:
Ring A is phenyl or a 5-6 membered heteroaryl comprising 1, 2 or 3 heteroatoms selected from N, O and S, wherein said phenyl or heteroaryl is substituted with R¹ and further optionally substituted with one or two substituents independently selected from C₁₋₆alkyl, halo and haloalkyl;
R¹ is selected from —CO₂H, —C(O)NH₂, —CO₂alkyl, —CH₂CH₂CO₂H, —CH₂CH₂CO₂alkyl, —NHC(O)CH₃, —N(C(O)CH₃)₂, —N(SO₂CF₃)₂, —OCF₃ and an acid equivalent group
Z¹ is —CH₂—, —CO—, —NH—, —S—, —SO— or —SO₂—;
a is 0 or 1;
Ring B is selected from 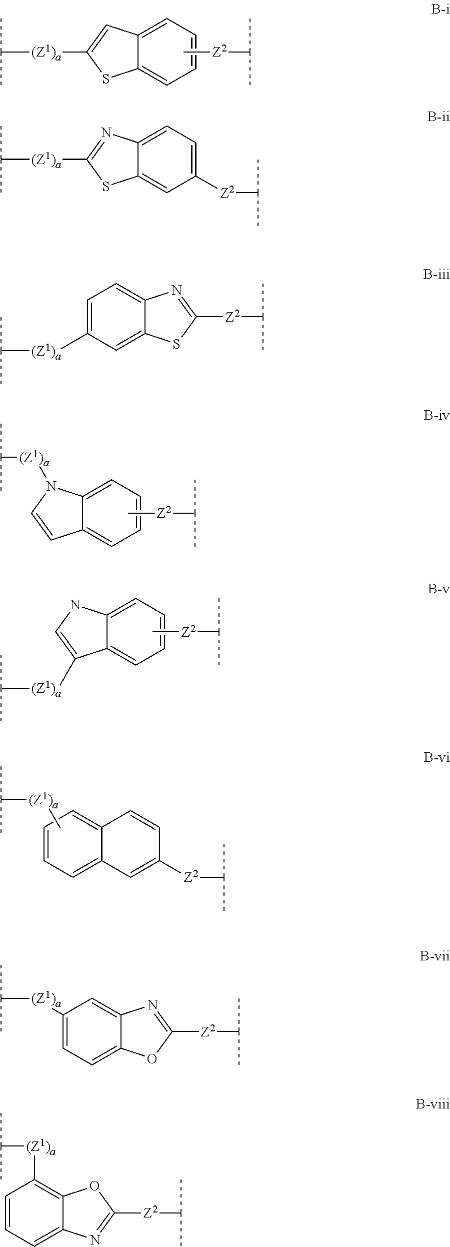 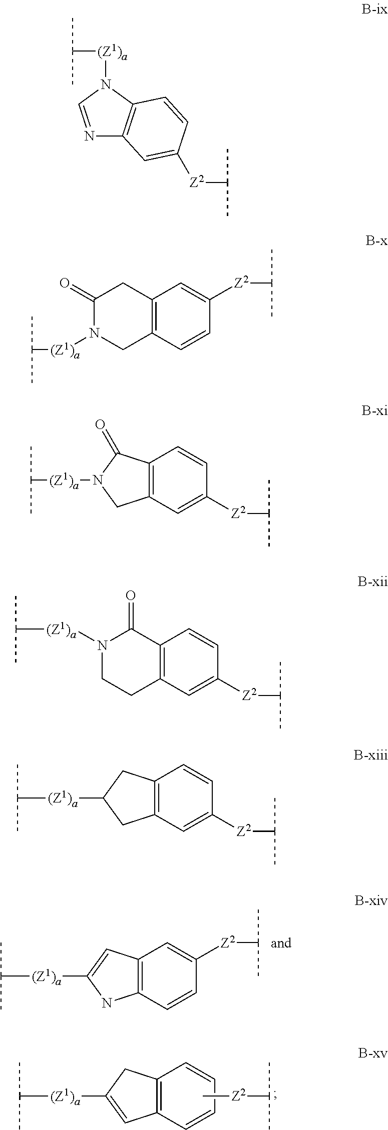
Z² is —O—, —S—, —CH₂— or —N(R⁵)—, wherein R⁵ is H or alkyl;
R⁶ is selected from alkyl, 2,2,2-trifluoroethyl, C₃₋₆cycloalkyl, alkenyl, C₃₋₆cycloalkenyl and fluoro-substituted C₃₋₆cycloalkyl;
R⁷ is —C₁₋₃alkylene-;
Z³ is —O—, —S(O)_{c}—, or —NH—, where c is 0, 1 or 2;
d and e are both 0 or d is 1 and e is 0 or 1; and
Ring D is selected from C₃₋₆cycloalkyl and a moiety of formula D-i, D-ii, D-iii, D-iv or D-v 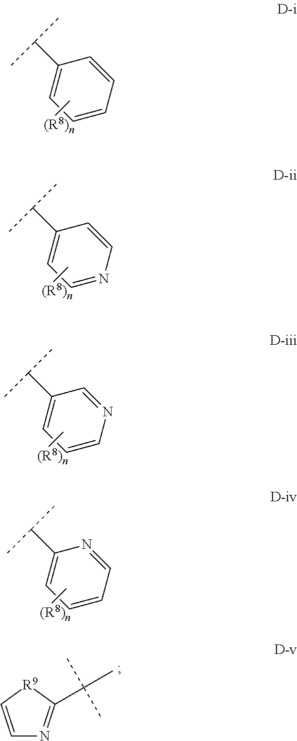
wherein n is 0, 1, 2 or 3;
each R⁹ is the same or different and is independently selected from halo, alkyl, alkenyl, —O-alkyl, haloalkyl, hydroxyl substituted alkyl, and —OCF₃;
R⁹ is —O—, —NH— or —S—;
or a pharmaceutically acceptable salt thereof.

2. The compound according to claim 1 wherein Ring A is A-i: 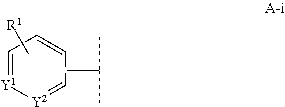
wherein:
Y¹ is selected from CR², N;
Y² is selected from CR², N; and
R² is selected from H, C₁₋₆ alkyl, halo, haloalkyl.

3. The compound according to claim 1 wherein Ring A is 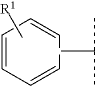
substituted with R¹ and further optionally substituted with C₁₋₆alkyl, halo or haloalkyl.

4. The compound according to claim 1 wherein Ring A is A-iii: 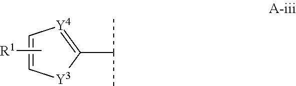
wherein Y³ is selected from O, S, or NH; and
Y⁴ is selected from CH or N.

5. The compound according to claim 1 wherein R¹ is —CO₂H.

6. The compound according to claim 1 wherein a is 0.

7. The compound according to claim 1 wherein Ring B is B-iv:

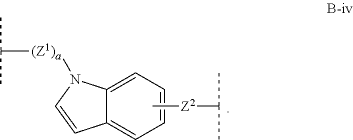

8. The compound according to claim 1 where Ring B is

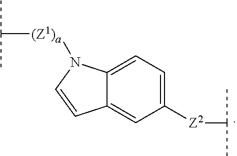

9. The compound according to claim 1 wherein Z² is —O—.

10. The compound according to claim 1 wherein R⁶ is alkyl, 2,2,2-trifluoroethyl or C₃₋₆cycloalkyl.

11. The compound according to claim 1 wherein R⁶ is isopropyl.

12. The compound according to claim 1 wherein d and e are both 0.

13. The compound according to claim 1 wherein d is 1 and R⁷ is methylene or ethylene.

14. The compound according to claim 1 wherein Ring D is a moiety of formula D-i

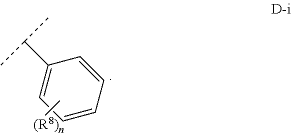

15. The compound according to claim 1 wherein Ring D is a moiety of formula D-i, n is 2 or 3 and each R⁸ is the same or different and is independently selected from halo and alkyl.

16. The compound according to claim 1 wherein n is 2.

17. The compound according to claim 1 wherein Ring D is a moiety of formula D-i, n is 2 and each R⁸ is the same and is halo or alkyl.

18. The compound according to claim 1 wherein n is 1, 2 or 3 and each R⁸ is the same or different and is independently selected from halo and alkyl.

19. A compound selected from the group consisting of
3-{[5-({[3-{[(2,6-Dimethylphenyl)oxy]methyl}-5-(1-methylethyl)-4-isoxazolyl]methyl}oxy)-1H-indol-1-yl]methyl}benzoic acid;
3-[(5-{[(5-(1-Methylethyl)-3-{[(2,4,6-trifluorophenyl)oxy]methyl}-4-isoxazolyl)methyl]oxy}-1H-indol-1-yl)methyl]benzoic acid;
3-[(5-{[(5-(1-Methylethyl)-3-{[(2,4,6-trichlorophenyl)oxy]methyl}-4-isoxazolyl)methyl]oxy}-1H-indol-1-yl)methyl]benzoic acid;
3-{[5-({[3-{[(2,6-Dichlorophenyl)amino]methyl}-5-(1-methylethyl)-4-isoxazolyl]methyl}oxy)-1H-indol-1-yl]methyl}benzoic acid;
3-{[5-({[3-{[(2,6-Dibromophenyl)oxy]methyl}-5-(1-methylethyl)-4-isoxazolyl]methyl}oxy)-1H-indol-1-yl]methyl}benzoic acid;
3-({5-[({5-(1-Methylethyl)-3-[(1,3-thiazol-2-ylthio)methyl]-4-isoxazolyl}methyl)oxy]-1H-indol-1-yl}methyl)benzoic acid;
3-[(5-{[(5-(1-Methylethyl)-3-{2-[(trifluoromethyl)oxy]phenyl}-4-isoxazolyl)methyl]oxy}-1H-indol-1-yl)methyl]benzoic acid;
3-{[6-({[3-(3,5-Dichloro-4-pyridinyl)-5-(1-methylethyl)-4-isoxazolyl]methyl}oxy)-1H-indol-1-yl]methyl}benzoic acid;
4-[6-({[3-(2,6-Dichlorophenyl)-5-(1-methylethyl)-4-isoxazolyl]methyl}oxy)-1-benzothien-2-yl]benzoic acid;
3-{[5-({[3-(2,6-Dichlorophenyl)-5-(1-methylethyl)-4-isoxazoly]methyl}oxy)-1H-indol-1-yl]carbonyl}benzoic acid;
3-[6-({[3-(2,6-Dichlorophenyl)-5-(1-methylethyl)-4-isoxazolyl]methyl}oxy)-1,3-benzothiazol-2-yl]benzoic acid;
5-({[3-(2,6-Dichlorophenyl)-5-(1-methylethyl)-4-isoxazolyl]methyl}oxy)-1-{[3-(1H-tetrazol-5-yl)phenyl]methyl}-1H-indole;
4-{[5-({[3-(2,6-Dichlorophenyl)-5-(1-methylethyl)-4-isoxazolyl]methyl}oxy)-1H-indol-1-yl]methyl}benzoic acid;
3-{[5-({[3-{[(2,6-Dichloro-4-fluorophenyl)oxy]methyl}-5-(1-methylethyl)-4-isoxazolyl]methyl}oxy)-1H-indol-1-yl]methyl}benzoic acid;
3-{[5-({[3-{[(2,6-Dichlorophenyl)oxy]methyl}-5-(1-methylethyl)-4-isoxazolyl]methyl}oxy)-1H-indol-1-yl]methyl}benzoic acid;
4-[5-({[3-(2,6-Dichlorophenyl)-5-(1-methylethyl)-4-isoxazolyl]methyl}oxy)-1-benzothien-2-yl]benzoic acid;
5-[6-({[3-(2,6-Dichlorophenyl)-5-(1-methylethyl)-4-isoxazolyl]methyl}oxy)-2-naphthalenyl]-3-pyridinecarboxylic acid;
6-[6-({[3-(2,6-Dichlorophenyl)-5-(1-methylethyl)-4-isoxazolyl]methyl}oxy)-2-naphthalenyl]-2-pyridinecarboxylic acid;
5-[6-({[3-(2,6-Dichlorophenyl)-5-(1-methylethyl)-4-isoxazolyl]methyl}oxy)-2-naphthalenyl]-2-pyridinecarboxylic acid;
4-[6-({[3-(2,6-Dichlorophenyl)-5-(1-methylethyl)-4-isoxazolyl]methyl}oxy)-2-naphthalenyl]-2-pyridinecarboxylic acid;
2-[6-({[3-(2,6-Dichlorophenyl)-5-(1-methylethyl)-4-isoxazolyl]methyl}oxy)-2-naphthalenyl]-4-pyridinecarboxylic acid;
4-[6-({[3-(2,6-Dichlorophenyl)-5-(1-methylethyl)-4-isoxazolyl]methyl}oxy)-2-naphthalenyl]benzoic acid;
1-{3-[6-({[3-(2,6-Dichlorophenyl)-5-(1-methylethyl)-4-isoxazolyl]methyl}oxy)-2-naphthalenyl]phenyl}methanamine;
3-{4-[6-({[3-(2,6-Dichlorophenyl)-5-(1-methylethyl)-4-isoxazolyl]methyl}oxy)-2-naphthalenyl]phenyl}propanoic acid;
6-[6-({[3-(2,6-Dichlorophenyl)-5-(1-methylethyl)-4-isoxazolyl]methyl}oxy)-2-naphthalenyl]-3-pyridinecarboxylic acid;
5-[6-({[3-(2,6-Dichlorophenyl)-5-(1-methylethyl)-4-isoxazolyl]methyl}oxy)-2-naphthalenyl]-2-thiophenecarboxylic acid;
N-{3-[6-({[3-(2,6-Dichlorophenyl)-5-(1-methylethyl)-4-isoxazolyl]methyl}oxy)-2-naphthalenyl]phenyl}-1,1,1-trifluoro-N-[(trifluoromethyl)sulfonyl]methanesulfonamide;
N-Acetyl-N-{3-[6-({[3-(2,6-dichlorophenyl)-5-(1-methylethyl)-4-isoxazolyl]methyl}oxy)-2-naphthalenyl]phenyl}acetamide;
N-{3-[6-({[3-(2,6-Dichlorophenyl)-5-(1-methylethyl)-4-isoxazolyl]methyl}oxy)-2-naphthalenyl]phenyl}-1,1,1-trifluoromethanesulfonamide;
N-{3-[6-({[3-(2,6-Dichlorophenyl)-5-(1-methylethyl)-4-isoxazolyl]methyl}oxy)-2-naphthalenyl]phenyl}acetamide;
3-(2,6-Dichlorophenyl)-5-(1-methylethyl)-4-{[(6-{3-[(trifluoromethyl)oxy]phenyl}-2-naphthalenyl)oxy]methyl}isoxazole;
N-{4-[6-({[3-(2,6-Dichlorophenyl)-5-(1-methylethyl)-4-isoxazolyl]methyl}oxy)-2-naphthalenyl]phenyl}acetamide;
N-{4-[6-({[3-(2,6-dichlorophenyl)-5-(1-methylethyl)-4-isoxazolyl]methyl}oxy)-2-naphthalenyl]phenyl}-1,1,1-trifluoromethanesulfonamide;
3-[7-({[3-(2,6-Dichlorophenyl)-5-(1-methylethyl)-4-isoxazolyl]methyl}oxy)-2-naphthalenyl]benzoic acid;
2-Chloro-5-[6-({[3-(2,6-dichlorophenyl)-5-(1-methylethyl)-4-isoxazolyl]methyl}oxy)-2-naphthalenyl]benzoic acid;
5-[6-({[3-(2,6-Dichlorophenyl)-5-(1-methylethyl)-4-isoxazolyl]methyl}oxy)-2-naphthalenyl]-2-fluorobenzoic acid;
3-[6-({[3-(2,6-Dichlorophenyl)-5-(1-methylethyl)-4-isoxazolyl]methyl}oxy)-1-benzothien-2-yl]benzoic acid;
3-[2-({[3-(2,6-Dichlorophenyl)-5-(1-methylethyl)-4-isoxazolyl]methyl}amino)-1,3-benzothiazol-6-yl]benzoic acid;
3-[6-({[3-(2,6-Dichlorophenyl)-5-(1-methylethyl)-4-isoxazolyl]methyl}oxy)-2-naphthalenyl]benzoic acid;
3-(2-{2-[3-(2,6-Dichlorophenyl)-5-(1-methylethyl)-4-isoxazolyl]ethyl}-1,3-benzoxazol-5-yl)benzoic acid;
3-{[6-({[3-(2,6-Dichlorophenyl)-5-(1-methylethyl)-4-isoxazolyl]methyl}oxy)-1-naphthalenyl]amino}benzoic acid;
3-[(2-{2-[3-(2,6-Dichlorophenyl)-5-(1-methylethyl)-4-isoxazolyl]ethyl}-1,3-benzoxazol-7-yl)amino]benzoic acid;
3-{[5-({[3-(2,6-Dichlorophenyl)-5-(1-methylethyl)-4-isoxazolyl]methyl}oxy)-1H-benzimidazol-1-yl]methyl}benzoic acid;
3-{[5-({[3-(2,6-Dichlorophenyl)-5-(1-methylethyl)-4-isoxazolyl]methyl}oxy)-1H-indol-1-yl]sulfonyl}benzoic acid;
3-[5-({[3-(2,6-Dichlorophenyl)-5-(1-methylethyl)-4-isoxazolyl]methyl}oxy)-1-oxo-1,3-dihydro-2H-isoindol-2-yl]benzoic acid;
3-[6-({[3-(2,6-Dichlorophenyl)-5-(1-methylethyl)-4-isoxazolyl]methyl}oxy)-3-oxo-3,4-dihydro-2(1H)-isoquinolinyl]benzoic acid;
5-({[3-(2,6-Dichlorophenyl)-5-(1-methylethyl)-4-isoxazolyl]methyl}oxy)-1-{[4-(1H-tetrazol-5-yl)phenyl]methyl}-1H-indole;
2-{[5-({[3-(2,6-Dichlorophenyl)-5-(1-methylethyl)-4-isoxazolyl]methyl}oxy)-1H-indol-1-yl]methyl}-1,3-oxazole-4-carboxylic acid;
5-[([5-({[3-(2,6-Dichlorophenyl)-5-(1-methylethyl)-4-isoxazolyl]methyl]oxy)-1H-indol-1-yl]methyl}-2-methylbenzoic acid;
6-{[5-({[3-(2,6-Dichlorophenyl)-5-(1-methylethyl)-4-isoxazolyl]methyl}oxy)-1H-indol-1-yl]methyl}-2-pyridinecarboxylic acid;
3-{[5-({[3-(2,6-Dichlorophenyl)-5-(1-methylethyl)-4-isoxazolyl]methyl}oxy)-1H-indol-3-yl]methyl}benzoic acid;
2-{[5-({[3-(2,6-Dichlorophenyl)-5-(1-methylethyl)-4-isoxazolyl]methyl}oxy)-1H-indol-1-yl]methyl}-1,3-thiazole-4-carboxylic acid;
3-{[6-({[3-(2,6-Dichlorophenyl)-5-(1-methylethyl)-4-isoxazolyl]methyl}oxy)-1H-indol-3-yl]methyl}benzoic acid;
(3R)-1-{[5-({[3-(2,6-Dichlorophenyl)-5-(1-methylethyl)-4-isoxazolyl]methyl}oxy)-1H-indol-1-yl]carbonyl}-3-pyrrolidinecarboxylic acid;
3-[5-({[3-(2,6-Dichlorophenyl)-5-(1-methylethyl)-4-isoxazolyl]methyl}oxy)-1-benzothien-2-yl]benzoic acid;
(3S)-1-{[5-({[3-(2,6-Dichlorophenyl)-5-(1-methylethyl)-4-isoxazolyl]methyl}oxy)-1H-indol-1-yl]carbonyl}-3-pyrrolidinecarboxylic acid;
3-[5-({[3-(2,6-Dichlorophenyl)-5-(1-methylethyl)-4-isoxazolyl]methyl}oxy)-1H-indol-2-yl]benzoic acid;
3-[5-({[3-(2,6-Dichlorophenyl)-5-(1-methylethyl)-4-isoxazolyl]methyl}oxy)-2,3-dihydro-1H-inden-2-yl]benzoic acid;
3-[6-({[3-(2,6-Dichlorophenyl)-5-(1-methylethyl)-4-isoxazolyl]methyl}oxy)-1H-inden-2-yl]benzoic acid;
3-[5-({[3-(2,6-Dichlorophenyl)-5-(1-methylethyl)-4-isoxazolyl]methyl}oxy)-1H-inden-2-yl]benzoic acid;
3-{[5-({[3-(2,6-Dichlorophenyl)-5-(1-methylethyl)-4-isoxazolyl]methyl}oxy)-1H-indol-1-yl]methyl}benzoic acid; and
3-[6-({[3-(2,6-Dichlorophenyl)-5-(1-methylethyl)-4-isoxazolyl]methyl}oxy)-1-oxo-3,4-dihydro-2(1H)-isoquinolinyl]benzoic acid;
and pharmaceutically acceptable salts thereof.

20. 3-[5-({[3-(2,6-Dichlorophenyl)-5-(1-methylethyl)-4-isoxazolyl]methyl}oxy)-1-benzothien-2-yl]benzoic acid or a pharmaceutically acceptable salt thereof.

21. 3-{[5-({[3-(2,6-dichlorophenyl)-5-(1-methylethyl)-4-isoxazolyl]methyl}oxy)-1H-indol-1-yl]methyl}benzoic acid or a pharmaceutically acceptable salt thereof.

22. A pharmaceutical composition comprising a compound according to claim 1 and a pharmaceutically acceptable carrier or diluent.

23.-24. (canceled)

25. A method for the treatment of a condition selected from metabolic syndrome, cholestatic liver disease, organ fibrosis and liver fibrosis in a subject in need thereof comprising administering to said subject a therapeutically effective amount of a compound according to claim 1.

26. (canceled)

27. A process for preparing a compound according to claim 1 comprising the step of:
reacting a compound of formula (II) 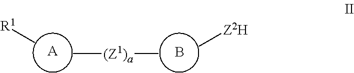
with a compound of formula (XLII) 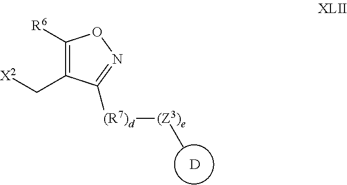
wherein: a is 0;
Z² is —O—, —NH— or —S—;
X² is chloride, iodide, bromide, triflate, tosylate, nosylate, besylate or mesylate;
R¹ is —CO₂alkyl, —CH₂CH₂CO₂alkyl, —NHC(O)CH₃, or —OCF₃; and
all other variables are as defined above for formula (I)
to prepare a compound of formula (I).

28.-31. (canceled)
